(19) United States
(12) Patent Application Publication
(10) Pub. No.: US 2005/0214844 A1
(43) Pub. Date: Sep. 29, 2005

(54) 86 human secreted proteins

(75) Inventors: Paul A. Moore, North Bethesda, MD (US); Yanggu Shi, Gaithersburg, MD (US); Craig A. Rosen, Laytonsville, MD (US); Steven M. Ruben, Brookeville, MD (US); David W. Lafleur, Washington, DC (US); Henrik S. Olsen, Gaithersburg, MD (US); Reinhard Ebner, Gaithersburg, MD (US); Laurie A. Brewer, St. Paul, MN (US); Paul E. Young, Gaithersburg, MD (US); John M. Greene, Gaithersburg, MD (US); Ann M. Ferrie, Painted Post, NY (US); Guo-Liang Yu, Berkeley, CA (US); Jian Ni, Germantown, MD (US); Ping Feng, Germantown, MD (US)

Correspondence Address: HUMAN GENOME SCIENCES INC;INTELLECTUAL PROPERTY DEPT., 14200 SHADY GROVE ROAD, ROCKVILLE, MD, 20850, US

(73) Assignee: Human Genome Sciences, Inc., Rockville, MD (US)

(21) Appl. No.: 11/111,953

(22) Filed: Apr. 22, 2005

Related U.S. Application Data
(63) Continuation of application No. 10/219,793, filed on Aug. 16, 2002.
Application No. 10/219,793 is a continuation of application No. 09/209,462, filed on Dec. 11, 1998, now abandoned.
Application No. 09/209,462 is a continuation-in-part of application No. PCT/US98/12125, filed on Jun. 11, 1998.
(60) Provisional application No. 60/049,547, filed on Jun. 13, 1997.
(60) Provisional application No. 60/049,548, filed on Jun. 13, 1997.
(60) Provisional application No. 60/049,549, filed on Jun. 13, 1997.
(60) Provisional application No. 60/049,550, filed on Jun. 13, 1997.
(60) Provisional application No. 60/049,566, filed on Jun. 13, 1997.
(60) Provisional application No. 60/049,606, filed on Jun. 13, 1997.
(60) Provisional application No. 60/049,607, filed on Jun. 13, 1997.
(60) Provisional application No. 60/049,608, filed on Jun. 13, 1997.
(60) Provisional application No. 60/049,609, filed on Jun. 13, 1997.
(60) Provisional application No. 60/049,610, filed on Jun. 13, 1997.
(60) Provisional application No. 60/049,611, filed on Jun. 13, 1997.
(60) Provisional application No. 60/050,901, filed on Jun. 13, 1997.
(60) Provisional application No. 60/052,989, filed on Jun. 13, 1997.
(60) Provisional application No. 60/051,919, filed on Jul. 8, 1997.
(60) Provisional application No. 60/055,984, filed on Aug. 18, 1997.
(60) Provisional application No. 60/058,665, filed on Sep. 12, 1997.
(60) Provisional application No. 60/058,668, filed on Sep. 12, 1997.
(60) Provisional application No. 60/058,669, filed on Sep. 12, 1997.
(60) Provisional application No. 60/058,750, filed on Sep. 12, 1997.
(60) Provisional application No. 60/058,971, filed on Sep. 12, 1997.
(60) Provisional application No. 60/058,972, filed on Sep. 12, 1997.
(60) Provisional application No. 60/058,975, filed on Sep. 12, 1997.
(60) Provisional application No. 60/060,834, filed on Oct. 2, 1997.
(60) Provisional application No. 60/060,841, filed on Oct. 2, 1997.
(60) Provisional application No. 60/060,844, filed on Oct. 2, 1997.
(60) Provisional application No. 60/060,865, filed on Oct. 2, 1997.
(60) Provisional application No. 60/061,059, filed on Oct. 2, 1997.
(60) Provisional application No. 60/061,060, filed on Oct. 2, 1997.

(51) Int. Cl. C12Q 1/68; C07H 21/04; C12N 9/00; C12N 15/09; C07K 14/47; C07K 16/18

(52) U.S. Cl. 435/6; 435/69.1; 435/183; 435/320.1; 435/325; 530/350; 536/23.2; 530/388.1

(57) ABSTRACT

The present invention relates to novel human secreted proteins and isolated nucleic acids containing the coding regions of the genes encoding such proteins. Also provided are vectors, host cells, antibodies, and recombinant methods for producing human secreted proteins. The invention further relates to diagnostic and therapeutic methods useful for diagnosing and treating disorders related to these novel human secreted proteins.

### CROSS-REFERENCE TO RELATED APPLICATIONS

This application is a continuation of U.S. patent application Ser. No. 10/219,793, filed Aug. 16, 2002; which is a continuation of U.S. patent application Ser. No. 09/209,462, filed on Dec. 11, 1998; which is a continuation-in-part of United States Patent Application No. PCT/US98/12125, filed Jun. 11, 1998, which claims benefit under 35 U.S.C. § 119(e) based on U.S. Provisional Applications: 60/049,547, 60/049,548, 60/049,549, 60/049,550, 60/049,566, 60/049,606, 60/049,607, 60/049,608, 60/049,609, 60/049,610, 60/049,611, 60/050,901, and 60/052,989, filed Jun. 13, 1997; U.S. Provisional Application 60/051,919, filed Jul. 8, 1997; U.S. Provisional Application 60/055,984, filed Aug. 18, 1997; U.S. Provisional Application 60/058,665, 60/058,668, 60/058,669, 60/058,750, 60/058,971, 60/058,972, and 60/058,975, filed Sep. 12, 1997; and U.S. Provisional Application 60/060,834, 60/060,841, 60/060,844, 60/060,865, 60/061,059, and 60/061,060, filed Oct. 2, 1997. All above-mentioned Applications are hereby incorporated by reference.

### REFERENCE TO SEQUENCE LISTING ON COMPACT DISC

This application refers to a “Sequence Listing” listed below, which is provided as an electronic document on two identical compact discs (CD-R), labeled “Copy 1” and “Copy 2.” These compact discs each contain the file “PZ008P1C2 SeqList.txt” (created Apr. 12, 2005, byte size=579,231 bytes), which is hereby incorporated by reference in its entirety.

### FIELD OF THE INVENTION

This invention relates to newly identified polynucleotides and the polypeptides encoded by these polynucleotides, uses of such polynucleotides and polypeptides, and their production.

### BACKGROUND OF THE INVENTION

Unlike bacterium, which exist as a single compartment surrounded by a membrane, human cells and other eukaryotes are subdivided by membranes into many functionally distinct compartments. Each membrane-bounded compartment, or organelle, contains different proteins essential for the function of the organelle. The cell uses “sorting signals,” which are amino acid motifs located within the protein, to target proteins to particular cellular organelles.

One type of sorting signal, called a signal sequence, a signal peptide, or a leader sequence, directs a class of proteins to an organelle called the endoplasmic reticulum (ER). The ER separates the membrane-bounded proteins from all other types of proteins. Once localized to the ER, both groups of proteins can be further directed to another organelle called the Golgi apparatus. Here, the Golgi distributes the proteins to vesicles, including secretory vesicles, the cell membrane, lysosomes, and the other organelles.

Proteins targeted to the ER by a signal sequence can be released into the extracellular space as a secreted protein. For example, vesicles containing secreted proteins can fuse with the cell membrane and release their contents into the extracellular space—a process called exocytosis. Exocytosis can occur constitutively or after receipt of a triggering signal. In the latter case, the proteins are stored in secretory vesicles (or secretory granules) until exocytosis is triggered. Similarly, proteins residing on the cell membrane can also be secreted into the extracellular space by proteolytic cleavage of a “linker” holding the protein to the membrane.

Despite the great progress made in recent years, only a small number of genes encoding human secreted proteins have been identified. These secreted proteins include the commercially valuable human insulin, interferon, Factor VIII, human growth hormone, tissue plasminogen activator, and erythropoietin. Thus, in light of the pervasive role of secreted proteins in human physiology, a need exists for identifying and characterizing novel human secreted proteins and the genes that encode them. This knowledge will allow one to detect, to treat, and to prevent medical disorders by using secreted proteins or the genes that encode them.

### SUMMARY OF THE INVENTION

The present invention relates to novel polynucleotides and the encoded polypeptides. Moreover, the present invention relates to vectors, host cells, antibodies, and recombinant methods for producing the polypeptides and polynucleotides. Also provided are diagnostic methods for detecting disorders related to the polypeptides, and therapeutic methods for treating such disorders. The invention further relates to screening methods for identifying binding partners of the polypeptides.

### DETAILED DESCRIPTION

Definitions

The following definitions are provided to facilitate understanding of certain terms used throughout this specification.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide.

In the present invention, a “secreted” protein refers to those proteins capable of being directed to the ER, secretory vesicles, or the extracellular space as a result of a signal sequence, as well as those proteins released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage.

As used herein, a “polynucleotide” refers to a molecule having a nucleic acid sequence contained in SEQ ID NO:X or the cDNA contained within the clone deposited with the ATCC™. For example, the polynucleotide can contain the nucleotide sequence of the full length cDNA sequence, including the 5′ and 3′ untranslated sequences, the coding region, with or without the signal sequence, the secreted protein coding region, as well as fragments, epitopes, domains, and variants of the nucleic acid sequence. Moreover, as used herein, a “polypeptide” refers to a molecule having the translated amino acid sequence generated from the polynucleotide as broadly defined.

In the present invention, the full length sequence identified as SEQ ID NO:X was often generated by overlapping sequences contained in multiple clones (contig analysis). A representative clone containing all or most of the sequence for SEQ ID NO:X was deposited with the American Type Culture Collection (“ATCC™”). As shown in Table 1, each clone is identified by a cDNA Clone ID (Identifier) and the ATCC™ Deposit Number. The ATCC™ is located at 10801 University Boulevard, Manassas, Va. 20110-2209, USA. The ATCC™ deposit was made pursuant to the terms of the Budapest Treaty on the international recognition of the deposit of microorganisms for purposes of patent procedure.

A “polynucleotide” of the present invention also includes those polynucleotides capable of hybridizing, under stringent hybridization conditions, to sequences contained in SEQ ID NO:X, the complement thereof, or the cDNA within the clone deposited with the ATCC™. “Stringent hybridization conditions” refers to an overnight incubation at 42° C. in a solution comprising 50% formamide, 5× SSC (750 mM NaCl, 75 mM sodium citrate), 50 mM sodium phosphate (pH 7.6), 5× Denhardt's solution, 10% dextran sulfate, and 20 μg/ml denatured, sheared salmon sperm DNA, followed by washing the filters in 0.1× SSC at about 65° C.

Also contemplated are nucleic acid molecules that hybridize to the polynucleotides of the present invention at lower stringency hybridization conditions. Changes in the stringency of hybridization and signal detection are primarily accomplished through the manipulation of formamide concentration (lower percentages of formamide result in lowered stringency); salt conditions, or temperature. For example, lower stringency conditions include an overnight incubation at 37° C. in a solution comprising 6× SSPE (20× SSPE=3M NaCl; 0.2M NaH₂PO₄; 0.02M EDTA, pH 7.4), 0.5% SDS, 30% formamide, 100 ug/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5× SSC).

Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Of course, a polynucleotide which hybridizes only to polyA+ sequences (such as any 3′ terminal polyA+ tract of a cDNA shown in the sequence listing), or to a complementary stretch of T (or U) residues, would not be included in the definition of “polynucleotide,” since such a polynucleotide would hybridize to any nucleic acid molecule containing a poly (A) stretch or the complement thereof (e.g., practically any double-stranded cDNA clone).

The polynucleotide of the present invention can be composed of any polyribonucleotide or polydeoxyribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the polynucleotide can be composed of triple-stranded regions comprising RNA or DNA or both RNA and DNA. A polynucleotide may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The polypeptide of the present invention can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W.H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182: 626-646 (1990); Rattan et al., Ann NY Acad Sci 663: 48-62 (1992).)

“SEQ ID NO:X” refers to a polynucleotide sequence while “SEQ ID NO:Y” refers to a polypeptide sequence, both sequences identified by an integer specified in Table 1.

“A polypeptide having biological activity” refers to polypeptides exhibiting activity similar, but not necessarily identical to, an activity of a polypeptide of the present invention, including mature forms, as measured in a particular biological assay, with or without dose dependency. In the case where dose dependency does exist, it need not be identical to that of the polypeptide, but rather substantially similar to the dose-dependence in a given activity as compared to the polypeptide of the present invention (i.e., the candidate polypeptide will exhibit greater activity or not more than about 25-fold less and, preferably, not more than about tenfold less activity, and most preferably, not more than about three-fold less activity relative to the polypeptide of the present invention.)

Polynucleotides and Polypeptides of the Invention

Features of Protein Encoded by Gene No: 1

The translation product of this gene shares sequence homology with LIM-homeobox domain proteins, such as T-cell translocation protein, which are thought to be important in development and leukemogenesis. In addition, the translation product of this gene shares homology with the human breast tumor autoantigen (See Genbank Accession No. gi|1914877). In specific embodiments, polypeptides of the invention comprise the following amino acid sequence:

| | |
|---|---|
| MNGSHKDPLLPFPASARTPSLPPAPPAQAPLPWKPSGFARISPPPPLAILQYRGKADHGESGQQLAAAPGDGRLPLLEAVRRLRGQ | (SEQ ID NO:221) |
| DCGPLSALCHGQLLAQPVPQVLLLPGAXGDIGTSCYTKSGMILCRNDYIRLFGNSGACSACGQSIPASELVMRAQGNVYHLKCFTC | |
| STCRNRLVPGDRFHYINGSLFCEHDRPTALINGHLNSLQSNPLLPDQKVCKVRVMQNACLHLRFVHHRWIPCXFSRQVTFVASTSA | |
| SSMPLHLL, | |
| MARTRTPSSPFLLLRELPPSLQLRQPRRPFPGSRAASLAFHRRRLSQYCNIGEKQTMVNPGSSSQPPPVTAGSLSWKRCAGCGGKI | (SEQ ID NO:222) |
| ADRFLLYA, | |
| LFGNSGACSACGQSIPASELVMRA, | (SEQ ID NO:223) |
| HDRPTALINGHLNSLQSNP, | (SEQ ID NO:224) |
| and/or | |
| LVPGDRFHYING. | (SEQ ID NO:225) |

This gene is expressed primarily in fetal brain, osteosarcoma, IL-1/TNF treated synovial, and estradiol treated endometrial stromal cells, and to a lesser extent in chondrosarcoma and smooth muscle. Therefore, polynucleotides and polypeptides of the invention are useful as reagents for differential identification of the tissue(s) or cell type(s) present in a biological sample and for diagnosis of diseases and conditions which include, but are not limited to, developmental defects or leukemia. Similarly, polypeptides and antibodies directed to these polypeptides are useful in providing immunological probes for differential identification of the tissue(s) or cell type(s). For a number of disorders of the above tissues or cells, particularly of the hematopoietic system and immune system, expression of this gene at significantly higher or lower levels may be routinely detected in certain tissues or cell types (e.g., immune, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 116 as residues: Pro-4 to Pro-11, Asp-40 to Cys-47. The tissue distribution in fetal brain and stromal cells, combined with the homology to the LIM-homeodomain containing proteins, such as T-cell translocation factor, indicates that polynucleotides and polypeptides corresponding to this gene are useful for the diagnosis and intervention of leukemia and other developmental defects. Because of the importance of the LIM-homeodomain proteins in development and their correlation to a number of leukemic diseases, the molecule can be either used as a diagnositic or prognostic indicator for leukemia progression or a therapeutic target. In addition, polynucleotides and polypeptides corresponding to this gene are useful for the detection/treatment of neurodegenerative disease states and behavioral disorders such as Alzheimers Disease, Parkinsons Disease, Huntingtons Disease, schizophrenia, mania, dementia, paranoia, obsessive compulsive disorder, panic disorder, and autism. In addition, the gene or gene product may also play a role in the treatment and/or detection of developmental disorders associated with the developing embryo, sexually-linked disorders, or disorders of the cardiovascular system. Furthermore, homology to the breast auto-antigen may suggest that this gene is useful in the detection, prevention, and or treatment of breast cancer and/or other proliferative disorders where expression is indicated. Protein, as well as, antibodies directed against the protein may show utility as a tissue-specific marker and/or immunotherapy target for the above listed tissues.

Many polynucleotide sequences, such as EST sequences, are publicly available and accessible through sequence databases. Some of these sequences are related to SEQ ID NO:11 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1373 of SEQ ID NO:11, b is an integer of 15 to 1387, where both a and b correspond to the positions of nucleotide residues shown in SEQ ID NO:11, and where b is greater than or equal to a+14.

Features of Protein Encoded by Gene No: 2

The translation product of this gene has homology to a highly conserved member of the human calpain family of proteases, Calpain large subunit 1 gene (See Genbank Accession No. T32454). Calpains are thought to play a defining role in protein regulation, particularly during development. In specific embodiments, polypeptides of the invention comprise the following amino acid sequence:

| | |
|---|---|
| MKYMGGCAKVMCKYYVILYQGLEYPLLXSGDPETSPPWILRADCIVLSSRNFHSNXGRLTINKIYVIGGGKYRGEVTNGAK, | (SEQ ID NO:226) |
| MGQSELYSSILRNLGVLFLVYTRGGFLLSPLLHGTLTCAHS, | (SEQ ID NO:227) |
| MVLLLLTVASYTVFWMIGDVLDILFLWNFEYTTLY, | (SEQ ID NO:228) |
| MELYNSLCPICYFSTVLTTTYYIYFVYSQSSXIRMKVP, | (SEQ ID NO:229) |
| MQIVIVLYCVRNKDKKKVCTCSVQTQFFFPIFPILGCLNGCRTQE, | (SEQ ID NO:230) |
| MKYMGGCAKVMCKYYVILYQGLEYPLLX, | (SEQ ID NO:231) |
| LEYPLLXSGDPETSPPWILRADCIVLSSRNFHSNX, | (SEQ ID NO:232) |
| RNFHSNXGRLTINKIYVIGGGKYRGEVTNGAK, | (SEQ ID NO:233) |
| and/or | |
| MLLLTILILLCNRTPELIP. | (SEQ ID NO:234) |

encoding these polypeptides are also encompassed by the invention.

This gene is expressed primarily in caudate nucleus, dermatofibrosarcoma protuberance and apoptotic T-cells, and to a lesser extent in eosinophils, brain and smooth muscle. Therefore, polynucleotides and polypeptides of the invention are useful as reagents for differential identification of the tissue(s) or cell type(s) present in a biological sample and for diagnosis of diseases and conditions which include, but are not limited to, neurodegenerative diseases or immune disorders. Similarly, polypeptides and antibodies directed to these polypeptides are useful in providing immunological probes for differential identification of the tissue(s) or cell type(s). For a number of disorders of the above tissues or cells, particularly of the nervous system or immune system, expression of this gene at significantly higher or lower levels may be routinely detected in certain tissues or cell types (e.g., neurodegenerative, immune, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in caudate nucleus and apoptic T-cells indicates that the protein product of this gene is useful for the detection or intervention of neurodegenerative diseases and behavioral disorders such as Alzheimers Disease, Parkinsons Disease, Huntinton's disease, schizophrenia, mania, dementia, paranoia, obsessive compulsive disorder, panic disorder or immune disorders, because the elevated level of the molecule in cells undergoing cell death may be the cause or consequence of these degenerative conditions. In addition, the gene or gene product may also play a role in the treatment and/or detection of developmental disorders associated with the developing embryo, or disorders of the cardiovascular system. Protein, as well as, antibodies directed against the protein may show utility as a tissue-specific marker and/or immunotherapy target for the above listed tissues.

Many polynucleotide sequences, such as EST sequences, are publicly available and accessible through sequence databases. Some of these sequences are related to SEQ ID NO:12 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1925 of SEQ ID NO:12, b is an integer of 15 to 1939, where both a and b correspond to the positions of nucleotide residues shown in SEQ ID NO:12, and where b is greater than or equal to a+14.

Features of Protein Encoded by Gene No: 3

The gene encoding the disclosed cDNA is thought to reside on chromosome 15. Accordingly, polynucleotides related to this invention are useful as a marker in linkage analysis for chromosome 15. In specific embodiments, polypeptides of the invention comprise the following amino acid sequence:

| | |
|---|---|
| VTNEMSQGRGKYDFYIGLGLAMSSSIFIGGSFILKKKGLLRLARKGSMRAGQGGHAYLKEWLWWAGLLSMGAGEVANFAAYAFAPA | (SEQ ID NO:235) |
| TLVTPLGALSVLVSAILSSYFLNERLNLHGKIGCLLSILG STVMVIHAPKEEEIETLNE, | |
| VTNEMSQGRGKYDFYIGLGLAMSSSIFIGGSFILKKKGLLRLARKGSMRAGQGGHAYLKEWLWWAGLLSMGAGEVANF, | (SEQ ID NO:236) |
| NFAAYAFAPATLVTPLGALSVLVSAILSSY, | (SEQ ID NO:237) |
| and/or | |
| ERLNLHGKIGCLLSILGSTVMVIHAPKEEEIETLNE. | (SEQ ID NO:238) |

This gene is expressed primarily in colon carcinoma cell line, and to a lesser extent in aorta endothelial cells, T-cells, human erythroleukemia cells (HEL), and stromal cells (TF274). Therefore, polynucleotides and polypeptides of the invention are useful as reagents for differential identification of the tissue(s) or cell type(s) present in a biological sample and for diagnosis of diseases and conditions which include, but are not limited to, colon carcinoma. Similarly, polypeptides and antibodies directed to these polypeptides are useful in providing immunological probes for differential identification of the tissue(s) or cell type(s). For a number of disorders of the above tissues or cells, particularly of colon carcinoma tissues, expression of this gene at significantly higher or lower levels may be routinely detected in certain tissues or cell types (e.g., colon, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 118 as residues: Asn-191 to Ser-196, Asn-208 to Gly-214. The tissue distribution in colon carcinoma indicates that the protein product of this gene is useful for the detection and intervention of colon carcinoma and/or tumors of other tissues where expression is indicated. Additionally, the significant presence in T-cell populations may indicate the involvement of the function of the gene product in cancer immunosurveillance. Furthermore, the tissue distribution indicates that polynucleotides and polypeptides corresponding to this gene are useful for the diagnosis and treatment of cancer and other proliferative disorders, in general. The expression in hematopoietic cells and tissues indicates that this protein may play a role in the proliferation, differentiation, and/or survival of hematopoietic cell lineages. Thus, this gene may be useful in the treatment of lymphoproliferative disorders, and in the maintenance and differentiation of various hematopoietic lineages from early hematopoietic stem and committed progenitor cells. Protein, as well as, antibodies directed against the protein may show utility as a tissue-specific marker and/or immunotherapy target for the above listed tissues.

Many polynucleotide sequences, such as EST sequences, are publicly available and accessible through sequence databases. Some of these sequences are related to SEQ ID NO: 13 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 2588 of SEQ ID NO:13, b is an integer of 15 to 2602, where both a and b correspond to the positions of nucleotide residues shown in SEQ ID NO:13, and where b is greater than or equal to a+14.

Features of Protein Encoded by Gene No: 4

This gene is expressed primarily in ovary. Therefore, polynucleotides and polypeptides of the invention are useful as reagents for differential identification of the tissue(s) or cell type(s) present in a biological sample and for diagnosis of diseases and conditions which include, but are not limited to, reproductive or endocrine disorders. Similarly, polypeptides and antibodies directed to these polypeptides are useful in providing immunological probes for differential identification of the tissue(s) or cell type(s). For a number of disorders of the above tissues or cells, particularly of the reproductive or endocrine systems, expression of this gene at significantly higher or lower levels may be routinely detected in certain tissues or cell types (e.g. reproductive, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 119 as residues: Pro-20 to Ser-25. The tissue distribution in ovary indicates that the protein product of this gene is useful for assessing reproductive dysfunction or endocrine disorders, because factors secreted by ovary may be involved in reproductive processes, and in such cases have global hormonal effects. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:14 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 794 of SEQ ID NO:14, b is an integer of 15 to 808, where both a and b correspond to the positions of nucleotide residues shown in SEQ ID NO:14, and where b is greater than or equal to a+14.

Features of Protein Encoded by Gene No: 5

In specific embodiments, polypeptides of the invention comprise the following amino acid sequence:

| | |
|---|---|
| PTAHLANPSPSTVNSGSAGPRPPAGAVVAAPGGALASVSFDSRDSKMAAQSAPKVVLKSTTKMSLNERFTNMLKNKQPTPVNIRAS | (SEQ ID NO:239) |
| MQQQQQLASARNRRLAQQMENRPSVQAALKLKQKSLKQRLGKSNIQARLGRPIGALARGAIGGRGLPIIQRGLPRGGLRGGRATRT | |
| LLRGGMSLRGQNLLRGGRAVAPRMGLRRGGVRGRGGPGRGGLGRGAMGRGGIGGRGRGMIGRGRGGFGGRGRGRGRGRGALARPVL | |
| TKEQLDNQLDAYMSKTKGHLDAELDAYMAQTDPETND, | |
| PPAGAVVAAPGGALASVSFDSRD, | (SEQ ID NO:240) |
| AQSAPKVVLKSTTKMSLNERFTNMLKNKQ, | (SEQ ID NO:241) |
| RNRRLAQQMENRPSVQAALKLKQ, | (SEQ ID NO:242) |
| GLPRGGLRGGRATRTLLRGGMSLRG, | (SEQ ID NO:243) |
| GGRAVAPRMGLRRGGVRGRGGPGR, | (SEQ ID NO:244) |
| RGMIGRGRGGFGGRGRGRGRGRGALA, | (SEQ ID NO:245) |
| and/or | |
| QLDNQLDAYMSKTKGHLDAELDAYMAQT. | (SEQ ID NO:246) |

Polynucleotides encoding these polypeptides are also encompassed by the invention.

The gene encoding the disclosed cDNA is thought to reside on chromosome 1. Accordingly, polynucleotides related to this invention are useful as a marker in linkage analysis for chromosome 1. This gene is expressed primarily in tissues in the central nervous system, including pineal gland, frontal cortex, and dura mater, and to a lesser extent in bladder, lung, T-cells and liver. Therefore, polynucleotides and polypeptides of the invention are useful as reagents for differential identification of the tissue(s) or cell type(s) present in a biological sample and for diagnosis of diseases and conditions which include, but are not limited to, neurodegenerative diseases, endocrine disorders, and immune disorders. Similarly, polypeptides and antibodies directed to these polypeptides are useful in providing immunological probes for differential identification of the tissue(s) or cell type(s). For a number of disorders of the above tissues or cells, particularly of the nervous and endocrine systems, expression of this gene at significantly higher or lower levels may be routinely detected in certain tissues or cell types (e.g., immune, endocrine, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The primary tissue distribution in tissues of the central nervous system indicates that the protein product of this gene is useful for the detection and intervention of neurodegenerative diseases or endocrine disorders, because extracellular proteins in these tissues may function as a neurotrophic factor, a matrix protein for tissue integrity, a neuroguidance factor or as a hormone. Furthermore, the tissue distribution indicates that the protein product of this gene is useful for the detection, treatment, and/or prevention of various endocrine disorders and cancers, particularly Addison's disease, Cushing's Syndrome, and disorders and/or cancers of the pancreas (e.g. diabetes mellitus), adrenal cortex, ovaries, pituitary (e.g., hyper-, hypopituitarism), thyroid (e.g. hyper-, hypothyroidism), parathyroid (e.g. hyper-, hypoparathyroidism), hypothalamus, and testes. Additionally, the tissue distribution indicates that the protein product of this gene is useful for the detection/treatment of neurodegenerative disease states and behavioral disorders such as Alzheimers Disease, Parkinsons Disease, Huntingtons Disease, Tourette Syndrome, schizophrenia, mania, dementia, paranoia, obsessive compulsive disorder, panic disorder, learning disabilities, ALS, psychoses, autism, and altered behaviors, including disorders in feeding, sleep patterns, balance, and perception. In addition, the gene or gene product may also play a role in the treatment and/or detection of developmental disorders associated with the developing embryo, or sexually-linked disorders. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:15 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 2129 of SEQ ID NO:15, b is an integer of 15 to 2143, where both a and b correspond to the positions of nucleotide residues shown in SEQ ID NO:15, and where b is greater than or equal to a+14.

Features of Protein Encoded by Gene No: 6

The gene encoding the disclosed cDNA is thought to reside on chromosome 5. Accordingly, polynucleotides related to this invention are useful as a marker in linkage analysis for chromosome 5. This gene is expressed primarily in spleen, resting T-cells, colorectal tumor and pancreatic carcinoma, and to a lesser extent in a number of tissues including prostate, synovial hypoxia, osteosarcoma, ulcerative colitis, myeloid progenitor cells, lung and placenta. Therefore, polynucleotides and polypeptides of the invention are useful as reagents for differential identification of the tissue(s) or cell type(s) present in a biological sample and for diagnosis of diseases and conditions which include, but are not limited to, inflammation, immunosurveillance of cancers, and immune and gastrointestinal disorders. Similarly, polypeptides and antibodies directed to these polypeptides are useful in providing immunological probes for differential identification of the tissue(s) or cell type(s). For a number of disorders of the above tissues or cells, particularly in carcinogenesis or the immune system, expression of this gene at significantly higher or lower levels may be routinely detected in certain tissues or cell types (e.g., immune, gastrointestinal, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 121 as residues: Arg-29 to Pro-37, Gln-46 to Val-56. The primary tissue distribution in lymphatic tissues such as T-cells and spleen, as well as tumors and ulcerative tissues, indicates that the protein product of this gene is involved in the immuno-response to or immunosurveillance of carcinogenesis and/or inflammatory conditions. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:16 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 2347 of SEQ ID NO:16, b is an integer of 15 to 2361, where both a and b correspond to the positions of nucleotide residues shown in SEQ ID NO:16, and where b is greater than or equal to a+14.

Features of Protein Encoded by Gene No: 7

The translation product of this gene shares very weak sequence homology with voltage dependent sodium channel protein and Bowman-Birk proteinase inhibitor which is thought to be important in membrane signaling or extracellular signaling cascades (See Genbank Accession No. gnl|PID|d1020763 (AB000216)). The translation product of this gene also shares homology with Cca3, which is a rat liver protein thought to be involved in DNA synthesis (See Genbank Accession No. gnl|PID|g2104558 (AB000216)). In specific embodiments, polypeptides of the invention comprise the following amino acid sequence: RFKTLMTNKSEQDGDSSKTIEISDMKYHIFQ (SEQ ID NO:247), and/or LVEGKLFYAHKVLLVTXSNR (SEQ ID NO:248). Polynucleotides encoding these polypeptides are also encompassed by the invention.

This gene is expressed primarily in prostate cancer. Therefore, polynucleotides and polypeptides of the invention are useful as reagents for differential identification of the tissue(s) or cell type(s) present in a biological sample and for diagnosis of diseases and conditions which include, but are not limited to, prostate cancer. Similarly, polypeptides and antibodies directed to these polypeptides are useful in providing immunological probes for differential identification of the tissue(s) or cell type(s). For a number of disorders of the above tissues or cells, particularly of prostate cancer tissue, expression of this gene at significantly higher or lower levels may be routinely detected in certain tissues or cell types (e.g., prostate, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 122 as residues: Glu-30 to Ser-35. The tissue distribution in the prostate, combined with the homology to a sodium channel or proteinase inhibitor as well as Cca3 suggest that the protein product of this gene is useful for the intervention of cancer progression, because the gene product may be involved in multidrug resistance by altering the drug kinetics by serving the function as a channel transporter. Alternatively, the proteinase inhibitor like function may facilitate tumor metastasis. By targeting these functions, either through vaccine or small molecules, therapeutics may be rationally designed to slow the cancer progression. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:17 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 789 of SEQ ID NO:17, b is an integer of 15 to 803, where both a and b correspond to the positions of nucleotide residues shown in SEQ ID NO:17, and where b is greater than or equal to a+14.

Features of Protein Encoded by Gene No: 8

In specific embodiments, polypeptides of the invention comprise the following amino acid sequence: MTVKPCLVFSCAFGLLVSRLS (SEQ ID NO:249). Polynucleotides encoding these polypeptides are also encompassed by the invention. This gene is expressed primarily in ovary, and to a lesser extent in the adrenal gland. Therefore, polynucleotides and polypeptides of the invention are useful as reagents for differential identification of the tissue(s) or cell type(s) present in a biological sample and for diagnosis of diseases and conditions which include, but are not limited to, female infertility and endocrine disorders. Similarly, polypeptides and antibodies directed to these polypeptides are useful in providing immunological probes for differential identification of the tissue(s) or cell type(s). For a number of disorders of the above tissues or cells, particularly of the female reproductive system and the endocrine system, expression of this gene at significantly higher or lower levels may be routinely detected in certain tissues or cell types (e.g., endocrine, reproductive, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution of this gene in ovary and adrenal gland indicates that the protein product of this gene is useful for the treatment/diagnosis of female infertility, endocrine disorders, ovarian function, amenorrhea, ovarian cancer and metabolic disorders. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:18 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1780 of SEQ ID NO:18, b is an integer of 15 to 1794, where both a and b correspond to the positions of nucleotide residues shown in SEQ ID NO:18, and where b is greater than or equal to a+14.

Features of Protein Encoded by Gene No: 9

This gene is expressed only in prostate cancer. Therefore, polynucleotides and polypeptides of the invention are useful as reagents for differential identification of the tissue(s) or cell type(s) present in a biological sample and for diagnosis of diseases and conditions which include, but are not limited to, prostate disorders including cancer. Similarly, polypeptides and antibodies directed to these polypeptides are useful in providing immunological probes for differential identification of the tissue(s) or cell type(s). For a number of disorders of the above tissues or cells, particularly of the endocrine and male reproductive system, expression of this gene at significantly higher or lower levels may be routinely detected in certain tissues or cell types (e.g., prostate, endocrine, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution of this gene only in prostate cancerous tissue indicates that the protein product of this gene is useful for the treatment/diagnosis of male infertility, metabolic disorders, and prostate disorders including benign prostate hyperplasia and prostate cancer. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:19 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1023 of SEQ ID NO:19, b is an integer of 15 to 1037, where both a and b correspond to the positions of nucleotide residues shown in SEQ ID NO:19, and where b is greater than or equal to a+14.

Features of Protein Encoded by Gene No: 10

The gene encoding the disclosed cDNA is thought to reside on chromosome 5. Accordingly, polynucleotides related to this invention are useful as a marker in linkage analysis for chromosome 5. The translation product of this gene shares homology with HVH2, which is thought to be a dual specific phosphatase which may function in vivo as a MAP kinase phosphatase (Genbank Accession No. PID|g773355|accession U21108). This gene is expressed primarily in placenta, and to a lesser extent in ovary. Therefore, polynucleotides and polypeptides of the invention are useful as reagents for differential identification of the tissue(s) or cell type(s) present in a biological sample and for diagnosis of diseases and conditions which include, but are not limited to, female infertility, pregnancy disorders, developmental disorders, and ovarian cancer. Similarly, polypeptides and antibodies directed to these polypeptides are useful in providing immunological probes for differential identification of the tissue(s) or cell type(s). For a number of disorders of the above tissues or cells, particularly of the reproductive system and developing embryo, expression of this gene at significantly higher or lower levels may be routinely detected in certain tissues or cell types (e.g., reproductive, developmental, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 125 as residues: Gln-39 to Pro-65. The tissue distribution of this gene in placenta and ovary indicates that the protein product of this gene is useful for the treatment/diagnosis of female infertility, endocrine disorders, fetal deficiencies, ovarian failure, amenorrhea, and ovarian cancer. Furthermore, the tissue distribution indicates that the protein product of this gene is useful for the diagnosis and/or treatment of disorders of the placenta. Specific expression within the placenta indicates that this gene product may play a role in the proper establishment and maintenance of placental function. Alternately, this gene product may be produced by the placenta and then transported to the embryo, where it may play a crucial role in the development and/or survival of the developing embryo or fetus. Expression of this gene product in a vascular-rich tissue such as the placenta also indicates that this gene product may be produced more generally in endothelial cells or within the circulation. In such instances, it may play more generalized roles in vascular function, such as in angiogenesis. It may also be produced in the vasculature and have effects on other cells within the circulation, such as hematopoietic cells. It may serve to promote the proliferation, survival, activation, and/or differentiation of hematopoietic cells, as well as other cells throughout the body.

Many polynucleotide sequences, such as EST sequences, are publicly available and accessible through sequence databases. Some of these sequences are related to SEQ ID NO:20 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1259 of SEQ ID NO:20, b is an integer of 15 to 1273, where both a and b correspond to the positions of nucleotide residues shown in SEQ ID NO:20, and where b is greater than or equal to a+14.

Features of Protein Encoded by Gene No: 11

This gene shares homology with the gene for the Human 3′ apolipoprotein B SAR element gene Rh32 (See Genbank Accession No. T31530). This gene is expressed primarily in prostate and in the pancreas. Therefore, polynucleotides and polypeptides of the invention are useful as reagents for differential identification of the tissue(s) or cell type(s) present in a biological sample and for diagnosis of diseases and conditions which include, but are not limited to, prostate and pancreatic disorders. Similarly, polypeptides and antibodies directed to these polypeptides are useful in providing immunological probes for differential identification of the tissue(s) or cell type(s). For a number of disorders of the above tissues or cells, particularly of the endocrine system, expression of this gene at significantly higher or lower levels may be routinely detected in certain tissues or cell types (e.g., endocrine, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution of this gene in prostate and pancreas indicates that the protein product of this gene is useful for the treatment/diagnosis of male infertility, prostate disorders including benign prostate hyperplasia, prostate cancer, pancreatic cancer, type I and type II diabetes and hypoglycemia. Homology to a known human apolipoprotein may suggest this gene is useful for the detection, prevention, or treatment of various metabolic disorders, particularly those secondary to lipoprotein disorders such as atherosclerosis, coronary heart disease, stroke, and hyperlipidemias. Furthermore, the tissue distribution indicates that the protein product of this gene is useful for the detection, treatment, and/or prevention of various endocrine disorders and cancers, particularly Addison's disease, Cushing's Syndrome, and disorders and/or cancers of the pancreas (e.g. diabetes mellitus), adrenal cortex, ovaries, pituitary (e.g., hyper-, hypopituitarism), thyroid (e.g. hyper-, hypothyroidism), parathyroid (e.g. hyper-, hypoparathyroidism), hypothalamus, and testes. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:21 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1067 of SEQ ID NO:21, b is an integer of 15 to 1081, where both a and b correspond to the positions of nucleotide residues shown in SEQ ID NO:21, and where b is greater than or equal to a+14.

Features of Protein Encoded by Gene No: 12

This gene has homology to a conserved Beta-casein, which is an abundant milk protein (See Genbank Accession No. Q37894). This gene is expressed primarily in stomach, thyroid, and kidney. Therefore, polynucleotides and polypeptides of the invention are useful as reagents for differential identification of the tissue(s) or cell type(s) present in a biological sample and for diagnosis of diseases and conditions which include, but are not limited to, disorders of the digestive tract and/or mammary glands, endocrine disorders. Similarly, polypeptides and antibodies directed to these polypeptides are useful in providing immunological probes for differential identification of the tissue(s) or cell type(s). For a number of disorders of the above tissues or cells, particularly of the digestive system, breast, and endocrine disorders, expression of this gene at significantly higher or lower levels may be routinely detected in certain tissues or cell types (e.g., gastrointestinal, endocrine, mammary, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution of this gene in stomach tissue indicates a role in the treatment/diagnosis of digestive disorders, including stomach cancer and ulceration. Furthermore, the homology to conserved beta-casein may indicate this gene as having utility in the diagnosis and prevention of mammary gland disorders, or other glands of the endocrine system, particularly the thyroid. Additionally, the tissue distribution indicates that the protein product of this gene is useful for the detection, treatment, and/or prevention of various endocrine disorders and cancers, particularly Addison's disease, Cushing's Syndrome, and disorders and/or cancers of the pancreas (e.g. diabetes mellitus), adrenal cortex, ovaries, pituitary (e.g., hyper-, hypopituitarism), thyroid (e.g. hyper-, hypothyroidism), parathyroid (e.g. hyper-, hypoparathyroidism), hypothalamus, and testes. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:22 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 793 of SEQ ID NO:22, b is an integer of 15 to 807, where both a and b correspond to the positions of nucleotide residues shown in SEQ ID NO:22, and where b is greater than or equal to a+14.

Features of Protein Encoded by Gene No: 13

This gene is expressed in brain and lung. Therefore, polynucleotides and polypeptides of the invention are useful as reagents for differential identification of the tissue(s) or cell type(s) present in a biological sample and for diagnosis of diseases and conditions which include, but are not limited to, neurodegenerative disease states, behavioral abnormalities and pulmonary disorders. Similarly, polypeptides and antibodies directed to these polypeptides are useful in providing immunological probes for differential identification of the tissue(s) or cell type(s). For a number of disorders of the above tissues or cells, particularly of the immune, nervous, and pulmonary systems, expression of this gene at significantly higher or lower levels may be routinely detected in certain tissues or cell types (e.g., brain, lung, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in brain and lung tissue indicates that the protein product of this gene is useful for the detection/treatment of neurodegenerative disease states and behavioral disorders such as Alzheimers Disease, Parkinsons Disease, Huntington's Disease, schizophrenia, mania, dementia, paranoia, obsessive compulsive disorder and panic disorder. In addition, it could be used in the detection and treatment of pulmonary disease states such as lung lymphoma or sarcoma formation, pulmonary edema and embolism, bronchitis, cystic fibrosis, and disorders associated with developing lungs, particularly in premature infants where the lungs are the last tissues to develop. Protein, as well as, antibodies directed against the protein may show utility as a tumor marker and immunotherapy targets for the above listed tumors and tissues.

Many polynucleotide sequences, such as EST sequences, are publicly available and accessible through sequence databases. Some of these sequences are related to SEQ ID NO:23 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 618 of SEQ ID NO:23, b is an integer of 15 to 632, where both a and b correspond to the positions of nucleotide residues shown in SEQ ID NO:23, and where b is greater than or equal to a+14.

Features of Protein Encoded by Gene No: 14

In specific embodiments, polypeptides of the invention comprise the following amino acid sequence: IYTHKYYKFRESDQPQLFLFEVGERNQKSE (SEQ ID NO:250). Polynucleotides encoding these polypeptides are also encompassed by the invention.

This gene is expressed exclusively in T-cells. Therefore, polynucleotides and polypeptides of the invention are useful as reagents for differential identification of the tissue(s) or cell type(s) present in a biological sample and for diagnosis of diseases and conditions which include, but are not limited to, immune disorders. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in T-cells indicates that the protein product of this gene is useful for the treatment/detection of immune disorders such as arthritis, asthma, immune deficiency diseases such as AIDS, and leukemia. Additionally, the expression in hematopoietic cells and tissues indicates that this protein may play a role in the proliferation, differentiation, and/or survival of hematopoietic cell lineages. Thus, this gene may be useful in the treatment of lymphoproliferative disorders, and in the maintenance and differentiation of various hematopoietic lineages from early hematopoietic stem and committed progenitor cells. Furthermore, this gene product may be involved in the regulation of cytokine production, antigen presentation, or other processes that may also suggest a usefulness in the treatment of cancer (e.g. by boosting immune responses). Expression of this gene product in T cells also strongly indicates a role for this protein in immune function and immune surveillance. Since the gene is expressed in cells of lymphoid origin, the gene or protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:24 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1344 of SEQ ID NO:24, b is an integer of 15 to 1358, where both a and b correspond to the positions of nucleotide residues shown in SEQ ID NO:24, and where b is greater than or equal to a+14.

Features of Protein Encoded by Gene No: 15

In specific embodiments, polypeptides of the invention comprise the following amino acid sequence: NSLVRSVTEWCANVRGNPCAAALSCPQAVLDAGK (SEQ ID NO:251). Polynucleotides encoding these polypeptides are also encompassed by the invention.

This gene is expressed primarily in T-cells. Therefore, polynucleotides and polypeptides of the invention are useful as reagents for differential identification of the tissue(s) or cell type(s) present in a biological sample and for diagnosis of diseases and conditions which include, but are not limited to, immune disorders. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 130 as residues: Ala-46 to Asp-51. The tissue distribution in T-cells indicates that the protein product of this gene is useful for the diagnosis and treatment of immune disorders including: leukemias, lymphomas, auto-immunities, immunodeficiencies (e.g. AIDS), immunosuppressive conditions (transplantation) and hematopoeitic disorders. Furthermore, this gene product may be involved in the regulation of cytokine production, antigen presentation, or other processes that may also suggest a usefulness in the treatment of cancer (e.g. by boosting immune responses). Expression of this gene product in T cells also strongly indicates a role for this protein in immune function and immune surveillance. Since the gene is expressed in cells of lymphoid origin, the gene or protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:25 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1362 of SEQ ID NO:25, b is an integer of 15 to 1376, where both a and b correspond to the positions of nucleotide residues shown in SEQ ID NO:25, and where b is greater than or equal to a+14.

Features of Protein Encoded by Gene No: 16

This gene is expressed primarily in endometrial tumors. Therefore, polynucleotides and polypeptides of the invention are useful as reagents for differential identification of the tissue(s) or cell type(s) present in a biological sample and for diagnosis of diseases and conditions which include, but are not limited to, cancer, particularly endometrial. Similarly, polypeptides and antibodies directed to these polypeptides are useful in providing immunological probes for differential identification of the tissue(s) or cell type(s). For a number of disorders of the above tissues or cells, particularly of the female reproductive system, expression of this gene at significantly higher or lower levels may be routinely detected in certain tissues or cell types (e.g., reproductive, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in endometrial tumors indicates that the protein product of this gene is useful for the treatment and diagnosis of ovarian and other endometrial cancers, as well as reproductive dysfunction, pre-natal disorders or fetal deficiencies. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:26 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 2909 of SEQ ID NO:26, b is an integer of 15 to 2923, where both a and b correspond to the positions of nucleotide residues shown in SEQ ID NO:26, and where b is greater than or equal to a+14.

Features of Protein Encoded by Gene No: 17

The gene encoding the disclosed cDNA is thought to reside on chromosome 9. Accordingly, polynucleotides related to this invention are useful as a marker in linkage analysis for chromosome 9. This gene is expressed primarily in a variety of osteoclastic cells: osteoclastoma stromal cells, osteosarcoma, chondrosarcoma and stromal cell culture. To a lesser extent, it is also seen in a variety of fetal and embryonic cell and tissue types. Therefore, polynucleotides and polypeptides of the invention are useful as reagents for differential identification of the tissue(s) or cell type(s) present in a biological sample and for diagnosis of diseases and conditions which include, but are not limited to, bone cancer. Similarly, polypeptides and antibodies directed to these polypeptides are useful in providing immunological probes for differential identification of the tissue(s) or cell type(s). For a number of disorders of the above tissues or cells, particularly of the skeletal and developmental systems, expression of this gene at significantly higher or lower levels may be routinely detected in certain tissues or cell types (e.g., skeletal, developmental, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 132 as residues: Arg-43 to Asp-48, Asn-61 to Trp-67, Asp-94 to Gly-105, Ser-110 to Thr-119, Pro-138 to Ala-163, Thr-186 to Trp-196, His-236 to Arg-243, Arg-269 to Cys-279. The tissue distribution in osteoclastic cells indicates that the protein product of this gene is useful for the treatment and detection of a variety of disorders and conditions affecting bone, as well as the skeletal system, including: osteoporosis, fracture, osteosarcoma, osteoclastoma, chondrosarcoma, ossification and osteonecrosis, arthritis, tendonitis, chrondomalacia and inflammation. Furthermore, the tissue distribution indicates that the protein product of this gene is useful for the diagnosis and/or treatment of disorders of the developing embryo. Specific expression within the placenta indicates that this gene product may play a role in the proper establishment and maintenance of placental function. Alternately, this gene product may be produced by the placenta and then transported to the embryo, where it may play a crucial role in the development and/or survival of the developing embryo or fetus. Expression of this gene product in a vascular-rich tissue such as the placenta also indicates that this gene product may be produced more generally in endothelial cells or within the circulation. In such instances, it may play more generalized roles in vascular function, such as in angiogenesis. It may also be produced in the vasculature and have effects on other cells within the circulation, such as hematopoietic cells. It may serve to promote the proliferation, survival, activation, and/or differentiation of hematopoietic cells, as well as other cells throughout the body.

Many polynucleotide sequences, such as EST sequences, are publicly available and accessible through sequence databases. Some of these sequences are related to SEQ ID NO:27 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 2940 of SEQ ID NO:27, b is an integer of 15 to 2954, where both a and b correspond to the positions of nucleotide residues shown in SEQ ID NO:27, and where b is greater than or equal to a+14.

Features of Protein Encoded by Gene No: 18

This gene is expressed primarily in smooth muscle. Therefore, polynucleotides and polypeptides of the invention are useful as reagents for differential identification of the tissue(s) or cell type(s) present in a biological sample and for diagnosis of diseases and conditions which include, but are not limited to, cardiovascular disorders including lymphatic system disorders. Similarly, polypeptides and antibodies directed to these polypeptides are useful in providing immunological probes for differential identification of the tissue(s) or cell type(s). For a number of disorders of the above tissues or cells, particularly of the cardiovascular and lymphatic systems, expression of this gene at significantly higher or lower levels may be routinely detected in certain tissues or cell types (e.g., cardiovascular, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in smooth muscle indicates that the protein product of this gene is useful for the diagnosis and treatment of conditions and pathologies of the cardiovascular system, such as heart disease, restenosis, atherosclerosis, stoke, angina, thrombosis, and wound healing. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:28 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 520 of SEQ ID NO:28, b is an integer of 15 to 534, where both a and b correspond to the positions of nucleotide residues shown in SEQ ID NO:28, and where b is greater than or equal to a+14.

Features of Protein Encoded by Gene No: 19

The translation product of this gene shares sequence homology with 5′-nucleotidase (See Genbank Accession No. 2668557), as well as the gene for alpha-1 collagen type X (See Genbank Accession No. gb|X67348|MMCOL10A).

In specific embodiments, polypeptides of the invention comprise the following amino acid sequence:

| | |
|---|---|
| MAQHFSLAACDVVGFDLDHTLCRYNLPESAPLIYNSFAQFLVKEKGYDKELLNVTPEDWDFCCKGLALDLEDGNFLKLANNGTVLR | (SEQ ID NO:255) |
| ASHGTKMMTPEVLAEAYGKKEWKHFLSDTGMACRSGKYYFYDNYFDLPGALLCARVVDYLTKLNNGQKTFDFWKDIVAAIQHNYKM | |
| SAFKENCGIYFPEIKRDPGRYLHSCPESVKKWLRQLKNAGKILLLITSSHSDYCRLLCEYILGNDFTDLFDIVITNALKPGFFSHL | |
| PSQRPFRTLENDEEQEALPSLDKPGWYSQGNAVHLYELLKKMTGKPEPKVVYFGDSMHSDIFPARHYSNWETVLILEELRGDEGTR | |
| SQRPEESEPLEKKGKYEGPKAKPLNTSSKKWGSFFIDSVLGLENTEDSLVYTWSCKRISTYSTIAIPSIEAIAELPLDYKFTRFSS | |
| SNSKTAGYYPNPPLVLSSDETLISK, | |
| TSSHSDYCRLLCEYILGNDFTDLFDIV, | (SEQ ID NO:256) |
| MAQHFSLAACDVVGFDLDHTLCRYNLPESAPLIYNSFAQFLVKEKGYDK, | (SEQ ID NO:257) |
| ELLNVTPEDWDFCCKGLALDLEDGNFLKLANNGTVLRASHGTKMMTPEVLAE, | (SEQ ID NO:258) |
| AYGKKEWKHFLSDTGMACRSGKYYFYDNYFDLPGALLCARVVDYLTKLN, | (SEQ ID NO:259) |
| NGQKTFDFWKDIVAAIQHNYKMSAFKENCGIYFPEIKRDPGRYLHSCPESVK, | (SEQ ID NO:260) |
| KWLRQLKNAGKILLLITSSHSDYCRLLCEYILGNDFTDLFDIVITNALKPGFFS, | (SEQ ID NO:261) |
| HLPSQRPFRTLENDEEQEALPSLDKPGWYSQGNAVHLYELLKKMTGKPEP, | (SEQ ID NO:262) |
| KVVYFGDSMHSDIFPARHYSNWETVLILEELRGDEGTRSQRPEESEPLEKKG, | (SEQ ID NO:263) |
| KYEGPKAKPLNTSSKKWGSFFIDSVLGLENTEDSLVYTWSCKRISTYSTIA, | (SEQ ID NO:264) |
| and/or | |
| IPSIEAIAELPLDYKFTRFSSSNSKTAGYYPNPPLVLSSDETLISK. | (SEQ ID NO:265) |

encompassed by the invention. Additionally, in specific embodiments, polypeptides of the invention comprise the following amino acid sequence::

| | |
|---|---|
| CCTTAAAAGCTGACATTTTATAATTGTGTTGTATAGCAGCAACTATATCCTTCCAAAAATCAAATGTTTTTTGACCATTGTTCAGT | (SEQ ID NO:252) |
| T, | |
| CCTTA AAAGCTGACATTTTATAATTGTGTTGTATAGCA, | (SEQ ID NO:253) |
| and/or | |
| CTTCCAAAAATCAAATGTTTTTTGACCATTGTTCAGTT. | (SEQ ID NO:254) |

encompassed by the invention.

The gene encoding the disclosed cDNA is thought to reside on chromosome 6. Accordingly, polynucleotides related to this invention are useful as a marker in linkage analysis for chromosome 6. This gene is expressed primarily in prostate and smooth muscle. Therefore, polynucleotides and polypeptides of the invention are useful as reagents for differential identification of the tissue(s) or cell type(s) present in a biological sample and for diagnosis of diseases and conditions which include, but are not limited to, prostate cancer and cardiovascular disorders. Similarly, polypeptides and antibodies directed to these polypeptides are useful in providing immunological probes for differential identification of the tissue(s) or cell type(s). For a number of disorders of the above tissues or cells, particularly of the prostate and cardiovascular system, expression of this gene at significantly higher or lower levels may be routinely detected in certain tissues or cell types (e.g., prostate, cardiovascular, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in prostate indicates that the protein product of this gene is useful for the treatment and diagnosis of prostate cancer and reproductive disorders. In addition, the expression in smooth muscle would suggest a role for this gene product in the treatment and diagnosis of cardiovascular disorders such as hypertension, restenosis, atherosclerosis, stoke, angina, thrombosis, and other aspects of heart disease and respiration. Protein, as well as, antibodies directed against the protein may show utility as a tissue-specific marker and/or immunotherapy target for the above listed tissues.

Many polynucleotide sequences, such as EST sequences, are publicly available and accessible through sequence databases. Some of these sequences are related to SEQ ID NO:29 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1813 of SEQ ID NO:29, b is an integer of 15 to 1827, where both a and b correspond to the positions of nucleotide residues shown in SEQ ID NO:29, and where b is greater than or equal to a+14.

Features of Protein Encoded by Gene No: 20

This gene is expressed primarily in endometrial tissue, and to a lesser extent in synovium. Therefore, polynucleotides and polypeptides of the invention are useful as reagents for differential identification of the tissue(s) or cell type(s) present in a biological sample and for diagnosis of diseases and conditions which include, but are not limited to, endometrial cancer and arthritis. Similarly, polypeptides and antibodies directed to these polypeptides are useful in providing immunological probes for differential identification of the tissue(s) or cell type(s). For a number of disorders of the above tissues or cells, particularly of the reproductive and skeletal systems, expression of this gene at significantly higher or lower levels may be routinely detected in certain tissues or cell types (e.g., reproductive, skeletal, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 135 as residues: Ser-19 to His-24, Pro-36 to Arg-43, Ala-61 to Gly-67, Pro-86 to Ala-95. The tissue distribution endometrial tissue indicates that the protein product of this gene is useful for the diagnosis and treatment of endometrial cancers, as well as reproductive and developmental disorders (fetal deficiencies and other pre-natal conditions). In addition, the expression of this gene product in synovium would suggest a role in the detection and treatment of disorders and conditions affecting the skeletal system, in particular the connective tissues (e.g. arthritis, trauma, tendonitis, chrondomalacia and inflammation), such as in the diagnosis or treatment of various autoimmune disorders such as rheumatoid arthritis, lupus, scleroderma, and dermatomyositis as well as dwarfism, spinal deformation, and specific joint abnormalities as well as chondrodysplasias (i.e. spondyloepiphyseal dysplasia congenita, familial osteoarthritis, Atelosteogenesis type II, metaphyseal chondrodysplasia type Schmid). Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:30 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1465 of SEQ ID NO:30, b is an integer of 15 to 1479, where both a and b correspond to the positions of nucleotide residues shown in SEQ ID NO:30, and where b is greater than or equal to a+14.

Features of Protein Encoded by Gene No: 21

The gene encoding the disclosed cDNA is thought to reside on chromosome 6. Accordingly, polynucleotides related to this invention are useful as a marker in linkage analysis for chromosome 6. This gene is expressed primarily in keratinocytes, fetal tissue (especially fetal brain) and leukocytic cell types and tissues (e.g., B-cell, macrophages, Jurkat T-Cell, T cell helper cells, spleen, thymus and lymphoma). Therefore, polynucleotides and polypeptides of the invention are useful as reagents for differential identification of the tissue(s) or cell type(s) present in a biological sample and for diagnosis of diseases and conditions which include, but are not limited to, integument and immune systems, as well as developmental disorders. Similarly, polypeptides and antibodies directed to these polypeptides are useful in providing immunological probes for differential identification of the tissue(s) or cell type(s). For a number of disorders of the above tissues or cells, particularly of the skin, immune and central nervous systems, expression of this gene at significantly higher or lower levels may be routinely detected in certain tissues or cell types (e.g., skin, immune, CNS, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 136 as residues: Gln-58 to Val-63. The tissue distribution in leukocytic cell types and tissues indicates that the protein product of this gene is useful for the diagnosis and treatment of immune disorders including leukemias, lymphomas, auto-immunities, immunodeficiencies (e.g. AIDS), immunosuppressive conditions (transplantation) and hematopoeitic disorders. Expression in keratinocytes would suggest a role for the gene product in the diagnosis and treatment of skin disorders such as cancers (melanomas), eczema, psoriasis, wound healing and grafts. In addition, the expression in fetal brain might implicate this gene product in the detection and treatment of developmental and neurodegenerative diseases of the brain and nervous system, or behavioral or nervous system disorders, such as depression, schizophrenia, Alzheimer's disease, Parkinson's disease, Huntington's disease, mania, dementia; paranoia, addictive behavior and sleep disorders. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:31 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 973 of SEQ ID NO:31, b is an integer of 15 to 987, where both a and b correspond to the positions of nucleotide residues shown in SEQ ID NO:31, and where b is greater than or equal to a+14.

Features of Protein Encoded by Gene No: 22

Translation product of this gene shares significant homology with the conserved YME1 PROTEIN from Saccharomyces cerevisiae, which is a putative ATP-dependent protease thought to regulate the assembly of key respiratory chains within the mitochondria (See Genbank Accession No. P32795). Furthermore, the translation product of this gene also shares significant homology with a metalloproteinase from mouse (See Genbank Accession No. AF090430).

In specific embodiments, polypeptides of the invention comprise the following amino acid sequence:

| | |
|---|---|
| MKTKNIPEAHQDAFKTGFAEGFLKAQALTQKTNDSLRRTRLILFVLLLFGIYGLLKNPFLSVRFRTTTGLDSAVDPVQMKNVTFEH | (SEQ ID NO:266) |
| VKGVEEAKQELQEVVEFLKNPQKFTILGGKLPKGILLVGPPGTGKTLLARAVAGEADVPFYYASGSEFDEMFVGVGASRIRNLFRE | |
| AKANAPCVIFIDELDSVGGKRIESPMHPYSRQTINQLLAEMDGFKPNEGVIIIGATNFPEALDNALIRPGRFDMQVTVPRPDVKGR | |
| TEILKWYLNKIKFDXSVDPEIIARGTVGFSGAELENLVNQAALKAAVDGKEMVTMKELGVFQRQNSNGA, | |
| MKTKNIPEAHQDAFKTGFAEG, | (SEQ ID NO:267) |
| PVQMKNVTFEHVKGVEEAKQELQ, | (SEQ ID NO:268) |
| SRQTINQLLAEMDGFKPNEGVII, | (SEQ ID NO:269) |
| and/or | |
| FSGAELENLVNQAALKAAVDG KEM. | (SEQ ID NO:270) |

invention.

The gene encoding the disclosed cDNA is thought to reside on chromosome 10. Accordingly, polynucleotides related to this invention are useful as a marker in linkage analysis for chromosome 10. This gene is expressed primarily in T-cells. Therefore, polynucleotides and polypeptides of the invention are useful as reagents for differential identification of the tissue(s) or cell type(s) present in a biological sample and for diagnosis of diseases and conditions which include, but are not limited to, immune and hematopoeitic disorders. Similarly, polypeptides and antibodies directed to these polypeptides are useful in providing immunological probes for differential identification of the tissue(s) or cell type(s). For a number of disorders of the above tissues or cells, particularly of the immune and hematopoeitic systems, expression of this gene at significantly higher or lower levels may be routinely detected in certain tissues or cell types (e.g., immune, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in T-cells indicates that the protein product of this gene is useful for the diagnosis and treatment of immune disorders, including leukemias, lymphomas, auto-immunities, immunodeficiencies (e.g. AIDS), immunosuppressive conditions (transplantation) and hematopoeitic disorders. Additionally, this gene product may be involved in the regulation of cytokine production, antigen presentation, or other processes that may also suggest a usefulness in the treatment of cancer (e.g. by boosting immune responses). Expression of this gene product in T cells also strongly indicates a role for this protein in immune function and immune surveillance. Since the gene is expressed in cells of lymphoid origin, the gene or protein, as well as, antibodies directed against the protein may show utility as a tumor marker and/or immunotherapy targets for the above listed tissues. Furthermore, the homology of this gene indicates that it may play an important role in disorders affecting metabolism.

Many polynucleotide sequences, such as EST sequences, are publicly available and accessible through sequence databases. Some of these sequences are related to SEQ ID NO:32 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 2919 of SEQ ID NO:32, b is an integer of 15 to 2933, where both a and b correspond to the positions of nucleotide residues shown in SEQ ID NO:32, and where b is greater than or equal to a+14.

Features of Protein Encoded by Gene No: 23

This gene is expressed primarily in human chronic synovitis. Therefore, polynucleotides and polypeptides of the invention are useful as reagents for differential identification of the tissue(s) or cell type(s) present in a biological sample and for diagnosis of diseases and conditions which include, but are not limited to, synovial and other inflammatory disorders. Similarly, polypeptides and antibodies directed to these polypeptides are useful in providing immunological probes for differential identification of the tissue(s) or cell type(s). For a number of disorders of the above tissues or cells, particularly of the synovial tissue and immune system, expression of this gene at significantly higher or lower levels may be routinely detected in certain tissues or cell types (e.g., synovial, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in human chronic synovitis indicates that the protein product of this gene is useful for the study, diagnosis and treatment of inflammatory disorders such as chronic synovitis. In addition, the expression of this gene product in synovium indicates a role in the detection and treatment of disorders and conditions affecting the skeletal system, in particular osteoporosis as well as disorders afflicting connective tissues (e.g. arthritis, trauma, tendonitis, chrondomalacia and inflammation), such as in the diagnosis or treatment of various autoimmune disorders such as rheumatoid arthritis, lupus, scleroderma, and dermatomyositis as well as dwarfism, spinal deformation, and specific joint abnormalities as well as chondrodysplasias (i.e. spondyloepiphyseal dysplasia congenita, familial osteoarthritis, Atelosteogenesis type II, metaphyseal chondrodysplasia type Schmid). Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:33 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1352 of SEQ ID NO:33, b is an integer of 15 to 1366, where both a and b correspond to the positions of nucleotide residues shown in SEQ ID NO:33, and where b is greater than or equal to a+14.

Features of Protein Encoded by Gene No: 24

This gene is expressed primarily in pituitary, breast cancer, and bone marrow, and to a lesser extent in breast, prostate, uterine cancer and cerebellum. Therefore, polynucleotides and polypeptides of the invention are useful as reagents for differential identification of the tissue(s) or cell type(s) present in a biological sample and for diagnosis of diseases and conditions which include, but are not limited to, endocrine, reproductive disorders and cancers. Similarly, polypeptides and antibodies directed to these polypeptides are useful in providing immunological probes for differential identification of the tissue(s) or cell type(s). For a number of disorders of the above tissues or cells, particularly of the reproductive, metabolic and endocrine systems, expression of this gene at significantly higher or lower levels may be routinely detected in certain tissues or cell types (e.g., endocrine, reproductive, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 139 as residues: Asp-32 to Gln-38, Lys-88 to Ile-97. The tissue distribution pituitary indicates that the protein products of this gene are useful for the study, treatment and diagnosis of various endocrine disorders, particularly Addison's disease, Cushing's Syndrome, and disorders and/or cancers of the pancreas (e.g. diabetes mellitus), adrenal cortex, ovaries, pituitary (e.g., hyper-, hypopituitarism), thyroid (e.g. hyper-, hypothyroidism), parathyroid (e.g. hyper-, hypoparathyroidism), hypothalamus, and testes, as well as reproductive diseases and disorders. Alternatively, the tissue distribution to breast cancer tissue indicates that the protein product of this gene is useful for the diagnosis and intervention of these tumors, as well as other tumors where expression is indicated. Protein, as well as, antibodies directed against the protein may show utility as a tissue-specific marker and/or immunotherapy target for the above listed tissues.

Many polynucleotide sequences, such as EST sequences, are publicly available and accessible through sequence databases. Some of these sequences are related to SEQ ID NO:34 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 653 of SEQ ID NO:34, b is an integer of 15 to 667, where both a and b correspond to the positions of nucleotide residues shown in SEQ ID NO:34, and where b is greater than or equal to a+14.

Features of Protein Encoded by Gene No: 25

The translation product of this gene shares sequence homology with androgen withdrawal apoptosis protein in rat which is thought to be important in programmed cell death. In specific embodiments, polypeptides of the invention comprise the following amino acid sequence:

| | |
|---|---|
| LPMWQVTAFLDHNIVTAQTTWKGLWMSCVVQSTGHMQCKVYDSVLALSTEVQAARALTVSAVLLAFVALFVTLAGAQCTTCVAPGP | (SEQ ID NO:271) |
| AKARVALTGGVLYLFCGLLALVPLCWFANIVVREFYDPSVPVSQKYELGAXLYIGWAATALLMVGGCLLCCGAWVCTGRPDLSFPV | |
| KYSAPRRPTATGDYDKKNYV, | |
| EVRQTRGANGLGPRAGSAGAKAPGPAQGAAQHGLGGSAGLRVRVSPLA, | (SEQ ID NO:272) |
| RGANGLGPRAGSAGAKAPGPAQG, | (SEQ ID NO:273) |
| RLRAARGSVIGCGTMTRARIGCFGPGGRARGTESAPEPSKRVPPGRXWQTLGGATDPRGKRTGAKSRERGRKGTRARPGRRAAR | (SEQ ID NO:274) |
| PWGFCGPSGARLASSHGVRSVGDPGPGAVPGGLGGSDPGVRAAHVAGDRLPGPQHRDGADHLEGAVDVVRGAEHXAHAVQSVRLGA | |
| GSEHRGAGGAGAHRERRAAGVRCALRDPGGRAVHHLRGPGPGQGACGPHGRRALPVLRAAGARATLLVRQHCRPRVLRPVCARVAE | |
| VRAGRXLYIGWAATALLMVGGCLLCCGAWVCTGRPDLSFPVKYSAPRRPTATGDYDKKNYV, | |
| GCFGPGGRARGTESAPEPSKRVPPGRX, | (SEQ ID NO:275) |
| GGATDPRGKRTGAKSRERGRKGTRARP, | (SEQ ID NO:276) |
| GPSGARLASSHGVRSVGDPGPGAVP, | (SEQ ID NO:277) |
| AAHVAGDRLPGPQHRDGADHLEGAVD, | (SEQ ID NO:278) |
| AVQSVRLGAGSEHRGAGGAGAHR, | (SEQ ID NO:279) |
| CALRDPGGRAVHHLRGPGPGQGACGPH, | (SEQ ID NO:280) |
| RATLLVRQHCRPRVLRPVCARVAEVRA, | (SEQ ID NO:281) |
| LYIGWAATALLMVGGCLLCCGAWVCTG, | (SEQ ID NO:282) |
| MGSAALE, | (SEQ ID NO:283) |
| ACGLPMWQVTAFLDHNIVTAQTTWKGLWMSCVVQSTG, | (SEQ ID NO:284) |
| HMQCKVYDSVLALSTEVQAAR, | (SEQ ID NO:285) |
| GAQCTTCVAPGPAKARVALT, | (SEQ ID NO:286) |
| GGVLYLFCGLLALVPLCWFANIVVREFYDPSVPVSQKYELGA, | (SEQ ID NO:287) |
| and/or | |
| LYIGWAATA. | (SEQ ID NO:288) |

polypeptide is expected to contain multiple transmembrane domains. The extracellular portion of the polypeptide is expected to comprise residues 1-51 of the foregoing amino acid sequence. Therefore, particularly preferred polypeptides encoded by this gene comprise residues 1-51 of the foregoing amino acid sequence. Polynucleotides encoding these polypeptides are also encompassed by the invention. This gene is expressed primarily in human adult pulmonary and brain (striatum) tissue, and to a lesser extent, in thymus, synovium and testis. Therefore, polynucleotides and polypeptides of the invention are useful as reagents for differential identification of the tissue(s) or cell type(s) present in a biological sample and for diagnosis of diseases and conditions which include, but are not limited to, reproductive, metabolic, pulmonary, or neurodegenerative disorders. Similarly, polypeptides and antibodies directed to these polypeptides are useful in providing immunological probes for differential identification of the tissue(s) or cell type(s). For a number of disorders of the above tissues or cells, particularly of the reproductive, nervous, respiratory and metabolic systems expression of this gene at significantly higher or lower levels may be routinely detected in certain tissues or cell types (e.g., pulmonary, neural, endocrine, skeletal, reproductive, and cancerous and wounded tissues) or bodily fluids (e.g., lymph, serum, plasma, seminal fluid, pulmonary surfactant or sputum, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in testes, combined with the homology to the androgen withdrawal apoptosis rat protein indicates that polynucleotides and polypeptides corresponding to this gene are useful for the study, diagnosis and treatment of disorders in which the mechanisms controlling programmed cell death are instrumental. This could include reproductive, neurodegenerative, and various metabolic disorders and diseases such as cancer. Moreover, the protein product of this gene is useful for the detection/treatment of neurodegenerative disease states, behavioral disorders, or inflammatory conditions which include, but are not limited to Alzheimer's Disease, Parkinson's Disease, Huntington's Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, depression, panic disorder, learning disabilities, ALS, psychoses, autism, and altered behaviors, including disorders in feeding, sleep patterns, balance, and perception. In addition, elevated expression of this gene product in regions of the brain indicates that it plays a role in normal neural function. Potentially, this gene product is involved in synapse formation, neurotransmission, learning, cognition, homeostasis, or neuronal differentiation or survival. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:35 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1696 of SEQ ID NO:35, b is an integer of 15 to 1710, where both a and b correspond to the positions of nucleotide residues shown in SEQ ID NO:35, and where b is greater than or equal to a+14.

Features of Protein Encoded by Gene No: 26

The translation product of this gene shares homology with both ubiquitin and a G-protein coupled receptor TM3 consensus polypeptide (see Genbank accession Nos. gnl|PID|e331456 (AJ000657) and R50664, respectively).

In specific embodiments, polypeptides of the invention comprise the following amino acid sequence:

| | |
|---|---|
| LHYFALSFVLILTEICLVSSGMGF, | (SEQ ID NO:289) |
| QLRNGIPPGRKALFCSGKPRLFTLGQGRTCA, | (SEQ ID NO:290) |
| WSGLWVTTWNGSSGERTPSPWRRKRASQSAGRIASWMSF, | (SEQ ID NO:291) |
| WRTQGP, | (SEQ ID NO:292) |
| PVGRVTGQGPAGRWVRRLPCSRRAGGERGPHWGVWAGPQMSCGLXFGPWFVPMLLMSHSLLPSWSGLWVTTWNGSSGERTPSPWRR | (SEQ ID NO:293) |
| KRASQSAGRIASWMSF, | |
| WVRRLPCSRRAGGERGPHWGVWAGPQMSC, | (SEQ ID NO:294) |
| WSGLWVTTWNGSSGERTPSPWRRKRA, | (SEQ ID NO:295) |
| TLAVNLCGQAVWPLVGMAGIWALPLGGIPACLVPVCTGKNSNMHLAPFTILPYPLCSFLLKSVLSAQEWDSPRKESTFLFWEAQTV | (SEQ ID NO:296) |
| HFGAGTNMCLVNLLENSHHLLPPSLYSEKPD, | |
| LVGMAGIWALPLGGIPACLVPVC, | (SEQ ID NO:297) |
| FLLKSVLSAQEWDSPRKESTFL, | (SEQ ID NO:298) |
| and/or | |
| AGTNMCLVNLLENSHHLLPPSLYSEK. | (SEQ ID NO:299) |

invention.

The gene encoding the disclosed cDNA is believed to reside on chromosome 1. Accordingly, polynucleotides related to this invention are useful as a marker in linkage analysis for chromosome 1. This gene is expressed primarily in activated T cells, and to a lesser extent, in CD34 depleted buffy coat. Therefore, polynucleotides and polypeptides of the invention are useful as reagents for differential identification of the tissue(s) or cell type(s) present in a biological sample and for diagnosis of diseases and conditions which include, but are not limited to, immune and hemopoietic disorders. Similarly, polypeptides and antibodies directed to these polypeptides are useful in providing immunological probes for differential identification of the tissue(s) or cell type(s). For a number of disorders of the above tissues or cells, particularly of the hemopoietic and immune system, expression of this gene at significantly higher or lower levels may be routinely detected in certain tissues or cell types (e.g., immune, hemopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 141 as residues: Thr-15 to His-21, Gly-30 to Lys-39, Arg-113 to Met-118, Arg-178 to Ala-187. The tissue distribution in activated T-cells indicates that polynucleotides and polypeptides corresponding to this gene are useful for the treatment and diagnosis of hematopoetic related disorders such as anemia, pancytopenia, leukopenia, thrombocytopenia or leukemia since stromal cells are important in the production of cells of hematopoietic lineages; The uses include bone marrow cell ex vivo culture, bone marrow transplantation, bone marrow reconstitution, radiotherapy or chemotherapy of neoplasia. The gene product may also be involved in lymphopoiesis, therefore, it can be used in immune disorders such as infection, inflammation, allergy, immunodeficiency etc. Furthermore, the homology to G-coupled proteins as well as to ubiquitin may implicate this gene as being important in regulation of gene expression and protein sorting—both of which are vital to development and would healing models. Therefore, the gene may provide utility in the diagnosis, prevention, and/or treatment of various developmental disorders which include, but are not limited to Tay-Sachs disease, phenylkenonuria, galactosemia, hyperlipidemias, porphyrias, and Hurler's syndrome. The tissue distribution within CD34 depleted buffy coat cells further implicates the invention as having a utility in the treatment, detection, and/or prevention of developmental disorders. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:36 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1082 of SEQ ID NO:36, b is an integer of 15 to 1096, where both a and b correspond to the positions of nucleotide residues shown in SEQ ID NO:36, and where b is greater than or equal to a+14.

Features of Protein Encoded by Gene No: 27

The translation product of this gene was found to have homology to the F38H4.7 protein of Caenorhabditis elegans, in addition to the muscle-specific human sarcosin protein (See Genbank Accession Nos. gnl|PID|e255903 and gi|3047308 (AF056929), respectively) which are thought to play a role in regulating certain aspects of cellular metabolism.

In specific embodiments, polypeptides of the invention comprise the following amino acid sequence: VGKGLSSQR (SEQ ID NO:300),

| | |
|---|---|
| KNHLGAWSEEDTPPMALGRTQVPGVTGQKHAGWVGAWPEWGGAVPQHLGVAPTAPGGLGQQYVQASSAPGPGRGRLGPHSAAGELW | (SEQ ID NO:301) |
| GTGSESHLGLQGPRGPSREAGQRGPLSGPIRWMRTDTANRSPPGPGLGLSGRGRPHTESCVRRTQAGIQAVGTPRRLGLGGLGHVT | |
| GGARVLINGQEQKERKVWDPRLWEQRHPGWLLAEKWESQMHCNVQ, | |
| TPPMALGRTQVPGVTGQKHAGWV, | (SEQ ID NO:302) |
| YVQASSAPGPGRGRLGPHSAAG, | (SEQ ID NO:303) |
| HLGLQGPRGPSREAGQRGPLSGPIRW, | (SEQ ID NO:304) |
| LGLSGRGRPHTESCVRRTQAG, | (SEQ ID NO:305) |
| PRRLGLGGLGHVTGGARVLINGQEQ, | (SEQ ID NO:306) |
| GAAVQCRCPLVRGRVSAAAAAGDAREQAEGSGQGPGPHSLPAHDHRGVRCRSRTVGHPGGPRGGQPLLHFTVNPKPRVEFIDRPRC | (SEQ ID NO:307) |
| CLRGKECSINRFQQVESRWGYXGTSDRIRFSVNKRIFVVGFGLYGSIHGPTDYQVNIQIIHTDSNTVLGQNDTGFSCDGSASTFRV | |
| MFKEPVEVLPNVNYTACATLKGPDSHYGTKGLRKVTHESPTTGAKTCFTFCYAAGNNNGTSVEDGQIPEVIFYT, | |
| EQAEGSGQGPGPHSLPAHDHRGVR, | (SEQ ID NO:308) |
| GPRGGQPLLHFTVNPKPRVEFID, | (SEQ ID NO:309) |
| ECSINRFQQVESRWGYXGTSDRIRFSV, | (SEQ ID NO:310) |
| YGSIHGPTDYQVNIQIIHTDSNTVL, | (SEQ ID NO:311) |
| SCDGSASTFRVMFKEPVEVLPNVN, | (SEQ ID NO:312) |
| VTHESPTTGAKTCFTFCYAAGNNNGT, | (SEQ ID NO:313) |
| HSASDQRTTALNSRTSRMPSVSRSRTATSVSRSMSVKPSAVMASAVFLSMFSRHRLASCGSSKSRACVSSMKALSARRFFFRNSTQ | (SEQ ID NO:314) |
| WASSAGTAYFLAVYXVVITVSGPICTS SE, | |
| TTALNSRTSRMPSVSRSRTATSVSRS, | (SEQ ID NO:315) |
| and/or | |
| SRHRLASCGSSKSRACVSSMKALSA. | (SEQ ID NO:316) |

invention.

This gene is expressed primarily in activated T cells, and to a lesser extent, in fetal kidney. Therefore, polynucleotides and polypeptides of the invention are useful as reagents for differential identification of the tissue(s) or cell type(s) present in a biological sample and for diagnosis of diseases and conditions which include, but are not limited to, immune, developmental, renal, and metabolic diseases. Similarly, polypeptides and antibodies directed to these polypeptides are useful in providing immunological probes for differential identification of the tissue(s) or cell type(s). For a number of disorders of the above tissues or cells, particularly of the immune and metabolic systems, expression of this gene at significantly higher or lower levels may be routinely detected in certain tissues or cell types (e.g., immune, developmental, renal, metabolic, and cancerous and wounded tissues) or bodily fluids (e.g., lymph, serum, amniotic fluid, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in activated T-cells and fetal kidney indicates that polynucleotides and polypeptides corresponding to this gene are useful for the study and treatment of diseases and disorders of the immune, metabolic, and endocrine systems; such as renal diseases and T cell dysfunctions. Since the gene is expressed in cells of lymphoid origin, the natural gene product may be involved in immune functions. Therefore it may be also used as an agent for immunological disorders including arthritis, asthma, immune deficiency diseases such as AIDS, and leukemia. Moreover, this gene or gene product could be used in the treatment and/or detection of kidney diseases including renal failure, nephritus, renal tubular acidosis, proteinuria, pyuria, edema, pyelonephritis, hydronephritis, nephrotic syndrome, crush syndrome, glomerulonephritis, hematuria, renal colic and kidney stones, in addition to Wilm's Tumor Disease, and congenital kidney abnormalities such as horseshoe kidney, polycystic kidney, and Falconi's syndrome. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:37 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 2265 of SEQ ID NO:37, b is an integer of 15 to 2279, where both a and b correspond to the positions of nucleotide residues shown in SEQ ID NO:37, and where b is greater than or equal to a+14.

Features of Protein Encoded by Gene No: 28

The translation product of this gene shares sequence homology with Cystatin-related epididymal specific protein in mouse, which is thought to be important in reproductive system function/regulation (See Genbank accession no.bbs|118813). Based on the structural similarity between these proteins, the translation product of this gene, hereinafter “Cystatin G”, is expected to share biological activities with cystatin related proteins and other cysteine protease inhibitors. Such activities are known in the art and are described elsewhere herein.

In specific embodiments, polypeptides of the invention comprise the following amino acid sequence:

(SEQ ID NO:319)

MPRCRWLSLILLTIPLALVARKDPKKNETGVLRKLKPVNASNANVKQCLW
FAMQEYNKESEDKYVFLVVKTLQAQLQVTNLLEYLIDVEIARSDCRKPLS
TNEICAIQENSKLKRKLSCSFLVGALPWNGEFTVMEKKCEDA,

(SEQ ID NO:321)

ARKDPKKNETGVLRKLKPVNASNANVKQCLWFAMQEYNKESEDKYVFLVV
KTLQAQLQVTNLLEYLIDVEIARSDCRKPLSTNEICAIQENSKLKRKLSCSFLVGA
LPWNGEFTVMEKKCEDA,

(SEQ ID NO:320)

CLWFAMQEYNKESEDKYVFLVVKTLQAQLQVTNLLEYLIDVEIARSDCRK
PLSTNEICAIQENSKLKRKLSCSFLVGALPWNGEFTVMEKKC

(SEQ ID NO:317)

EYNKESEDKYVFLV,

(SEQ ID NO:322)

THSSSCCGHPILPTAPALRRRGGESTLPLAQGT

(SEQ ID NO:)

IDVEIARSDCRKPL,

(SEQ ID NO:323)

SWACSVLTTRKTYLSSLSLLYSCMAKPXTLLPRWXLKALTGFNF
LSTPVSFFFGSFLATRARGMVRRIRESHRHLGMVPWARGKVDSPPRRLRA
GAVGRMGWPQLLLWVQESSSHTLPPVSVSPAL,
and/or

(SEQ ID NO:324)

SWACSVLTTRKTYLSSLSLLYSCMAKPXTLLPRWXLKALTGFNFLSTPVS
FFFGSFLATRARGMVRRIRESHRHLGMVPWARGKVDSPPRRLRAGAVGRM
GWPQLLLWVQESSSHTLPPVSVSPAL.

encompassed by the invention.

Preferred cystatin polypeptide fragments are shown to be active in the following assays: The methods used for active site titration of papain, titration of the molar enzyme inhibitory concentration in cystatin G preparations, and for determination of equilibrium constants for dissociation (Ki) of complexes between cystatin G and cysteine peptidases are described in detail in Hall et al., Biochem. J., 291: 123-29 (1993) and Abrahamson, Methods Enzymol., 244: 685-700 (1994), both of which are hereby incorporated herein by reference. The enzymes used for equilibrium assays are papain (EC 3.4.22.2; from Sigma, St Lois, Mo.) and cathepsin B (EC 3.4.22.1; from Calbiochem, La Jolla, Calif.). The fluorogenic substrate used was Z-Phe-Arg-NHMec (10 mM; from Bachem Feinchemikalien, Bubendorf, Switzerland) and the assay buffer was 100 mM Na-phosphate buffer (pH 6.5 and 6.0 for papain and cathepsin B, respectively), containing 1 mM dithiothreitol and 2 mM EDTA. Steady state velocities are measured and Ki values were calculated according to Henderson, Biochem J., 127: 321-333 (1972), incorporated herein by reference. Corrections for substrate competition are made using Km values of 150=B5M for cathepsins B (Barrett and Kirschke, Methods Enzymol., 80: 535-561 (1981) and 60=B5M for papain (Hall et al., Biochem. J., 291: 123-29 (1992)), both of which are hereby incorporated herein by reference. This gene is expressed primarily in human testes. Therefore, polynucleotides and polypeptides of the invention are useful as reagents for differential identification of the tissue(s) or cell type(s) present in a biological sample and for diagnosis of diseases and conditions which include, but are not limited to, reproductive disorders and cancer. Similarly, polypeptides and antibodies directed to these polypeptides are useful in providing immunological probes for differential identification of the tissue(s) or cell type(s). For a number of disorders of the above tissues or cells, particularly of the reproductive system, expression of this gene at significantly higher or lower levels may be routinely detected in certain tissues or cell types (e.g., reproductive, testicular, and cancerous and wounded tissues) or bodily fluids (e.g., lymph, serum, plasma, seminal fluid, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 143 as residues: Arg-21 to Thr-29. The tissue distribution in testes, combined with the homology to the mouse cystatin-related epididymal specific protein indicates that polynucleotides and polypeptides corresponding to this gene are useful for the study, diagnosis, treatment, and/or prevention of reproductive diseases and disorders. The protein may also show utility in the development and use as a contraceptive. Cysteine proteinase inhibitors of the cystatin superfamily are ubiquitous in the body and are generally tight-binding inhibitors of papain-like cysteine proteinases, such as cathepsins B, H, L, S, and K (for review, see Ref. 1). They should therefore serve a protective function to regulate the activities of such endogenous proteinases, which otherwise may cause uncontrolled proteolysis and tissue damage. Cysteine proteinase activity can normally not be measured in body fluids, but can been detected extracellularly in conditions like endotoxin-induced sepsis (2), metastasizing cancer (3), and at local inflammatory processes in rheumatoid arthritis (4), purulent bronchiectasis (5) and periodontitis (6), which indicates that a tight cystatin regulation is a necessity in the normal state. A deficiency state in which the levels of the intracellular cystatin, cystatin B, are lowered due to mutations has recently been shown to segregate with a form of progressive myoclonus epilepsy (7), which points to additional specialized functions of cystatins. Moreover, results showing that chicken cystatin inhibits polio virus replication (8), human cystatin C inhibits corona and herpes simplex virus replication (9,10), and human cystatin a inhibits rhabdovirus-induced apoptosis (11) in cell cultures indicates that cystatins play additional roles in the human defense system. The cystatins constitute a superfamily of evolutionary related proteins, all composed of at least one 100-120 residue domain with conserved sequence motifs (12). The previously well characterized single-domain human members of superfamily could be grouped in two protein families. The Family 1 members, cystatins (or stefins) a and B, contain approximately 100 amino acid residues, lack disulfide bridges, and are not synthesized as preproteins with signal peptides. The Family 2 cystatins (cystatins C, D, S, SN, and SA) are secreted proteins of approx. 120 amino acid residues (Mr 13,000-14,000) and have two characteristic intrachain disulfide bonds. Recently, we identified an additional human cystatin superfamily member by EST1 sequencing in epithelial cell derived cDNA libraries which we named cystatin E (13). The same cystatin was independently discovered by differential display experiments as a mRNA species down-regulated in breast tumor tissue, but present in the surrounding epithelium and reported under the name cystatin M (14). Cystatin E/M is an atypical, secreted low-Mr cystatin in that it is a glycoprotein and just shows 30-35% sequence identity in alignments with the human Family 2 cystatins, which shows that additional cystatin families are yet to be identified (13). The cystatin E/M gene has been localized to chromosome 2 (15), whereas all human Family 2 cystatin genes are clustered on the short arm of chromosome 20 (16), which further stresses that cystatin E/M is just distantly related to the other secreted human low-Mr cystatins. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:38 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 731 of SEQ ID NO:38, b is an integer of 15 to 745, where both a and b correspond to the positions of nucleotide residues shown in SEQ ID NO:38, and where b is greater than or equal to a+14.

Features of Protein Encoded by Gene No: 29

The translation product of this gene shares sequence homology with the leukocyte-associated Ig-like receptor-1, a putative inhibitory receptor which is thought to be important in the regulation of various physiological and cellular functions (See Genbank Accession No. gi|2352941 (AF013249).

In specific embodiments, polypeptides of the invention comprise the following amino acid sequence:

(SEQ ID NO:325)

DSPDTEPGSSAGPTQRPSDNSHNEHAPASQGLKAEHLYILIGVS,

(SEQ ID NO:326)

HRQNQIKQGPPRSKDEEQKPQQRPDLAVDVLERTADKATVNGLPEKDRET
DTSALAAGSSQEVTYAQLDHWALTQRTARAVSPQSTKPMAESITYAAVARH,

(SEQ ID NO:327)

MSPHPTALLGLVLCLAQTIHTQEEDLPRPSISAEPGTVIPLGSHVTFVCR
GPVGVQTFRLERESRSTYNDTEDVSQASPSESEARFRIDSVSEGNAGPYRCIYYKPPKWSEQSDY,

(SEQ ID NO:328)

TALLGLVLCLAQTIHTQE,

(SEQ ID NO:329)

LPRPSISAEPGTVI,

(SEQ ID NO:330)

CRGPVGVQTFRLERE,

(SEQ ID NO:331)

VLERTADKATVNGLPEKDRETDTSALAAGSS,

(SEQ ID NO:332)

AGGPRAAHPVCLCLLQSSVLALVRLRPGCTAGTWA,

(SEQ ID NO:333)

VKEMPGLIAASIISPLNGLSRVTTGAAGERNLWRPGLPGHRARLLSWTHA
EAVGQQSQ,

(SEQ ID NO:334)

LNGLSRVTTGAAGERNLWRPGLPGH,

(SEQ ID NO:335)

VKETSGGXDSPDTEPGSSAGPTQRPSDNSHNEHAPASQGLKAEHLYILIG
VS,

(SEQ ID NO:336)

TEPGSSAGPTQRPSDNSHNEHAPASQGLK,

(SEQ ID NO:337)

HRQNQIKQGPPRSKDEEQKPQQRPDLAVDVLERTADKATVNGLPEKDRET
DTS,

(SEQ ID NO:338)

PRSKDEEQKPQQRPDLAVDVLERTADKAT,

(SEQ ID NO:339)

ALAAGSSQEVTYAQLDHWALTQRTARAVSPQSTKPMAESITYAAVARH,

(SEQ ID NO:340)

AQLDHWALTQRTARAVSPQSTK,

(SEQ ID NO:341)

MSPHPTALLGLVLCLAQTIHTQEEDLPRPSISAEPGTVIPLGSHVTFVCR
GPVGVQTFRL,

(SEQ ID NO:342)

TIHTQEEDLPRPSISAEPGTVIPLGSHVTFV,

(SEQ ID NO:343)

ERESRSTYNDTEDVSQASPSESEARFRIDSVSEGNAGPYRCIYYKPPKWS
EQSDY,
and/or

(SEQ ID NO:344)

DTEDVSQASPSESEARFRIDSVSEGN.

invention

This gene is expressed primarily in macrophages and T-cells, and to a lesser extent, in human fetal heart. Therefore, polynucleotides and polypeptides of the invention are useful as reagents for differential identification of the tissue(s) or cell type(s) present in a biological sample and for diagnosis of diseases and conditions which include, but are not limited to, developmental, hematopoietic, vascular, or immune disorders. Similarly, polypeptides and antibodies directed to these polypeptides are useful in providing immunological probes for differential identification of the tissue(s) or cell type(s). For a number of disorders of the above tissues or cells, particularly of the growth and inflammatory systems, expression of this gene at significantly higher or lower levels may be routinely detected in certain tissues or cell types (e.g., developmental, hematopoietic, vascular, immune, and cancerous and wounded tissues) or bodily fluids (e.g., lymph, serum, plasma, amniotic fluid, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 144 as residues: His-20 to Arg-28, Glu-61 to Val-74, Ser-78 to Ala-84, Lys-105 to Ser-117. The tissue distribution in fetal heart indicates that polynucleotides and polypeptides corresponding to this gene are useful for the study, diagnosis and treatment of functional disorders of the developing fetal heart, including circulatory, vascular, and inflammatory disorders. In addition, expression in macrophages and lymphocytes, combined with the homology to a putative leukocyte inhibitory receptor indicates a role in the treatment/detection of immune disorders including disorders such as arthritis, asthma, immune deficiency diseases such as AIDS, and leukemia. Moreover, the expression within fetal tissue indicates that this protein may play a role in the regulation of cellular division, and may show utility in the diagnosis and treatment of cancer and other proliferative disorders. Similarly, developmental tissues rely on decisions involving cell differentiation and/or apoptosis in pattern formation. Thus, this protein may also be involved in apoptosis or tissue differentiation and could again be useful in cancer therapy. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:39 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1704 of SEQ ID NO:39, b is an integer of 15 to 1718, where both a and b correspond to the positions of nucleotide residues shown in SEQ ID NO:39, and where b is greater than or equal to a+14.

Features of Protein Encoded by Gene No: 30

The translation product of this gene shares sequence homology with erythroid cell specific transcription factor—murine which is thought to be important in normal physiological function of erythroid cells, or in the maintenance of cellular metabolism. In addition, the translation product of this gene also shares homology with the conserved 3-phosphoglycerate dehydrogenase gene which is an essential component of metabolic biosynthetic pathways (See Genbank Accession No. gi|2674062).

In specific embodiments, polypeptides of the invention comprise the following amino acid sequence:

(SEQ ID NO:345)

MNTPNGNSLSAAELTCGMIMCLARQIPQATASMKDGKWERKKFMGTELNG
KTLGILGLGRIGREVATRMQSFGMKTIGYDPIISPEVSASFGVQQLPLEE
IWPLCDFITVHTPLLPSTTGLLNDNTFAQCKKGVRVVNCARGGIVDEGAL
LRALQSGQCAGAALDVFTEEPPRDRALVDHENVISCPHLGASTKEAQSRC
GEEIAVQFVDMVKGKSLTGVVNAQALTSAFSPHTKPWIGLAEALGTLMRA
WAGSPKGTIQVITQGTSLKNAGNCLSPAVIVGLLKEASKQADVNLVNAKL
LVKEAGLNVTTSHSPAAPGEQGFGECLLAVALAGAPYQAVGLVQGTTPVL
QGLNGAVFRPEVPLRRDLPLLLFRTQTSDPAMLPTMIGLLAEAGVRLLSY
QTSLVSDGETWHVMGISSLLPSLEAWKQHVTEAFQFHF,
MAFANLRKVLISDSLDPCCRKILQ, (SEQ ID NO:346)

(SEQ ID NO:347)

GGLQVVEKQNLSKEELIA,

(SEQ ID NO:348)

MCLARQIPQATASMKDGKWERKKFMGTEL,

(SEQ ID NO:349)

ALTSAFSPHTKPWIGLAEALGTLMRAWAG,

(SEQ ID NO:350)

EVPLRRDLPLLLFRTQTSDPAMLPTMIGLLAEAGVR,

(SEQ ID NO:351)

GNSLSAAELTCGMIMCLARQIPQ,

(SEQ ID NO:352)

TASMKDGKWERKKFMGTELNGKTLGI,

(SEQ ID NO:353)

TRMQSFGMKTIGYDPIISPEVSASF,

(SEQ ID NO:354)

EEIWPLCDFITVHTPLLPSTTGLLND,

(SEQ ID NO:355)

RVVNCARGGIVDEGALLRALQSG,

(SEQ ID NO:356)

EPPRDRALVDHENVISCPHLGAST,

(SEQ ID NO:357)

VDMVKGKSLTGVVNAQALTSAFSPHT,

(SEQ ID NO:358)

GTLMRAWAGSPKGTIQVITQGTSL,

(SEQ ID NO:359)

PAVIVGLLKEASKQADVNLVNAKLL,

(SEQ ID NO:360)

HSPAAPGEQGFGECLLAVALAGAPY,

(SEQ ID NO:361)

TPVLQGLNGAVFRPEVPLRRDLPLLLF,

(SEQ ID NO:362)

TSDPAMLPTMIGLLAEAGVRLLSYQTSL,

(SEQ ID NO:363)

ACRSTLVDPKNSARAGERDXRILPSYSSAPGRTAASWLRYFYSQRPIPRP
TPA,

(SEQ ID NO:364)

VDPKNSARAGERDXRILPSYSSAPGRT,

(SEQ ID NO:365)

CRTVKALLFALPPR,

(SEQ ID NO:366)

MAFANLRKVLISDSLDPCCRKILQDGGLQVVEKQNLSKEELIAD,
and/or

(SEQ ID NO:367)

KVLISDSLDPCCRKILQDGGLQVVE.

The gene encoding the disclosed cDNA is thought to reside on chromosome 1. Accordingly, polynucleotides related to this invention are useful as a marker in linkage analysis for chromosome 1. This gene is expressed primarily in IL-1 induced smooth muscle and fetal kidney, and to a lesser extent, in myoloid progenitor cell line and bone marrow. Therefore, polynucleotides and polypeptides of the invention are useful as reagents for differential identification of the tissue(s) or cell type(s) present in a biological sample and for diagnosis of diseases and conditions which include, but are not limited to, immune, hemopoietic, or vascular disorders. Similarly, polypeptides and antibodies directed to these polypeptides are useful in providing immunological probes for differential identification of the tissue(s) or cell type(s). For a number of disorders of the above tissues or cells, particularly of the hemopoietic and immune system, expression of this gene at significantly higher or lower levels may be routinely detected in certain tissues or cell types (e.g., immune, hemopoietic, vascular, renal, metabolic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 145 as residues: Ala-15 to Ser-20. The tissue distribution in myeloid progenitor cell line and bone marrow, combined with the homology to an erythroid cell specific murine transcription factor indicates that polynucleotides and polypeptides corresponding to this gene are useful for the study, diagnosis and treatment of disorders and diseases involving the hemopoietic and immune systems; the maturation of progenitor cells; and the development or disorders of various smooth muscle tissues (e.g. atherosclerosis, embolism, stroke, aneurysm, microvascular disease, etc.). In addition, tissue distribution in kidney, combined with the homology to a key biosynthetic protein implicates this the protein product of this gene as being important in metabolism. Therefore, the protein may show utility in the diagnosis, prevention, and/or treatment of metabolic disorders and conditions. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:40 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1952 of SEQ ID NO:40, b is an integer of 15 to 1966, where both a and b correspond to the positions of nucleotide residues shown in SEQ ID NO:40, and where b is greater than or equal to a+14.

Features of Protein Encoded by Gene No: 31

In specific embodiments, polypeptides of the invention comprise the following amino acid sequence:

(SEQ ID NO:368)

HEPSGPPDKAQDVHIHCILDPVQVK,

(SEQ ID NO:369)

ARAKWSPRQGSGCPHPLHPGPCAGEDVPTHAYSSFACHHFSNHHSSSLLR
CVRRRRRRHRRCRRRCCNHQQRXQNXRQIPHSHSVFRNPHRSQKMSQLHR
VPFFDQEDPDSYLEEEDNLPFPYPKYPRRGWGGFYQRAGLPPMWGCGATR
VYPGQSATTLSLPVT,

(SEQ ID NO:370)

SGCPHPLHPGPCAGEDVPTHAYSSFA,

(SEQ ID NO:371)

VFRNPHRSQKMSQLHRVPFFDQEDPDSYLEE,
and/or

(SEQ ID NO:372)

YQRAGLPPMWGCGATRVYPGQS.

invention.

This gene is expressed primarily in human adult testes. Therefore, polynucleotides and polypeptides of the invention are useful as reagents for differential identification of the tissue(s) or cell type(s) present in a biological sample and for diagnosis of diseases and conditions which include, but are not limited to, reproductive disorders, particularly of the male genitalia. Similarly, polypeptides and antibodies directed to these polypeptides are useful in providing immunological probes for differential identification of the tissue(s) or cell type(s). For a number of disorders of the above tissues or cells, particularly of the reproductive system, expression of this gene at significantly higher or lower levels may be routinely detected in certain tissues or cell types (e.g., reproductive, cancerous and wounded tissues) or bodily fluids (e.g., lymph, serum, plasma, seminal fluid, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 146 as residues: Met-1 to Pro-8, Ser-45 to Thr-50. The tissue distribution in testes indicates that polynucleotides and polypeptides corresponding to this gene are useful for the study, diagnosis, treatment, and possibly prevention of various male reproductive disorders and diseases including male impotence, failed lebido and male secondary sex characteristics, infertility, and testicular cancer. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:41 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 958 of SEQ ID NO:41, b is an integer of 15 to 972, where both a and b correspond to the positions of nucleotide residues shown in SEQ ID NO:41, and where b is greater than or equal to a+14.

Features of Protein Encoded by Gene No: 32

In specific embodiments, polypeptides of the invention comprise the following amino acid sequence:

(SEQ ID NO:373)

WLSNPGGSGGALWFFCKEMKSGKQTMIKHVSIRDLSFGE,

SEQ ID NO:374)

ISCTPKTPSAPMAAFLSPLSLSRLSLSPHFSLSSFLDLYYVDKNPSNLLS

(SEQ ID NO:374)
LLSIASPTRIILKKEARIRHSDQKSSSLXSXGVREFRNIHTLVILVPVTV
TSLYQDPCYYLQTIFHLS,
(SEQ ID NO:375)

KTPSAPMAAFLSPLSLSRLSLSPHFS,

(SEQ ID NO:376)

NLLSLLSIASPTRIILKKEARIRHS,

(SEQ ID NO:377)

RGDTMGAHHGETQPAKQTKAKLVMGRCRMPDVRLFQGVNLLKRHRMKQCV
WKHLVNWLALYMWXIIXEKCDQTKRFHPLSWVSAIRQFIFLATASLSGPF
SXTLHGPCELFTLFLRSKSSLRKVSYVYLTPTVVTSVKGSHSGVPGXLLL
NDHEILKEELPRXAASLGVGQELLIL,

(SEQ ID NO:378)

AKQTKAKLVMGRCRMPDVRLFQGVNLLK,

(SEQ ID NO:379)

LSWVSAIRQFIFLATASLSGPFSXTLH,
and/or

(SEQ ID NO:380)

KGSHSGVPGXLLLNDHEILKEELP.

by the invention.

This gene is expressed primarily in human adult testes. Therefore, polynucleotides and polypeptides of the invention are useful as reagents for differential identification of the tissue(s) or cell type(s) present in a biological sample and for diagnosis of diseases and conditions which include, but are not limited to, reproductive disorders and cancers of the male reproductive system. Similarly, polypeptides and antibodies directed to these polypeptides are useful in providing immunological probes for differential identification of the tissue(s) or cell type(s). For a number of disorders of the above tissues or cells, particularly of the reproductive system, expression of this gene at significantly higher or lower levels may be routinely detected in certain tissues or cell types (e.g., reproductive, and cancerous and wounded tissues) or bodily fluids (e.g., lymph, serum, plasma, seminal fluid, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in testes indicates that polynucleotides and polypeptides corresponding to this gene are useful for the study, diagnosis, treatment, and possibly prevention of various male reproductive disorders and diseases including male impotence, failed libido and male secondary sex characteristics, infertility, and testicular cancer. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:42 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1522 of SEQ ID NO:42, b is an integer of 15 to 1536, where both a and b correspond to the positions of nucleotide residues shown in SEQ ID NO:42, and where b is greater than or equal to a+14.

Features of Protein Encoded by Gene No: 33

The translation product of this gene shares homology to the W09D10.1 protein of Caenorhabditis elegans and the phosphatidylinositol-3,4,5-triphosphate binding protein of Bos taurus (See Genbank Accession No. gnl|PID|d1020948). In addition, the gene also shares homology with the human protein hRIP, a protein known to be critical for HIV replication (See Genbank Accession Nos. gnl|PID|e1186472 and W12713).

In specific embodiments, polypeptides of the invention comprise the following amino acid sequence:

(SEQ ID NO:381)

FGTRFLANLLLEEDNKFCADCQSKGPRWASWNIGVFICIRCAXIHRNLGV
HISRVKSVNLDQWTQVQIQCMQXMGNGKANRLYEAYLPETFRRPQIDPAV
EGFIRDXYE,

(SEQ ID NO:385)

MDLLGLDAPVACSIANSKTSNTLEKDLDLLASVPSPSSSGSRKVVGSMPT
AGSAGSVPENLNLFPEPGSKSEEIGKKQLSKDSILSLYGSQTXQMPTQAM
FMAPAQMAYPTAYPSFPGVTPPNSIMGSMMPPPVGMVAQPGASGMVAPMA
MPAGYMGGMQASMMGVPNGMMTTQQAGYMAGMAAMPQTVYGVQPAQQLQW
NLTQMTQQMAGMNFYGANGMMNYGQSMSGGNGQAANQTLSPQMWK,

(SEQ ID NO:387)

MQXMGNGKANRLYEAYLPETFRRPQIDPAVEGFIRDXYE,

(SEQ ID NO:382)

EEDNKFCADCQSKGPRWASWN,

(SEQ ID NO:383)

GVFICIRCAXIHRNLGVHIS

(SEQ ID NO:384)

SVNLDQWTQVQIQCMQXMGNGKA,

(SEQ ID NO:388)

TQLLKDLFETXMRRRNTWTEVWDINAFRKEKDDKWKRGSEPVPEKKLEPV
VFEKVKMPQKKEDPQLPRKSSPKSTAPV,

(SEQ ID NO:389)

VWDPYSERMESLESCFLPISSDLLPGSGNRFRFSGTEPALPAVGMEPTTF
LEPEEEGDGTEANRSKSFSRVLLVLLFAMEQATGASRPNKSMTGAVDFGE
LFRGSCGSSFFCGIFTFSKTTGSNFFSGTGSLPLFHLSSFSFLKALMSQT
SVHVFLLLIXVSNKSFNSWVYLRSPKGLRKIGFIKSVCLSISHXLHALNL
YLSPLVEVN,

(SEQ ID NO:390)

LESCFLPISSDLLPGSGNRFRFSGTE,

(SEQ ID NO:391)

GTEANRSKSFSRVLLVLLFAME,

(SEQ ID NO:392)

GTGSLPLFHLSSFSFLKALMSQTS,

(SEQ ID NO:393)

RKIGFIKSVCLSISHXLHALNLY,

(SEQ ID NO:394)

LPFSLPFSRSADHSFMPVYPGCSVSPPFPGVRGRGPGQLSSQSLFTPLEN
SDKKTLLKRFHVWEPQFLAAFLLCMCKFLNKYCREGQ,

(SEQ ID NO:395)

MPVYPGCSVSPPFPGVRGRGPGQ,

(SEQ ID NO:396)

FGTRFLANLLLEEDNKFCADCQSKGPRWASWNIGVFICIRCAXIHRNLG
VHISRVKSVNLDQWTQVQIQC,
and/or

(SEQ ID NO:386)

MDLLGLDAPVACSIANSKTSNTLEKDLDLLASVPSPSSSGSRKVVGSMPT
AGSAGSVPENLNLFPEPGSKSEEIGKKQLSKDSILSLYGSQTXQMPTQAM
FMAPAQMAYPTAYPSFPGVTPPNSIMGSMMPPPVGMVAQPGASGMVAPMA
MPAGYMGGMQASMMGVPNGMMTTQQAGYMAGMAAMPQTVYGVQPAQQLQW
NLTQMTQQMAGMNFYGANGMMNYGQSMSGGNGQAANQTLSPQMWKFGTRF
LANLLLEEDNKFCADCQSKGPRWASWNIGVFICIRCAXIHRNLGVHISRV
KSVNLDQWTQVQIQC.

encoding these polypeptides are also encompassed by the invention.

This gene is expressed primarily in lymphoid tumors. Therefore, polynucleotides and polypeptides of the invention are useful as reagents for differential identification of the tissue(s) or cell type(s) present in a biological sample and for diagnosis of diseases and conditions which include, but are not limited to, immune and inflammatory disorders. Similarly, polypeptides and antibodies directed to these polypeptides are useful in providing immunological probes for differential identification of the tissue(s) or cell type(s). For a number of disorders of the above tissues or cells, particularly of the immune, hematopoietic and inflammatory, expression of this gene at significantly higher or lower levels may be routinely detected in certain tissues or cell types (e.g., immune, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 148 as residues: Cys-21 to Trp-28. The tissue distribution in lymphoid tumors, combined with the homology to the phosphatidylinositol-3,4,5-triphosphate binding protein indicates that the protein products of this gene are useful for the study, diagnosis and treatment of various immune disorders and diseases, including self-recognition and rejection functions of the immune system, hematopoietic disorders, and inflammatory disorders, in addition to proliferative conditions of immune cells and tissues. Homology to the W09D10.1 of C. elegans and the hRIP implicates this gene as playing a role as an essential receptor for host-viral interactions including, but not limited to retroviral infections such as AIDS. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:43 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 2527 of SEQ ID NO:43, b is an integer of 15 to 2541, where both a and b correspond to the positions of nucleotide residues shown in SEQ ID NO:43, and where b is greater than or equal to a+14.

Features of Protein Encoded by Gene No: 34

The translation product of this gene shares homology to an Arabidopsis thaliana recombination and DNA-damage resistance/repair protein (See Genbank Accession No. gi|166694).

In specific embodiments, polypeptides of the invention comprise the following amino acid sequence:

(SEQ ID NO:397)

KYGKVGKCVIFEIPGAPDDEAVRIFLEFERVESAIKAVVDLNGRYFGGR
VVKACFYNLDKFRVLDLA,

(SEQ ID NO:398)

KAVDLGRYFGGR,

(SEQ ID NO:399)

EAVRIEFRE,

(SEQ ID NO:400)

DLGLSCSSYTGGGMAALEDCDRGLESSS,

(SEQ ID NO:401)

TRFPGDTPFSVASPTMILPPRLLVF,

(SEQ ID NO:402)

MILCATVPPMLARKELLGPVGDLGLS,

(SEQ ID NO:403)

DLAEQV,

(SEQ ID NO:404)

EAVRIFLEFERVESAIKAVVDLNGRYFGGRV,

(SEQ ID NO:405)

QGLGKHEQGLSTALSVEKTSKRGGKIIVGDATEKGVSPGKRVTRGKGLAP
SISDMASLDPHVSAGGQ,

(SEQ ID NO:406)

LVEKDKELPRDFPYEEDSRPRSQSSK,

(SEQ ID NO:407)

LANMGGTVAHKIMQKYGFREGQGLGKHEQGLST,

(SEQ ID NO:408)

RVTRGKGLAPSISDMASLDPHVS,

(SEQ ID NO:409)

GYITSLTDASKKSDSNPLTEILKCPTKVVLLRNMVGAGEVDEDLXS,
and/or

(SEQ ID NO:410)

SLTDASKKSDSNPLTEILKCPTKVVL.

encompassed by the invention.

This gene is expressed primarily in ovarian and other cancers. Therefore, polynucleotides and polypeptides of the invention are useful as reagents for differential identification of the tissue(s) or cell type(s) present in a biological sample and for diagnosis of diseases and conditions which include, but are not limited to, cancer, particularly of the female reproductive system. Similarly, polypeptides and antibodies directed to these polypeptides are useful in providing immunological probes for differential identification of the tissue(s) or cell type(s). For a number of disorders of the above tissues or cells, particularly of the reproductive system, expression of this gene at significantly higher or lower levels may be routinely detected in certain tissues or cell types (e.g., reproductive, ovarian, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 149 as residues: Thr-11 to Trp-19, Ala-40 to Gln-47, Lys-58 to Arg-66, Asp-98 to Lys-110, Arg-114 to Glu-121. The tissue distribution in tumors of ovarian origins combined with the homology to a known DNA damage repair enzyme indicates that polynucleotides and polypeptides corresponding to this gene are useful for the diagnosis and intervention of tumors. Protein, as well as, antibodies directed against the protein may show utility as a tumor marker and/or immunotherapy targets for the above listed tumors and tissues.

Many polynucleotide sequences, such as EST sequences, are publicly available and accessible through sequence databases. Some of these sequences are related to SEQ ID NO:44 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 2404 of SEQ ID NO:44, b is an integer of 15 to 2418, where both a and b correspond to the positions of nucleotide residues shown in SEQ ID NO:44, and where b is greater than or equal to a+14.

Features of Protein Encoded by Gene No: 35

The translation product of this gene shares homology with the conserved human P1.11659_{—}4 gene which encodes the XRCC9 DNA repair gene, in addition to the F30A10.5 protein of Caenorhabditis elegans (See Genbank Accession Nos. gi|2984585 and gnl|PID|e276130, respectively).

In specific embodiments, polypeptides of the invention comprise the following amino acid sequence:

(SEQ ID NO:411)

RMGRFHRILEPGLNILIPVLDRIRYVQSLKEIVINVPEQSAVTLDNVTLQ
IDGVLYLRIMDPYKASYGVEDPEYAVTQLAQTTMRSELGKLSLDKVFRER
ESLNASIVDAINQAADCWGIRCLRYEIKDIHVPPRVKESMQMQVEAERRK
RATVLESEGTRESAINVAEGKKQAQILASEAEKAEQINQAAGEASAVLAK
AKAKAEAIRILAAALTQHNGDAAASLTVAEQYVSAFSKLAKDSNTILLPS
NPGDVTSMVAQAMGVYGALTKAPVPGTPDSLSSGSSRDVQGTDASLDEEL
DRVKMS,

(SEQ ID NO:412)

ASYGVEDPEYAVTQLAQTTMRSELGK,

(SEQ ID NO:413)

MQMQVEAERRKRATVLESEGTRESAIN,

(SEQ ID NO:414)

LTVAEQYVSAFSKLAKDSNTILLPSN,

(SEQ ID NO:415)

LLGATAPLVSLVPEVAAAVGNAGARGAXHWGPFAEGLSTGFWPR
SARASSGLPRNTVVLFVPQQEAWVVE,

(SEQ ID NO:416)

PRRASSGLPRNTVVLFVPQQEAWVVERMGRFHRILEPGLNILIPVLDRIR
YVQSLKEIVI,

(SEQ ID NO:417)

VPQQEAWVVERMGRFHRILEPGLNILIP,

(SEQ ID NO:418)

NVPEQSAVTLDNVTLQIDGVLYLRIMDPYKASYGVEDPEYAVTQLAQTTM
RSELGKLSLD,

(SEQ ID NO:419)

IDGVLYLRIMDPYKASYGVEDPEYAVTQLA,

(SEQ ID NO:420)
KVFRERESLNASIVDAINQAADCWGIRCLRYEIKDIHVPPRVKESMQMQV
,
(SEQ ID NO:421)

ASIVDAINQAADCWGIRCLRYEIKD,

(SEQ ID NO:422)

EAERRKRATVLESEGTRESAINVAEGKKQAQILASEAEKAEQINQ,

(SEQ ID NO:423)

LESEGTRESAINVAEGKKQAQILA,

(SEQ ID NO:424)

TQHNGDAAASLTVAEQYVSAFSKLAKDSNTILLPSNPGDVTSMVA,

(SEQ ID NO:425)

LTVAEQYVSAFSKLAKDSNTILLP,

(SEQ ID NO:426)

QAMGVYGALTKAPVPGTPDSLSSGSSRDVQGTDASLDEELDRVKMS,

(SEQ ID NO:427)

KAPVPGTPDSLSSGSSRDVQGTDASL,

(SEQ ID NO:429)

NAGARGAXHWGPFAEGLSTGFWPRSARAS,

(SEQ ID NO:430)

STHASGATSLLRSSRWFRRSLRRWE,
and/or

(SEQ ID NO:428)

AAAVGNAGARGAXHWGPFAEGLSTGFWPRS.

encompassed by the invention.

The gene encoding the disclosed cDNA is believed to reside on chromosome 9. Accordingly, polynucleotides related to this invention are useful as a marker in linkage analysis for chromosome 9. This gene is expressed primarily in activated T-cells. Therefore, polynucleotides and polypeptides of the invention are useful as reagents for differential identification of the tissue(s) or cell type(s) present in a biological sample and for diagnosis of diseases and conditions which include, but are not limited to, immune disorders, particularly proliferative conditions such as leukemia. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, hematopoieitic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 150 as residues: Arg-23 to Pro-33, Pro-184 to Ser-189, Ala-196 to Arg-201, Glu-208 to Ser-213, Glu-230 to Ile-237, Gly-326 to Leu-331, Gly-334 to Gln-340. The tissue distribution in activated T-cells, combined with the strong homology, to the human XRCC9 DNA repair gene indicates that the protein products of this gene are useful for the treatment and diagnosis of immune or hematopoetic related disorders such as anemia, pancytopenia, leukopenia, thrombocytopenia or leukemia, since stromal cells are important in the production of cells of hematopoietic lineages. The uses include bone marrow cell ex vivo culture, bone marrow transplantation, bone marrow reconstitution, radiotherapy or chemotherapy of neoplasia. The gene product may also be involved in lymphopoiesis, therefore, it can be used in immune disorders such as infection, inflammation, allergy, immunodeficiency etc. In addition, the homology to known intestinal antigens may suggest that the protein is important in the diagnosis, treatment, and/or prevention of gastrointestinal disorders. Moreover, the protein may be useful as a detection, treatment, or possibly the prevention of a variety of immune disorders, particularly proliferative conditions, since there is a strong correlation between a cells DNA repair capacity with the incidence of aberrant cellular metabolism and regulation. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:45 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1323 of SEQ ID NO:45, b is an integer of 15 to 1337, where both a and b correspond to the positions of nucleotide residues shown in SEQ ID NO:45, and where b is greater than or equal to a+14.

Features of Protein Encoded by Gene No: 36

The translation product of this gene has homology to a human estrogen receptor variant from human breast cancer. In specific embodiments, polypeptides of the invention comprise the following amino acid sequence:

(SEQ ID NO:431)

RMWRNGTHFWECKIVQPLWKTVWWFPRKLSIELPENLAILIGTYFK,

(SEQ ID NO:432)

LKRHFPKEANKHVKRCSTSLDIREIQIKIKMRY,

(SEQ ID NO:433)

HVLVRMWRNGTHFW,

(SEQ ID NO:434)

GTSGSYYLMWPIGLKPITIKEXXXXTTSNSIXK,

(SEQ ID NO:435)

AKNLKRHFPKEANKHVKRCSTSLDIREIQIKIKMRYQFILRWL,

(SEQ ID NO:436)

TMWYIPRGEYYSALKRNEVLVHATKVDEPQKHAE
and/or

(SEQ ID NO:437)

PKEANKHVKRCSTSLDIREIQIK.

This gene is expressed primarily in ulcerative colitis. Therefore, polynucleotides and polypeptides of the invention are useful as reagents for differential identification of the tissue(s) or cell type(s) present in a biological sample and for diagnosis of diseases and conditions which include, but are not limited to, gastrointestinal or metabolic disorders, in addition to intestinal ulcers, inflammatory conditions and cancers, particular of the breast. Similarly, polypeptides and antibodies directed to these polypeptides are useful in providing immunological probes for differential identification of the tissue(s) or cell type(s). For a number of disorders of the above tissues or cells, particularly of the gastrointestinal system, expression of this gene at significantly higher or lower levels may be routinely detected in certain tissues or cell types (e.g., gastrointestinal, metabolic, breast, and cancerous and wounded tissues) or bodily fluids (e.g., lymph, serum, chyme, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in colon and breast origins, combined with the homology to the human estrogen receptor variant indicates that polynucleotides and polypeptides corresponding to this gene are useful for the diagnosis and intervention of tumors or other conditions within these tissues, in addition to tumors and conditions of other tissues. Protein, as well as, antibodies directed against the protein may show utility as a tumor marker and/or immunotherapy targets for the above listed tumors and tissues.

Many polynucleotide sequences, such as EST sequences, are publicly available and accessible through sequence databases. Some of these sequences are related to SEQ ID NO:46 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1262 of SEQ ID NO:46, b is an integer of 15 to 1276, where both a and b correspond to the positions of nucleotide residues shown in SEQ ID NO:46, and where b is greater than or equal to a+14.

Features of Protein Encoded by Gene No: 37

This gene is expressed primarily in epithelial cells. Therefore, polynucleotides and polypeptides of the invention are useful as reagents for differential identification of the tissue(s) or cell type(s) present in a biological sample and for diagnosis of diseases and conditions which include, but are not limited to, integumentary disorders, particularly melanoma, in addition to cancers. Similarly, polypeptides and antibodies directed to these polypeptides are useful in providing immunological probes for differential identification of the tissue(s) or cell type(s). For a number of disorders of the above tissues or cells, particularly of the skin and other epithelia, expression of this gene at significantly higher or lower levels may be routinely detected in certain tissues or cell types (e.g., integumentary, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 152 as residues: Met-1 to Tyr-6. The tissue distribution in epithelial tissue indicates that polynucleotides and polypeptides corresponding to this gene are useful for diagnosis and intervention of tumors of this tissue. Moreover, the protein product of this gene is useful for the treatment, diagnosis, and/or prevention of various skin disorders including congenital disorders (i.e. nevi, moles, freckles, Mongolian spots, hemangiomas, port-wine syndrome), integumentary tumors (i.e. keratoses, Bowen's disease, basal cell carcinoma, squamous cell carcinoma, malignant melanoma, Paget's disease, mycosis fungoides, and Kaposi's sarcoma), injuries and inflammation of the skin (i.e. wounds, rashes, prickly heat disorder, psoriasis, dermatitis), atherosclerosis, uticaria, eczema, photosensitivity, autoimmune disorders (i.e. lupus erythematosus, vitiligo, dermatomyositis, morphea, scleroderma, pemphigoid, and pemphigus), keloids, striae, erythema, petechiae, purpura, and xanthelasma. In addition, such disorders may predispose increased susceptibility to viral and bacterial infections of the skin (i.e. cold sores, warts, chickenpox, molluscum contagiosum, herpes zoster, boils, cellulitis, erysipelas, impetigo, tinea, althletes foot, and ringworm). Moreover, the protein product of this gene may also be useful for the treatment or diagnosis of various connective tissue disorders such as arthritis, trauma, tendonitis, chrondomalacia and inflammation, autoimmune disorders such as rheumatoid arthritis, lupus, scleroderma, and dermatomyositis as well as dwarfism, spinal deformation, and specific joint abnormalities as well as chondrodysplasias (i.e. spondyloepiphyseal dysplasia congenita, familial osteoarthritis, Atelosteogenesis type II, metaphyseal chondrodysplasia type Schmid). Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:47 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1268 of SEQ ID NO:47, b is an integer of 15 to 1282, where both a and b correspond to the positions of nucleotide residues shown in SEQ ID NO:47, and where b is greater than or equal to a+14.

Features of Protein Encoded by Gene No: 38

This gene is expressed primarily in adult retina. Therefore, polynucleotides and polypeptides of the invention are useful as reagents for differential identification of the tissue(s) or cell type(s) present in a biological sample and for diagnosis of diseases and conditions which include, but are not limited to, diseases of the eye. Similarly, polypeptides and antibodies directed to these polypeptides are useful in providing immunological probes for differential identification of the tissue(s) or cell type(s). For a number of disorders of the above tissues or cells, particularly of the eye, expression of this gene at significantly higher or lower levels may be routinely detected in certain tissues or cell types (e.g., visual, and cancerous and wounded tissues) or bodily fluids (e.g., lymph, serum, aqueous humor, vitreous humor, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 153 as residues: Cys-14 to Lys-21. The tissue distribution in the adult retina indicates that polynucleotides and polypeptides corresponding to this gene are useful for the treatment and diagnosis of disorders of the eye. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:48 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 631 of SEQ ID NO:48, b is an integer of 15 to 645, where both a and b correspond to the positions of nucleotide residues shown in SEQ ID NO:48, and where b is greater than or equal to a+14.

Features of Protein Encoded by Gene No: 39

In specific embodiments, polypeptides of the invention comprise the following amino acid sequence:

(SEQ ID NO:438)

VEMRRWPPSKNFTFTSSLSLSSLTALLQEKLLLSVSRRHAALSVCVLTHH
LDGRILVTTETTFYKSKLKGTASSSVLGRGG,

(SEQ ID NO:439)

PPSKNFTFTSSLSLSSLTALLQ,

(SEQ ID NO:440)

DGRILVTTETTFYKSKLKGTA,
and/or

(SEQ ID NO:441)

VENSRKA.

This gene is expressed primarily in bone marrow and fetal liver. Therefore, polynucleotides and polypeptides of the invention are useful as reagents for differential identification of the tissue(s) or cell type(s) present in a biological sample and for diagnosis of diseases and conditions which include, but are not limited to, immune, haemopoietic, or hepatatic disorders. Similarly, polypeptides and antibodies directed to these polypeptides are useful in providing immunological probes for differential identification of the tissue(s) or cell type(s). For a number of disorders of the above tissues or cells, particularly of the haemopoietic system, expression of this gene at significantly higher or lower levels may be routinely detected in certain tissues or cell types (e.g., immune, haemopoietic, hepatatic, metabolic, and cancerous and wounded tissues) or bodily fluids (e.g., lymph, serum, bile, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in bone marrow and fetal liver indicates that polynucleotides and polypeptides corresponding to this gene are useful for the treatment and diagnosis of disorders of the haemopoietic system. Moreover, the protein product of this gene is useful for the treatment and diagnosis of hematopoietic related disorders such as anemia, pancytopenia, leukopenia, thrombocytopenia or leukemia since stromal cells are important in the production of cells of hematopoietic lineages. The uses include bone marrow cell ex vivo culture, bone marrow transplantation, bone marrow reconstitution, radiotherapy or chemotherapy of neoplasia. The gene product may also be involved in lymphopoiesis, therefore, it can be used in immune disorders such as infection, inflammation, allergy, immunodeficiency etc. In addition, this gene product may have commercial utility in the expansion of stem cells and committed progenitors of various blood lineages, and in the differentiation and/or proliferation of various cell types. Expression within fetal tissue indicates that this protein may play a role in the regulation of cellular division, and may show utility in the diagnosis and treatment of cancer and other proliferative disorders. Similarly, developmental tissues rely on decisions involving cell differentiation and/or apoptosis in pattern formation. Thus this protein may also be involved in apoptosis or tissue differentiation and could again be useful in cancer therapy. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:49 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1481 of SEQ ID NO:49, b is an integer of 15 to 1495, where both a and b correspond to the positions of nucleotide residues shown in SEQ ID NO:49, and where b is greater than or equal to a+14.

Features of Protein Encoded by Gene No: 40

This gene is expressed primarily in lymph node, fetal liver, and brain. Therefore, polynucleotides and polypeptides of the invention are useful as reagents for differential identification of the tissue(s) or cell type(s) present in a biological sample and for diagnosis of diseases and conditions which include, but are not limited to, haemopoietic or immune disorders, and disorders of the CNS. Similarly, polypeptides and antibodies directed to these polypeptides are useful in providing immunological probes for differential identification of the tissue(s) or cell type(s). For a number of disorders of the above tissues or cells, particularly of the haemopoietic and CNS, expression of this gene at significantly higher or lower levels may be routinely detected in certain tissues or cell types (e.g., haemopoietic, immune, neural, and cancerous and wounded tissues) or bodily fluids (e.g., lymph, serum, amniotic fluid, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in fetal tissue and other cellular sources marked by proliferating cells indicates that this protein may play a role in the regulation or cellular division. Additionally, the expression in hematopoietic cells and tissues indicates that this protein may play a role in the proliferation, differentiation, and/or survival of hematopoietic cell lineages.

Thus, this gene may be useful in the treatment of lymphoproliferative disorders, and in the maintenance and differentiation of various hematopoietic lineages from early hematopoietic stem and committed progenitor cells. In addition, polynucleotides and polypeptides corresponding to this gene are useful for the detection treatment of neurodegenerative disease states and behavioral disorders such as Alzheimers Disease, Parkinsons Disease, Huntingtons Disease, schizophrenia, mania, dementia, paranoia, obsessive compulsive disorder, panic disorder, and autism. In addition, the gene or gene product may also play a role in the treatment and/or detection of developmental disorders associated with the developing embryo, sexually-linked disorders, or disorders of the cardiovascular system. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:50 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1616 of SEQ ID NO:50, b is an integer of 15 to 1630, where both a and b correspond to the positions of nucleotide residues shown in SEQ ID NO:50, and where b is greater than or equal to a+14.

Features of Protein Encoded by Gene No: 41

The translation product of this gene shares sequence homology with fibropellin and epidermal growth factors, and the rat Notch2 protein which are thought to be important in growth and regeneration of epidermal cells (See Genbank Accession Nos. WI 1719, gi|310660, and pir|A49128|A49128, respectively).

In specific embodiments, polypeptides of the invention comprise the following amino acid sequence:

(SEQ ID NO:442)

GTRPGESHANDLECSGKGKCTTKPSEATFSCTCEEQYVGTFCEEYDACQR
KPCQNNASCIDANEKQDGSNFTCVCLPGYTGELCQSKIDYCILDPCRNGA
TCISSLSGFTCQDPEGYFGSACEEKVDPCASSPCQNNGTCYVDGVHFTCN
CSPGFTGPTCAQLIDFCALSPCAHGTCRSVGTSYKCLCDPGYHGLYCEEE
YNECLSAPCLNAATCRDLVNGYECVCLAEYKGTHCELYKDPCANVSCLNG
ATCDSDGLNGTCICAPGFTGEECDIDINECDSNPCHHGGSCLDQPNGYNC
HCPHGWVGANCEIHLQWKSGHMAESLTN.

(SEQ ID NO:443)

GKCTTKPSEATFSCTCEEQYVGTFC,

(SEQ ID NO:444)

CAHGTCRSVGTSYKCLCDPGYH,

(SEQ ID NO:445)

CANVSCLNGATCDSDGLNGTCICAPGFTGEECD,

(SEQ ID NO:446)

HANDLECSGKGKCTTKPSEATFSCTCE,

(SEQ ID NO:447)

YVGTFCEEYDACQRKPCQNNASCIDA,

(SEQ ID NO:448)

GYTGELCQSKIDYCILDPCRNGA,

(SEQ ID NO:449)

CPEGYFGSACEEKVDPCASSPCQNN,

(SEQ ID NO:450)

CSPGFTGPTCAQLIDFCALSPC,

(SEQ ID NO:451)

GTCRSVGTSYKCLCDPGYHGLYCEE,

(SEQ ID NO:452)

DQPNGYNCHCPHGWVGANCEIHLQWK,

(SEQ ID NO:453)

PPFRLQGDWSSWRE,

(SEQ ID NO:454)

THAEMEQHAFPVSVDSPASVQKDTSDLLVKKRWTPAPRLRARTTAPAMWT
GYTLPATAARASQGRPVPSLLTSVPSAPVLMARAAAWAPATNASVIQVTM
ASTVRRNIMSASPLHA,

(SEQ ID NO:455)

EQHAFPVSVDSPASVQKDTSDL,

(SEQ ID NO:456)

PAPRLRARTTAPAMWTGYTLPA,

(SEQ ID NO:457)

VPSLLTSVPSAPVLMARAA,

(SEQ ID NO:458)

VIQVTMASTVRRNIMSASPLH,

(SEQ ID NO:459)

EMLALGNNHFIGFVNDSVTKSIVALRLTLVVKVST,

(SEQ ID NO:460)

SGKGKCTTKPSEATFSCTCEEQYVGTFCEEYDACQRKPCQNNASCIDANE
KQDGSNFTCV,

(SEQ ID NO:461)

ATFSCTCEEQYVGTFCEEYDACQRKPCQNNA,

(SEQ ID NO:462)

CLPGYTGELCQSKIDYCILDPCRNGATCISSLSGFTCQCPEGYFGSACEE
KVDPCASSPC,

(SEQ ID NQ:463)

YCILDPCRNGATCISSLSGFTCQCPEGY,

(SEQ ID NO:464)

QNNGTCYVDGVHTCNCSPGFTGPTCAQLIDFCALSPCAHGTCRSVGTSYK
CLCDPGYHG,

(SEQ ID NO:465)

CNCSPGFTGTCAQLIDFCALSPCAHG,

(SEQ ID NO:466)

LYCEEEYNECLSAPCLNAATCRDLVNGYECVCLAEYKGTHCELYKDPCAN
VSCLNGATCD,

(SEQ ID NO:467)

PCLNAATCRDLVNGYECVCLAEYK,

(SEQ ID NO:468)

SDGLNGTCICAPGFTGEECDIDINECDSNPCHHGGSCLDQPNGYNXHCPH
GWVGANCEIH,

(SEQ ID NO:469)

GEECDIDINECDSNPCHHGGSCL,

(SEQ ID NO:470)

FYNCRSLDSEFSNAIASIRHARFGKKSRPAMYDVSPIAYEDYSPDDKPLV
TLIKTKDL,

(SEQ ID NO:471)

ASLRHARFGKKSRPAMYDVSPIAYEDY,
and/or

(SEQ ID NO:472)

QQCSLIDGRSVTPLQASGGLVLLE.

Polynucleotides encoding these polypeptides are also encompassed by the invention.

The gene encoding the disclosed cDNA is believed to reside on chromosome 2. Accordingly, polynucleotides related to this invention are useful as a marker in linkage analysis for chromosome 2. This gene is expressed primarily in brain and kidney. Therefore, polynucleotides and polypeptides of the invention are useful as reagents for differential identification of the tissue(s) or cell type(s) present in a biological sample and for diagnosis of diseases and conditions which include, but are not limited to, disorders of the neural and renal systems, particularly growth disorders such as cancer. Similarly, polypeptides and antibodies directed to these polypeptides are useful in providing immunological probes for differential identification of the tissue(s) or cell type(s). For a number of disorders of the above tissues or cells, particularly of the neural and renal systems, expression of this gene at significantly higher or lower levels may be routinely detected in certain tissues or cell types (e.g., renal, neural, developmental, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in brain and kidney, combined with the homology to epidermal growth factor and notch2 protein indicates that polynucleotides and polypeptides corresponding to this gene are useful for the diagnosis and treatment of growth disorders, especially in the neural and renal systems. In addition, polynucleotides and polypeptides corresponding to this gene are useful for the detection/treatment of neurodegenerative disease states and behavioral disorders such as Alzheimers Disease, Parkinsons Disease, Huntingtons Disease, schizophrenia, mania, dementia, paranoia, obsessive compulsive disorder, panic disorder, and autism. In addition, the gene or gene product may also play a role in the treatment and/or detection of developmental disorders associated with the developing embryo, sexually-linked disorders, or disorders of the cardiovascular system. Moreover, the protein may also show utility in the treatment, detection, and/or prevention of a variety of integumentary disorders, particularly those which derive from aberrant structure or function of the apical lamina, the extracellular matrix, or the basal lamina, in addition to their relation to proper development, particularly during gastrulation. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:51 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 2406 of SEQ ID NO:51, b is an integer of 15 to 2420, where both a and b correspond to the positions of nucleotide residues shown in SEQ ID NO:51, and where b is greater than or equal to a+14.

Features of Protein Encoded by Gene No: 42

In specific embodiments, polypeptides of the invention comprise the following amino acid sequence: PNGSSNVCVSLCVFVCVCALKTSNSLEAWGGIPALPLACL (SEQ ID NO:473), and/or LCVFVCVCALKTSNSLEAWGGIP (SEQ ID NO:474). Polynucleotides encoding these polypeptides are also encompassed by the invention.

The gene encoding the disclosed cDNA is believed to reside on chromosome 15. Accordingly, polynucleotides related to this invention are useful as a marker in linkage analysis for chromosome 15. This gene is expressed primarily in brain, kidney and stromal cells. Therefore, polynucleotides and polypeptides of the invention are useful as reagents for differential identification of the tissue(s) or cell type(s) present in a biological sample and for diagnosis of diseases and conditions which include, but are not limited to, immune, haemopoietic, renal, or neural disorders. Similarly, polypeptides and antibodies directed to these polypeptides are useful in providing immunological probes for differential identification of the tissue(s) or cell type(s). For a number of disorders of the above tissues or cells, particularly of the haemopoietic, renal and central nervous system, expression of this gene at significantly higher or lower levels may be routinely detected in certain tissues or cell types (e.g., immune, haemopoietic, neural, renal, urogenital, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 157 as residues: Lys-71 to Trp-76, Glu-99 to Gly-108, Arg-142 to Ser-149. The tissue distribution in brain indicates that polynucleotides and polypeptides corresponding to this gene are useful for the detection/treatment of neurodegenerative disease states and behavioral disorders such as Alzheimers Disease, Parkinsons Disease, Huntingtons Disease, schizophrenia, mania, dementia, paranoia, obsessive compulsive disorder, panic disorder, and autism. In addition, the gene or gene product may also play a role in the treatment and/or detection of developmental disorders associated with the developing embryo, sexually-linked disorders, or disorders of the cardiovascular system. Moreover, the protein product of this gene is useful for the treatment and diagnosis of hematopoetic related disorders such as anemia, pancytopenia, leukopenia, thrombocytopenia or leukemia since stromal cells are important in the production of cells of hematopoietic lineages. The uses include bone marrow cell ex vivo culture, bone marrow transplantation, bone marrow reconstitution, radiotherapy or chemotherapy of neoplasia. The gene product is thought to be involved in lymphopoiesis, therefore, it can be used in immune disorders to modulate infection, inflammation, allergy, immunodeficiency, etc. Alternatively, this gene or gene product could be used in the treatment and/or detection of kidney diseases including renal failure, nephritus, renal tubular acidosis, proteinuria, pyuria, edema, pyelonephritis, hydronephritis, nephrotic syndrome, crush syndrome, glomerulonephritis, hematuria, renal colic and kidney stones, in addition to Wilm's Tumor Disease, and congenital kidney abnormalities such as horseshoe kidney, polycystic kidney, and Falconi's syndrome. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:52 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1158 of SEQ ID NO:52, b is an integer of 15 to 1172, where both a and b correspond to the positions of nucleotide residues shown in SEQ ID NO:52, and where b is greater than or equal to a+14.

Features of Protein Encoded by Gene No: 43

The gene product above share sequence similarity with prohibitin, in addition to the human B-cell receptor associated protein (See Genbank Accession No. gi|1922935). Thus, these polypeptides are expected to share biological activities with prohibitin. These activities are known in the art include, though are not limited to, the following: antiproliferative activities, particularly for neoplasias.

In specific embodiments, polypeptides of the invention comprise the following amino acid sequence:

(SEQ ID NO:475)

MAQNLKDLAGRLPAGRGMGTALKLLLGAGAVAYGVRESVFTVEGGHRAIF
FNRIGGVQQDTILAEGLHFRIPWFQYPIIYDIRARPRKISSPTGSKDLQM
VNISLRVLSRPNAQELPSMYQRLGLDYEERVLPSIVNEVLKSVVAKFNAS
QLLTQRAQVSLLLRRELTERAKDFSLILDDVAITELSFSREYTAAVEAKQ
VAQQEAQRAQFLVEKAKQEQRQKIVQAEGEAEAAKMLGEALSKNPGYIKL
RKIRAAQNISKTIATSQNRIYLTADNLVLNLQDESFVRGSDSLIKGKK,

(SEQ ID NO:476)

PPVPPASRSD,

(SEQ ID NO:477)

MAQNLKDLAGRLPAGPR,

(SEQ ID NO:478)

YGVRESVFTVEGGHRALFFNRIGGVQQ,

(SEQ ID NO:479)

DTILAEGLHFRIPWFQYPIIYDIRARPRKISSTGSKDLQMVNISLRVLSR
PNAQELPSM,

(SEQ ID NO:480)

WFQYPIIYDIRARPRKISSPTGSKDLQMV,

(SEQ ID NO:481)

YQRLGLDYEERVLPSIVNEVLKSVVAKFNASQLITQRAQVSLLIRRELTE
RAKDFSLILD,

(SEQ ID NO:482)

VNEVLKSVVAKFNASQLITQRAQVSL,

(SEQ ID NO:483)

DVAITELSFSREYT,

(SEQ ID NO:484)

FLVEKAKQEQRQKIVQAEGEAEAAKMLGE,

(SEQ ID NO:485)

ALSKNPGYIKLRKIRAAQNISKTIATSQNRIYLTADNLVLNLQDESFTRG
SDSLIKGKK,
and/or

(SEQ ID NO:486)

KIRAAQNISKTIATSQNRIYLTADNLV.

invention.

The gene encoding the disclosed cDNA is believed to reside on chromosome 12. Accordingly, polynucleotides related to this invention are useful as a marker in linkage analysis for chromosome 12. This gene is expressed primarily in fetal brain. Therefore, polynucleotides and polypeptides of the invention are useful as reagents for differential identification of the tissue(s) or cell type(s) present in a biological sample and for diagnosis of diseases and conditions which include, but are not limited to, neural diseases, particularly proliferative conditions such as cancers or tumors. Similarly, polypeptides and antibodies directed to these polypeptides are useful in providing immunological probes for differential identification of the tissue(s) or cell type(s). For a number of disorders of the above tissues or cells, particularly of the nervous system, expression of this gene at significantly higher or lower levels may be routinely detected in certain tissues or cell types (e.g., neural, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 158 as residues: Ala-85 to Ser-91, Pro-93 to Asp-98, Glu-167 to Lys-173, Gln-205 to Ala-210. The tissue distribution in fetal brain, combined with the homology to prohibitin and the B-cell receptor associated protein indicates that the protein products of this gene are useful for the detection/treatment of neurodegenerative disease states and behavioral disorders such as Alzheimers Disease, Parkinsons Disease, Huntingtons Disease, schizophrenia, mania, dementia, paranoia, obsessive compulsive disorder, panic disorder, and autism. In addition, the gene or gene product may also play a role in the treatment and/or detection of developmental disorders associated with the developing embryo, sexually-linked disorders, and/or disorders of the cardiovascular system. Similarly, expression within fetal tissue indicates that this protein may play a role in the regulation of cellular division, and may show utility in the diagnosis and treatment of cancer and other proliferative disorders. Similarly, developmental tissues rely on decisions involving cell differentiation and/or apoptosis in pattern formation. Thus this protein may also be involved in apoptosis or tissue differentiation and could again be useful in cancer therapy. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:53 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1575 of SEQ ID NO:53, b is an integer of 15 to 1589, where both a and b correspond to the positions of nucleotide residues shown in SEQ ID NO:53, and where b is greater than or equal to a+14.

Features of Protein Encoded by Gene No: 44

The translation product of this gene shares sequence homology with the F44G4.1 gene of the c. elegans genome which has no known function (See Genbank Accession No. gnl|PID|e236516). Moreover, the translation product of this gene also shares sequence homology with the human torsionA and torsionB gene products, a gene candidate for the Torsion Dystonia disease locus (See Genbank Accession Nos. gi|2358279 (AF007871) and gi|2358281 (AF007872)).

(SEQ ID NO:487)

KALALSFHGWSGTGKNFV,

(SEQ ID NO:488)

NLIDYFLPFLPLEYRHVRLCAR,

(SEQ ID NO:489),

NLIDYFIPFLPLEYRHVRLC,

(SEQ ID NO:490)

CHQTLFIFDEAEKLHPGLLEVLGPHL,

(SEQ ID NO:491)

PEKALALSFHGWSGTGKNFVA,

(SEQ ID NO:492)

GSLARPAAGWSRSSGPA,

(SEQ ID NO:493)

DAKETIWSVIISPWDLLSSHMAFFNHLAHFLQPHSTLECVSLRHQVILRM
GSVFEFSNPSSYLSRWDLQKKEKGLAWLLM,

(SEQ ID NO:494)

NHLAHFLQPHSTLECVSLRHQVILRMG,

(SEQ ID NO:495)

MSRPPIVFEKVPPPPPKSVDHKSWPTLTWFVKYLPLCTFPFSLRLLADSU
ITEAAWLSSGSTNLKAHQPANLECILHPWVTELRSPRLCNPRTLQPLRPN
TQALPCRRAEMLRRPSGVS,
and/or

(SEQ ID NQ:496)

HQPANLECLLHPWVTELRSPRLCNPRTLQP.

Polynucleotides encoding these polypeptides are also encompassed by the invention.

This gene is expressed primarily in tonsils. Therefore, polynucleotides and polypeptides of the invention are useful as reagents for differential identification of the tissue(s) or cell type(s) present in a biological sample and for diagnosis of diseases and conditions which include, but are not limited to, immune or hematopoietic disorders, particularly tonsillitis or adnoiditis. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in tonsils, combined with the homology to F44G4.1 gene of the C. elegans and the torsion a and B proteins indicates that polynucleotides and polypeptides corresponding to this gene are useful for the treatment and detection of conditions affecting the tonsils. The tonsils have not been thoroughly studied and the actual function of this organ is not known, but this gene could be used in determining what may trigger tonsillitis, especially in children, where the tonsils seem to be most active. Furthermore, due to the homology of this gene, it may display potential utility in the detection, diagnosis, and/or treatment for Torsion Dystonia disease. Additionally, considering the high conservation of the torsian a and B proteins, in addition to their homology to ATP binding domains and heat shock protein resemblance, an essential function may be attributed to the current invention—potentially within the context of a developmental, metabolic, or signaling role in various tissues, which specifically includes immune cells and tissues. The protein may also show utility in the treatment, detection, and/or prevention of a variety of muscular degenerative or neurodegenerative conditions. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:54 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 2060 of SEQ ID NO:54, b is an integer of 15 to 2074, where both a and b correspond to the positions of nucleotide residues shown in SEQ ID NO:54, and where b is greater than or equal to a+14.

Features of Protein Encoded by Gene No: 45

The translation product of this gene was found to have homology to a protein from Schizosaccharomyces pombe, which, based upon its level of conservation, may be attributed to having an essential cellular function (See Genbank Accession No. gnl|PID|e339908).

In specific embodiments, polypeptides of the invention comprise the following amino acid sequence:

(SEQ ID NO:496)

NSXRAWRRHVPCGGGREIAWTSGRPRTA.

(SEQ ID NO:498)

VSIFSTDIHPNNKSNTIAIEFTIINGTLITVLTDRIRFISCDLIPGSSPN
PPIPWSVPQPMFLRNRSSCSTAMALTQSRXACSSEYRVKAMAVRGRPEVQ
AISLPPPQGTWRLHARXEF,
and/or

(SEQ ID NO:499)

QPMFLRNRSSCSTAMALTQSRXACSSEYRV.

Polynucleotides encoding these polypeptides are also encompassed by the invention.

The gene encoding the disclosed cDNA is believed to reside on chromosome 18. Accordingly, polynucleotides related to this invention are useful as a marker in linkage analysis for chromosome 18. This gene is expressed primarily in osteoclastoma stromal cells, and to a lesser extent, in T-cells. Therefore, polynucleotides and polypeptides of the invention are useful as reagents for differential identification of the tissue(s)- or cell type(s) present in a biological sample and for diagnosis of diseases and conditions which include, but are not limited to, immune, haemopoietic, or skeletal disorders, particularly proliferative conditions, such as leukemia. Similarly, polypeptides and antibodies directed to these polypeptides are useful in providing immunological probes for differential identification of the tissue(s) or cell type(s). For a number of disorders of the above tissues or cells, particularly of the haemo-lymphoid system, expression of this gene at significantly higher or lower levels may be routinely detected in certain tissues or cell types (e.g., immune, hematopoietic, skeletal, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in stromal and T-cells indicates that polynucleotides and polypeptides corresponding to this gene are useful for the diagnosis, treatment, and/or prevention of immune or hematopoietic diseases, such as leukemia. Moreover, the protein product of this gene is useful for the treatment and diagnosis of hematopoietic related disorders such as anemia, pancytopenia, leukopenia, thrombocytopenia or leukemia since stromal cells are important in the production of cells of hematopoietic lineages. The uses include bone marrow cell ex vivo culture, bone marrow transplantation, bone marrow reconstitution, radiotherapy or chemotherapy of neoplasia. The gene product may also be involved in lymphopoiesis, therefore, it can be used in immune disorders such as infection, inflammation, allergy, immunodeficiency etc. In addition, this gene product may have commercial utility in the expansion of stem cells and committed progenitors of various blood lineages, and in the differentiation and/or proliferation of various cell types. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:55 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1469 of SEQ ID NO:55, b is an integer of 15 to 1483, where both a and b correspond to the positions of nucleotide residues shown in SEQ ID NO:55, and where b is greater than or equal to a+14.

Features of Protein Encoded by Gene No: 46

In specific embodiments, polypeptides of the invention comprise the following amino acid sequence:

(SEQ ID NO:500)

KLPRERCGRMKWRQHEFLSPCHMFSFPXLXSKRVRYIFCNSDNFLPLCR.

Polynucleotides encoding these polypeptides are also encompassed by the invention.

This gene is expressed primarily in activated monocytes. Therefore, polynucleotides and polypeptides of the invention are useful as reagents for differential identification of the tissue(s) or cell type(s) present in a biological sample and for diagnosis of diseases and conditions which include, but are not limited to, immune or hematopoietic disorders including leukemia and allergies. Similarly, polypeptides and antibodies directed to these polypeptides are useful in providing immunological probes for differential identification of the tissue(s) or cell type(s). For a number of disorders of the above tissues or cells, particularly of the lymphoid system, expression of this gene at significantly higher or lower levels may be routinely detected in certain tissues or cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 161 as residues: Met-1 to Gly-7. The tissue distribution in monocytes indicates that the protein product of this gene is particularly useful for the treatment in tissue repair and modeling since monocytes engage in the synthesis and secretion of many cytokines which are soluble proteins that regulate highly diverse aspects of cellular biology. Monocytes are also important in the fact that their expression of Major Histocompatibility Factor II (MHCII) enable them to select and stimulate the appropriate lymphocytes to combat specific antigens in the blood. Since the gene is expressed in cells of lymphoid origin, the natural gene product may be involved in immune functions. Therefore it may be also used as an agent for immunological disorders including arthritis, asthma, immune deficiency diseases such as AIDS, and leukemia. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:56 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1109 of SEQ ID NO:56, b is an integer of 15 to 1123, where both a and b correspond to the positions of nucleotide residues shown in SEQ ID NO:56, and where b is greater than or equal to a+14.

Features of Protein Encoded by Gene No: 47

The translation product of this gene has homology to the Na+/H+-exchanging protein: Na+/H+ antiporter in Methanobacterium thermoautotrophicum, as well as, the Na+/H+ antiporter cdu2′ in Clostridium difficile (See Genbank Accession Nos. gi|2621849 (AE000854) and pir|JC53431|JC5343, respectively). Thus, it is likely that this gene has similar Na+/H+ antiporter activity.

In specific embodiments, polypeptides of the invention comprise the following amino acid sequence:

(SEQ ID NO:501)

NLKEKIFISFAWLPKATVQAAIG,

(SEQ ID NO:502)

WLPKATVQAAIGSVALD,

(SEQ ID NO:503)

VLIRILTTFLMVCFAGFNLKEKIFISFAWLPKATVQAAIGSVALDTARXH
GEKQLEDYG,

(SEQ ID NO:504)

FLMVCFAGFNLKEKIFISFAWLPKATVQAAIGSV,

(SEQ ID NO:505)

LKRKDIYFFCMASKGHSSGCNRICGFGHSKVTWRETIRRLWNGCVDSGIF
VHPHHSPNWKSAYWFTGPQASAES,

(SEQ ID NO:506)

GHSSGCNRICGFGHSKVTWRETLRRL

(SEQ ID NO:507)

TAFLALALSMWVVMIYITNLNLSAFFFKHPFIIHLNLHKDISTLMYYHSL
QLMWERPASVSSSARIALTFNFHSFLIHICPGSDALNWFWLSFLDLFLLL
NIIMQSLINFYHHLP,

(SEQ ID NO:508)

LSAFFFKHPFIIHLNLHKDISTLM,
and/or

(SEQ ID NO:509)

HICPGSDALNWFWLSFLDLFLLLN.

This gene is expressed primarily in osteoclastoma cells. Therefore, polynucleotides and polypeptides of the invention are useful as reagents for differential identification of the tissue(s) or cell type(s) present in a biological sample and for diagnosis of diseases and conditions which include, but are not limited to, immune, hematopoietic, or skeletal disorders, particularly, osteoporosis or leukemia. Similarly, polypeptides and antibodies directed to these polypeptides are useful in providing immunological probes for differential identification of the tissue(s) or cell type(s). For a number of disorders of the above tissues or cells, particularly of the lymphoid system, expression of this gene at significantly higher or lower levels may be routinely detected in certain tissues or cell types (e.g., immune, hematopoietic, skeletal, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 162 as residues: His-35 to Gln-43. The tissue distribution predominantly in osteoclastoma cells (the site of hematopoieisis) indicates that polynucleotides and polypeptides corresponding to this gene are useful for the diagnosis and treatment of bone related diseases including osteoporosis, osteopetrosis and leukemia. Furthermore, its homology to known transporter proteins may suggest the protein is useful in the diagnosis, treatment, and prevention of various developmental and metabolic disorders, particularly those based upon ion and proton transport. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:57 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1225 of SEQ ID NO:57, b is an integer of 15 to 1239, where both a and b correspond to the positions of nucleotide residues shown in SEQ ID NO:57, and where b is greater than or equal to a+14.

Features of Protein Encoded by Gene No: 48

In specific embodiments, polypeptides of the invention comprise the following amino acid sequence:

(SEQ ID NO:510)

RAGGQGACTHAKGSETPPPASPQTSEPAPSPLPPHLTGGPGMYSSEAKLP
NSFSCLGLAGTGAGI,

(SEQ ID NO:511)

GSETPPPASPQTSEPAPSPLPPHLTGGP,

(SEQ ID NO:512)

PVAAGCLPHQPLKGWGAGGMHPCKRLRNSPSGKPSDFGACAFPPTASPHR
RARHVFLRGETAKLFLLSWVGWHWGGHLGYSLCSWHWASSSSTCVHPQLG
QPSALRWGPKHLPFRLASLGLG,
and/or

(SEQ ID NO:513)

LRNSPSGKPSDFGACAFPPTASPHRRARHVFLR.

This gene is expressed primarily in amygdala and to a lesser extent in amniotic cells. Therefore, polynucleotides and polypeptides of the invention are useful as reagents for differential identification of the tissue(s) or cell type(s) present in a biological sample and for diagnosis of diseases and conditions which include, but are not limited to, neural, reproductive, or developmental disorders, particularly depression and other emotional behavioral problems. Similarly, polypeptides and antibodies directed to these polypeptides are useful in providing immunological probes for differential identification of the tissue(s) or cell type(s). For a number of disorders of the above tissues or cells, particularly of the nervous system, expression of this gene at significantly higher or lower levels may be routinely detected in certain tissues or cell types (e.g., neural, reproductive, developmental, endocrine, cancerous and wounded tissues) or bodily fluids (e.g., lymph, amniotic fluid, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 163 as residues: Pro-74 to Gly-83, Pro-88 to Thr-95. The tissue distribution in amygdala indicates that polynucleotides and polypeptides corresponding to this gene are useful for the diagnosis and treatment of mental problems associated with emotional behavior and neurodegenerative states such as Alzheimers Disease, Parkinsons Disease, Huntingtons Disease, schizophrenia, mania, dementia, paranoia, obsessive compulsive disorder and panic disorders, and depression. The amygdala processes sensory information and relays this to other areas of the brain including the endocrine and autonomic domains of the hypothalamus and the brain stem. The protein may also be useful in the diagnosis, treatment, and/or prevention of a variety of developmental disorders or even in the amelioration of immune or endocrine conditions since the efficacy of the immune system has been shown to be indirectly related to an individual's emotional condition. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:58 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 789 of SEQ ID NO:58, b is an integer of 15 to 803, where both a and b correspond to the positions of nucleotide residues shown in SEQ ID NO:58, and where b is greater than or equal to a+14.

Features of Protein Encoded by Gene No: 49

This gene is expressed primarily in stromal cells. Therefore, polynucleotides and polypeptides of the invention are useful as reagents for differential identification of the tissue(s) or cell type(s) present in a biological sample and for diagnosis of diseases and conditions which include, but are not limited to, immune or hematopoietic disorders, particularly leukemia and other cancers and disorders deriving from hematopoietic cells. Similarly, polypeptides and antibodies directed to these polypeptides are useful in providing immunological probes for differential identification of the tissue(s) or cell type(s). For a number of disorders of the above tissues or cells, particularly of the lymphoid system, expression of this gene at significantly higher or lower levels may be routinely detected in certain tissues or cell types (e.g., immune, hematopoietic cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in stromal cells indicates that polynucleotides and polypeptides corresponding to this gene are useful for the treatment and diagnosis of hematopoetic related disorders such as anemia, pancytopenia, leukopenia, thrombocytopenia or leukemia since stromal cells are important in the production of cells of hematopoietic lineages. The uses include bone marrow cell ex vivo culture, bone marrow transplantation, bone marrow reconstitution, radiotherapy or chemotherapy of neoplasia. The gene product may also be involved in lymphopoiesis, therefore, it can be used in immune disorders such as infection, inflammation, allergy, immunodeficiency etc. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:59 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 981 of SEQ ID NO:59, b is an integer of 15 to 995, where both a and b correspond to the positions of nucleotide residues shown in SEQ ID NO:59, and where b is greater than or equal to a+14.

Features of Protein Encoded by Gene No: 50

In specific embodiments, polypeptides of the invention comprise the following amino acid sequence: FFTWRTYLPLD (SEQ ID NO:514). Polynucleotides encoding these polypeptides are also encompassed by the invention. The gene encoding the disclosed cDNA is believed to reside on chromosome 9. Accordingly, polynucleotides related to this invention are useful as a marker in linkage analysis for chromosome 9. This gene is expressed primarily in tumors, particularly skin and adrenal gland tumors, and to a lesser extent in bone marrow stromal cells and activated T cells. Therefore, polynucleotides and polypeptides of the invention are useful as reagents for differential identification of the tissue(s) or cell type(s) present in a biological sample and for diagnosis of diseases and conditions which include, but are not limited to, cancer, hematopoietic or immune disorders. Similarly, polypeptides and antibodies directed to these polypeptides are useful in providing immunological probes for differential identification of the tissue(s) or cell type(s). For a number of disorders of the above tissues or cells, particularly of the skin, adrenal gland, and immune system, expression of this gene at significantly higher or lower levels may be routinely detected in certain tissues or cell types (e.g., hematopoietic, immune, endocrine, integumentary, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 165 as residues: Glu-13 to Arg-22, Ser-58 to Trp-63. The tissue distribution in tumors indicates that polynucleotides and polypeptides corresponding to this gene are useful for the detection and treatment of cancer. Elevated levels of expression of this gene in a variety of tumors suggest that it may play a role in cell proliferation, the induction of angiogenesis, destruction of the basal lamina, or a variety of other physiological processes that support the growth and development of tumors and cancer. Alternatively, its expression in the hematopoietic compartment, particularly in the bone marrow stroma and by activated T cells suggests that it may represent a soluble factor capable of influencing a variety of hematopoietic lineages. Therefore, this gene product may have commercial utility in the expansion of stem cells and committed progenitors of various blood lineages, and in the differentiation and/or proliferation of blood cells. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:60 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 952 of SEQ ID NO:60, b is an integer of 15 to 966, where both a and b correspond to the positions of nucleotide residues shown in SEQ ID NO:60, and where b is greater than or equal to a+14.

Features of Protein Encoded by Gene No: 51

(SEQ ID NO:515)

LQVYIQMHR,

(SEQ ID NO:516)

KFIQNKDCQRMLNLGRGRFDGGTELSNKVSKFLLTNYPLIAKGKTITIHW
RNWTSYPSDQN,
and/or

(SEQ ID NO:517)

RGRFDGGTELSNKVSKFLLTNYPLIAKG.

invention.

This gene is expressed primarily in benign human breast tissue. Therefore, polynucleotides and polypeptides of the invention are useful as reagents for differential identification of the tissue(s) or cell type(s) present in a biological sample and for diagnosis of diseases and conditions which include, but are not limited to, reproductive, proliferative, or endocrine disorders, particularly breast cancer. Similarly, polypeptides and antibodies directed to these polypeptides are useful in providing immunological probes for differential identification of the tissue(s) or cell type(s). For a number of disorders of the above tissues or cells, particularly of the breast and reproductive tissues, expression of this gene at significantly higher or lower levels may be routinely detected in certain tissues or cell types (e.g., reproductive, proliferative, endocrine, and cancerous and wounded tissues) or bodily fluids (e.g., lymph, serum, breast milk, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in benign human breast tissue indicates that polynucleotides and polypeptides corresponding to this gene are useful for the treatment and/or diagnosis of breast cancer. Alternately, this protein may play an important role in lactation or represent a critical component secreted into the milk, which may have an important function in the immunoprotection, health, and/or nourishment of the infant upon breastfeeding. Protein, as well as, antibodies directed against the protein may show utility as a tumor marker and/or immunotherapy targets for the above listed tumors and tissues

Many polynucleotide sequences, such as EST sequences, are publicly available and accessible through sequence databases. Some of these sequences are related to SEQ ID NO:61 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 248 of SEQ ID NO:61, b is an integer of 15 to 262, where both a and b correspond to the positions of nucleotide residues shown in SEQ ID NO:61, and where b is greater than or equal to a+14.

Features of Protein Encoded by Gene No: 52

The translation product of this gene has homology with the conserved human ring finger proteins (See Genbank Accession No. gnl|PID|e351238 (AJ001019)), which are thought to be important in facilitating and regulating signal transduction pathways in eukaryotic cells.

(SEQ ID NO:518)

HDRTMQDIVYKLVPGLQE,

(SEQ ID NO:519)

FASHDRTMQDIVYKLVPGLQEGE,

(SEQ ID NO:520)

GTGSFASHDRT,

(SEQ ID NO:521)

PRSRPALRPGRQRPPSHSATSGVLRPRKKPDP,

(SEQ ID NO:522)

SLRSQPGLCSPCSPGSLVLGWEAPFLLAESPSSHPCSQPNFISLAGLFFR
LRCVXIFLPLMCIHSASMDRMFSTWXPQGSTQLLPLPGPCLXPGPHLLSQ
ACLPSSHSASFPTTEEQHVGIAGSWCF,
and/or

(SEQ ID NO:523)

CSPCSPGSLVLGWEAPFLLAESPSSHPCSQPNFISLA.

This gene is expressed primarily in adult whole brain. Therefore, polynucleotides and polypeptides of the invention are useful as reagents for differential identification of the tissue(s) or cell type(s) present in a biological sample and for diagnosis of diseases and conditions which include, but are not limited to, neural disorders, particularly neurodegenerative disorders; Schizophrenia; Alzheimers; tumors of a brain or neuronal cell origin. Similarly, polypeptides and antibodies directed to these polypeptides are useful in providing immunological probes for differential identification of the tissue(s) or cell type(s). For a number of disorders of the above tissues or cells, particularly of the CNS and/or peripheral nervous system, expression of this gene at significantly higher or lower levels may be routinely detected in certain tissues or cell types (e.g., neural, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 167 as residues: Phe-39 to Gly-44. The tissue distribution in adult whole brain indicates that polynucleotides and polypeptides corresponding to this gene are useful for the detection/treatment of neurodegenerative disease states and behavioral disorders such as Alzheimers Disease, Parkinsons Disease, Huntingtons Disease, schizophrenia, mania, dementia, paranoia, obsessive compulsive disorder and panic disorder. In addition, considering the homology to the conserved ring finger proteins may suggest that the gene or gene product may also play a role in the treatment and/or detection of developmental disorders associated with the developing embryo. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:62 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 739 of SEQ ID NO:62, b is an integer of 15 to 753, where both a and b correspond to the positions of nucleotide residues shown in SEQ ID NO:62, and where b is greater than or equal to a+14.

Features of Protein Encoded by Gene No: 53

The translation product of this gene shares homology with the human conserved Lst-1 gene product, a member of the TNF family of proteins (See Genbank Accession No. gi|1127546).

(SEQ ID NO:524)

LVLSLGAWGWPSTCLWW,

(SEQ ID NO:525)

GWQNAYRD,

(SEQ ID NO:526)

LHLKHWPSLXKLQEAVGKVIINATTCTVTCGLGYKEETVCEVGPDGVRRK
CQTRRLECLTNWICGMLHFTILIGKEFELSCLSSDILEFGQEAFRFTXXL
ARGVISTDDEVFKPFQANSHFVKFKYAQEYDSGTYRCDVQLVKNLRLVKR
LYFGLRVLPPNLVNLNFHQSLTEDQD,

(SEQ ID NO:527)

ANSHFVKFKYAQEYDSGTYRCDVQLVKNLRLVKRLYFGLRVLPPNLV,

(SEQ ID NO:528)

LMEIQIHQVRRKDPQPKIEPLDESQVFYQLHITAICPRVILLSIFKLHKV
GVGLKGFEDLIVSGDDTSSKXXGEPESFLSKLQDV,
and/or

(SEQ ID NO:529)

LDESQVFYQLHITAICPRVILLSIFKL.

encompassed by the invention.

This gene is expressed primarily in human 6-week old embryo. Therefore, polynucleotides and polypeptides of the invention are useful as reagents for differential identification of the tissue(s) or cell type(s) present in a biological sample and for diagnosis of diseases and conditions which include, but are not limited to, developmental disorders, particularly abnormal cell proliferation; defects in terminal tissue differentiation. Similarly, polypeptides and antibodies directed to these polypeptides are useful in providing immunological probes for differential identification of the tissue(s) or cell type(s). For a number of disorders of the above tissues or cells, particularly of the embryo, expression of this gene at significantly higher or lower levels may be routinely detected in certain tissues or cell types (e.g., developmental, and cancerous and wounded tissues) or bodily fluids (e.g., lymph, amniotic fluid, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in embryonic tissues indicates that polynucleotides and polypeptides corresponding to this gene are useful for the treatment and/or diagnosis of fetal disorders. Alternately, the expression may reflect a role for this protein in proliferating cells. In such an event, this gene product may be useful in the treatment or diagnosis of abnormal cell proliferation, such as that involved in cancer. Similarly, embryonic development also involves decisions involving cell differentiation and/or apoptosis involved in pattern formation. Thus, this protein may also be involved in apoptosis or tissue differentiation, and could again be useful in cancer therapy. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:63 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 725 of SEQ ID NO:63, b is an integer of 15 to 739, where both a and b correspond to the positions of nucleotide residues shown in SEQ ID NO:63, and where b is greater than or equal to a+14.

Features of Protein Encoded by Gene No: 54

This gene is expressed primarily in human epithelioid sarcoma. Therefore, polynucleotides and polypeptides of the invention are useful as reagents for differential identification of the tissue(s) or cell type(s) present in a biological sample and for diagnosis of diseases and conditions which include, but are not limited to, integumentary disorders, particularly epithelial sarcoma; tumors of an epithelial cell origin including the underlying integument. Similarly, polypeptides and antibodies directed to these polypeptides are useful in providing immunological probes for differential identification of the tissue(s) or cell type(s). For a number of disorders of the above tissues or cells, particularly of the skin and epithelial tissue layers, expression of this gene at significantly higher or lower levels may be routinely detected in certain tissues or cell types (e.g., integumenary, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 169 as residues: Met-1 to Tyr-6, Thr-24 to Cys-36. The tissue distribution in epithelioid sarcoma tissue indicates that polynucleotides and polypeptides corresponding to this gene are useful for the treatment and/or diagnosis of epithelial cancer. This gene product displays enhanced expression in epithelial cell sarcoma, and thus may be involved in cell proliferation, apoptosis, or in the control of angiogenesis. Similarly, expression within epithelial tissues indicates the protein product of this gene is useful for the treatment, diagnosis, and/or prevention of various skin disorders including congenital disorders (i.e. nevi, moles, freckles, Mongolian spots, hemangiomas, port-wine syndrome), integumentary tumors (i.e. keratoses, Bowen's disease, basal cell carcinoma, squamous cell carcinoma, malignant melanoma, Paget's disease, mycosis fungoides, and Kaposi's sarcoma), injuries and inflammation of the skin (i.e. wounds, rashes, prickly heat disorder, psoriasis, dermatitis), atherosclerosis, uticaria, eczema, photosensitivity, autoimmune disorders (i.e. lupus erythematosus, vitiligo, dermatomyositis, morphea, scleroderma, pemphigoid, and pemphigus), keloids, striae, erythema, petechiae, purpura, and xanthelasma. In addition, such disorders may predispose increased susceptibility to viral and bacterial infections of the skin (i.e. cold sores, warts, chickenpox, molluscum contagiosum, herpes zoster, boils, cellulitis, erysipelas, impetigo, tinea, althletes foot, and ringworm). Moreover, the protein product of this gene may also be useful for the treatment or diagnosis of various connective tissue disorders such as arthritis, trauma, tendonitis, chrondomalacia and inflammation, autoimmune disorders such as rheumatoid arthritis, lupus, scleroderma, and dermatomyositis as well as dwarfism, spinal deformation, and specific joint abnormalities as well as chondrodysplasias (i.e. spondyloepiphyseal dysplasia congenita, familial osteoarthritis, Atelosteogenesis type II, metaphyseal chondrodysplasia type Schmid). Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:64 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 462 of SEQ ID NO:64, b is an integer of 15 to 476, where both a and b correspond to the positions of nucleotide residues shown in SEQ ID NO:64, and where b is greater than or equal to a+14.

Features of Protein Encoded by Gene No: 55

In specific embodiments, polypeptides of the invention comprise the following amino acid sequence: NSARED (SEQ ID NO:530). Polynucleotides encoding these polypeptides are also encompassed by the invention. This gene is expressed primarily in endometrial tumors. Therefore, polynucleotides and polypeptides of the invention are useful as reagents for differential identification of the tissue(s) or cell type(s) present in a biological sample and for diagnosis of diseases and conditions which include, but are not limited to, endometrial cancer including other cancers of the female reproductive system. Similarly, polypeptides and antibodies directed to these polypeptides are useful in providing immunological probes for differential identification of the tissue(s) or cell type(s). For a number of disorders of the above tissues or cells, particularly of the endometrium and reproductive system, expression of this gene at significantly higher or lower levels may be routinely detected in certain tissues or cell types (e.g., reproductive, cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution indicates that polynucleotides and polypeptides corresponding to this gene are useful for the diagnosis and treatment of cancers, particularly those of the endometrium and other reproductive organs. Protein, as well as, antibodies directed against the protein may show utility as a tumor marker and/or immunotherapy targets for the above listed tumors and tissues

Many polynucleotide sequences, such as EST sequences, are publicly available and accessible through sequence databases. Some of these sequences are related to SEQ ID NO:65 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 682 of SEQ ID NO:65, b is an integer of 15 to 696, where both a and b correspond to the positions of nucleotide residues shown in SEQ ID NO:65, and where b is greater than or equal to a+14.

Features of Protein Encoded by Gene No: 56

This gene is expressed primarily in metastatic melanoma, and to a lesser extent, in fetal lung. Therefore, polynucleotides and polypeptides of the invention are useful as reagents for differential identification of the tissue(s) or cell type(s) present in a biological sample and for diagnosis of diseases and conditions which include, but are not limited to, integumentary, developmental, or pulmonary disorders, particularly melanoma, ARDS, emphysema, or cystic fibrosis. Similarly, polypeptides and antibodies directed to these polypeptides are useful in providing immunological probes for differential identification of the tissue(s) or cell type(s). For a number of disorders of the above tissues or cells, particularly of the skin, expression of this gene at significantly higher or lower levels may be routinely detected in certain tissues or cell types (e.g., developmental, integumentary, pulmonary, and cancerous and wounded tissues) or bodily fluids (e.g., lymph, serum, pulmonary sputum or surfactant, amniotic fluid, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 171 as residues: Asp-20 to Lys-25. The tissue distribution in metastatic melanoma indicates that polynucleotides and polypeptides corresponding to this gene are useful for the diagnosis and treatment of cancer, particularly melanoma, and more particularly, metastasizing melanomas. In addition, the tissue distribution also indicates that polynucleotides and polypeptides corresponding to this gene are useful for the diagnosis and treatment of cancer and other proliferative disorders. Expression in embryonic tissue and other cellular sources marked by proliferating cells indicates that this protein may play a role in the regulation of cellular division. Moreover, the protein product of this gene is useful for the treatment, diagnosis, and/or prevention of various skin disorders including congenital disorders (i.e. nevi, moles, freckles, Mongolian spots, hemangiomas, port-wine syndrome), integumentary tumors (i.e. keratoses, Bowen's disease, basal cell carcinoma, squamous cell carcinoma, malignant melanoma, Paget's disease, mycosis fungoides, and Kaposi's sarcoma), injuries and inflammation of the skin (i.e. wounds, rashes, prickly heat disorder, psoriasis, dermatitis), atherosclerosis, uticaria, eczema, photosensitivity, autoimmune disorders (i.e. lupus erythematosus, vitiligo, dermatomyositis, morphea, scleroderma, pemphigoid, and pemphigus), keloids, striae, erythema, petechiae, purpura, and xanthelasma. In addition, such disorders may predispose increased susceptibility to viral and bacterial infections of the skin (i.e. cold sores, warts, chickenpox, molluscum contagiosum, herpes zoster, boils, cellulitis, erysipelas, impetigo, tinea, althletes foot, and ringworm). Moreover, the protein product of this gene may also be useful for the treatment or diagnosis of various connective tissue disorders such as arthritis, trauma, tendonitis, chrondomalacia and inflammation, autoimmune disorders such as rheumatoid arthritis, lupus, scleroderma, and dermatomyositis as well as dwarfism, spinal deformation, and specific joint abnormalities as well as chondrodysplasias (i.e. spondyloepiphyseal dysplasia congenita, familial osteoarthritis, Atelosteogenesis type II, metaphyseal chondrodysplasia type Schmid). Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:66 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1876 of SEQ ID NO:66, b is an integer of 15 to 1890, where both a and b correspond to the positions of nucleotide residues shown in SEQ ID NO:66, and where b is greater than or equal to a+14.

Features of Protein Encoded by Gene No: 57

In specific embodiments, polypeptides of the invention comprise the following amino acid sequence:

| | |
|---|---|
| EGTGTRFGLACSLPASHALLRPGSESRLLPVMPPIQEPRFFSKTRPVPFSTAASQQRAPGSPRSQLWLWTTWLRPLGLQSLHWVYLGL | (SEQ ID NO:531) |
| IHSWSQGWGFTCEHQTDLLASRAVDSLMKALVRRKHSVLRLLCNRFVI, | |
| PPIQEPRFFSKTRPVPFSTAASQQRAPGSP, | (SEQ ID NO:532) |
| TCEHQ TDLLASRAVDSLMKALVRR, | (SEQ ID NO:533) |
| QCKLCNPRGRSHVVQSHSWDLGDPGALCWEAAVEKGTGRVLLKNRGSCMGGITGRRRLSLPGLSRAWLAGRLHASPNRVPVPSHRA | (SEQ ID NO:534) |
| DGSCGSHGEGEXLGALLRSRXL, | |
| and/or | |
| MGGITGRRRLSLPGLSRAWLAGRLHASPNRVP. | (SEQ ID NO:535) |

Polynucleotides encoding these polypeptides are also encompassed by the invention.

This gene is expressed primarily in T-cell lymphoma. Therefore, polynucleotides and polypeptides of the invention are useful as reagents for differential identification of the tissue(s) or cell type(s) present in a biological sample and for diagnosis of diseases and conditions which include, but are not limited to, immune or hematopoietic disorders, particularly lymphomas and other immune-derived cancers. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 172 as residues: Met-1 to Asn-7. The tissue distribution in T-cell lymphoma indicates that polynucleotides and polypeptides corresponding to this gene are useful for the diagnosis and treatment of lymphomas, particularly T cell lymphomas, and other cancers. In addition, the tissue distribution indicates that polynucleotides and polypeptides corresponding to this gene are useful for the diagnosis and treatment of cancer and other proliferative disorders. Moreover, the expression in hematopoietic cells and tissues indicates that this protein may play a role in the proliferation, differentiation, and/or survival of hematopoietic cell lineages. Thus, this gene may be useful in the treatment of lymphoproliferative disorders, and in the maintenance and differentiation of various hematopoietic lineages from early hematopoietic stem and committed progenitor cells. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:67 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1600 of SEQ ID NO:67, b is an integer of 15 to 1614, where both a and b correspond to the positions of nucleotide residues shown in SEQ ID NO:67, and where b is greater than or equal to a+14.

Features of Protein Encoded by Gene No: 58

In specific embodiments, polypeptides of the invention comprise the following amino acid sequence: WRYSVFFFSFKQRKK (SEQ ID NO:536). Polynucleotides encoding these polypeptides are also encompassed by the invention. The gene encoding the disclosed cDNA is believed to reside on chromosome 7. Accordingly, polynucleotides related to this invention are useful as a marker in linkage analysis for chromosome 7. This gene is expressed primarily in brain, and to a lesser extent, in spinal cord. Therefore, polynucleotides and polypeptides of the invention are useful as reagents for differential identification of the tissue(s) or cell type(s) present in a biological sample and for diagnosis of diseases and conditions which include, but are not limited to, CNS and peripheral nervous system diseases and disorders, particularly neurodegenerative conditions. Similarly, polypeptides and antibodies directed to these polypeptides are useful in providing immunological probes for differential identification of the tissue(s) or cell type(s). For a number of disorders of the above tissues or cells, particularly of the nervous system, expression of this gene at significantly higher or lower levels may be routinely detected in certain tissues or cell types (e.g., neural, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 173 as residues: Tyr-14 to Ala-30. The tissue distribution in brain and spinal cord indicates that polynucleotides and polypeptides corresponding to this gene are useful for the detection/treatment of neurodegenerative disease states and behavioral disorders such as Alzheimers Disease, Parkinsons Disease, Huntingtons Disease, schizophrenia, mania, dementia, paranoia, obsessive compulsive disorder, panic disorder, and autism. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:68 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 582 of SEQ ID NO:68, b is an integer of 15 to 596, where both a and b correspond to the positions of nucleotide residues shown in SEQ ID NO:68, and where b is greater than or equal to a+14.

Features of Protein Encoded by Gene No: 59

The translation product of this gene shares homology to the conserved C. elegans protein FER-1, which, based upon its conservation, may be attributed to playing a vital role in cellular metabolism (See Genbank Accession No. gi|1373333).

| | |
|---|---|
| QGKLQMWVDVFPKSL, | (SEQ ID NO:537) |
| PPFNITPRKAKKYYLR, | (SEQ ID NO:538) |
| KTDVHYRSLDGEGNFNWRF, | (SEQ ID NO:539) |
| PRLIIQIWDNDKFSLDDYLGFLELDL, | (SEQ ID NO:540) |
| PEFPGVFDPSGTLHSTFQPNISQGKLQMWVDVFPKSLGPPGPPFNITPRKAKKYYLRVIIWNTKDVILDEKSITGEEMSDIYVKGW | (SEQ ID NO:541) |
| IPGNEENKQKTDVHYRSLDGEGNFNWRFVFPFDYLPAEQLCIVAKKEHFWSIDQTEFRIPPRLIIQIWDNDKFSLDDYLGFLELDL | |
| RHTIIPAKSPEKCRLDMIPDLKAMNPLKAKTASLFEQKSMKGWWPCYAEKDGARVMAGKVEMTLEILNEKEADERPAGKGRDEPNM | |
| NPKLDLPNRPETSFLWFTNPCKT, | |
| QGKLQMWVDVFPKSLGPPGPPFNITPRK, | (SEQ ID NO:542) |
| VHYRSLDGEGNFNWRFVFPFDYLPAEQLCIVAKK, | (SEQ ID NO:543) |
| FSLDD YLGFLELDLRHTIIPAKSPEKCRLD, | (SEQ ID NO:544) |
| PAGKGRDEPNMNPKLDLPNRPETSFLWF, | (SEQ ID NO:545) |
| AEVHERMVAMLRRERWRPRNGWESGDDIGNPQREGGRREASREGAGRTQHEPQAGLTKSTRNLLPLVHQPMQDHEVHRVAPL, | (SEQ ID NO:546) |
| GDDIGNPQREGGRREASREGAGRTQHEPQA, | (SEQ ID NO:547) |
| ETQVVIQRKLVIVPYLNDQPGWDSKFRLVNTPEMLFFRNDTELFGWKVVKRENKSPVKIPFTIQRSVMDICFLFVFFIARNPAFDV | (SEQ ID NO:548) |
| DVTHFLSCDAFLVQDNVLGVPDDHTQVVFLGFPGCDVERRAWWPQTLGENIHPHLKFSLGNVGLEGAVQSPGRVEHTREFR, | |
| FFRNDTELFGWKVVKRENKSPVKIPFTIQ, | (SEQ ID NO:549) |
| and/or | |
| QVVFLGFPGCDVERRAWWPQTLGENIHPH. | (SEQ ID NO:550) |

The gene encoding the disclosed cDNA is believed to reside on chromosome 10. Accordingly, polynucleotides related to this invention are useful as a marker in linkage analysis for chromosome 10. This gene is expressed primarily in synovial fibroblasts, and to a lesser extent, in synovial hypoxia. Therefore, polynucleotides and polypeptides of the invention are useful as reagents for differential identification of the tissue(s) or cell type(s) present in a biological sample and for diagnosis of diseases and conditions which include, but are not limited to, skeletal disorders, particularly degenerative joint conditions, such as arthritis, synovial inflammation and other diseases of the joints. Similarly, polypeptides and antibodies directed to these polypeptides are useful in providing immunological probes for differential identification of the tissue(s) or cell type(s). For a number of disorders of the above tissues or cells, particularly of the synovium, expression of this gene at significantly higher or lower levels may be routinely detected in certain tissues or cell types (e.g., skeletal, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in synovial tissues indicates that polynucleotides and polypeptides corresponding to this gene are useful for the diagnosis and treatment of diseases affecting the synovium of the joints, such as rheumatoid arthritis, osteoarthritis, other inflammatory conditions affecting the joints, as well as in the detection and treatment of disorders and conditions affecting the skeletal system, in particular the connective tissues (e.g. trauma, tendonitis, chrondomalacia and inflammation). Furthermore, the homology to a conserved C. elegans protein may suggest that this protein is important in human development, and thus is beneficial in the diagnosis, prevention, and treatment of developmental disorders. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:69 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1510 of SEQ ID NO:69, b is an integer of 15 to 1524, where both a and b correspond to the positions of nucleotide residues shown in SEQ ID NO:69, and where b is greater than or equal to a+14.

Features of Protein Encoded by Gene No: 60

In specific embodiments, polypeptides of the invention comprise the following amino acid sequence:

| | |
|---|---|
| LPPQAWRRRPRSPAAPQPFNDSIEWSGYNKPERKGPLALFLVFLFLDTPPLQGDL. | (SEQ ID NO:551) |

Polynucleotides encoding these polypeptides are also encompassed by the invention.

This gene is expressed primarily in endothelial cells, and to a lesser extent, in brain. Therefore, polynucleotides and polypeptides of the invention are useful as reagents for differential identification of the tissue(s) or cell type(s) present in a biological sample and for diagnosis of diseases and conditions which include, but are not limited to, inflammation and other disorders of the integumentary or vascular system, such as stroke, in addition to neurodegenerative and nervous system disorders. Similarly, polypeptides and antibodies directed to these polypeptides are useful in providing immunological probes for differential identification of the tissue(s) or cell type(s). For a number of disorders of the above tissues or cells, particularly of the endothelial, circulatory, and nervous systems, expression of this gene at significantly higher or lower levels may be routinely detected in certain tissues or cell types (e.g., vascular, endothelial, neural, integumentary, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 175 as residues: Ser-4 to Gly-13. The tissue distribution in endothelial cells indicates that polynucleotides and polypeptides corresponding to this gene are useful for the diagnosis and treatment of inflammatory diseases primarily mediated through endothelial cells, such as sepsis, inflammatory bowel disease, psoriasis, and Crohn's disease, as well as stroke. Similarly, the protein would also be useful for the treatment, detection, and/or prevention of a variety of vascular disorders, which include atherosclerosis, embolism, aneurysm, or microvascular disease. In addition, the tissue distribution indicates that polynucleotides and polypeptides corresponding to this gene are useful for the detection/treatment of neurodegenerative disease states and behavioral disorders such as Alzheimers Disease, Parkinsons Disease, Huntingtons Disease, schizophrenia, mania, dementia, paranoia, obsessive compulsive disorder and panic disorder. In addition, the gene or gene product may also play a role in the treatment and/or detection of developmental disorders associated with the developing embryo, or disorders of the cardiovascular system. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:70 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 805 of SEQ ID NO:70, b is an integer of 15 to 819, where both a and b correspond to the positions of nucleotide residues shown in SEQ ID NO:70, and where b is greater than or equal to a+14.

Features of Protein Encoded by Gene No: 61

In specific embodiments, polypeptides of the invention comprise the following amino acid sequence: LLGMSLLWLFLSLIPLRNQPL (SEQ ID NO:552). Polynucleotides encoding these polypeptides are also encompassed by the invention.

This gene is expressed primarily in fetal brain. Therefore, polynucleotides and polypeptides of the invention are useful as reagents for differential identification of the tissue(s) or cell type(s) present in a biological sample and for diagnosis of diseases and conditions which include, but are not limited to, CNS and peripheral nervous system disorders, or developmental disorders. Similarly, polypeptides and antibodies directed to these polypeptides are useful in providing immunological probes for differential identification of the tissue(s) or cell type(s). For a number of disorders of the above tissues or cells, particularly of the nervous system, expression of this gene at significantly higher or lower levels may be routinely detected in certain tissues or cell types (e.g., neural, developmental, and cancerous and wounded tissues) or bodily fluids (e.g., lymph, serum, amniotic fluid, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 176 as residues: Lys-24 to Ser-30. The tissue distribution in fetal brain indicates that polynucleotides and polypeptides corresponding to this gene are useful for the diagnosis and treatment of neural disorders such as Alzheimer's disease, depression, paranoia, schizophrenia, autism, and particularly developmental brain disorders. Moreover, the expression within fetal tissue indicates that this protein may play a role in the regulation of cellular division, and may show utility in the diagnosis and treatment of cancer and other proliferative disorders. Similarly, developmental tissues rely on decisions involving cell differentiation and/or apoptosis in pattern formation. Thus this protein may also be involved in apoptosis or tissue differentiation and could again be useful in cancer therapy. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:71 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1428 of SEQ ID NO:71, b is an integer of 15 to 1442, where both a and b correspond to the positions of nucleotide residues shown in SEQ ID NO:71, and where b is greater than or equal to a+14.

Features of Protein Encoded by Gene No: 62

The translation product of this gene shares homology with the conserved 4-nitrophenylphosphatase from Schizosaccharomyces pombe (See Genbank Accession No. gi|1938421). In specific embodiments, polypeptides of the invention comprise the following amino acid sequence:

| | |
|---|---|
| AVMIGDDCRDDVGGA, | (SEQ ID NO:553) |
| ILVKTGKYRASDEEKIN, | (SEQ ID NO:554) |
| EFPSCCGPHSAAPIVKQCVHLKQLE, | (SEQ ID NO:555) |
| PEEAVMIGDDCRDDVGGAQDVGMLGILVKTGKYRASDEEKINPPPYLTCESFPHAVDHILQHLL, | (SEQ ID NO:556) |
| and/or | |
| RDDVGGAQDVGMLGILVKTGKYRASDEEKIN. | (SEQ ID NO:557) |

the invention.

The gene encoding the disclosed cDNA is thought to reside on chromosome 18. Accordingly, polynucleotides related to this invention are useful as a marker in linkage analysis for chromosome 18. This gene is expressed primarily in endometrial tumor, and to a lesser extent, in leukemia and lymphoma. Therefore, polynucleotides and polypeptides of the invention are useful as reagents for differential identification of the tissue(s) or cell type(s) present in a biological sample and for diagnosis of diseases and conditions which include, but are not limited to, immune, hematopoetic, or reproductive system disorders, particularly cancers. Similarly, polypeptides and antibodies directed to these polypeptides are useful in providing immunological probes for differential identification of the tissue(s) or cell type(s). For a number of disorders of the above tissues or cells, particularly of the endometrium and white blood cells, expression of this gene at significantly higher or lower levels may be routinely detected in certain tissues or cell types (e.g., immune, hematopoetic, reproductive, and cancerous and wounded tissues) or bodily fluids (e.g., lymph, amniotic fluid, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 177 as residues: Val-19 to Cys-24. The tissue distribution in endometrial tumors, leukemia, and lymphoma tissue indicates that polynucleotides and polypeptides corresponding to this gene are useful for the detection, diagnosis, and treatment of cancers, particularly those cancers affecting endometrial tissues and the lymphatic system. In addition, the tissue distribution indicates that polynucleotides and polypeptides corresponding to this gene are useful for the treatment and diagnosis of hematopoetic related disorders such as anemia, pancytopenia, leukopenia, thrombocytopenia or leukemia, since stromal cells are important in the production of cells of hematopoietic lineages. The uses include bone marrow cell ex vivo culture, bone marrow transplantation, bone marrow reconstitution, radiotherapy or chemotherapy of neoplasia. The gene product may also be involved in lymphopoiesis, and therefore it can be used in immune disorders such as infection, inflammation, allergy, immunodeficiency, etc. Furthermore, homology to a conserved S. pombe protein may suggest that this protein is important in development. Therefore, this protein may be beneficial in the diagnosis, prevention, and treatment of developmental disorders. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:72 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1209 of SEQ ID NO:72, b is an integer of 15 to 1223, where both a and b correspond to the positions of nucleotide residues shown in SEQ ID NO:72, and where b is greater than or equal to a+14.

Features of Protein Encoded by Gene No: 63

The translation product of this gene shares sequence homology with the CP24 protein from Brucella melitensis, which is thought to be a ribosomal releasing factor thought to be important in protein synthesis (See Genbank Accession No. gi|1674445).

In specific embodiments, polypeptides of the invention comprise the following amino acid sequence:

| | |
|---|---|
| QALYLDLLLRSAFCLILFSGCFQGLSSV, | (SEQ ID NO:558) |
| VQAFCQQQVXSESHPPAQSRSRKRMNMSECSPGRLGLTRTGFCQHGDSLLCRWGRERSPNVPPMGSSRNWALEYCWPQWTFIQGVL | (SEQ ID NO:559) |
| WSSLPDACPVLPLCHRPFG, | |
| NVPPMGSSRNWALEYCWPQWTFIQGVLWSSLPD, | (SEQ ID NO:560) |
| TCLSVPLEGWALQGQASVSMVTVFCVDGGGREVPMCHPWDPAETGLWSIAGPSGLSSKEFFGLHCQMPVQFCHCVIGHLADLFLY, | (SEQ ID NO:561) |
| and/or | |
| HPWDPAETGLWSIAGPSGLSSKEFFGLHCQMPV. | (SEQ ID NO:562) |

polypeptides are also encompassed by the invention.

This gene is expressed primarily in pancreas tumor, placenta, testis, ovarian cancer, adipocytes, spleen, and fetal liver and heart. Therefore, polynucleotides and polypeptides of the invention are useful as reagents for diagnosis of diseases and conditions which include, but are not limited to, immune, hematopoietic, metabolic, reproductive, developmental, cardiovascular, or vascular diseases. Similarly, polypeptides and antibodies directed to these polypeptides are useful in providing immunological probes for differential identification of the tissue(s) or cell type(s). For a number of disorders of the above tissues or cells, particularly of the immune system, cardiovascular system, digestive system and reproductive system. expression of this gene at significantly higher or lower levels may be routinely detected in certain tissues or cell types (e.g., immune, hematopoietic, reproductive, developmental, metabolic, cardiovascular, vascular, cancerous and wounded tissues) or bodily fluids (e.g., lymph, serum, plasma, amniotic fluid, seminal fluid, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 178 as residues: Glu-36 to His-41, Thr-57 to Thr-70, Glu-87 to Met-92, Lys-100 to Lys-105, Ala-197 to Ser-227. The tissue distribution in pancreas, reproductive tissues, and spleen, combined with the homology to ribosomal releasing factor indicates that polynucleotides and polypeptides corresponding to this gene are useful for the treatment and diagnosis of many diseases, especially cancers and immune, vascular, or reproductive diseases. Moreover, the homology to the ribosome releasing factor indicates that the protein may serve as an anticancer agent or an agent that may be useful for the inhibition of proliferative cells. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:73 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1800 of SEQ ID NO:73, b is an integer of 15 to 1814, where both a and b correspond to the positions of nucleotide residues shown in SEQ ID NO:73, and where b is greater than or equal to a+14.

Features of Protein Encoded by Gene No: 64

The translation product of this gene shares sequence homology with a secretory protein containing thrombospondin motifs from Mus musculus called ADAMTS-1, which is thought to be important in the activation of proteins and the processes of thrombopoiesis and metabolism (See Genbank Accession No. gnl|PID|d1011748). ADAMTS-1 is a new type of ADAM family protein with TSP type I motifs. The TSP homologous domain containing the TSP type I motif of ADAMTS-1 is functional for binding to heparin. ADAMTS-1 mRNA could be induced by stimulating colon 26 cells with an inflammatory cytokine, interleukin-1, in vitro. Moreover, intravenous administration of lipopolysaccharide in mice selectively induced ADAMTS-1 mRNA in kidney and heart. These data suggest that ADAM-TS-1 may be a gene whose expression is associated with various inflammatory processes as well as development of cancer cachexia.

Thrombospondin is a 450 kDa, multifunctional adhesive glycoprotein released from activated platelets and secreted by growing cells. It binds to components of the cell surface and extracellular milieu. Thrombospondin probably modulates a number of processes, including aggregation of platelets, formation and lysis of fibrin, adhesion and migration of cells, and progression of cells through the growth cycle. Some data indicate that tumor cell production of TSP1 can exert a significant inhibitory effect on tumor progression, which may be attributable in part to a reduction in angiogenesis. Other studies show that activation of latent TGF-beta is mediated by two sequences present in the type I repeats of TSP1, a sequence (GGWSHW) that binds active TGF-beta and potentially orients the TSP molecule, and a second sequence (RFK) that activates latent TGF-beta. Peptides based on these sites have potential therapeutic applications for the modulation of TGF-beta activation. TSP is a homotrimer with a number of functional domains, at least four of which might serve as receptor recognizing regions. The amino-terminal heparin binding domain interacts with heparin, other glycosaminoglycans and glycolipids and likely recognizes specific cell surface proteoglycans. The central disulfide cross-linked region, 210 kDa non-reduced and 70 kDa reduced, contains a peptide motif CSVTCG which is apparently responsible for binding to glycoprotein IV (CD36) with high affinity. Immediately adjacent to the calcium binding region of TSP, which undergoes considerable molecular relaxation in the absence of calcium, is an RGDA sequence. TSP has been demonstrated to bind to integrins of the alpha v beta 3 and alpha IIb beta 3 class. The carboxy-terminal region of TSP also contains at least one binding epitope for a cell receptor. There are 2 well characterized genes for TSP and truncated forms of TSP have been detected which have inhibitory effects on angiogenesis. Finally, TSP can interact with fibrinogen and fibronectin, perhaps on cellular surfaces, which might serve as secondary receptor-like mechanisms for TSP binding and subsequent mediation of cell adhesion. Hemorrhagic toxins are metalloproteases found in some snake venoms which, depending on the member of the family, proteolyze extracellular matrix or basement membrane proteins. This degradation of the blood vessel wall leads to leakage of RBCs to the surrounding tissues. One member also has fibrin as a substrate. These characteristics combined with the TSR could make this gene important in coagulation, either formation or lysis. There also could be wound healing and tumor metastasis implications.

In specific embodiments, polypeptides of the invention comprise the following amino acid sequence:

| | |
|---|---|
| QSRAGQRGXALPTRTKAPSLRALLRAVSPGAP, | (SEQ ID NO:563) |
| KLVVCFTLFLSKGFSIIVWTVLKVTGSVAHSFAFTVLPSILWDWVVTFPHRVLDSSTARSRTVSPRPPHSPASMKRRIKMLSVGFV | (SEQ ID NO:564) |
| HTLAVDTPFPFSTAGAIRPREVFGLTHQHPTRGAGTPQGATCAGCIRAVFGVLPKCKLALPVGIVRGAREIAWELYGILRLVHQTF | |
| PMTIIQK, | |
| TLAVDTPFPFSTAGAIRPREVFGLTHQHPTR, | (SEQ ID NO:565) |
| FLAPGFTLQ, | (SEQ ID NO:566) |
| DLAHCFYSGTVNGD, | (SEQ ID NO:567) |
| SAAALSLCEGVRGAFYL, | (SEQ ID NO:568) |
| RYVETMLVADQSMA, | (SEQ ID NO:569) |
| FHGSGLKHYLLTLFSVAAR, | (SEQ ID NO:570) |
| EQKGPEVTSNAALTLRNFC, | (SEQ ID NO:571) |
| EHYDTAILFTRQDLCGS, | (SEQ ID NO:572) |
| MADVGTVCDPSRSCSVIEDDGLQAAFTTAHELGHVFNMPHDDAK, | (SEQ ID NO:573) |
| LDHSQPWSPCSAYM, | (SEQ ID NO:574) |
| TSFLDNGHGECLMDKPQNPI, | (SEQ ID NO:575) |
| YDANRQCQFT, | (SEQ ID NO:576) |
| SKHCPDAASTC, | (SEQ ID NO:577) |
| TLWCTGTSGG, | (SEQ ID NO:578) |
| LVCQTKHFPWADGTSCGEGKWC, | (SEQ ID NO:579) |
| WGPWGDCSRTCGGGVQYTMRECDNPVPKNGGKYCEGKRVRYRSCN, | (SEQ ID NO:580) |
| DCPDNNGKTFREEQCEAHNEFSKASFG, | (SEQ ID NO:581) |
| PKYAGVSPKDRCKL, | (SEQ ID NO:582) |
| AKGIGYFF, | (SEQ ID NO:583) |
| VLQPKVVDGTPCSPDSTSVCVQGQCVKAGCDRIIDSKKKFDKCGVCGGNGSTCKK, | (SEQ ID NO:584) |
| RNQRGSRNNGSFLAI, | (SEQ ID NO:585) |
| GATNIEVK, | (SEQ ID NO:586) |
| AADGTYILNG, | (SEQ ID NO:587) |
| VLRYSGSSAALERIRSFSPL KEPLTIQVL, | (SEQ ID NO:588) |
| WVIEEWGECSK, | (SEQ ID NO:589) |
| PASECAKEVKPASTRPCAD, | (SEQ ID NO:590) |
| CSKTCGKGYKKR, | (SEQ ID NO:591) |
| and/or | |
| ESCDPLKKPKH. | (SEQ ID NO:592) |

expressed in bladder, kidney, and ovary. Therefore, polynucleotides and polypeptides of the invention are useful as reagents for differential identification of the tissue(s) or cell type(s) present in a biological sample and for diagnosis of diseases and conditions which include, but are not limited to, urogenital, renal, immune, or reproductive disorders, particularly cancers. Similarly, polypeptides and antibodies directed to these polypeptides are useful in providing immunological probes for differential identification of the tissue(s) or cell type(s). For a number of disorders of the above tissues or cells, particularly of the immune and gastrointestinal systems, expression of this gene at significantly higher or lower levels may be routinely detected in certain tissues or cell types (e.g., urogenital, renal, reproductive, immune, hematopoietic, and cancerous and wounded tissues) or bodily fluids (e.g., serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 179 as residues: Gly-8 to Leu-14, Met-18 to Phe-30. The tissue distribution in bladder, kidney, and ovary combined with the homology to the highly conserved thrombospondin protein indicates that the protein product of this gene is useful for the treatment and diagnosis of a variety of blood-related diseases, particularly immune responses to proliferative disorders of urogenital, renal, or reproductive tissues. Moreover, the tissue distribution in kidney indicates that this gene or gene product could be used in the treatment and/or detection of kidney diseases including renal failure, nephritus, renal tubular acidosis, proteinuria, pyuria, edema, pyelonephritis, hydronephritis, nephrotic syndrome, crush syndrome, glomerulonephritis, hematuria, renal colic and kidney stones, in addition to Wilm's Tumor Disease, and congenital kidney abnormalities such as horseshoe kidney, polycystic kidney, and Falconi's syndrome. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:74 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 4698 of SEQ ID NO:74, b is an integer of 15 to 4712, where both a and b correspond to the positions of nucleotide residues shown in SEQ ID NO:74, and where b is greater than or equal to a+14.

Features of Protein Encoded by Gene No: 65

In specific embodiments, polypeptides of the invention comprise the following amino acid sequence:

| | |
|---|---|
| THASGQESLYKICKAYSVYDEDIGYCQGQSFLAAVLLLHMPEEQAFCVLVKIMYDYGLRDLYRNNFEDLHCKFYQLERLMQEQLPD | (SEQ ID NO:593) |
| LHSHFSDLNLEAH, | |
| YSVYDEDIGYCQGQSFLAAVLLLH, | (SEQ ID NO:594) |
| and/or | |
| LYRNNFEDLHCKFYQLERLMQEQLPD. | (SEQ ID NO:595) |

invention.

The gene encoding the disclosed cDNA is believed to reside on chromosome 1. Accordingly, polynucleotides related to this invention are useful as a marker in linkage analysis for chromosome 1. This gene is expressed primarily in tonsil, placenta, and fetal tissues. Therefore, polynucleotides and polypeptides of the invention are useful as reagents for differential identification of the tissue(s) or cell type(s) present in a biological sample and for diagnosis of diseases and conditions which include, but are not limited to, immune, developmental, reproductive, or vascular disorders. Similarly, polypeptides and antibodies directed to these polypeptides are useful in providing immunological probes for differential identification of the tissue(s) or cell type(s). For a number of disorders of the above tissues or cells, particularly of the immune and reproductive systems, expression of this gene at significantly higher or lower levels may be routinely detected in certain tissues or cell types (e.g., immune, developmental, reproductive, vascular, and cancerous and wounded tissues) or bodily fluids (e.g., serum, amniotic fluid, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 180 as residues: Ala-72 to Arg-77, Val-88 to Tyr-117. The tissue distribution in tonsil indicates that the protein product of this gene is useful for the diagnosis and treatment of diseases of the immune system including many cancers such as lymphomas, leukemias, lymphocytomas, and the like. Moreover, the expression within fetal tissue and other cellular sources marked by proliferating cells indicates that this protein may play a role in the regulation of cellular division, and may show utility in the diagnosis and treatment of cancer and other proliferative disorders. Similarly, developmental tissues rely on decisions involving cell differentiation and/or apoptosis in pattern formation. Thus this protein may also be involved in apoptosis or tissue differentiation and could again be useful in cancer therapy. The protein may also be useful for the treatment, detection, and/or prevention of a variety of vascular disorders which include, but are not limited to, embolism, atherosclerosis, microvascualr disease, stroke, or aneurysm. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:75 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1633 of SEQ ID NO:75, b is an integer of 15 to 1647, where both a and b correspond to the positions of nucleotide residues shown in SEQ ID NO:75, and where b is greater than or equal to a+14.

Features of Protein Encoded by Gene No: 66

Polypeptides encoded by this gene share homology to steroid/thyroid hormone orphan nuclear receptor and to several additional orphan nuclear receptors isolated from different tissues, such as in T-cells and the brain. Sequence analysis of the 5′ flanking region surrounding +1 revealed several possible response elements such as a hexanucleotide glucocorticoid binding site, a cAMP-response element, a CArG box, and two c-Jun-binding sites.

In specific embodiments, polypeptides of the invention comprise the following amino acid sequence:

| | |
|---|---|
| QKMYETMKLDACXHQQRPTLQAGPKLLTLAPREEPRGQSGRGSELTARQRHSTGDPQGEQALPRAGCVTGPPATPHRPSEPQLLRT | (SEQ ID NO:596) |
| HPDARPKSAMAQTFVHQGPVALQQLTTNRRVETSMSSDGHGQNPTPSPWADVCASRADAVAFPASGXCHSPWLMXPSSHPLNPHSP | |
| LNLPPPSFHCKDPVMTLHPQTLVTQGHLSTSGRLT, | |
| GPKLLTLAPREEPRGQSGRGSELTARQR, | (SEQ ID NO:597) |
| PKSAMAQTFVHQGPVALQQLTTNRRVETS, | (SEQ ID NO:598) |
| RADAVAFPASGXCHSPWLMXPSSHPLNPH, | (SEQ ID NQ:599) |
| and/or | |
| PPSFHCKDPVMTLHPQTLVTQGHLSTSG. | (SEQ ID NO:600) |

invention.

This gene is expressed primarily in testis. Therefore, polynucleotides and polypeptides of the invention are useful as reagents for differential identification of the tissue(s) or cell type(s) present in a biological sample and for diagnosis of testicular tumors, impotence, and other reproductive disorders. Similarly, polypeptides and antibodies directed to these polypeptides are useful in providing immunological probes for differential identification of the tissue(s) or cell type(s). For a number of disorders of the above tissues or cells, particularly of the reproductive system, expression of this gene at significantly higher or lower levels may be routinely detected in certain tissues or cell types (e.g., reproductive, testicular, and cancerous and wounded tissues) or bodily fluids (e.g., lymph, serum, plasma, seminal fluid, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in testes, combined with the homology to steroid/thyroid hormone orphan nuclear receptors indicates that the protein product of this gene is useful for the treatment and diagnosis of diseases in the male reproductive system such as tumors of the testis and other reproductive disorders. As nuclear receptors have the ability to regulate expression of other genes, the protein product of this gene may be useful as a contraceptive by inhibiting the expression of a key determinant in sperm maturation. Furthermore, the protein may serve to inhibit other vital functions in cells of varying tissues and cell types. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:76 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 876 of SEQ ID NO:76, b is an integer of 15 to 890, where both a and b correspond to the positions of nucleotide residues shown in SEQ ID NO:76, and where b is greater than or equal to a+14.

Features of Protein Encoded by Gene No: 67

The translation product of this gene has a high degree of sequence identity with CTGF-4. The protein product of this gene was shown to inhibit adhesion to peripheral blood mononuclear cells.

In specific embodiments, polypeptides of the invention comprise the following amino acid sequence:

| | |
|---|---|
| MDSMPEPASRCLLLLPLLLLLLLLLPAPELGPSQAGAEENDWVRLPSKCEVCKYVAVELKVKPLRKRQDTEVIGTVYGILDQKASG | (SEQ ID NO:601) |
| VKYTKSDLRLIEVTETICKRLLDYSLHKERTGSXRFAKGMSETFETLHXLVHKGVKVVMDIPYELWNETSAEVADLKKQCDVLVEEFE | |
| EVIEDWYRNHQEEDLTEFLCANHVLKGKDTSCLAEQWSGKKGDTAALGGKKSKKKSIRAKAAGGRSSSSKQRKELGGLEGDPSPEE | |
| DEGIQKASPLTHSPPDEL, | |
| LLSWLLGGTCAVPEAARGNCSARAAGGGTARSFRVRARPG, | (SEQ ID NO:602) |
| EGMPSGCPHPPRGWGLPQGHPAPSFVCCCYSCRLLPWPXCSSSWTSSLPGQLCLPSCRTTALPGNWCLFPSARGWRRGIQSGLPPG | (SEQ ID NO:603) |
| GXCTSPRSPPQTLPPAHHTAS, | |
| RTTALPGNWCLFPSARGWRRGIQSGL, | (SEQ ID NO:604) |
| LLSFKIRGLRTEDAGWAQSSSGGLCVRGDAFWMPSSSSGLGSPSRPPSSFLCLLLLLLPPAALALXLFFLDFFPPRAAVSPFLPDH | (SEQ ID NO:605) |
| CSARQLVSFPFSTWLAQRNSVRSSSWWFLYQSSITSSNSSTSTSHCFLRSATSAEVSFHSS, | |
| AAVSPFLPDHCSARQLVSFPFSTWLAQRNSVRS, | (SEQ ID NO:606) |
| TLGQERNTWGGERTAWATHDGSEFFLGLVSFLGRRGHSQGKEGLKAAGPSRPARAMPLTPQLQNQGSQDRGCWVGSELIRGAVCER | (SEQ ID NO:607) |
| GCLLDALILLGAGVSLKATQLLPLFAAATPAACCLGPXALLLGLLPSQGSCVSLLAGPLLCQATGVFSLQHVVGAEEFSQVFLLVV | |
| XVPVLDHLLKLFHQHITLLLEVSHLCRSLVPQLIGDVHHHLDPFVYQVV, | |
| TWGGERTAWATHDGSEFFLGLVSFLG, | (SEQ ID NO:608) |
| TAWATHDGSEFFLGLVSFLGRRGH, | (SEQ ID NO:609) |
| ARAMPLTPQLQNQGSQDRGCWVGSELIRGAVCE, | (SEQ ID NO:610) |
| SQGSCVSLLAGPLLCQATGVFSLQHVV, | (SEQ ID NO:611) |
| EPFARGSWIIACTRRGPAAIDLPRACQRPLRHYTTWYTKGSRW, | (SEQ ID NO:612) |
| and/or | |
| GERLGSPAQQMRRDLRLIEVTENHLQEAPGL. | (SEQ ID NO:613) |

the invention.

This gene is expressed in fetal liver/spleen, amniotic cells, and human testes. Therefore, polynucleotides and polypeptides of the invention are useful as reagents for differential identification of the tissue(s) or cell type(s) present in a biological sample and for the diagnosis of cancers, immunological disorders, and neural diseases (such as spinocerebellar ataxia, bipolar affective disorder, schizophrenia, and autism), and other diseases featuring anticipation, neurodegeneration, or abnormalities of neurodevelopment. Similarly, polypeptides and antibodies directed to these polypeptides are useful in providing immunological probes for differential identification of the tissue(s) or cell type(s). For a number of disorders of the above tissues or cells, particularly of the nerve system, immune system, expression of this gene at significantly higher or lower levels may be routinely detected in certain tissues or cell types (e.g., immune, neural, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 182 as residues: Ser-3 to Ser-9, Gly-36 to Val-43, Leu-45 to Gly-51. The tissue distribution in fetal liver/spleen combined with the homology to the CTGF-4 protein indicates that the protein product of this gene is useful for the diagnosis and treatment of a variety of immune system disorders. Expression of this gene product indicates a role in regulating the proliferation; survival; differentiation; and/or activation of hematopoietic cell lineages, including blood stem cells. This gene product may be involved in the regulation of cytokine production, antigen presentation, or other processes that may also suggest a usefulness in the treatment of cancer (e.g. by boosting immune responses). Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues. In addition, this gene product may have commercial utility in the expansion of stem cells and committed progenitors of various blood lineages, and in the differentiation and/or proliferation of various cell types. Moreover, the expression within fetal tissue and other cellular sources marked by proliferating cells indicates that this protein may play a role in the regulation of cellular division, and may show utility in the diagnosis and treatment of cancer and other proliferative disorders. Similarly, developmental tissues rely on decisions involving cell differentiation and/or apoptosis in pattern formation. Thus this protein may also be involved in apoptosis or tissue differentiation and could again be useful in cancer therapy. Moreover, the protein product of this gene may also show utility in the treatment, diagnosis, or prevention of a variety of neural disorders, which include, but are not limited to anticipation, neurodegeneration, or abnormalities of neurodevelopment. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:77 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1643 of SEQ ID NO:77, b is an integer of 15 to 1657, where both a and b correspond to the positions of nucleotide residues shown in SEQ ID NO:77, and where b is greater than or equal to a+14.

Features of Protein Encoded by Gene No: 68

Polypeptides encoded by polynucleotides comprising this gene contain a zinc finger homology domain. Such motifs are believed to be important for protein interactions, particularly with regard to gene regulation. In specific embodiments, polypeptides of the invention comprise the following amino acid sequence: WPRLKGWRRC (SEQ ID NO:614). Polynucleotides encoding these polypeptides are also encompassed by the invention. This gene is expressed primarily in T cells and the colon and, to a lesser extent, in the testes and placenta. Therefore, polynucleotides and polypeptides of the invention are useful as reagents for differential identification of the tissue(s) or cell type(s) present in a biological sample and for diagnosis of many immune, reproductive, or digestive disorders. Similarly, polypeptides and antibodies directed to these polypeptides are useful in providing immunological probes for differential identification of the tissue(s) or cell type(s). For a number of disorders of the above tissues or cells, particularly of the immune and digestive systems, expression of this gene at significantly higher or lower levels may be routinely detected in certain tissues or cell types (e.g., immune, reproductive, digestive, and cancerous and wounded tissues) or bodily fluids (e.g., lymph, serum, bile, seminal fluid, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 183 as residues: Pro-12 to Lys-33, Asn-41 to His-46, Pro-48 to Ser-58, Gly-71 to Asp-78, Ala-94 to Gly-102, Ser-133 to Ser-140, Arg-197 to Lys-202. The tissue distribution of this gene in T-cells indicates a potential role in the treatment and detection of immune disorders such as arthritis, asthma, immune deficiency diseases (such as AIDS), and leukemia. Expression of this gene in the colon indicates a potential role in the treatment and detection of colon disorders such as ulcers and colon cancer, in addition to digestive disorders in general. Moreover, the protein product of this gene may be useful on the detection, treatment, and/or prevention of normal testicular function. In addition, this gene product may be useful in the treatment of male infertility, and/or could be used as a male contraceptive. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:78 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 2001 of SEQ ID NO:78, b is an integer of 15 to 2015, where both a and b correspond to the positions of nucleotide residues shown in SEQ ID NO:78, and where b is greater than or equal to a+14.

Features of Protein Encoded by Gene No: 69

The translation product of this gene shares sequence homology with neuroendocrine protein, which is thought to be important in neuronal development and differentiation.

In specific embodiments, polypeptides of the invention comprise the following amino acid sequence:

| | |
|---|---|
| MDGQKKNWKDKVVDLLYWRDIKKTGVVFGASLFLLLSLTVFSIVSVTAYIALALLSVTISFRIYKGVIQAIQKSDEGHPFRAYLES | (SEQ ID NO:615) |
| EVAISEELVQKYSNSALGHVNCTIKELRRLFLVDDLVDSLKFAVLMWVFTYVGALFNGLTLLILALISLFSVPVIYERHQAQIDHYL | |
| GLANKNVKDAMAKIQAKIPGLKRKAE, | |
| KNWKDKVVDLLYWRDIKKTGVVFGASLFLLLS, | (SEQ ID NO:616) |
| RAYLESEVAISEELVQKYSNSALGHV, | (SEQ ID NO:617) |
| LISLFSVPVIYERHQAQIDHYLGLANKNV, | (SEQ ID NO:618) |
| QEMDGQKKNWKDKVVDLLYWRDIKKTGVVFGASLFLLLSLTVFSIVSVTAYIALALLSVTISFRIYKGVIQAIQKSDEGHPFRAYLE | (SEQ ID NO:619) |
| SEVAISEELVQKYSNSALGHVNCTIKELRRLFLVDDLVDSLKFAVLMWVFIYVGALFNGLTLLILALISLFSVPVIYERHQAQIDH | |
| YLGLANKNVKDAMAKIQAKIPGLKRKAE, | |
| DIKKTGVVFGASLFLLLSLTVFSIVSVTAYIALA, | (SEQ ID NO:620) |
| DEGHPFRAYLESEVAISEELVQKYSNSA, | (SEQ ID NO:621) |
| and/or | |
| NGLTLLILALISLFSVPVIYERHQAQIDHYL. | (SEQ ID NO:622) |

Polynucleotides encoding these polypeptides are also encompassed by the invention.

This gene is expressed primarily in brain, and, to a lesser extent, in fetal tissue, placenta, bone marrow, and stromal cells. Therefore, polynucleotides and polypeptides of the invention are useful as reagents for diagnosis of neurodegenerative diseases, immune, hematopoietic, or developmental disorders. Similarly, polypeptides and antibodies directed to these polypeptides are useful in providing immunological probes for differential identification of the tissue(s) or cell type(s). For a number of disorders of the above tissues or cells, particularly of the nervous system and during development, expression of this gene at significantly higher or lower levels may be routinely detected in certain tissues or cell types (e.g., neural, immune, hematopoietic, developmental, and cancerous and wounded tissues) or bodily fluids (e.g., lymph, serum, amniotic fluid, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 184 as residues: Gln-47 to Gly-52, Leu-169 to Glu-174. The predominant tissue distribution in brain, combined with the homology to the neuroendocrine protein indicates that the protein product of this gene is useful for the diagnosis and treatment of neurodegenerative diseases and behavioral disorders such as Alzheimer's Disease, Parkinson's Disease, Huntington's Disease, schizophrenia, mania, dementia, paranoia, obsessive-compulsive disorder and panic disorder. Moreover, the expression within fetal tissue and other cellular sources marked by proliferating cells (i.e. placenta) indicates that this protein may play a role in the regulation of cellular division, and may show utility in the diagnosis and treatment of cancer and other proliferative disorders. Similarly, developmental tissues rely on decisions involving cell differentiation and/or apoptosis in pattern formation. Thus, this protein may also be involved in apoptosis or tissue differentiation and could again be useful in cancer therapy. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:79 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1199 of SEQ ID NO:79, b is an integer of 15 to 1213, where both a and b correspond to the positions of nucleotide residues shown in SEQ ID NO:79, and where b is greater than or equal to a+14.

Features of Protein Encoded by Gene No: 70

Polypeptides encoded by polynucleotides comprising this gene share sequence identity with human hepatoma-derived growth factor (WPI 95-069304/10). As such, polynucleotides comprising this gene can be used for the recombinant production of the protein, which can be used to encourage the growth of various animal cells, and for the purification of receptors.

| | |
|---|---|
| MAVTLSLLLGGRVCA; | (SEQ ID NO:623) |
| PSLAVGSRPGGWRAQALLAGSRTPIPTGSRRNGS | (SEQ ID NO:624) |
| CRRWRAP; | |
| and | |
| MAVTLSLLLGGRVCAPSLAVGSRPGGWRAQALLA | (SEQ ID NO:625) |
| GSRTPIPTGSRRNGSCRRWRAP, | |
| QRPTAEGGLRRHGGYPESLAGRARLRAVTRCGFA | (SEQ ID NO:626) |
| TRGVAGPGPIGREPDPDSDWEPEERELQEVESTL | |
| KRQKQAIRFQKIRRQMEAPGAPPRTLTWEAMEQI | |
| RYLHEEFPESWSVPRLAEGFDVSTDVIRRVLKSK | |
| FLPTLEQKLKQDQKVLKKAGLAHSLQHLRGSGNT | |
| SKLLPAGHSVSGSLLMPGHEASSKDPNHSTALKV | |
| IESDTHRTNTPRRRKGRNKEIQDLEESFVPVAAP | |
| LGHPRELQKYSSDSESPRGTGSGALPSGQKLEEL | |
| KAEEPDNFSSKVVQRGREFFDSNGNFLYRI, | |
| LAGRARLRAVTRCGFATRGVAGPGPI, | (SEQ ID NO:627) |
| APPRTLTWEAMEQIRYLHEEFPESWSVP, | (SEQ ID NO:628) |
| QKVLKKAGLAHSLQHLRGSGNTSKLLPAGHSV, | (SEQ ID NO:629) |
| and/or | |
| PVAAPLGHPRELQKYSSDSESPRGTGSG. | (SEQ ID NO:630) |

the invention.

The gene encoding the disclosed cDNA is believed to reside on chromosome 15. Accordingly, polynucleotides related to this invention are useful as a marker in linkage analysis for chromosome 15. This gene is expressed primarily in brain, and to a lesser extent, in endotheilium, T cell, and tumors. Therefore, polynucleotides and polypeptides of the invention are useful as reagents for differential identification of the tissue(s) or cell type(s) present in a biological sample and for diagnosis of many neurodegenerative diseases (for example, Alzheimer's Disease, ALS, and the like) and cancers (including, but not limited to neuroblastoma, glioblastoma, Schwannoma, astrocytoma, and the like). Similarly, polypeptides and antibodies directed to these polypeptides are useful in providing immunological probes for differential identification of the tissue(s) or cell type(s). For a number of disorders of the above tissues or cells, particularly of the nervous system, expression of this gene at significantly higher or lower levels may be routinely detected in certain tissues or cell types (e.g., neural, integumentary, immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 185 as residues: Thr-42 to Pro-56. The tissue distribution in brain indicates that the protein product of this gene is useful for the treatment and diagnosis of many neurodegenerative diseases and cancers. Similarly, the protein product of this gene is useful for the detection/treatment of neurodegenerative disease states, behavioral disorders, or inflammatory conditions which include, but are not limited to Alzheimer's Disease, Parkinson's Disease, Huntington's Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, depression, panic disorder, learning disabilities, ALS, psychoses, autism, and altered behaviors, including disorders in feeding, sleep patterns, balance, and perception. In addition, elevated expression of this gene product in regions of the brain indicates that it plays a role in normal neural function. Potentially, this gene product is involved in synapse formation, neurotransmission, learning, cognition, homeostasis, or neuronal differentiation or survival. Moreover, the expression within epithelium indicates that the protein product of this gene is useful for the treatment, diagnosis, and/or prevention of various skin disorders, which include, but are not limited to, congenital disorders (i.e. nevi, moles, freckles, Mongolian spots, hemangiomas, port-wine syndrome), integumentary tumors (i.e. keratoses, Bowen's disease, basal cell carcinoma, squamous cell carcinoma, malignant melanoma, Paget's disease, mycosis fungoides, and Kaposi's sarcoma), injuries and inflammation of the skin (i.e. wounds, rashes, prickly heat disorder, psoriasis, dermatitis), atherosclerosis, uticaria, eczema, photosensitivity, autoimmune disorders (i.e. lupus erythematosus, vitiligo, dermatomyositis, morphea, scleroderma, pemphigoid, and pemphigus), keloids, striae, erythema, petechiae, purpura, and xanthelasma. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:80 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1377 of SEQ ID NO:80, b is an integer of 15 to 1391, where both a and b correspond to the positions of nucleotide residues shown in SEQ ID NO:80, and where b is greater than or equal to a+14.

Features of Protein Encoded by Gene No: 71

The translation product of this gene shares sequence homology with acrosin, trypsin, as well as trypsinogen precursor, which are thought to be important in cell-cell recognition and proteinase activity for protein cleavage and degradation.

In specific embodiments, polynucleotides of the invention comprise the following sequence:

| | |
|---|---|
| gatgttacacagctctttaataatagtggccata | (SEQ ID NO:631) |
| gctgtaataacaatgacaacagtaggtaacggta | |
| gtcataccaacagtagggcagtgcattttatatt | |
| acaactggtttcttgctctagtaggcttggggat | |
| gggtgaagacggacagggctggcgcagacccttt | |
| ccttctcctctccagcccacagtgatctgggctt | |
| ttacaagacagcctgcttccattcagtagtgtgg | |
| gaaagttccttcttggcttagcaatacccctgag | |
| accttgttcagtgggctgtgtctctccctgggat | |
| gctgggagcaccaagtgtggccgagctagggctg | |
| ctgacttcctctgggcgcctctgggctgcgaggg | |
| tctcttataggaattgaggccctttgctgctcca | |
| agaaatgctgaggctgtgggcaragggktgtacc | |
| caaggggactcttgctctgtgtctgactttgggg | |
| ratcc, | |
| cacagctctttaataatagtggccatagctgtaa | (SEQ ID NO:632) |
| taacaatgacaacagtaggtaacg, | |
| tgtgtctctccctgggatgctgggagcaccaagt | (SEQ ID NO:633) |
| gtggccgagctagggctgctgactt, | |
| gcgagggtctcttataggaattgaggccctttgc | (SEQ ID NO:634) |
| tgctccaagaaatgctgaggctgtgggcaraggg | |
| ktgtacccaaggggact. | |

In specific embodiments, polypeptides of the invention comprise the following amino acid sequence:

| | |
|---|---|
| CCCPFAVLGAASFLPCPPGDXPKSDTEQESPWVX | (SEQ ID NO:635) |
| PXAHSLSISWSSKGPQFL, | |
| CTNLTSGMWWASLAGAMAAGARAPQEYTPRSQPI | (SEQ ID NO:636) |
| STGSTMSGRLSCNAAAPLQCWEPLPSCPAHLGIP | |
| QSQTQSKSPLGYXPLFTASAFLGAAKGLNSYKRP | |
| SQPRGAQRKSAALARPHLVLPASQGETQPTEQGL | |
| RGIAKPRRNFPTLLNGSRLSCKSPDHCGLERRRK | |
| GSAPALSVFTHPQAY, | |
| LQCWEPLPSCPAHLGIPQSQTQSKSPLGYXPL, | (SEQ ID NO:637) |
| PTEQGLRGIAKPRRNFPTLLNGSRLSCKS, | (SEQ ID NO:638) |
| PQLTMPTTCHWSDWYIRGPPLSPWQVSTPPSGMP | (SEQ ID NO:639) |
| AHIIFSVTSPWYASSAXHRVLSMTWTDACSSMSD | |
| IFPPFCFVKPHPMIQSGVAGVSSSSKKGRQMGLT | |
| VPEKVSGNCSFMRAMS, | |
| GPPLSPWQVSTPPSGMPAHIIFSVTSPWYAS, | (SEQ ID NO:640) |
| CFVKPHPMIQSGVAGVSSSSKKGR, | (SEQ ID NO:641) |
| AVMLLPLCSAGSRFLPALPTWGXPKVRHRARVPL | (SEQ ID NO:642) |
| GTXLCPQPQHFLEQQRASIPIRDPRSPEAPRGSQ | |
| QP, | |
| and/or | |
| AGSRFLPALPTWGXPKVRHRARVPLGTXLCPQPQ | (SEQ ID NO:643) |
| HF. | |

encompassed by the invention.

This gene is expressed primarily in cheek carcinoma, and to a lesser extent, in uterine and pancreatic cancers. Therefore, polynucleotides and polypeptides of the invention are useful as reagents for differential identification of the tissue(s) or cell type(s) present in a biological sample and for diagnosis of diseases and conditions which include, but are not limited to, integumentary, mucosal, gastrointestinal, digestive, or reproductive disorders, particularly cheek cancers or cancers of uterine and pancreatic origins. Similarly, polypeptides and antibodies directed to these polypeptides are useful in providing immunological probes for differential identification of the tissue(s) or cell type(s). For a number of disorders of the above tissues or cells, particularly of the neoplastic tissues, expression of this gene at significantly higher or lower levels may be routinely detected in certain tissues or cell types (e.g., integumentary, mucosal, gastrointestinal, digestive, reproductive, and cancerous and wounded tissues) or bodily fluids (e.g., lymph, serum, bile, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 186 as residues: Thr-39 to Gly-44. The tissue distribution in cheek, uterine, and pancreatic tumors, combined with the homology to acrosin and trypsin, indicates that polynucleotides and polypeptides corresponding to this gene are useful for the diagnosis and intervention of cancers. The homology to acrosin and trypsin may indicate the gene function in tumor metastasis or migration, since in both cases cell-cell interaction and extracellular matrix degradation may be involved. The gene product can also be used as a target for cancer immunotherapy or as a diagnostic marker. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:81 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 994 of SEQ ID NO:81, b is an integer of 15 to 1008, where both a and b correspond to the positions of nucleotide residues shown in SEQ ID NO:81, and where b is greater than or equal to a+14.

Features of Protein Encoded by Gene No: 72

In specific embodiments, polypeptides of the invention comprise the following amino acid sequence: PQDAGKAYSDRHMCSV (SEQ ID NO:644). Polynucleotides encoding these polypeptides are also encompassed by the invention. The gene encoding the disclosed cDNA is believed to reside on chromosome 3. Accordingly, polynucleotides related to this invention are useful as a marker in linkage analysis for chromosome 3. This gene is expressed primarily in T helper cells 1, T-cells stimulated with PHA for 24 hours, and in a placenta Nb2HP cDNA library. Therefore, polynucleotides and polypeptides of the invention are useful as reagents for differential identification of the tissue(s) or cell type(s) present in a biological sample and for diagnosis of many immunodeficiencies and disorders (especially autoimmune diseases), in addition to reproductive disorders. Similarly, polypeptides and antibodies directed to these polypeptides are useful in providing immunological probes for differential identification of the tissue(s) or cell type(s). For a number of disorders of the above tissues or cells, particularly of the immune system expression of this gene at significantly higher or lower levels may be routinely detected in certain tissues or cell types (e.g., immune, hematopoietic, reproductive, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in T-helper and T-cells indicates that the protein product of this gene is useful for the diagnosis and treatment of autoimmune diseases, immunodeficiencies, and other immune system disorders. This gene product may be involved in the regulation of cytokine production, antigen presentation, or other processes that may also suggest a usefulness in the treatment of cancer (e.g. by boosting immune responses). Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues. In addition, this gene product may have commercial utility in the expansion of stem cells and committed progenitors of various blood lineages, and in the differentiation and/or proliferation of various cell types. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:82 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1247 of SEQ ID NO:82, b is an integer of 15 to 1261, where both a and b correspond to the positions of nucleotide residues shown in SEQ ID NO:82, and where b is greater than or equal to a+14.

Features of Protein Encoded by Gene No: 73

When tested against Jurket cell lines, supernatants removed from cells containing this gene activated the GAS (gamma activating sequence) promoter element. Thus, it is likely that this gene activates immune cells, particularly T-cells, through the JAK-STAT signal transduction pathway. GAS (gamma activating sequence)—a promoter element found upstream of many genes which are involved in the Jak-STAT pathway. The Jak-STAT pathway is a large, signal transduction pathway involved in the differentiation and proliferation of cells. Therefore, activation of the Jak-STAT pathway, reflected by the binding of the GAS element, can be used to indicate proteins involved in the proliferation and differentiation of cells. The gene encoding the disclosed cDNA is believed to reside on chromosome 1. Accordingly, polynucleotides related to this invention are useful as a marker in linkage analysis for chromosome 1. This gene is expressed primarily in 7 week old early stage human, human chronic synovitis, and infant brain. Therefore, polynucleotides and polypeptides of the invention are useful as reagents for differential identification of the tissue(s) or cell type(s) present in a biological sample and for diagnosis of developmental, neural, or skeletal disorders, particularly chronic synovitis, or congenital defects. Similarly, polypeptides and antibodies directed to these polypeptides are useful in providing immunological probes for differential identification of the tissue(s) or cell type(s). For a number of disorders of the above tissues or cells, particularly of the synovium, expression of this gene at significantly higher or lower levels may be routinely detected in certain tissues or cell types (e.g., neural, skeletal, developmental, and cancerous and wounded tissues) or bodily fluids (e.g., lymph, amniotic fluid, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 188 as residues: Ser-44 to Pro-49. The tissue distribution in chronic synovitis, combined with the detected GAS biological activity indicates that the protein product of this gene is useful for the diagnosis and treatment of chronic synovitis and other disorders of the synovium, particularly inflammatory conditions. Moreover, the expression within brain indicates the protein product of this gene is useful for the detection/treatment of neurodegenerative disease states, behavioral disorders, or inflammatory conditions which include, but are not limited to Alzheimer's Disease, Parkinson's Disease, Huntington's Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, depression, panic disorder, learning disabilities, ALS, psychoses, autism, and altered behaviors, including disorders in feeding, sleep patterns, balance, and perception. In addition, elevated expression of this gene product in regions of the brain indicates that it plays a role in normal neural function. Potentially, this gene product is involved in synapse formation, neurotransmission, learning, cognition, homeostasis, or neuronal differentiation or survival. Moreover, the expression within embryonic tissue and other cellular sources marked by proliferating cells indicates that this protein may play a role in the regulation of cellular division, and may show utility in the diagnosis and treatment of cancer and other proliferative disorders. Similarly, developmental tissues rely on decisions involving cell differentiation and/or apoptosis in pattern formation. Thus, this protein may also be involved in apoptosis or tissue differentiation and could again be useful in cancer therapy. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:83 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1031 of SEQ ID NO:83, b is an integer of 15 to 1045, where both a and b correspond to the positions of nucleotide residues shown in SEQ ID NO:83, and where b is greater than or equal to a+14.

Features of Protein Encoded by Gene No: 74

Polypeptides encoded by polynucleotides comprising this gene exhibit sequence homology to a number of mucin-like extracellular or cell surface proteins.

In specific embodiments, polypeptides of the invention comprise the following amino acid sequence:

| | |
|---|---|
| MVGPVTLHKKIHTTTVLFIVQIHILLIQAITQ | (SEQ ID NO:645) |
| AK, | |
| LQMHLMILQMTGLSILALLGKSTTTIVEQKFHNG | (SEQ ID NO:646) |
| KNQKSGLKENRDKKKQTRWQSTASQKIGITEER; | |
| MVGPVTLHKKIHTTTVLFIVQIHILLIQAITQAK | (SEQ ID NO:647) |
| LQMHLMILQMTGLSILALLGKSTTTIVEQKFHNG | |
| KNQKSGLKENRDKKKQTRWQSTASQKIGITEER, | |
| GLQKRGDHRHEKMRDAGDPSPPNKMLRRSDSPEN | (SEQ ID NO:648) |
| KYSDSTGHSKAKNVHTHRVRERDGGTSYSPQENS | |
| HNHSALHSSNSHSSNPSNPSKTSDAPYDSADDWS | |
| EHISSSGKKYYYNCRTEVSQWEKPKEWLEREQRQ | |
| KEANKMAVNSFPKDRDYRREVMQATATSGFASGM | |
| EDKHSSDASSLLPQNILSQTSRHNDRDYRLPRAE | |
| THSSSTPVQHPIKPVVHPTATPSTVPSSPFTLQS | |
| DHQPKKSFDANGASTLSKLPTPTSSVPAQKTERK | |
| ESTSGDKPVSHSCTTPSTSSASGLNPTSAPPTSA | |
| SAVPVSPVPQSPIPPLLQDPNLLRQLLPALQATL | |
| QLNNSNVDISKINEVLTAAVTQASLQSIIHKFLT | |
| AGPSAFNITSLISQAAQLSTQAQPSNQSPMSLTS | |
| DASSPRSYVSPRISTPQTNTVPIKPLISTPPVSS | |
| QPKVSTPVVKQGPVSQSATQQPVTADKXQGHEPV | |
| SPRSLQRSSSQRSPSPGPNHTSNSSNASNATVVP | |
| QNSSARSTCSLTPALAAHFSENLIKHVQGWPADH | |
| AEKQASRLREEAHNMGTLHMSEICTELKNLRSLV | |
| RVCEIQATLREQRDTIFETTN, | |
| DAGDPSPPNKMLRRSDSPENKYSDSTGHSK, | (SEQ ID NO:649) |
| NHSALHSSNSHSSNPSNNPSKTSDAPYDSADDW, | (SEQ ID NO:650) |
| ANKMAVNSFPKDRDYRREVMQATATSGFASGME | (SEQ ID NO:651) |
| DK, | |
| VVHPTATPSTVPSSPFTLQSDHQPKKSFDANGA | (SEQ ID NO:652) |
| ST, | |
| SGDKPVSHSCTTPSTSSASGLNPTSAPPTSAS | (SEQ ID NO:653) |
| AV, | |
| ISKINEVLTAAVTQASLQSIIHKFLTAGPSAFNI | (SEQ ID NO:654) |
| TSL, | |
| PGPNHTSNSSNASNATVVPQNSSARSTCSLTP | (SEQ ID NO:655) |
| AL, | |
| LREEAHNMGTIHMSEICTELKNLRSLVRVC, | (SEQ ID NO:656) |
| NSARDKMAVNSFPKDRDYRREVITDMKRCETPEI | (SEQ ID NO:657) |
| LHHQIKCCGDLIVLKTNTVTAQVTVRPKMCILTE | |
| LERGMVGPVTLHKKIHTTTVLFIVQIHILLIQAI | |
| TQAKLQMHLMILQMTGLSILALLGKSTTTIVEQK | |
| FHNGKNQKSGLKENRDKKKQTRWQSTASQKIGIT | |
| EER, | |
| VITDMKRCETPEILHHQIKCCGDLIVLKTNTV, | (SEQ ID NO:658) |
| TVLFIVQIHILLIQAITQAKLQMHLMILQMT, | (SEQ ID NO:659) |
| STTTIVEQKFHNGKNQKSGLKENRDKKKQTRW | (SEQ ID NO:660) |
| QS, | |
| CKQQPLVGLPVEWKTSIPVMPVVCSHRIFCLKQA | (SEQ ID NO:661) |
| DTMTETTDCQEQRLTVVLRQYSTPSNQWFIQLLP | |
| QALFLLVHLRYSLITSQRNHLMLMEHLLYQNCLH | |
| PHLLSLHRKQKEKNLHQETNPYHILAQLLPRLLP | |
| LD, | |
| SIPVMPVVCSHRIFCLKQADTMTETTDCQEQ, | (SEQ ID NO:662) |
| PSNQWFIQLLPQALFLLVHLRYSLITSQR, | (SEQ ID NO:663) |
| and/or | |
| YQNCLHPHLLSLHRKQKEKNLHQETN. | (SEQ ID NO:664) |

invention.

The gene encoding the disclosed cDNA is believed to reside on chromosome 10. Accordingly, polynucleotides related to this invention are useful as a marker in linkage analysis for chromosome 10. This gene is expressed primarily in ovarian cancer, endometrial tumor, B-cell lymphoma, brain-medulloblastoma, hepatocellular tumor, osteosarcoma, and T- and B-cells. Therefore, polynucleotides and polypeptides of the invention are useful as reagents for differential identification of the tissue(s) or cell type(s) present in a biological sample and for diagnosis of diseases and conditions which include, but are not limited to, ovarian cancer, endometrial tumors, B-cell lymphoma, brain medulloblastoma, hepatocellular tumor, and osteosarcoma, and proliferative conditions in other tissues. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., reproductive, immune, hematopoietic, neural, skeletal, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 189 as residues: Lys-74 to Ala-101. The tissue distribution in proliferative tissues indicates that the protein product of this gene is useful for the diagnosis and treatment of ovarian cancer, endometrial tumors, B-cell lymphoma, brain medulloblastoma, hepatocellular tumor, and osteosarcoma. Expression within cellular sources marked by proliferating cells indicates that this protein may play a role in the regulation of cellular division, and may show utility in the diagnosis and treatment of cancer and other proliferative disorders. Similarly, developmental tissues rely on decisions involving cell differentiation and/or apoptosis in pattern formation. Thus this protein may also be involved in apoptosis or tissue differentiation and could again be useful in cancer therapy. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:84 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 2863 of SEQ ID NO:84, b is an integer of 15 to 2877, where both a and b correspond to the positions of nucleotide residues shown in SEQ ID NO:84, and where b is greater than or equal to a+14.

Features of Protein Encoded by Gene No: 75

In specific embodiments, polypeptides of the invention comprise the following amino acid sequence:

(SEQ ID NO:665)

MQTCPLVGTLLTRNMDGYTCAVVTSTSFWIISAWXLWKGSPSTSMPTMPE
TPLRTLCCTKMPSIFSSLMTDGRA.

Polynucleotides encoding these polypeptides are also encompassed by the invention. This polypeptide sequence has sequence homology with a Drosophila melanogaster male germ-line specific transcript which encodes a putative protamine molecule (see, gi|608696). The gene encoding the disclosed cDNA is believed to reside on chromosome 14. Accordingly, polynucleotides related to this invention are useful as a marker in linkage analysis for chromosome 14. This gene is expressed primarily in breast tissue, and to a lesser extent in, fetal and adult cells and tissues, especially those comprising endocrine organs. Therefore, polynucleotides and polypeptides of the invention are useful as reagents for differential identification of the tissue(s) or cell type(s) present in a biological sample and for diagnosis of diseases and conditions which include, but are not limited to, developmental and reproductive disorders, or defects. Similarly, polypeptides and antibodies directed to these polypeptides are useful in providing immunological probes for differential identification of the tissue(s) or cell type(s). For a number of disorders of the above tissues or cells, particularly of the female reproductive system, expression of this gene at significantly higher or lower levels may be routinely detected in certain tissues or cell types (e.g., reproductive, endocrine, reproductive, and cancerous and wounded tissues) or bodily fluids (e.g., lymph, serum, breast milk, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 190 as residues: Ser-100 to Gln-106. The tissue distribution in breast and fetal tissues indicates that the protein product of this gene is useful for the study and treatment of developmental, reproductive and growth and metabolic disorders. Similarly, the expression within embryonic tissue and other cellular sources marked by proliferating cells indicates that this protein may play a role in the regulation of cellular division, and may show utility in the diagnosis and treatment of cancer and other proliferative disorders. Similarly, developmental tissues rely on decisions involving cell differentiation and/or apoptosis in pattern formation. Thus, this protein may also be involved in apoptosis or tissue differentiation and could again be useful in cancer therapy. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:85 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1353 of SEQ ID NO:85, b is an integer of 15 to 1367, where both a and b correspond to the positions of nucleotide residues shown in SEQ ID NO:85, and where b is greater than or equal to a+14.

Features of Protein Encoded by Gene No: 76

In specific embodiments, polypeptides of the invention comprise the following amino acid sequence:

(SEQ ID NO:666)

MTLIQNCWYSWLFFGFFFHFLRKSISIFSIFLVCFRILALGPTCFLVWFW
KAFFRHILIFICLSREVFRPRCFLVYFR.

Polynucleotides encoding these polypeptides are also encompassed by the invention.

This polypeptide sequence has sequence homology with the MURF4 protein of Herpetomonas muscarum (See Genbank Accession No. S43288). Such RNA-editing enzymes may be useful as molecular targets in the intervention of the life cycle of trypanosomes and other protozoa. This gene is expressed primarily in fetal liver and spleen, osteosarcoma and bone marrow. Therefore, polynucleotides and polypeptides of the invention are useful as reagents for differential identification of the tissue(s) or cell type(s) present in a biological sample and for diagnosis of immune, hematopoietic, hepatic, or skeletal disorders, particularly liver tumors, osteosarcoma, and other cancers. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, hematopoietic, hepatic, skeletal, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in fetal liver and osteosarcoma, indicates that the protein product of this gene is useful for diagnosis of cancers such as liver tumor and osteosarcoma. Moreover, the expression within fetal liver/spleen and bone marrow indicates that the protein product of this gene is useful for the treatment and diagnosis of hematopoietic related disorders such as anemia, pancytopenia, leukopenia, thrombocytopenia or leukemia since stromal cells are important in the production of cells of hematopoietic lineages. The uses include bone marrow cell ex vivo culture, bone marrow transplantation, bone marrow reconstitution, radiotherapy or chemotherapy of neoplasia. The gene product may also be involved in lymphopoiesis, therefore, it can be used in immune disorders such as infection, inflammation, allergy, immunodeficiency etc. In addition, this gene product may have commercial utility in the expansion of stem cells and committed progenitors of various blood lineages, and in the differentiation and/or proliferation of various cell types. Furthermore, the expression within fetal tissue and other cellular sources marked by proliferating cells indicates that this protein may play a role in the regulation of cellular division, and may show utility in the diagnosis and treatment of cancer and other proliferative disorders. Similarly, developmental tissues rely on decisions involving cell differentiation and/or apoptosis in pattern formation. Thus this protein may also be involved in apoptosis or tissue differentiation and could again be useful in cancer therapy. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:86 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 996 of SEQ ID NO:86, b is an integer of 15 to 1010, where both a and b correspond to the positions of nucleotide residues shown in SEQ ID NO:86, and where b is greater than or equal to a+14.

Features of Protein Encoded by Gene No: 77

In specific embodiments, polypeptides of the invention comprise the following amino acid sequence:

| | |
|---|---|
| DFIYIYTHTHTHPKSFYIIKLSYYY, | (SEQ ID NO:667) |
| VLVCLGKKELIFKKSRLLHPCIFLCMLLKSL, | (SEQ ID NO:668) |
| and/or | |
| VMIRFYIIYTHTHTPQKLLYNQVVXLLLSFGLLR | (SEQ ID NO:669) |
| EERXNF. | |

encompassed by the invention.

The gene encoding the disclosed cDNA is believed to reside on chromosome 22. Accordingly, polynucleotides related to this invention are useful as a marker in linkage analysis for chromosome 22. This gene is expressed primarily in T cell lymphoma and monocytes. Therefore, polynucleotides and polypeptides of the invention are useful as reagents for differential identification of the tissue(s) or cell type(s) present in a biological sample and for diagnosis of immune or hematopoietic disorders, particularly T-cell lymphoma. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, hematopoietic and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in T-cell lymphoma and monocytes indicates that the protein product of this gene is useful for the diagnosis and treatment of proliferative conditions of blood cells, particularly T-cell lymphoma. Moreover, the expression of this gene product indicates a role in regulating the proliferation, survival, differentiation, and/or activation of hematopoietic cell lineages, including blood stem cells. This gene product may be involved in the regulation of cytokine production, antigen presentation, or other processes that may also suggest a usefulness in the treatment of cancer (e.g. by boosting immune responses). Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues. In addition, this gene product may have commercial utility in the expansion of stem cells and committed progenitors of various blood lineages, and in the differentiation and/or proliferation of various cell types. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:87 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1353 of SEQ ID NO:87, b is an integer of 15 to 1367, where both a and b correspond to the positions of nucleotide residues shown in SEQ ID NO:87, and where b is greater than or equal to a+14.

Features of Protein Encoded by Gene No: 78

| | |
|---|---|
| ILVDSFKLKL, | (SEQ ID NO:670) |
| LPFFLIHQVLTPLYLMTCTFRTAEYFPFYPCLHC | (SEQ ID NO:671) |
| SITFIQFLYGSSERXDSEDPSLKKQTIKCIHSDQ | |
| SKKRHIPSPLHTEKFGILRSP, | |
| MTCTFRTAEYFPFYPCLHCSITF, | (SEQ ID NO:672) |
| and/or | |
| SDQSKKRHIPSPLHTEKFGIL. | (SEQ ID NO:673) |

Polynucleotides encoding these polypeptides are also encompassed by the invention.

The gene encoding the disclosed cDNA is believed to reside on chromosome 8. Accordingly, polynucleotides related to this invention are useful as a marker in linkage analysis for chromosome 8. This gene is expressed primarily in tonsils, and a bone marrow cell line. Therefore, polynucleotides and polypeptides of the invention are useful as reagents for differential identification of the tissue(s) or cell type(s) present in a biological sample and for diagnosis of immunological or hematopoietic disorders. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in tonsils and bone marrow cell lines indicates that the protein product of this gene is useful for the diagnosis and treatment of immunological disorders. Moreover, the protein product of this gene is useful for the treatment and diagnosis of hematopoietic related disorders such as anemia, pancytopenia, leukopenia, thrombocytopenia or leukemia since stromal cells are important in the production of cells of hematopoietic lineages. The uses include bone marrow cell ex vivo culture, bone marrow transplantation, bone marrow reconstitution, radiotherapy or chemotherapy of neoplasia. The gene product may also be involved in lymphopoiesis, therefore, it can be used in immune disorders such as infection, inflammation, allergy, immunodeficiency etc. In addition, this gene product may have commercial utility in the expansion of stem cells and committed progenitors of various blood lineages, and in the differentiation and/or proliferation of various cell types. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:88 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1074 of SEQ ID NO:88, b is an integer of 15 to 1088, where both a and b correspond to the positions of nucleotide residues shown in SEQ ID NO:88, and where b is greater than or equal to a+14.

Features of Protein Encoded by Gene No: 79

In specific embodiments, polypeptides of the invention comprise the following amino acid sequence:

| | |
|---|---|
| MGTRAQVTPGRLPIPPPAPGLPFSAXEPLQGQLR | (SEQ ID NO:674) |
| RVSSSRGGFPGLALQLLRSETVKAYVNNEINILA | |
| SFF, | |
| MLVRTRPSQPLPLPGVGLGGPRSGDPPESTELRK | (SEQ ID NO:675) |
| GPGFLA, | |
| RRVSSSRGGFPGLALQLLRSETVKAYVNN, | (SEQ ID NO:676) |
| and/or | |
| YIYLIVYISFYSFRPQQL. | (SEQ ID NO:677) |

invention.

This gene is expressed primarily in brain, placenta, bone marrow, keratinocyte, fetal liver, and spleen. Therefore, polynucleotides and polypeptides of the invention are useful as reagents for differential identification of the tissue(s) or cell type(s) present in a biological sample and for diagnosis of neural, integumentary, immune, or hematopoietic disorders, particularly neurodegenerative conditions, and skin related diseases. Similarly, polypeptides and antibodies directed to these polypeptides are useful in providing immunological probes for differential identification of the tissue(s) or cell type(s). For a number of disorders of the above tissues or cells, particularly of the immune and skin system, expression of this gene at significantly higher or lower levels may be routinely detected in certain tissues or cell types (e.g., neural, integumentary, immune, hematopoietic, hepatic, developmental, reproductive, and cancerous and wounded tissues) or bodily fluids (e.g., lymph, serum, amniotic fluid, bile, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in brain and keratinocytes indicates that the protein product of this gene is useful for the diagnosis and treatment of many neural and skin related diseases. Alternatively, the expression within placenta and fetal tissues indicates that the protein product of this gene may be useful in the treatment, detection, or prevention of a variety of developmental or reproductive disorders. Moreover, the secreted protein can also be used to determine biological activity, to raise antibodies, as tissue markers, to isolate cognate ligands or receptors, to identify agents that modulate their interactions and as nutritional supplements. It may also have a very wide range of biological activities. Typical of these are cytokine, cell proliferation/differentiation modulating activity or induction of other cytokines; immunostimulating/immunosuppressant activities (e.g. for treating human immunodeficiency virus infection, cancer, autoimmune diseases and allergy); regulation of hematopoiesis (e.g. for treating anemia or as adjunct to chemotherapy); stimulation or growth of bone, cartilage, tendons, ligaments and/or nerves (e.g. for treating wounds, stimulation of follicle stimulating hormone (for control of fertility); chemotactic and chemokinetic activities (e.g. for treating infections, tumors); hemostatic or thrombolytic activity (e.g. for treating hemophilia, cardiac infarction etc.); anti-inflammatory activity (e.g. for treating septic shock, Crohn's disease); as antimicrobials; for treating psoriasis or other hyperproliferative diseases; for regulation of metabolism, and behavior. Also contemplated is the use of the corresponding nucleic acid in gene therapy procedures. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:89 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1847 of SEQ ID NO:89, b is an integer of 15 to 1861, where both a and b correspond to the positions of nucleotide residues shown in SEQ ID NO:89, and where b is greater than or equal to a+14.

Features of Protein Encoded by Gene No: 80

The translation product of this gene shares sequence homology with both the human and mouse RNA Polymerase I which is thought to be important in gene transcription and DNA repair processes (See Genbank Accession No. gi|2266929).

| | |
|---|---|
| LRCQLWLWRRSWCTIIHPLFR, | (SEQ ID NO:678) |
| PQRTVREPQRLIXFQRALSDQCMMISSSLSCGLA | (SEQ ID NO:679) |
| KKLTCSCTVSRALAKIMPSFHQWQQPVTGSCQTS | |
| PCLSPWKGRQLRS, | |
| IMPSFHQWQQPVTGSCQTSPCLSP, | (SEQ ID NO:680) |
| NSARARGEIEDGGFSGGGGNADRVVLGEFGVRNV | (SEQ ID NO:681) |
| HTTDFPGNYSGYDDAWDQDRFEKNFRVDVVHMDE | |
| NSLEFDMVGIDAAIANAFRRILLAEVPTMAVEKV | |
| LVYNNTSIVQDEILAHRLGLIPIHADPRLFEYRN | |
| QGDEEGTEIDTLQFRLQVRCTRNPHAAKDSSDPN | |
| ELYVNHKG, | |
| VLGEFGVRNVHTTDFPGNYSGYDDAWDQDRF, | (SEQ ID NO:682) |
| AEVPTMAVEKVLVYNNTSIVQDEILAHRLGLIPI | (SEQ ID NO:683) |
| HA, | |
| LQFRLQVRCTRNPHAAKDSSDPN, | (SEQ ID NO:684) |
| TMDVLLYTRTFSTAIVGTSASRIRRKALAMAASI | (SEQ ID NO:685) |
| PTMSNSSEFSSMCTTSTRKFFSKRSWSQASS, | |
| TAIVGTSASRIRRKALAMAASIP, | (SEQ ID NO:686) |
| PTMSNSSEFSSMCTTSTRKFFS, | (SEQ ID NO:687) |
| IRHELVERLKMAASQAVEEMRTAWFWGSLGFAMS | (SEQ ID NO:688) |
| ILLTFPVTIPVMMMPGTRTASRRISVWM, | |
| VERLKMAASQAVEEMRTAWFW, | (SEQ ID NO:689) |
| SILLTFPVTIPVMMMPGTRTASRRI, | (SEQ ID NO:690) |
| TTKADLFPEGTIRPVHDDILIAQLRPGQEIDLLM | (SEQ ID NO:691) |
| HCVKGIGKDHAKFSPVATASYRXLPDITLLEPVE | |
| GEAAEELSRCFSXGVIEVQEVQGKKVARVANPRL | |
| DTFSREIFRNEKLKKVVRLARVRDHYIFSVESTG | |
| VLPPDVLVSEAIKVLMGKCRRFLDELDAVQMD, | |
| PEGTIRPVHDDILIAQLRPGQEI, | (SEQ ID NO:692) |
| AKFSPVATASYRXLPDITLLEPV, | (SEQ ID NO:693) |
| KLKKVVRLARVRDHYIFSVE, | (SEQ ID NO:694) |
| and/or | |
| DVLVSEAIKVLMGKCRRFLDELDAV. | (SEQ ID NO:695) |

encoding these polypeptides are also encompassed by the invention.

This gene is expressed primarily in HEL cell lines and aorta endothelial cells and to a lesser extent in Jurkat T-cells. Therefore, polynucleotides and polypeptides of the invention are useful as reagents for differential identification of the tissue(s) or cell type(s) present in a biological sample and for diagnosis and treatment of vascular, immune, or hematopoietic disorders, particularly cancers and autoimmune diseases. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., vascular, immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 195 as residues: Lys-25 to Arg-32. The tissue distribution in endothelial and T-cells, combined with the strong homology to the human and mouse RNA polymerase I protein indicates that the protein product of this gene is useful for the treatment of immune diseases and cardiovascular diseases, which include, but are not limited to embolism, atherosclerosis, aneurysm, stroke, or microvascular disease. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:90 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1245 of SEQ ID NO:90, b is an integer of 15 to 1259, where both a and b correspond to the positions of nucleotide residues shown in SEQ ID NO:90, and where b is greater than or equal to a+14.

Features of Protein Encoded by Gene No: 81

The translation product of this gene shares sequence homology with human trichohylin, in addition to, a human DNA-binding protein (See Genbank Accession No. gi|2588783) which are thought to be important in gene regulation. In specific embodiments, polypeptides of the invention comprise the following amino acid sequence:

| | |
|---|---|
| MCPVCGRALSSPGSLGRHLLIHSEDQRSNCAVCGARFTSHATFNSEKLPEVLNMESLPTVHNEGPSSAEGKDIAFSPPVYPAGILL | (SEQ ID NO:696) |
| VCNNCAAYRKXLEAQTPSVXKWALRRQNEPLEVRLQRLERERTAKKSRRDNETPEEREVRRMRDREAKRLQRMQETDEQRARRLQR | |
| DREAMRLKRANETPEKRQARLIREREAKRLKRRLEKMDMMLRAQFGQDPSAMAALAAEMNFFQLPVSGVELDXQLLGKMAFEEQNS | |
| SXLH, | |
| IWNPYPQST, | (SEQ ID NO:697) |
| TLPVSPLPARLRDREQLIAHVYQHTAAVVSAKSY, | (SEQ ID NO:698) |
| SSPGSLGRHLLIHSEDQRSNCA, | (SEQ ID NO:699) |
| FNSEKLPEVLNMESLPTVHNEGPS, | (SEQ ID NO:700) |
| PAGILLVCNNCAAYRKXLEAQTPS, | (SEQ ID NO:701) |
| LEVRLQRLERERTAKKSRRDNE, | (SEQ ID NO:702) |
| EAKRLQRMQETDEQRARRLQRDR, | (SEQ ID NO:703) |
| KRQARLIREREAKRLKRRLEKMD, | (SEQ ID NO:704) |
| PLPARLRDREQLIAHVYQHTAAV, | (SEQ ID NO:705) |
| ARSLLTPSSCSHMQGLLRSTCNKPRPASGSKDRASWVAAALVLVAVGVGGWVGRWEGGQAGGCGSVQXXAVLLFKGHLAQKLXVQLHP | (SEQ ID NO:706) |
| TYRQLEEVHFSC, | |
| PASGSKDRASWVAAALVLVAVGVGGWVGRWE, | (SEQ ID NO:707) |
| ARNIMSIFSSLLLSRLASRSRMSRACRFSGVSLARFSLMASRSRCSRRARCSSVSCMRCKRLASRSLMRLTSRSSGVSLSRRLFLA | (SEQ ID NO:708) |
| VRSRSSRCSRTSKGSFCRRRAHLXTLGVWASSXLR, | |
| and/or | |
| RCKRLASRSLMRLTSRSSGVSLSRRLFLAVR. | (SEQ ID NO:709) |

encompassed by the invention.

This gene is expressed primarily in brain tissue, and to a lesser extent, in apoptopic T-cell and B-cell lymphoma. Therefore, polynucleotides and polypeptides of the invention are useful as reagents for differential identification of the tissue(s) or cell type(s) present in a biological sample and for diagnosis and treatment of growth disorders, neurodegenerative diseases, immune, hematopoietic, or endocrine disorders. Similarly, polypeptides and antibodies directed to these polypeptides are useful in providing immunological probes for differential identification of the tissue(s) or cell type(s). For a number of disorders of the above tissues or cells, particularly of the neural and immune systems, expression of this gene at significantly higher or lower levels may be routinely detected in certain tissues or cell types (e.g., growth disorders, neurodegenerative diseases, immune, hematopoietic, endocrine, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in brain, combined with the homology to proteins involved in gene regulation indicates that the protein product of this gene is useful for the diagnosis and treatment of immune and neurological diseases. Moreover, the protein may show utility in the detection, treatment, or prevention of a variety of proliferative disorders, particularly within the above tissues, such as brain or immune system cancers. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:91 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1552 of SEQ ID NO:91, b is an integer of 15 to 1566, where both a and b correspond to the positions of nucleotide residues shown in SEQ ID NO:91, and where b is greater than or equal to a+14.

Features of Protein Encoded by Gene No: 82

The translation product of this gene was found to have homology to the human high-affinity copper uptake protein, hctr1, (See Genbank Accession No. gi|2315987), which is thought to be important in the regulation of vital cellular processes indirectly, since an overabundance of intracellular copper can lead to toxic conditions within the cell, in addition to altering the function of cellular proteins via copper ion substitution. The ctrl gene was identified in the process of copper uptake, by complementation of the yeast high-affinity copper uptake mutant, ctrl. Besides complementing ctrl growth defect on nonfermentable media, the human gene also rescues iron transport and SOD1 defects in ctrl yeast. Overexpression of the gene in yeast leads to vulnerability to the toxicity of copper overload. In addition, its expression in ctrl yeast significantly increases the level of cellular copper, as demonstrated by atomic absorption. This gene is proposed to be a candidate for high-affinity copper uptake in humans. The hCTR1 and yeast CTR1 predicted transmembrane proteins are 29% identical, but the human protein is substantially smaller in both the extracellular metal-binding and intracellular domains. An additional human gene similar to hCTR1, here named hCTR2, was identified in a database search. Both hCTR1 and hCTR2 are expressed in all human tissues examined, and both genes are located in 9q31/32. These studies, together with the previously recognized functional and sequence similarity between the Menkes/Wilson copper export proteins and CCC2 in yeast, demonstrate that similar copper homeostatic mechanisms are used in these evolutionarily divergent organisms. The hcrt1 gene was found by another group subsequent to our initial filing (See for example, Proc. Natl. Acad. Sci. U.S.A. 94 (14), 7481-7486 (1997)).

In specific embodiments, polypeptides of the invention comprise the following amino acid sequence:

| | |
|---|---|
| MDHSHHMGMSYMDSNSTMQPSHHHPTTSASHSHGGGDSSMMMMPMTFYFGFKNVELLFSGLVINTAGEMAGAFVAVFLLAMFYEGL | (SEQ ID NO:710) |
| KIARESLLRKSQVSIRYNSMPVPGPNGTILMETHKTVGQQMLSFPHLLQTVLHIIQVVISYFLMLIFMTYNGYLCIAXAAGAGTGY | |
| FLFSWKKAVVVDITEHCH, | |
| GMSYMDSNSTMQPSHHHPITSA, | (SEQ ID NO:711) |
| GDSSMMMMPMTFYFGFKNVELLFSGL, | (SEQ ID NO:712) |
| VFLLAMFYEGLKIARESLLRKSQVSIRYNS, | (SEQ ID NO:713) |
| VPGPNGTILMETHKTVGQQMLSFPHLL, | (SEQ ID NO:714) |
| and/or | |
| FMTYNGYLCIAXAAGAGTGYFLFSWK. | (SEQ ID NO:715) |

Polynucleotides encoding these polypeptides are also encompassed by the invention.

The gene encoding the disclosed cDNA is believed to reside on chromosome 9. Accordingly, polynucleotides related to this invention are useful as a marker in linkage analysis for chromosome 9. This gene is expressed primarily in osteosarcoma, and to a lesser extent, in T-cell and bone marrow stromal cell. Therefore, polynucleotides and polypeptides of the invention are useful as reagents for differential identification of the tissue(s) or cell type(s) present in a biological sample and for treatment and diagnosis of skeletal, immune, or hematopoietic disorders, particularly osteosarcoma and copper and other metal uptake disorders. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., skeletal, immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 197 as residues: Ser-24 to Ser-29. The tissue distribution in osteosarcoma indicates that the protein product of this gene is useful for the prevention or treatment of osteosarcoma and copper or other metal uptake disorders. Alternatively, the expression within T-cell and bone marrow stromal cells indicates the protein product of this gene is useful for the treatment and diagnosis of hematopoietic related disorders such as anemia, pancytopenia, leukopenia, thrombocytopenia or leukemia since stromal cells are important in the production of cells of hematopoietic lineages. The uses include bone marrow cell ex vivo culture, bone marrow transplantation, bone marrow reconstitution, radiotherapy or chemotherapy of neoplasia. The gene product may also be involved in lymphopoiesis, therefore, it can be used in immune disorders such as infection, inflammation, allergy, immunodeficiency etc. In addition, this gene product may have commercial utility in the expansion of stem cells and committed progenitors of various blood lineages, and in the differentiation and/or proliferation of various cell types. Moreover, the overexpression or repression of this protein may show utility in the amelioration of cancer through the resulting changes in gene expression of important nuclear or cytoplasmic ion-dependent proteins required for normal cellular homeostasis. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:92 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1579 of SEQ ID NO:92, b is an integer of 15 to 1593, where both a and b correspond to the positions of nucleotide residues shown in SEQ ID NO:92, and where b is greater than or equal to a+14.

Features of Protein Encoded by Gene No: 83

| | |
|---|---|
| ACARAPGLTWRGKXQRVK, | (SEQ ID NO:716) |
| CGTSHQGCXHHGPAQATSWLCLPLPPQAHRSSGVLSGLAFILGQLQVTTSGRGGKRSQDTGQSEGHTPPTVLERGLHVSAPGGPGQ | (SEQ ID NO:717) |
| GGEDWGWGGGWTLQVLGPVVNREAKSLICSEAAAASPLNRHSKHLPAMPLCRRDANSDKGGQDGPQTRHPIFTLCXFPLQVSPGAL | |
| AQAQEGREAMQSDHTGPSALRAWAPRAEFGT, | |
| LSGLAFILGQLQVTTSGRGGKRSQDTGQ, | (SEQ ID NO:718) |
| CSEAAAASPLNRHSKHLPAMPLCRRDAN, | (SEQ ID NO:719) |
| and/or | |
| PGALAQAQEGREAMQSDHTGPSALRA. | (SEQ ID NO:720) |

Polynucleotides encoding these polypeptides are also encompassed by the invention.

This gene is expressed primarily in skin tumor, and to a lesser extent, in apoptotic T-cells. Therefore, polynucleotides and polypeptides of the invention are useful as reagents for differential identification of the tissue(s) or cell type(s) present in a biological sample and for diagnosis of diseases and conditions which include, but are not limited to, integumentary, immune, or hematopoietic disorders, particularly proliferative skin disorders, such as tumors. Similarly, polypeptides and antibodies directed to these polypeptides are useful in providing immunological probes for differential identification of the tissue(s) or cell type(s). For a number of disorders of the above tissues or cells, particularly of the skin, expression of this gene at significantly higher or lower levels may be routinely detected in certain tissues or cell types (e.g., integumentary, immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 198 as residues: Leu-51 to Gly-77, Ile-117 to Pro-125. The tissue distribution in skin and T-cells indicates that polynucleotides and polypeptides corresponding to this gene are useful for diagnosis the treatment of skin tumors. Similarly, the protein product of this gene is useful for the treatment, diagnosis, and/or prevention of various skin disorders including congenital disorders (i.e. nevi, moles, freckles, Mongolian spots, hemangiomas, port-wine syndrome), integumentary tumors (i.e. keratoses, Bowen's disease, basal cell carcinoma, squamous cell carcinoma, malignant melanoma, Paget's disease, mycosis fungoides, and Kaposi's sarcoma), injuries and inflammation of the skin (i.e. wounds, rashes, prickly heat disorder, psoriasis, dermatitis), atherosclerosis, uticaria, eczema, photosensitivity, autoimmune disorders (i.e. lupus erythematosus, vitiligo, dermatomyositis, morphea, scleroderma, pemphigoid, and pemphigus), keloids, striae, erythema, petechiae, purpura, and xanthelasma. In addition, such disorders may predispose increased susceptibility to viral and bacterial infections of the skin (i.e. cold sores, warts, chickenpox, molluscum contagiosum, herpes zoster, boils, cellulitis, erysipelas, impetigo, tinea, althletes foot, and ringworm). Moreover, the protein product of this gene may also be useful for the treatment or diagnosis of various connective tissue disorders such as arthritis, trauma, tendonitis, chrondomalacia and inflammation, autoimmune disorders such as rheumatoid arthritis, lupus, scleroderma, and dermatomyositis as well as dwarfism, spinal deformation, and specific joint abnormalities as well as chondrodysplasias (i.e. spondyloepiphyseal dysplasia congenita, familial osteoarthritis, Atelosteogenesis type II, metaphyseal chondrodysplasia type Schmid). The protein may also show utility in the treatment, detection, prevention, and/or amelioration of immune-directed responses to aberrant cellular skin conditions. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:93 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 956 of SEQ ID NO:93, b is an integer of 15 to 970, where both a and b correspond to the positions of nucleotide residues shown in SEQ ID NO:93, and where b is greater than or equal to a+14.

Features of Protein Encoded by Gene No: 84

In specific embodiments, polypeptides of the invention comprise the following amino acid sequence: GVSGEFHDEHLDLA (SEQ ID NO:721). Polynucleotides encoding these polypeptides are also encompassed by the invention.

This gene is expressed primarily in testis. Therefore, polynucleotides and polypeptides of the invention are useful as reagents for differential identification of the tissue(s) or cell type(s) present in a biological sample and for diagnosis of diseases and conditions which include, but are not limited to, reproductive, and/or endocrine disorders, particularly infertility. Similarly, polypeptides and antibodies directed to these polypeptides are useful in providing immunological probes for differential identification of the tissue(s) or cell type(s). For a number of disorders of the above tissues or cells, particularly of the reproductive system, expression of this gene at significantly higher or lower levels may be routinely detected in certain tissues or cell types (e.g., reproductive, endocrine, and cancerous and wounded tissues) or bodily fluids (e.g., lymph, serum, seminal fluid, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. The tissue distribution in testes indicates that polynucleotides and polypeptides corresponding to this gene are useful for the treatment of reproductive and endocrine diseases and disorders. Expression of this gene product in the testis may implicate this gene product in normal testicular function. In addition, this gene product may be useful in the treatment of male infertility, and/or could be used as a male contraceptive. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:94 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 920 of SEQ ID NO:94, b is an integer of 15 to 934, where both a and b correspond to the positions of nucleotide residues shown in SEQ ID NO:94, and where b is greater than or equal to a+14.

Features of Protein Encoded by Gene No: 85

In specific embodiments, polypeptides of the invention comprise the following amino acid sequence:

| | |
|---|---|
| MVQPCGACAKTXWKACSSCCSSPCCLQERWPXPXAXCPEXGPSSHPGIQALCAVAVVYLSPSSRLDWSLAPLFVPSLAAGETPLTQ | (SEQ ID NO:722) |
| PAWALTTNTLGHGQPAQDRLPALGHCAPISVLGLGSS, | |
| KCVLCGGVQVAGLXPPAAPGAAGLPLHPPXPGEQSKWLVIVMTV, | (SEQ ID NO:723) |
| QKEISTSWWHCYTAAACTRT, | (SEQ ID NO:724) |
| APCRWLKMCPLWRSTGGWPXSSCCSWSCWSASSPSXAWRTEQVAGDRDDSHESPGSRPELGLHGPGGSHGRGPQ, | (SEQ ID NO:725) |
| SSPSXAWRTEQVAGDRDDSHESPGSRPE, | (SEQ ID NO:726) |
| XGPSSHPGIQALCAVAVVYLSPSSR, | (SEQ ID NO:727) |
| and/or | |
| PLTQPAWALTTNTLGHGQPAQDRLPALGH. | (SEQ ID NO:728) |

invention.

This gene is expressed primarily in kidney cortex, frontal cortex, spinal cord and hippocampus. Therefore, polynucleotides and polypeptides of the invention are useful as reagents for differential identification of the tissue(s) or cell type(s) present in a biological sample and for diagnosis of diseases and conditions which include, but are not limited to, renal, urogenital, or neural disorders, particularly kidney fibrosis, schizophrenia and neurodegenerative disorders. Similarly, polypeptides and antibodies directed to these polypeptides are useful in providing immunological probes for differential identification of the tissue(s) or cell type(s). For a number of disorders of the above tissues or cells, particularly of the neural system, expression of this gene at significantly higher or lower levels may be routinely detected in certain tissues or cell types (e.g., renal, urogenital, neural, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 200 as residues: Cys-27 to Tyr-33, Thr-38 to Gly-43, Leu-125 to Gly-130. The tissue distribution in kidney cortex indicates that polynucleotides and polypeptides corresponding to this gene are useful for the treatment of kidney diseases. Similarly, this gene or gene product could be used in the treatment and/or detection of renal failure, nephritus, renal tubular acidosis, proteinuria, pyuria, edema, pyelonephritis, hydronephritis, nephrotic syndrome, crush syndrome, glomerulonephritis, hematuria, renal colic and kidney stones, in addition to Wilm's Tumor Disease, and congenital kidney abnormalities such as horseshoe kidney, polycystic kidney, and Falconi's syndrome. Moreover, the expression within various brain tissues strongly indicates that the protein product of this gene is useful for the detection/treatment of neurodegenerative disease states, behavioral disorders, or inflammatory conditions which include, but are not limited to Alzheimer's Disease, Parkinson's Disease, Huntington's Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, depression, panic disorder, learning disabilities, ALS, psychoses, autism, and altered behaviors, including disorders in feeding, sleep patterns, balance, and perception. In addition, elevated expression of this gene product in regions of the brain indicates that it plays a role in normal neural function. Potentially, this gene product is involved in synapse formation, neurotransmission, learning, cognition, homeostasis, or neuronal differentiation or survival. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:95 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1378 of SEQ ID NO:95, b is an integer of 15 to 1392, where both a and b correspond to the positions of nucleotide residues shown in SEQ ID NO:95, and where b is greater than or equal to a+14.

Features of Protein Encoded by Gene No: 86

| | |
|---|---|
| SELGQGHCEWAAPN, | (SEQ ID NO:729) |
| LQYGPIPGSTHASGEMQIKTVKCHFHFLDWQRVLCILLTVLNISSKQQMVSKYELRNLREMISLFQPADSFLQPV, | (SEQ ID NO:730) |
| EMQIKTVKCHFHFLDWQRVLCILLTVLNISSKQQ, | (SEQ ID NO:731) |
| HVKVKPMAELPPQHTGXQTGSCCLTFLNGLQPHLPXSVLRTMKLWSSLWTHHTTRRSKAMVTHPRVGPEENKALVLILTLTLSQDF | (SEQ ID NO:732) |
| DTEPKS, | |
| LWTHHTTRRSKAMVTHPRVGPEENKALV, | (SEQ ID NO:733) |
| IKVFTGDAHVSSSLCLYPTFIPTVLMVALSISPFSIPRYLPRNKPKSYYMLWHAXMMSSDEQPKPQGRDAFVKQDLWNITCESKAH | (SEQ ID NO:734) |
| GRTPSSAHGXADRKLLPHLPQWXTATSPQVSLKDNETLVFXVDTPHYQALQSHGDPPSGGS, | (SEQ ID NO:735) |
| FIPTVLMVALSISPFSIPRYLPRNKP, | |
| PKPQGRDAFVKQDLWNITCESKAH, | (SEQ ID NO:736) |
| and/or | |
| QVSLKDNETLVFXVDTPHYQALQSHG. | (SEQ ID NO:737) |

invention.

This gene is expressed primarily in resting T-cell. Therefore, polynucleotides and polypeptides of the invention are useful as reagents for differential identification of the tissue(s) or cell type(s) present in a biological sample and for diagnosis of diseases and conditions which include, but are not limited to, immune or hematopoietic disorders, particularly inflammatory or immunodeficiency conditions. Similarly, polypeptides and antibodies directed to these polypeptides are useful in providing immunological probes for differential identification of the tissue(s) or cell type(s). For a number of disorders of the above tissues or cells, particularly of the immune system, expression of this gene at significantly higher or lower levels may be routinely detected in certain tissues or cell types (e.g., immune, hematopoietic, and cancerous and wounded tissues) or bodily fluids (e.g., lymph, serum, plasma, urine, synovial fluid and spinal fluid) or another tissue or cell sample taken from an individual having such a disorder, relative to the standard gene expression level, i.e., the expression level in healthy tissue or bodily fluid from an individual not having the disorder. Preferred epitopes include those comprising a sequence shown in SEQ ID NO. 201 as residues: Thr-54 to Ile-59. The tissue distribution in T-cells indicates that polynucleotides and polypeptides corresponding to this gene are useful for the treatment of immune diseases. Moreover, expression of this gene product indicates a role in regulating the proliferation; survival; differentiation; and/or activation of hematopoietic cell lineages, including blood stem cells. This gene product may be involved in the regulation of cytokine production, antigen presentation, or other processes that may also suggest a usefulness in the treatment of cancer (e.g. by boosting immune responses). Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues. In addition, this gene product may have commercial utility in the expansion of stem cells and committed progenitors of various blood lineages, and in the differentiation and/or proliferation of various cell types. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:96 and may have been publicly available prior to conception of the present invention. Preferably, such related polynucleotides are specifically excluded from the scope of the present invention. To list every related sequence is cumbersome. Accordingly, preferably excluded from the present invention are one or more polynucleotides comprising a nucleotide sequence described by the general formula of a-b, where a is any integer between 1 to 1949 of SEQ ID NO:96, b is an integer of 15 to 1963, where both a and b correspond to the positions of nucleotide residues shown in SEQ ID NO:96, and where b is greater than or equal to a+14.

TABLE 1

| | | | | | | | | | 5′ NT | | | | First | |
|---|---|---|---|---|---|---|---|---|---|---|---|---|---|---|
| | | | | | | | | | of First AA | | First | Last | AA of | |
| Gene | cDNA | ATCC ™ Deposit | | NT SEQ | Total NT | 5′ NT of | 3′ NT of | 5′ NT of | of Signal | AA SEQ | AA of | AA of | Secreted | Last AA |
| No. | Clone ID | No: Z and Date | Vector | ID NO: X | Seq. | Clone Seq. | Clone Seq. | Start Codon | Pep | ID NO: Y | Sig Pep | Sig Pep | Portion | of ORF |
| 1 | HOAAE80 | 209012 Apr. 28, 1997 | Uni-ZAP XR | 11 | 1387 | 430 | 1387 | 340 | 340 | 116 | 1 | 30 | 31 | 165 |
| | | 209089 Jun. 05, 1997 | | | | | | | | | | | | |
| 1 | HOAAE80 | 209012 Apr. 28, 1997 | Uni-ZAP XR | 97 | 1220 | 264 | 1220 | 288 | 288 | 202 | 1 | 26 | 27 | 31 |
| | | 209089 Jun. 05, 1997 | | | | | | | | | | | | |
| 2 | HODDN92 | 209012 Apr. 28, 1997 | Uni-ZAP XR | 12 | 1939 | 294 | 1939 | | 434 | 117 | 1 | 26 | 27 | 35 |
| | | 209089 Jun. 05, 1997 | | | | | | | | | | | | |
| 3 | HOSBI96 | 209012 Apr. 28, 1997 | Uni-ZAP XR | 13 | 2602 | 672 | 1811 | 690 | 690 | 118 | 1 | 30 | 31 | 219 |
| | | 209089 Jun. 05, 1997 | | | | | | | | | | | | |
| 4 | HOVAI58 | 209012 Apr. 28, 1997 | pSport1 | 14 | 808 | 1 | 808 | 28 | 28 | 119 | 1 | 26 | 27 | 31 |
| | | 209089 Jun. 05, 1997 | | | | | | | | | | | | |
| 5 | HPBDD36 | 209012 Apr. 28, 1997 | pBLUESCRIPT ™ | 15 | 2143 | 1351 | 2095 | 1130 | 1130 | 120 | 1 | 38 | 39 | 95 |
| | | 209089 Jun. 05, 1997 | SK- | | | | | | | | | | | |
| 5 | HPBDD36 | 209012 Apr. 28, 1997 | pBLUESCRIPT ™ | 98 | 864 | 87 | 831 | 147 | 147 | 203 | 1 | 18 | 19 | 26 |
| | | 209089 Jun. 05, 1997 | SK- | | | | | | | | | | | |
| 6 | HPDDC77 | 209012 Apr. 28, 1997 | pBLUESCRIPT ™ | 16 | 2361 | 455 | 1442 | 510 | 510 | 121 | 1 | 29 | 30 | 131 |
| | | 209089 Jun. 05, 1997 | SK- | | | | | | | | | | | |
| 7 | HPEBD85 | 209012 Apr. 28, 1997 | Uni-ZAP XR | 17 | 803 | 1 | 803 | 81 | 81 | 122 | 1 | 20 | 21 | 64 |
| | | 209089 Jun. 05, 1997 | | | | | | | | | | | | |
| 8 | HPFCX38 | 209012 Apr. 28, 1997 | Uni-ZAP XR | 18 | 1794 | 1051 | 1757 | | 578 | 123 | 1 | | | 8 |
| | | 209089 Jun. 05, 1997 | | | | | | | | | | | | |
| 9 | HPFCY51 | 209012 Apr. 28, 1997 | Uni-ZAP XR | 19 | 1037 | 1 | 1037 | 467 | 467 | 124 | 1 | 30 | 31 | 50 |
| | | 209089 Jun. 05, 1997 | | | | | | | | | | | | |
| 9 | HPFCY51 | 209012 Apr. 28, 1997 | Uni-ZAP XR | 99 | 1052 | 1 | 1052 | 30 | 30 | 204 | 1 | | | 12 |
| | | 209089 Jun. 05, 1997 | | | | | | | | | | | | |
| 10 | HPMGQ80 | 209012 Apr. 28, 1997 | Uni-ZAP XR | 20 | 1273 | 168 | 1266 | 364 | 364 | 125 | 1 | 18 | 19 | 66 |
| | | 209089 Jun. 05, 1997 | | | | | | | | | | | | |
| 10 | HPMGQ80 | 209012 Apr. 28, 1997 | Uni-ZAP XR | 100 | 1309 | 157 | 1309 | 360 | 360 | 205 | 1 | 19 | 20 | 75 |
| | | 209089 Jun. 05, 1997 | | | | | | | | | | | | |
| 11 | HPRTG55 | 209012 Apr. 28, 1997 | pBLUESCRIPT ™ | 21 | 1081 | 55 | 1014 | 237 | 237 | 126 | 1 | 24 | 25 | 26 |
| | | 209089 Jun. 05, 1997 | | | | | | | | | | | | |
| 12 | HROAN56 | 209012 Apr. 28, 1997 | Uni-ZAP XR | 22 | 807 | 1 | 807 | 26 | 26 | 127 | 1 | 19 | 20 | 23 |
| | | 209089 Jun. 05, 1997 | | | | | | | | | | | | |
| 13 | HSABI42 | 209012 Apr. 28, 1997 | pBLUESCRIPT ™ | 23 | 632 | 1 | 596 | 190 | 190 | 128 | 1 | 15 | 16 | 21 |
| | | 209089 Jun. 05, 1997 | SK- | | | | | | | | | | | |
| 14 | HSAUW44 | 209012 Apr. 28, 1997 | Uni-ZAP XR | 24 | 1358 | 1 | 1358 | 372 | 372 | 129 | 1 | 30 | 31 | 54 |
| | | 209089 Jun. 05, 1997 | | | | | | | | | | | | |
| 15 | HSDES04 | 209012 Apr. 28, 1997 | Uni-ZAP XR | 25 | 1376 | 686 | 1376 | 146 | 146 | 130 | 1 | 33 | 34 | 318 |
| | | 209089 Jun. 05, 1997 | | | | | | | | | | | | |
| 15 | HSDES04 | 209089 Jun. 05, 1997 | Uni-ZAP XR | 101 | 929 | 57 | 929 | 291 | 291 | 206 | 1 | 28 | 29 | 60 |
| 16 | HSHBQ68 | 209012 Apr. 28, 1997 | Uni-ZAP XR | 26 | 2923 | 195 | 2642 | 211 | 211 | 131 | 1 | 23 | 24 | 58 |
| | | 209089 Jun. 05, 1997 | | | | | | | | | | | | |
| 17 | HSKBO20 | 209012 Apr. 28, 1997 | Uni-ZAP XR | 27 | 2954 | 337 | 2954 | 456 | 456 | 132 | 1 | 30 | 31 | 305 |
| | | 209089 Jun. 05, 1997 | | | | | | | | | | | | |
| 17 | HSKBO20 | 209012 Apr. 28, 1997 | Uni-ZAP XR | 102 | 775 | 1 | 501 | | 308 | 207 | 1 | 28 | 29 | 98 |
| | | 209089 Jun. 05, 1997 | | | | | | | | | | | | |
| 18 | HSKNM85 | 209012 Apr. 28, 1997 | pBLUESCRIPT ™ | 28 | 534 | 1 | 534 | 122 | 122 | 133 | 1 | 19 | 20 | 28 |
| | | 209089 Jun. 05, 1997 | | | | | | | | | | | | |
| 19 | HSKXJ37 | 209012 Apr. 28, 1997 | pBLUESCRIPT ™ | 29 | 1827 | 67 | 1634 | 311 | 311 | 134 | 1 | 21 | | 21 |
| | | 209089 Jun. 05, 1997 | | | | | | | | | | | | |
| 20 | HSKZE52 | 209012 Apr. 28, 1997 | Uni-ZAP XR | 30 | 1479 | 418 | 1453 | 555 | 555 | 135 | 1 | 18 | 19 | 111 |
| | | 209089 Jun. 05, 1997 | | | | | | | | | | | | |
| 21 | HWTAZ75 | 209012 Apr. 28, 1997 | Uni-ZAP XR | 31 | 987 | 448 | 963 | 133 | 133 | 136 | 1 | 29 | 30 | 114 |
| | | 209089 Jun. 05, 1997 | | | | | | | | | | | | |
| 22 | HSRBA90 | 209012 Apr. 28, 1997 | Uni-ZAP XR | 32 | 2933 | 1437 | 2933 | 1670 | 1670 | 137 | 1 | 19 | 20 | 21 |
| | | 209089 Jun. 05, 1997 | | | | | | | | | | | | |
| 23 | HSVAG05 | 209090 Jun. 05, 1997 | Uni-ZAP XR | 33 | 1366 | 1 | 1366 | 66 | 66 | 138 | 1 | 31 | 32 | 51 |
| 24 | HSVBF78 | 209090 Jun. 05, 1997 | Uni-ZAP XR | 34 | 667 | 141 | 621 | 64 | 64 | 139 | 1 | 28 | 29 | 99 |
| 25 | HSXBO51 | 209090 Jun. 05, 1997 | Uni-ZAP XR | 35 | 1710 | 388 | 1683 | 462 | 462 | 140 | 1 | 26 | 27 | 175 |
| 26 | HT3BE24 | 209090 Jun. 05, 1997 | Uni-ZAP XR | 36 | 1096 | 756 | 1091 | 422 | 422 | 141 | 1 | 15 | 16 | 187 |
| 26 | HT3BE24 | 209090 Jun. 05, 1997 | Uni-ZAP XR | 103 | 359 | 1 | 359 | 41 | 41 | 208 | 1 | 42 | 43 | 70 |
| 27 | HT4AI54 | 209090 Jun. 05, 1997 | Uni-ZAP XR | 37 | 2279 | 1387 | 2279 | 29 | 29 | 142 | 1 | 24 | 25 | 288 |
| 27 | HT4AI54 | 209090 Jun. 05, 1997 | Uni-ZAP XR | 104 | 952 | 1 | 952 | 199 | 199 | 209 | 1 | | | 9 |
| 28 | HTEHU93 | 209090 Jun. 05, 1997 | Uni-ZAP XR | 38 | 745 | 1 | 745 | 187 | 187 | 143 | 1 | 24 | 25 | 113 |
| 29 | HTGCQ82 | 209090 Jun. 05, 1997 | Uni-ZAP XR | 39 | 1718 | 70 | 1718 | 114 | 114 | 144 | 1 | 23 | 24 | 119 |
| 30 | HTLAB25 | 209090 Jun. 05, 1997 | Uni-ZAP XR | 40 | 1966 | 321 | 1966 | 1371 | 1371 | 145 | 1 | 38 | 39 | 78 |
| 31 | HTLAV68 | 209090 Jun. 05, 1997 | Uni-ZAP XR | 41 | 972 | 1 | 972 | 78 | 78 | 146 | 1 | 35 | 36 | 162 |
| 32 | HTLDQ11 | 209090 Jun. 05, 1997 | Uni-ZAP XR | 42 | 1536 | 1 | 1536 | 213 | 213 | 147 | 1 | 36 | 37 | 72 |
| 33 | HTOBX52 | 209090 Jun. 05, 1997 | Uni-ZAP XR | 43 | 2541 | 1743 | 2541 | | 3 | 148 | 1 | 4 | 5 | 123 |
| 34 | HTTCN24 | 209090 Jun. 05, 1997 | Uni-ZAP XR | 44 | 2418 | 918 | 2290 | 188 | 188 | 149 | 1 | 30 | 31 | 138 |
| 34 | HTTCN24 | 209090 Jun. 05, 1997 | Uni-ZAP XR | 105 | 1545 | 123 | 1545 | 345 | 345 | 210 | 1 | 39 | 40 | 49 |
| 35 | HTXCS21 | 209090 Jun. 05, 1997 | Uni-ZAP XR | 45 | 1337 | 657 | 1309 | 76 | 76 | 150 | 1 | 24 | 25 | 356 |
| 35 | HTXCS21 | 209090 Jun. 05, 1997 | Uni-ZAP XR | 106 | 1322 | 641 | 1293 | | 1203 | 211 | 1 | | | 12 |
| 36 | HUFAC49 | 209090 Jun. 05, 1997 | pSport1 | 46 | 1276 | 1 | 1276 | 105 | 105 | 151 | 1 | 17 | 18 | 39 |
| 37 | HAIDK60 | 209090 Jun. 05, 1997 | Uni-ZAP XR | 47 | 1282 | 1 | 1282 | 528 | 528 | 152 | 1 | 30 | 31 | 71 |
| 37 | HAIDK60 | 209090 Jun. 05, 1997 | Uni-ZAP XR | 107 | 276 | 1 | 276 | 14 | 14 | 212 | 1 | 25 | 26 | 37 |
| 38 | HARAG28 | 209090 Jun. 05, 1997 | pBLUESCRIPT ™ | 48 | 645 | 1 | 645 | 150 | 150 | 153 | 1 | 16 | 17 | 33 |
| | | | SK- | | | | | | | | | | | |
| 38 | HARAG28 | 209090 Jun. 05, 1997 | pBLUESCRIPT ™ | 108 | 381 | 1 | 381 | 154 | 154 | 213 | 1 | 18 | 19 | 33 |
| | | | SK- | | | | | | | | | | | |
| 39 | HBMBB80 | 209090 Jun. 05, 1997 | pBLUESCRIPT ™ | 49 | 1495 | 2 | 1495 | 23 | 23 | 154 | 1 | 30 | 31 | 78 |
| 39 | HBMBB80 | 209090 Jun. 05, 1997 | pBLUESCRIPT ™ | 109 | 638 | 1 | 638 | 196 | 196 | 214 | 1 | 16 | 17 | 25 |
| 40 | HCEGR33 | 209090 Jun. 05, 1997 | Uni-ZAP XR | 50 | 1630 | 1 | 1630 | 243 | 243 | 155 | 1 | 22 | 23 | 31 |
| 41 | HSXBP68 | 209090 Jun. 05, 1997 | Uni-ZAP XR | 51 | 2420 | 1009 | 2252 | 79 | 79 | 156 | 1 | 41 | 42 | 464 |
| 41 | HSXBP68 | 209090 Jun. 05, 1997 | Uni-ZAP XR | 110 | 2246 | 835 | 2079 | 985 | 985 | 215 | 1 | 32 | 33 | 104 |
| 42 | HFFAT33 | 209090 Jun. 05, 1997 | Lambda ZAP II | 52 | 1172 | 166 | 802 | 209 | 209 | 157 | 1 | 29 | 30 | 151 |
| 43 | HFGAG96 | 209090 Jun. 05, 1997 | Uni-ZAP XR | 53 | 1589 | 885 | 1446 | 189 | 189 | 158 | 1 | 33 | 34 | 299 |
| 43 | HFGAG96 | 209090 Jun. 05, 1997 | Uni-ZAP XR | 111 | 1105 | 1 | 1105 | | 247 | 216 | 1 | 17 | 18 | 63 |
| 44 | HETFJ05 | 209076 May 22, 1997 | Uni-ZAP XR | 54 | 2074 | 1 | 2065 | 75 | 75 | 159 | 1 | 24 | 25 | 397 |
| 45 | HLTEY63 | 209076 May 22, 1997 | Uni-ZAP XR | 55 | 1483 | 1 | 1280 | 86 | 86 | 160 | 1 | 18 | 19 | 82 |
| 46 | HMSJU68 | 209076 May 22, 1997 | Uni-ZAP XR | 56 | 1123 | 4 | 1123 | 272 | 272 | 161 | 1 | 31 | 32 | 49 |
| 47 | HOSCZ41 | 209076 May 22, 1997 | Uni-ZAP XR | 57 | 1239 | 117 | 1222 | 178 | 178 | 162 | 1 | 20 | 21 | 50 |
| 48 | HSHAV28 | 209076 May 22, 1997 | Uni-ZAP XR | 58 | 803 | 105 | 719 | 100 | 100 | 163 | 1 | 19 | 20 | 194 |
| 49 | HSQEA85 | 209076 May 22, 1997 | Uni-ZAP XR | 59 | 995 | 1 | 995 | 98 | 98 | 164 | 1 | 23 | 24 | 52 |
| 50 | HSTAG52 | 209076 May 22, 1997 | Uni-ZAP XR | 60 | 966 | 114 | 966 | 191 | 191 | 165 | 1 | 45 | 46 | 63 |
| 51 | HBNAJ22 | 209076 May 22, 1997 | Uni-ZAP XR | 61 | 262 | 1 | 262 | 28 | 28 | 166 | 1 | 23 | 24 | 32 |
| 52 | HBXGP76 | 209076 May 22, 1997 | ZAP Express | 62 | 753 | 1 | 753 | 34 | 34 | 167 | 1 | 34 | 35 | 94 |
| 53 | HE6GL64 | 209076 May 22, 1997 | Uni-ZAP XR | 63 | 739 | 1 | 739 | 132 | 132 | 168 | 1 | 32 | 33 | 57 |
| 54 | HESAL35 | 209076 May 22, 1997 | Uni-ZAP XR | 64 | 476 | 1 | 476 | 20 | 20 | 169 | 1 | 27 | 28 | 43 |
| 55 | HETBB70 | 209076 May 22, 1997 | Uni-ZAP XR | 65 | 696 | 1 | 696 | 81 | 81 | 170 | 1 | 25 | 26 | 57 |
| 55 | HETBB70 | 209076 May 22, 1997 | Uni-ZAP XR | 112 | 754 | 14 | 754 | | 263 | 217 | 1 | 17 | 18 | 17 |
| 56 | HLHAY19 | 209076 May 22, 1997 | Uni-ZAP XR | 66 | 1890 | 8 | 1890 | 18 | 18 | 171 | 1 | 22 | 23 | 28 |
| 57 | HLTER45 | 209076 May 22, 1997 | Uni-ZAP XR | 67 | 1614 | 557 | 1614 | 578 | 578 | 172 | 1 | 25 | 26 | 36 |
| 58 | HNHAL34 | 209076 May 22, 1997 | Uni-ZAP XR | 68 | 596 | 1 | 596 | 90 | 90 | 173 | 1 | 18 | 19 | 39 |
| 59 | HOSFF78 | 209076 May 22, 1997 | Uni-ZAP XR | 69 | 1524 | 791 | 1524 | 846 | 846 | 174 | 1 | 34 | 35 | 46 |
| 60 | HSKDV92 | 209076 May 22, 1997 | Uni-ZAP XR | 70 | 819 | 53 | 819 | | 158 | 175 | 1 | 32 | 33 | 33 |
| 61 | HFCCU63 | 209076 May 22, 1997 | Uni-ZAP XR | 71 | 1442 | 1 | 1442 | 1243 | 1243 | 176 | 1 | 16 | 17 | 39 |
| 62 | HLTCS34 | 209076 May 22, 1997 | Uni-ZAP XR | 72 | 1223 | 1 | 1223 | 227 | 227 | 177 | 1 | 17 | 18 | 24 |
| 63 | HPMCC16 | 209086 May 29, 1997 | Uni-ZAP XR | 73 | 1814 | 1024 | 1814 | 85 | 85 | 178 | 1 | 19 | 20 | 262 |
| 64 | HOUCQ17 | 209086 May 29, 1997 | Uni-ZAP XR | 74 | 4712 | 1 | 4693 | 508 | 508 | 179 | 1 | 51 | 52 | 967 |
| 65 | HTDAG66 | 209086 May 29, 1997 | pSport1 | 75 | 1647 | 780 | 1647 | 298 | 298 | 180 | 1 | 31 | 32 | 122 |
| 65 | HTDAG66 | 209086 May 29, 1997 | pSport1 | 113 | 1885 | 262 | 1885 | 369 | 369 | 218 | 1 | | | 17 |
| 66 | HTLBC79 | 209086 May 29, 1997 | Uni-ZAP XR | 76 | 890 | 1 | 890 | 276 | 276 | 181 | 1 | 28 | 29 | 57 |
| 67 | HTOFC34 | 209086 May 29, 1997 | Uni-ZAP XR | 77 | 1657 | 356 | 1645 | 434 | 434 | 182 | 1 | 31 | 32 | 54 |
| 68 | H2CBJ08 | 209086 May 29, 1997 | pBLUESCRIPT ™ | 78 | 2015 | 13 | 2015 | 70 | 70 | 183 | 1 | 17 | 18 | 435 |
| | | | SK- | | | | | | | | | | | |
| 69 | HAGFT48 | 209086 May 29, 1997 | Uni-ZAP XR | 79 | 1213 | 242 | 1213 | | 290 | 184 | 1 | 23 | 24 | 174 |
| 70 | HCE5M29 | 209086 May 29, 1997 | Uni-ZAP XR | 80 | 1391 | 23 | 1353 | 36 | 36 | 185 | 1 | 19 | 20 | 56 |
| 71 | HTPBQ83 | 209076 May 22, 1997 | Uni-ZAP XR | 81 | 1008 | 146 | 1008 | 14 | 14 | 186 | 1 | 15 | 16 | 144 |
| 72 | HCFNN01 | 209086 May 29, 1997 | pSport1 | 82 | 1261 | 154 | 1261 | 254 | 254 | 187 | 1 | 27 | 28 | 43 |
| 73 | HE7TF86 | 209086 May 29, 1997 | Uni-ZAP XR | 83 | 1045 | 241 | 986 | 426 | 426 | 188 | 1 | 23 | 24 | 58 |
| 74 | HGBAC11 | 209086 May 29, 1997 | Uni-ZAP XR | 84 | 2877 | 1 | 2272 | 188 | 188 | 189 | 1 | 25 | 26 | 111 |
| 75 | HHGAU81 | 209086 May 29, 1997 | Lambda ZAP II | 85 | 1367 | 747 | 1367 | 323 | 323 | 190 | 1 | 24 | 25 | 166 |
| 76 | HLCAA05 | 209086 May 29, 1997 | Uni-ZAP XR | 86 | 1010 | 1 | 1010 | 656 | 656 | 191 | 1 | 69 | 70 | 78 |
| 76 | HLCAA05 | 209086 May 29, 1997 | Uni-ZAP XR | 114 | 1009 | 1 | 1009 | 276 | 276 | 219 | 1 | | | 8 |
| 77 | HMSCD68 | 209086 May 29, 1997 | Uni-ZAP XR | 87 | 1367 | 1 | 1367 | 373 | 373 | 192 | 1 | 18 | 19 | 36 |
| 78 | HMWDZ81 | 209086 May 29, 1997 | Uni-Zap XR | 88 | 1088 | 1 | 883 | 214 | 214 | 193 | 1 | 22 | 23 | 30 |
| 79 | HMWGQ73 | 209086 May 29, 1997 | Uni-Zap XR | 89 | 1861 | 1 | 1861 | 789 | 789 | 194 | 1 | 21 | 22 | 34 |
| 80 | HOECN31 | 209086 May 29, 1997 | Uni-ZAP XR | 90 | 1259 | 34 | 1259 | 338 | 338 | 195 | 1 | 28 | 29 | 32 |
| 81 | HPTRF90 | 209086 May 29, 1997 | pBLUESCRIPT ™ | 91 | 1566 | 450 | 1552 | 593 | 593 | 196 | 1 | 28 | 29 | 83 |
| 82 | HSRDH01 | 209086 May 29, 1997 | Uni-ZAP XR | 92 | 1593 | 107 | 1593 | 379 | 379 | 197 | 1 | 22 | 23 | 122 |
| 83 | HSAWD74 | 209126 Jun. 19, 1997 | Uni-ZAP XR | 93 | 970 | 106 | 970 | 142 | 142 | 198 | 1 | 26 | 27 | 142 |
| 83 | HSTBE27 | 209086 May 29, 1997 | Uni-ZAP XR | 115 | 646 | 117 | 646 | 122 | 122 | 220 | 1 | 31 | 32 | 45 |
| 84 | HTEJO12 | 209086 May 29, 1997 | Uni-ZAP XR | 94 | 934 | 1 | 934 | 202 | 202 | 199 | 1 | 20 | 21 | 50 |
| 85 | HTLAB43 | 209086 May 29, 1997 | Uni-ZAP XR | 95 | 1392 | 199 | 1392 | 384 | 384 | 200 | 1 | 17 | 18 | 221 |
| 86 | HTWCT03 | 209086 May 29, 1997 | pSport1 | 96 | 1963 | 1 | 1963 | 334 | 334 | 201 | 1 | 26 | 27 | 101 |

Table 1 summarizes the information corresponding to each “Gene No.” described above. The nucleotide sequence identified as “NT SEQ ID NO:X” was assembled from partially homologous (“overlapping”) sequences obtained from the “cDNA clone ID” identified in Table I and, in some cases, from additional related DNA clones. The overlapping sequences were assembled into a single contiguous sequence of high redundancy (usually three to five overlapping sequences at each nucleotide position), resulting in a final sequence identified as SEQ ID NO:X.

The cDNA Clone ID was deposited on the date and given the corresponding deposit number listed in “ATCC™ Deposit No:Z and Date.” Some of the deposits contain multiple different clones corresponding to the same gene. “Vector” refers to the type of vector contained in the cDNA Clone ID.

“Total NT Seq.” refers to the total number of nucleotides in the contig identified by “Gene No.” The deposited clone may contain all or most of these sequences, reflected by the nucleotide position indicated as “5′ NT of Clone Seq.” and the “3′ NT of Clone Seq.” of SEQ ID NO:X. The nucleotide position of SEQ ID NO:X of the putative start codon (methionine) is identified as “5′ NT of Start Codon.” Similarly, the nucleotide position of SEQ ID NO:X of the predicted signal sequence is identified as “5′ NT of First AA of Signal Pep.”

The translated amino acid sequence, beginning with the methionine, is identified as “AA SEQ ID NO:Y,” although other reading frames can also be easily translated using known molecular biology techniques. The polypeptides produced by these alternative open reading frames are specifically contemplated by the present invention.

The first and last amino acid position of SEQ ID NO:Y of the predicted signal peptide is identified as “First AA of Sig Pep” and “Last AA of Sig Pep.” The predicted first amino acid position of SEQ ID NO:Y of the secreted portion is identified as “Predicted First AA of Secreted Portion.” Finally, the amino acid position of SEQ ID NO:Y of the last amino acid in the open reading frame is identified as “Last AA of ORF.”

SEQ ID NO:X and the translated SEQ ID NO:Y are sufficiently accurate and otherwise suitable for a variety of uses well known in the art and described further below. For instance, SEQ ID NO:X is useful for designing nucleic acid hybridization probes that will detect nucleic acid sequences contained in SEQ ID NO:X or the cDNA contained in the deposited clone. These probes will also hybridize to nucleic acid molecules in biological samples, thereby enabling a variety of forensic and diagnostic methods of the invention. Similarly, polypeptides identified from SEQ ID NO:Y may be used to generate antibodies which bind specifically to the secreted proteins encoded by the cDNA clones identified in Table 1.

Nevertheless, DNA sequences generated by sequencing reactions can contain sequencing errors. The errors exist as misidentified nucleotides, or as insertions or deletions of nucleotides in the generated DNA sequence. The erroneously inserted or deleted nucleotides cause frame shifts in the reading frames of the predicted amino acid sequence. In these cases, the predicted amino acid sequence diverges from the actual amino acid sequence, even though the generated DNA sequence may be greater than 99.9% identical to the actual DNA sequence (for example, one base insertion or deletion in an open reading frame of over 1000 bases).

Accordingly, for those applications requiring precision in the nucleotide sequence or the amino acid sequence, the present invention provides not only the generated nucleotide sequence identified as SEQ ID NO:X and the predicted translated amino acid sequence identified as SEQ ID NO:Y, but also a sample of plasmid DNA containing a human cDNA of the invention deposited with the ATCC™, as set forth in Table 1. The nucleotide sequence of each deposited clone can readily be determined by sequencing the deposited clone in accordance with known methods. The predicted amino acid sequence can then be verified from such deposits. Moreover, the amino acid sequence of the protein encoded by a particular clone can also be directly determined by peptide sequencing or by expressing the protein in a suitable host cell containing the deposited human cDNA, collecting the protein, and determining its sequence.

The present invention also relates to the genes corresponding to SEQ ID NO:X, SEQ ID NO:Y, or the deposited clone. The corresponding gene can be isolated in accordance with known methods using the sequence information disclosed herein. Such methods include preparing probes or primers from the disclosed sequence and identifying or amplifying the corresponding gene from appropriate sources of genomic material.

Also provided in the present invention are species homologs. Species homologs may be isolated and identified by making suitable probes or primers from the sequences provided herein and screening a suitable nucleic acid source for the desired homologue.

The polypeptides of the invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

The polypeptides may be in the form of the secreted protein, including the mature form, or may be a part of a larger protein, such as a fusion protein (see below). It is often advantageous to include an additional amino acid sequence which contains secretory or leader sequences, pro-sequences, sequences which aid in purification, such as multiple histidine residues, or an additional sequence for stability during recombinant production.

The polypeptides of the present invention are preferably provided in an isolated form, and preferably are substantially purified. A recombinantly produced version of a polypeptide, including the secreted polypeptide, can be substantially purified by the one-step method described in Smith and Johnson, Gene 67: 3140 (1988). Polypeptides of the invention also can be purified from natural or recombinant sources using antibodies of the invention raised against the secreted protein in methods which are well known in the art.

Signal Sequences

Methods for predicting whether a protein has a signal sequence, as well as the cleavage point for that sequence, are available. For instance, the method of McGeoch, Virus Res. 3: 271-286 (1985), uses the information from a short N-terminal charged region and a subsequent uncharged region of the complete (uncleaved) protein. The method of von Heinje, Nucleic Acids Res. 14: 4683-4690 (1986) uses the information from the residues surrounding the cleavage site, typically residues −13 to +2, where +1 indicates the amino terminus of the secreted protein. The accuracy of predicting the cleavage points of known mammalian secretory proteins for each of these methods is in the range of 75-80%. (von Heinje, supra.) However, the two methods do not always produce the same predicted cleavage point(s) for a given protein.

In the present case, the deduced amino acid sequence of the secreted polypeptide was analyzed by a computer program called SignalP (Henrik Nielsen et al., Protein Engineering 10: 1-6 (1997)), which predicts the cellular location of a protein based on the amino acid sequence. As part of this computational prediction of localization, the methods of McGeoch and von Heinje are incorporated. The analysis of the amino acid sequences of the secreted proteins described herein by this program provided the results shown in Table 1.

As one of ordinary skill would appreciate, however, cleavage sites sometimes vary from organism to organism and cannot be predicted with absolute certainty. Accordingly, the present invention provides secreted polypeptides having a sequence shown in SEQ ID NO:Y which have an N-terminus beginning within 5 residues (i.e., + or −5 residues) of the predicted cleavage point. Similarly, it is also recognized that in some cases, cleavage of the signal sequence from a secreted protein is not entirely uniform, resulting in more than one secreted species. These polypeptides, and the polynucleotides encoding such polypeptides, are contemplated by the present invention.

Moreover, the signal sequence identified by the above analysis may not necessarily predict the naturally occurring signal sequence. For example, the naturally occurring signal sequence may be further upstream from the predicted signal sequence. However, it is likely that the predicted signal sequence will be capable of directing the secreted protein to the ER. These polypeptides, and the polynucleotides encoding such polypeptides, are contemplated by the present invention.

Polynucleotide and Polypeptide Variants

“Variant” refers to a polynucleotide or polypeptide differing from the polynucleotide or polypeptide of the present invention, but retaining essential properties thereof. Generally, variants are overall closely similar, and, in many regions, identical to the polynucleotide or polypeptide of the present invention.

By a polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence encoding the polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence shown in Table 1, the ORF (open reading frame), or any fragment specified as described herein.

As a practical matter, whether any particular nucleic acid molecule or polypeptide is at least 90%, 95%, 96%, 97%, 98% or 99% identical to a nucleotide sequence of the presence invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6: 237-245). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the lenght of the subject nucleotide sequence, whichever is shorter.

If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for. No other manual corrections are to made for the purposes of the present invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, (indels) or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequences shown in Table 1 or to the amino acid sequence encoded by deposited DNA clone can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6: 237-245). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are to made for the purposes of the present invention.

The variants may contain alterations in the coding regions; non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred. Polynucleotide variants can be produced for a variety of reasons, e.g., to optimize codon expression for a particular host (change codons in the human mRNA to those preferred by a bacterial host such as E. coli).

Naturally occurring variants are called “allelic variants,” and refer to one of several alternate forms of a gene occupying a given locus on a chromosome of an organism. (Genes II, Lewin, B., ed., John Wiley & Sons, New York (1985).) These allelic variants can vary at either the polynucleotide and/or polypeptide level. Alternatively, non-naturally occurring variants may be produced by mutagenesis techniques or by direct synthesis.

Using known methods of protein engineering and recombinant DNA technology, variants may be generated to improve or alter the characteristics of the polypeptides of the present invention. For instance, one or more amino acids can be deleted from the N-terminus or C-terminus of the secreted protein without substantial loss of biological function. The authors of Ron et al., J. Biol. Chem. 268: 2984-2988 (1993), reported variant KGF proteins having heparin binding activity even after deleting 3, 8, or 27 amino-terminal amino acid residues. Similarly, Interferon gamma exhibited up to ten times higher activity after deleting 8-10 amino acid residues from the carboxy terminus of this protein. (Dobeli et al., J. Biotechnology 7: 199-216 (1988).)

Moreover, ample evidence demonstrates that variants often retain a biological activity similar to that of the naturally occurring protein. For example, Gayle and coworkers (J. Biol. Chem 268: 22105-22111 (1993)) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See, Abstract.) In fact, only 23 unique amino acid sequences, out of more than 3,500 nucleotide sequences examined, produced a protein that significantly differed in activity from wild-type.

Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N- or C-terminal residues of a protein retains such immunogenic activities can readily be determined by routine methods described herein and otherwise known in the art.

Thus, the invention further includes polypeptide variants which show substantial biological activity. Such variants include deletions, insertions, inversions, repeats, and substitutions selected according to general rules known in the art so as have little effect on activity: For example, guidance concerning how to make phenotypically silent amino acid substitutions is provided in Bowie, J. U. et al., Science 247: 1306-1310 (1990), wherein the authors indicate that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein.

The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) can be used. (Cunningham and Wells, Science 244: 1081-1085 (1989).) The resulting mutant molecules can then be tested for biological activity.

As the authors state, these two strategies have revealed that proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved. Moreover, tolerated conservative amino acid substitutions involve replacement of the aliphatic or hydrophobic amino acids Ala, Val, Leu and lie; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

Besides conservative amino acid substitution, variants of the present invention include (i) substitutions with one or more of the non-conserved amino acid residues, where the substituted amino acid residues may or may not be one encoded by the genetic code, or (ii) substitution with one or more of amino acid residues having a substituent group, or (iii) fusion of the mature polypeptide with another compound, such as a compound to increase the stability and/or solubility of the polypeptide (for example, polyethylene glycol), or (iv) fusion of the polypeptide with additional amino acids, such as an IgG Fe fusion region peptide, or leader or secretory sequence, or a sequence facilitating purification. Such variant polypeptides are deemed to be within the scope of those skilled in the art from the teachings herein.

For example, polypeptide variants containing amino acid substitutions of charged amino acids with other charged or neutral amino acids may produce proteins with improved characteristics, such as less aggregation. Aggregation of pharmaceutical formulations both reduces activity and increases clearance due to the aggregate's immunogenic activity. (Pinckard et al., Clin. Exp. Immunol. 2: 331-340 (1967); Robbins et al., Diabetes 36: 838-845 (1987); Cleland et al., Crit. Rev. Therapeutic Drug Carrier Systems 10: 307-377 (1993).)

Polynucleotide and Polypeptide Fragments

In the present invention, a “polynucleotide fragment” refers to a short polynucleotide having a nucleic acid sequence contained in the deposited clone or shown in SEQ ID NO:X. The short nucleotide fragments are preferably at least about 15 nt, and more preferably at least about 20 nt, still more preferably at least about 30 nt, and even more preferably, at least about 40 nt in length. A fragment “at least 20 nt in length,” for example, is intended to include 20 or more contiguous bases from the cDNA sequence contained in the deposited clone or the nucleotide sequence shown in SEQ ID NO:X. These nucleotide fragments are useful as diagnostic probes and primers as discussed herein. Of course, larger fragments (e.g., 50, 150, 500, 600, 2000 nucleotides) are preferred.

Moreover, representative examples of polynucleotide fragments of the invention, include, for example, fragments having a sequence from about nucleotide number 1-50, 51-100, 101-150, 151-200, 201-250, 251-300, 301-350, 351400, 401450, 451-500, 501-550, 551-600, 651-700, 701-750, 751-800, 800-850, 851-900, 901-950, 951-1000, 1001-1050, 1051-1100, 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750, 1751-1800, 1801-1850, 1851-1900, 1901-1950, 1951-2000, or 2001 to the end of SEQ ID NO:X or the cDNA contained in the deposited clone. In this context “about” includes the particularly recited ranges, larger or smaller by several (5, 4, 3, 2, or 1) nucleotides, at either terminus or at both termini. Preferably, these fragments encode a polypeptide which has biological activity. More preferably, these polynucleotides can be used as probes or primers as discussed herein.

In the present invention, a “polypeptide fragment” refers to a short amino acid sequence contained in SEQ ID NO:Y or encoded by the cDNA contained in the deposited clone. Protein fragments may be “free-standing,” or comprised within a larger polypeptide of which the fragment forms a part or region, most preferably as a single continuous region. Representative examples of polypeptide fragments of the invention, include, for example, fragments from about amino acid number 1-20, 21-40, 41-60, 61-80, 81-100, 102-120, 121-140, 141-160, or 161 to the end of the coding region. Moreover, polypeptide fragments can be about 20, 30, 40, 50, 60, 70, 80, 90, 100, 110, 120, 130, 140, or 150 amino acids in length. In this context “about” includes the particularly recited ranges, larger or smaller by several (5, 4, 3, 2, or 1) amino acids, at either extreme or at both extremes.

Preferred polypeptide fragments include the secreted protein as well as the mature form. Further preferred polypeptide fragments include the secreted protein or the mature form having a continuous series of deleted residues from the amino or the carboxy terminus, or both. For example, any number of amino acids, ranging from 1-60, can be deleted from the amino terminus of either the secreted polypeptide or the mature form. Similarly, any number of amino acids, ranging from 1-30, can be deleted from the carboxy terminus of the secreted protein or mature form. Furthermore, any combination of the above amino and carboxy terminus deletions are preferred. Similarly, polynucleotide fragments encoding these polypeptide fragments are also preferred.

Also preferred are polypeptide and polynucleotide fragments characterized by structural or functional domains, such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Polypeptide fragments of SEQ ID NO:Y falling within conserved domains are specifically contemplated by the present invention. Moreover, polynucleotide fragments encoding these domains are also contemplated.

Other preferred fragments are biologically active fragments. Biologically active fragments are those exhibiting activity similar, but not necessarily identical, to an activity of the polypeptide of the present invention. The biological activity of the fragments may include an improved desired activity, or a decreased undesirable activity.

Epitopes & Antibodies

In the present invention, “epitopes” refer to polypeptide fragments having antigenic or immunogenic activity in an animal, especially in a human. A preferred embodiment of the present invention relates to a polypeptide fragment comprising an epitope, as well as the polynucleotide encoding this fragment. A region of a protein molecule to which an antibody can bind is defined as an “antigenic epitope.” In contrast, an “immunogenic epitope” is defined as a part of a protein that elicits an antibody response. (See, for instance, Geysen et al., Proc. Natl. Acad. Sci. USA 81: 3998-4002 (1983).)

Fragments which function as epitopes may be produced by any conventional means. (See, e.g., Houghten, R. A., Proc. Natl. Acad. Sci. USA 82: 5131-5135 (1985) further described in U.S. Pat. No. 4,631,211.)

In the present invention, antigenic epitopes preferably contain a sequence of at least seven, more preferably at least nine, and most preferably between about 15 to about 30 amino acids. Antigenic epitopes are useful to raise antibodies, including monoclonal antibodies, that specifically bind the epitope. (See, for instance, Wilson et al., Cell 37: 767-778 (1984); Sutcliffe, J. G. et al., Science 219: 660-666 (1983).) Similarly, immunogenic epitopes can be used to induce antibodies according to methods well known in the art. (See, for instance, Sutcliffe et al., supra; Wilson et al., supra; Chow, M. et al., Proc. Natl. Acad. Sci. USA 82: 910-914; and Bittle, F. J. et al., J. Gen. Virol. 66: 2347-2354 (1985).) a preferred immunogenic epitope includes the secreted protein. The immunogenic epitopes may be presented together with a carrier protein, such as an albumin, to an animal system (such as rabbit or mouse) or, if it is long enough (at least about 25 amino acids), without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting.)

As used herein, the term “antibody” (Ab) or “monoclonal antibody” (Mab) is meant to include intact molecules as well as antibody fragments (such as, for example, Fab and F(ab′)2 fragments) which are capable of specifically binding to protein. Fab and F(ab′)2 fragments lack the Fc fragment of intact antibody, clear more rapidly from the circulation, and may have less non-specific tissue binding than an intact antibody. (Wahl et al., J. Nucl. Med. 24: 316-325 (1983).) Thus, these fragments are preferred, as well as the products of a FAB or other immunoglobulin expression library. Moreover, antibodies of the present invention include chimeric, single chain, and humanized antibodies.

Fusion Proteins

Any polypeptide of the present invention can be used to generate fusion proteins. For example, the polypeptide of the present invention, when fused to a second protein, can be used as an antigenic tag. Antibodies raised against the polypeptide of the present invention can be used to indirectly detect the second protein by binding to the polypeptide. Moreover, because secreted proteins target cellular locations based on trafficking signals, the polypeptides of the present invention can be used as targeting molecules once fused to other proteins.

Examples of domains that can be fused to polypeptides of the present invention include not only heterologous signal sequences, but also other heterologous functional regions. The fusion does not necessarily need to be direct, but may occur through linker sequences.

Moreover, fusion proteins may also be engineered to improve characteristics of the polypeptide of the present invention. For instance, a region of additional amino acids, particularly charged amino acids, may be added to the N-terminus of the polypeptide to improve stability and persistence during purification from the host cell or subsequent handling and storage. Also, peptide moieties may be added to the polypeptide to facilitate purification. Such regions may be removed prior to final preparation of the polypeptide. The addition of peptide moieties to facilitate handling of polypeptides are familiar and routine techniques in the art.

Moreover, polypeptides of the present invention, including fragments, and specifically epitopes, can be combined with parts of the constant domain of immunoglobulins (IgG), resulting in chimeric polypeptides. These fusion proteins facilitate purification and show an increased half-life in vivo. One reported example describes chimeric proteins consisting of the first two domains of the human CD4-polypeptide and various domains of the constant regions of the heavy or light chains of mammalian immunoglobulins. (EP a 394,827; Traunecker et al., Nature 331: 84-86 (1988).) Fusion proteins having disulfide-linked dimeric structures (due to the IgG) can also be more efficient in binding and neutralizing other molecules, than the monomeric secreted protein or protein fragment alone. (Fountoulakis et al., J. Biochem. 270: 3958-3964 (1995).)

Similarly, EP-A-O 464 533 (Canadian counterpart 2045869) discloses fusion proteins comprising various portions of constant region of immunoglobulin molecules together with another human protein or part thereof. In many cases, the Fc part in a fusion protein is beneficial in therapy and diagnosis, and thus can result in, for example, improved pharmacokinetic properties. (EP-A 0232 262.) Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5. (See, D. Bennett et al., J. Molecular Recognition 8: 52-58 (1995); K. Johanson et al., J. Biol. Chem. 270: 9459-9471 (1995).)

Moreover, the polypeptides of the present invention can be fused to marker sequences, such as a peptide which facilitates purification of the fused polypeptide. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86: 821-824 (1989), for instance, hexa-histidine provides for convenient purification of the fusion protein. Another peptide tag useful for purification, the “HA” tag, corresponds to an epitope derived from the influenza hemagglutinin protein. (Wilson et al., Cell 37: 767 (1984).)

Thus, any of these above fusions can be engineered using the polynucleotides or the polypeptides of the present invention.

Vectors, Host Cells, and Protein Production

The present invention also relates to vectors containing the polynucleotide of the present invention, host cells, and the production of polypeptides by recombinant techniques. The vector may be, for example, a phage, plasmid, viral, or retroviral vector. Retroviral vectors may be replication competent or replication defective. In the latter case, viral propagation generally will occur only in complementing host cells.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, 293, and Bowes melanoma cells; and plant cells. Appropriate culture mediums and conditions for the above-described host cells are known in the art.

Among vectors preferred for use in bacteria include pQE70, pQE60 and pQE-9, available from QIAGEN, Inc.; pBLUESCRIPT™ vectors, Phagescript vectors, pNH8A, pNH16a, pNH18a, pNH46A, available from Stratagene Cloning Systems, Inc.; and ptrc99a, pKK223-3, pKK233-3, pDR540, pRIT5 available from Pharmacia Biotech, Inc. Among preferred eukaryotic vectors are pWLNEO, pSV2CAT, pOG44, pXT1 and pSG available from STRATAGENE™; and pSVK3, pBPV, pMSG and pSVL available from PHARMACIA™. Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that the polypeptides of the present invention may in fact be expressed by a host cell lacking a recombinant vector.

A polypeptide of this invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

Polypeptides of the present invention, and preferably the secreted form, can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

Uses of the Polynucleotides

Each of the polynucleotides identified herein can be used in numerous ways as reagents. The following description should be considered exemplary and utilizes known techniques.

The polynucleotides of the present invention are useful for chromosome identification. There exists an ongoing need to identify new chromosome markers, since few chromosome marking reagents, based on actual sequence data (repeat polymorphisms), are presently available. Each polynucleotide of the present invention can be used as a chromosome marker.

Briefly, sequences can be mapped to chromosomes by preparing PCR primers (preferably 15-25 bp) from the sequences shown in SEQ ID NO:X. Primers can be selected using computer analysis so that primers do not span more than one predicted exon in the genomic DNA. These primers are then used for PCR screening of somatic cell hybrids containing individual human chromosomes. Only those hybrids containing the human gene corresponding to the SEQ ID NO:X will yield an amplified fragment.

Similarly, somatic hybrids provide a rapid method of PCR mapping the polynucleotides to particular chromosomes. Three or more clones can be assigned per day using a single thermal cycler. Moreover, sublocalization of the polynucleotides can be achieved with panels of specific chromosome fragments. Other gene mapping strategies that can be used include in situ hybridization, prescreening with labeled flow-sorted chromosomes, and preselection by hybridization to construct chromosome specific-cDNA libraries.

Precise chromosomal location of the polynucleotides can also be achieved using fluorescence in situ hybridization (FISH) of a metaphase chromosomal spread. This technique uses polynucleotides as short as 500 or 600 bases; however, polynucleotides 2,000-4,000 bp are preferred. For a review of this technique, see Verma et al., “Human Chromosomes: a Manual of Basic Techniques,” Pergamon Press, New York (1988).

For chromosome mapping, the polynucleotides can be used individually (to mark a single chromosome or a single site on that chromosome) or in panels (for marking multiple sites and/or multiple chromosomes). Preferred polynucleotides correspond to the noncoding regions of the cDNAs because the coding sequences are more likely conserved within gene families, thus increasing the chance of cross hybridization during chromosomal mapping.

Once a polynucleotide has been mapped to a precise chromosomal location, the physical position of the polynucleotide can be used in linkage analysis. Linkage analysis establishes coinheritance between a chromosomal location and presentation of a particular disease. (Disease mapping data are found, for example, in V. McKusick, Mendelian Inheritance in Man (available on line through Johns Hopkins University Welch Medical Library).) Assuming 1 megabase mapping resolution and one gene per 20 kb, a cDNA precisely localized to a chromosomal region associated with the disease could be one of 50-500 potential causative genes.

Thus, once coinheritance is established, differences in the polynucleotide and the corresponding gene between affected and unaffected individuals can be examined. First, visible structural alterations in the chromosomes, such as deletions or translocations, are examined in chromosome spreads or by PCR. If no structural alterations exist, the presence of point mutations are ascertained. Mutations observed in some or all affected individuals, but not in normal individuals, indicates that the mutation may cause the disease. However, complete sequencing of the polypeptide and the corresponding gene from several normal individuals is required to distinguish the mutation from a polymorphism. If a new polymorphism is identified, this polymorphic polypeptide can be used for further linkage analysis.

Furthermore, increased or decreased expression of the gene in affected individuals as compared to unaffected individuals can be assessed using polynucleotides of the present invention. Any of these alterations (altered expression, chromosomal rearrangement, or mutation) can be used as a diagnostic or prognostic marker.

In addition to the foregoing, a polynucleotide can be used to control gene expression through triple helix formation or antisense DNA or RNA. Both methods rely on binding of the polynucleotide to DNA or RNA. For these techniques, preferred polynucleotides are usually 20 to 40 bases in length and complementary to either the region of the gene involved in transcription (triple helix—see Lee et al., Nucl. Acids Res. 6: 3073 (1979); Cooney et al., Science 241: 456 (1988); and Dervan et al., Science 251: 1360 (1991)) or to the mRNA itself (antisense—Okano, J. Neurochem. 56: 560 (1991); Oligodeoxy-nucleotides as Antisense Inhibitors of Gene Expression, CRC Press, Boca Raton, Fla. (1988).) Triple helix formation optimally results in a shut-off of RNA transcription from DNA, while antisense RNA hybridization blocks translation of an mRNA molecule into polypeptide. Both techniques are effective in model systems, and the information disclosed herein can be used to design antisense or triple helix polynucleotides in an effort to treat disease.

Polynucleotides of the present invention are also useful in gene therapy. One goal of gene therapy is to insert a normal gene into an organism having a defective gene, in an effort to correct the genetic defect. The polynucleotides disclosed in the present invention offer a means of targeting such genetic defects in a highly accurate manner. Another goal is to insert a new gene that was not present in the host genome, thereby producing a new trait in the host cell.

The polynucleotides are also useful for identifying individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identifying personnel. This method does not suffer from the current limitations of “Dog Tags” which can be lost, switched, or stolen, making positive identification difficult. The polynucleotides of the present invention can be used as additional DNA markers for RFLP.

The polynucleotides of the present invention can also be used as an alternative to RFLP, by determining the actual base-by-base DNA sequence of selected portions of an individual's genome. These sequences can be used to prepare PCR primers for amplifying and isolating such selected DNA, which can then be sequenced. Using this technique, individuals can be identified because each individual will have a unique set of DNA sequences. Once an unique ID database is established for an individual, positive identification of that individual, living or dead, can be made from extremely small tissue samples.

Forensic biology also benefits from using DNA-based identification techniques as disclosed herein. DNA sequences taken from very small biological samples such as tissues, e.g., hair or skin, or body fluids, e.g., blood, saliva, semen, etc., can be amplified using PCR. In one prior art technique, gene sequences amplified from polymorphic loci, such as DQa class II HLA gene, are used in forensic biology to identify individuals. (Erlich, H., PCR Technology, Freeman and Co. (1992).) Once these specific polymorphic loci are amplified, they are digested with one or more restriction enzymes, yielding an identifying set of bands on a Southern blot probed with DNA corresponding to the DQa class II HLA gene. Similarly, polynucleotides of the present invention can be used as polymorphic markers for forensic purposes.

There is also a need for reagents capable of identifying the source of a particular tissue. Such need arises, for example, in forensics when presented with tissue of unknown origin. Appropriate reagents can comprise, for example, DNA probes or primers specific to particular tissue prepared from the sequences of the present invention. Panels of such reagents can identify tissue by species and/or by organ type. In a similar fashion, these reagents can be used to screen tissue cultures for contamination.

In the very least, the polynucleotides of the present invention can be used as molecular weight markers on Southern gels, as diagnostic probes for the presence of a specific mRNA in a particular cell type, as a probe to “subtract-out” known sequences in the process of discovering novel polynucleotides, for selecting and making oligomers for attachment to a “gene chip” or other support, to raise anti-DNA antibodies using DNA immunization techniques, and as an antigen to elicit an immune response.

Uses of the Polypeptides

Each of the polypeptides identified herein can be used in numerous ways. The following description should be considered exemplary and utilizes known techniques.

A polypeptide of the present invention can be used to assay protein levels in a biological sample using antibody-based techniques. For example, protein expression in tissues can be studied with classical immunohistological methods. (Jalkanen, M., et al., J. Cell. Biol. 101: 976-985 (1985); Jalkanen, M., et al., J. Cell. Biol. 105: 3087-3096 (1987).) Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (125I, 121I), carbon (14C), sulfur (35S), tritium (3H), indium (112In), and technetium (99mTc), and fluorescent labels, such as fluorescein and rhodamine, and biotin.

In addition to assaying secreted protein levels in a biological sample, proteins can also be detected in vivo by imaging. Antibody labels or markers for in vivo imaging of protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

A protein-specific antibody or antibody fragment which has been labeled with an appropriate detectable imaging moiety, such as a radioisotope (for example, 131I, 112In, 99mTc), a radio-opaque substance, or a material detectable by nuclear magnetic resonance, is introduced (for example, parenterally, subcutaneously, or intraperitoneally) into the mammal. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99 mTc. The labeled antibody or antibody fragment will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).)

Thus, the invention provides a diagnostic method of a disorder, which involves (a) assaying the expression of a polypeptide of the present invention in cells or body fluid of an individual; (b) comparing the level of gene expression with a standard gene expression level, whereby an increase or decrease in the assayed polypeptide gene expression level compared to the standard expression level is indicative of a disorder.

Moreover, polypeptides of the present invention can be used to treat disease. For example, patients can be administered a polypeptide of the present invention in an effort to replace absent or decreased levels of the polypeptide (e.g., insulin), to supplement absent or decreased levels of a different polypeptide (e.g., hemoglobin S for hemoglobin B), to inhibit the activity of a polypeptide (e.g., an oncogene), to activate the activity of a polypeptide (e.g., by binding to a receptor), to reduce the activity of a membrane bound receptor by competing with it for free ligand (e.g., soluble TNF receptors used in reducing inflammation), or to bring about a desired response (e.g., blood vessel growth).

Similarly, antibodies directed to a polypeptide of the present invention can also be used to treat disease. For example, administration of an antibody directed to a polypeptide of the present invention can bind and reduce overproduction of the polypeptide. Similarly, administration of an antibody can activate the polypeptide, such as by binding to a polypeptide bound to a membrane (receptor).

At the very least, the polypeptides of the present invention can be used as molecular weight markers on SDS-PAGE gels or on molecular sieve gel filtration columns using methods well known to those of skill in the art. Polypeptides can also be used to raise antibodies, which in turn are used to measure protein expression from a recombinant cell, as a way of assessing transformation of the host cell. Moreover, the polypeptides of the present invention can be used to test the following biological activities.

Biological Activities

The polynucleotides and polypeptides of the present invention can be used in assays to test for one or more biological activities. If these polynucleotides and polypeptides do exhibit activity in a particular assay, it is likely that these molecules may be involved in the diseases associated with the biological activity. Thus, the polynucleotides and polypeptides could be used to treat the associated disease.

Immune Activity

A polypeptide or polynucleotide of the present invention may be useful in treating deficiencies or disorders of the immune system, by activating or inhibiting the proliferation, differentiation, or mobilization (chemotaxis) of immune cells. Immune cells develop through a process called hematopoiesis, producing myeloid (platelets, red blood cells, neutrophils, and macrophages) and lymphoid (B and T lymphocytes) cells from pluripotent stem cells. The etiology of these immune deficiencies or disorders may be genetic, somatic, such as cancer or some autoimmune disorders, acquired (e.g., by chemotherapy or toxins), or infectious. Moreover, a polynucleotide or polypeptide of the present invention can be used as a marker or detector of a particular immune system disease or disorder.

A polynucleotide or polypeptide of the present invention may be useful in treating or detecting deficiencies or disorders of hematopoietic cells. A polypeptide or polynucleotide of the present invention could be used to increase differentiation and proliferation of hematopoietic cells, including the pluripotent stem cells, in an effort to treat those disorders associated with a decrease in certain (or many) types hematopoietic cells. Examples of immunologic deficiency syndromes include, but are not limited to: blood protein disorders (e.g. agammaglobulinemia, dysgammaglobulinemia), ataxia telangiectasia, common variable immunodeficiency, Digeorge Syndrome, HIV infection, HTLV-BLV infection, leukocyte adhesion deficiency syndrome, lymphopenia, phagocyte bactericidal dysfunction, severe combined immunodeficiency (SCIDs), Wiskott-Aldrich Disorder, anemia, thrombocytopenia, or hemoglobinuria.

Moreover, a polypeptide or polynucleotide of the present invention could also be used to modulate hemostatic (the stopping of bleeding) or thrombolytic activity (clot formation). For example, by increasing hemostatic or thrombolytic activity, a polynucleotide or polypeptide of the present invention could be used to treat blood coagulation disorders (e.g., afibrinogenemia, factor deficiencies), blood platelet disorders (e.g. thrombocytopenia), or wounds resulting from trauma, surgery, or other causes. Alternatively, a polynucleotide or polypeptide of the present invention that can decrease hemostatic or thrombolytic activity could be used to inhibit or dissolve clotting. These molecules could be important in the treatment of heart attacks (infarction), strokes, or scarring.

A polynucleotide or polypeptide of the present invention may also be useful in treating or detecting autoimmune disorders. Many autoimmune disorders result from inappropriate recognition of self as foreign material by immune cells. This inappropriate recognition results in an immune response leading to the destruction of the host tissue. Therefore, the administration of a polypeptide or polynucleotide of the present invention that inhibits an immune response, particularly the proliferation, differentiation, or chemotaxis of T-cells, may be an effective therapy in preventing autoimmune disorders.

Examples of autoimmune disorders that can be treated or detected by the present invention include, but are not limited to: Addison's Disease, hemolytic anemia, antiphospholipid syndrome, rheumatoid arthritis, dermatitis, allergic encephalomyelitis, glomerulonephritis, Goodpasture's Syndrome, Graves' Disease, Multiple Sclerosis, Myasthenia Gravis, Neuritis, Ophthalmia, Bullous Pemphigoid, Pemphigus, Polyendocrinopathies, Purpura, Reiter's Disease, Stiff-Man Syndrome, Autoimmune Thyroiditis, Systemic Lupus Erythematosus, Autoimmune Pulmonary Inflammation, Guillain-Barre Syndrome, insulin dependent diabetes mellitis, and autoimmune inflammatory eye disease.

Similarly, allergic reactions and conditions, such as asthma (particularly allergic asthma) or other respiratory problems, may also be treated by a polypeptide or polynucleotide of the present invention. Moreover, these molecules can be used to treat anaphylaxis, hypersensitivity to an antigenic molecule, or blood group incompatibility.

A polynucleotide or polypeptide of the present invention may also be used to treat and/or prevent organ rejection or graft-versus-host disease (GVHD). Organ rejection occurs by host immune cell destruction of the transplanted tissue through an immune response. Similarly, an immune response is also involved in GVHD, but, in this case, the foreign transplanted immune cells destroy the host tissues. The administration of a polypeptide or polynucleotide of the present invention that inhibits an immune response, particularly the proliferation, differentiation, or chemotaxis of T-cells, may be an effective therapy in preventing organ rejection or GVHD.

Similarly, a polypeptide or polynucleotide of the present invention may also be used to modulate inflammation. For example, the polypeptide or polynucleotide may inhibit the proliferation and differentiation of cells involved in an inflammatory response. These molecules can be used to treat inflammatory conditions, both chronic and acute conditions, including inflammation associated with infection (e.g., septic shock, sepsis, or systemic inflammatory response syndrome (SIRS)), ischemia-reperfusion injury, endotoxin lethality, arthritis, complement-mediated hyperacute rejection, nephritis, cytokine or chemokine induced lung injury, inflammatory bowel disease, Crohn's disease, or resulting from over production of cytokines (e.g., TNF or IL-1.)

Hyperproliferative Disorders

A polypeptide or polynucleotide can be used to treat or detect hyperproliferative disorders, including neoplasms. A polypeptide or polynucleotide of the present invention may inhibit the proliferation of the disorder through direct or indirect interactions. Alternatively, a polypeptide or polynucleotide of the present invention may proliferate other cells which can inhibit the hyperproliferative disorder.

For example, by increasing an immune response, particularly increasing antigenic qualities of the hyperproliferative disorder or by proliferating, differentiating, or mobilizing T-cells, hyperproliferative disorders can be treated. This immune response may be increased by either enhancing an existing immune response, or by initiating a new immune response. Alternatively, decreasing an immune response may also be a method of treating hyperproliferative disorders, such as a chemotherapeutic agent.

Examples of hyperproliferative disorders that can be treated or detected by a polynucleotide or polypeptide of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital.

Similarly, other hyperproliferative disorders can also be treated or detected by a polynucleotide or polypeptide of the present invention. Examples of such hyperproliferative disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

Infectious Disease

A polypeptide or polynucleotide of the present invention can be used to treat or detect infectious agents. For example, by increasing the immune response, particularly increasing the proliferation and differentiation of B and/or T cells, infectious diseases may be treated. The immune response may be increased by either enhancing an existing immune response, or by initiating a new immune response. Alternatively, the polypeptide or polynucleotide of the present invention may also directly inhibit the infectious agent, without necessarily eliciting an immune response.

Viruses are one example of an infectious agent that can cause disease or symptoms that can be treated or detected by a polynucleotide or polypeptide of the present invention. Examples of viruses, include, but are not limited to the following DNA and RNA viral families: Arbovirus, Adenoviridae, Arenaviridae, Arterivirus, Birnaviridae, Bunyaviridae, Caliciviridae, Circoviridae, Coronaviridae, Flaviviridae, Hepadnaviridae (Hepatitis), Herpesviridae (such as, Cytomegalovirus, Herpes Simplex, Herpes Zoster), Mononegavirus (e.g., Paramyxoviridae, Morbillivirus, Rhabdoviridae), Orthomyxoviridae (e.g., Influenza), Papovaviridae, Parvoviridae, Picornaviridae, Poxyiridae (such as Smallpox or Vaccinia), Reoviridae (e.g., Rotavirus), Retroviridae (HTLV-I, HTLV-II, Lentivirus), and Togaviridae (e.g., Rubivirus). Viruses falling within these families can cause a variety of diseases or symptoms, including, but not limited to: arthritis, bronchiollitis, encephalitis, eye infections (e.g., conjunctivitis, keratitis), chronic fatigue syndrome, hepatitis (A, B, C, E, Chronic Active, Delta), meningitis, opportunistic infections (e.g., AIDS), pneumonia, Burkitt's Lymphoma, chickenpox, hemorrhagic fever, Measles, Mumps, Parainfluenza, Rabies, the common cold, Polio, leukemia, Rubella, sexually transmitted diseases, skin diseases (e.g., Kaposi's, warts), and viremia. A polypeptide or polynucleotide of the present invention can be used to treat or detect any of these symptoms or diseases.

Similarly, bacterial or fungal agents that can cause disease or symptoms and that can be treated or detected by a polynucleotide or polypeptide of the present invention include, but not limited to, the following Gram-Negative and Gram-positive bacterial families and fungi: Actinomycetales (e.g., Corynebacterium, Mycobacterium, Norcardia), Aspergillosis, Bacillaceae (e.g., Anthrax, Clostridium), Bacteroidaceae, Blastomycosis, Bordetella, Borrelia, Brucellosis, Candidiasis, Campylobacter, Coccidioidomycosis, Cryptococcosis, Dermatocycoses, Enterobacteriaceae (Klebsiella, Salmonella, Serratia, Yersinia), Erysipelothrix, Helicobacter, Legionellosis, Leptospirosis, Listeria, Mycoplasmatales, Neisseriaceae (e.g., Acinetobacter, Gonorrhea, Menigococcal), Pasteurellacea Infections (e.g., Actinobacillus, Heamophilus, Pasteurella), Pseudomonas, Rickettsiaceae, Chlamydiaceae, Syphilis, and Staphylococcal. These bacterial or fungal families can cause the following diseases or symptoms, including, but not limited to: bacteremia, endocarditis, eye infections (conjunctivitis, tuberculosis, uveitis), gingivitis, opportunistic infections (e.g., AIDS related infections), paronychia, prosthesis-related infections, Reiter's Disease, respiratory tract infections, such as Whooping Cough or Empyema, sepsis, Lyme Disease, Cat-Scratch Disease, Dysentery, Paratyphoid Fever, food poisoning, Typhoid, pneumonia, Gonorrhea, meningitis, Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g., cellulitis, dermatocycoses), toxemia, urinary tract infections, wound infections. A polypeptide or polynucleotide of the present invention can be used to treat or detect any of these symptoms or diseases.

Moreover, parasitic agents causing disease or symptoms that can be treated or detected by a polynucleotide or polypeptide of the present invention include, but not limited to, the following families: Amebiasis, Babesiosis, Coccidiosis, Cryptosporidiosis, Dientamoebiasis, Dourine, Ectoparasitic, Giardiasis, Helminthiasis, Leishmaniasis, Theileriasis, Toxoplasmosis, Trypanosomiasis, and Trichomonas. These parasites can cause a variety of diseases or symptoms, including, but not limited to: Scabies, Trombiculiasis, eye infections, intestinal disease (e.g., dysentery, giardiasis), liver disease, lung disease, opportunistic infections (e.g., AIDS related), Malaria, pregnancy complications, and toxoplasmosis. A polypeptide or polynucleotide of the present invention can be used to treat or detect any of these symptoms or diseases.

Preferably, treatment using a polypeptide or polynucleotide of the present invention could either be by administering an effective amount of a polypeptide to the patient, or by removing cells from the patient, supplying the cells with a polynucleotide of the present invention, and returning the engineered cells to the patient (ex vivo therapy). Moreover, the polypeptide or polynucleotide of the present invention can be used as an antigen in a vaccine to raise an immune response against infectious disease.

Regeneration

A polynucleotide or polypeptide of the present invention can be used to differentiate, proliferate, and attract cells, leading to the regeneration of tissues. (See, Science 276: 59-87 (1997).) The regeneration of tissues could be used to repair, replace, or protect tissue damaged by congenital defects, trauma (wounds, burns, incisions, or ulcers), age, disease (e.g. osteoporosis, osteocarthritis, periodontal disease, liver failure), surgery, including cosmetic plastic surgery, fibrosis, reperfusion injury, or systemic cytokine damage.

Tissues that could be regenerated using the present invention include organs (e.g., pancreas, liver, intestine, kidney, skin, endothelium), muscle (smooth, skeletal or cardiac), vascular (including vascular endothelium), nervous, hematopoietic, and skeletal (bone, cartilage, tendon, and ligament) tissue. Preferably, regeneration occurs without or decreased scarring. Regeneration also may include angiogenesis.

Moreover, a polynucleotide or polypeptide of the present invention may increase regeneration of tissues difficult to heal. For example, increased tendon/ligament regeneration would quicken recovery time after damage. A polynucleotide or polypeptide of the present invention could also be used prophylactically in an effort to avoid damage. Specific diseases that could be treated include of tendinitis, carpal tunnel syndrome, and other tendon or ligament defects. A further example of tissue regeneration of non-healing wounds includes pressure ulcers, ulcers associated with vascular insufficiency, surgical, and traumatic wounds.

Similarly, nerve and brain tissue could also be regenerated by using a polynucleotide or polypeptide of the present invention to proliferate and differentiate nerve cells. Diseases that could be treated using this method include central and peripheral nervous system diseases, neuropathies, or mechanical and traumatic disorders (e.g., spinal cord disorders, head trauma, cerebrovascular disease, and stoke). Specifically, diseases associated with peripheral nerve injuries, peripheral neuropathy (e.g., resulting from chemotherapy or other medical therapies), localized neuropathies, and central nervous system diseases (e.g., Alzheimer's disease, Parkinson's disease, Huntington's disease, amyotrophic lateral sclerosis, and Shy-Drager syndrome), could all be treated using the polynucleotide or polypeptide of the present invention.

Chemotaxis

A polynucleotide or polypeptide of the present invention may have chemotaxis activity. A chemotaxic molecule attracts or mobilizes cells (e.g., monocytes, fibroblasts, neutrophils, T-cells, mast cells, eosinophils, epithelial and/or endothelial cells) to a particular site in the body, such as inflammation, infection, or site of hyperproliferation. The mobilized cells can then fight off and/or heal the particular trauma or abnormality.

A polynucleotide or polypeptide of the present invention may increase chemotaxic activity of particular cells. These chemotactic molecules can then be used to treat inflammation, infection, hyperproliferative disorders, or any immune system disorder by increasing the number of cells targeted to a particular location in the body. For example, chemotaxic molecules can be used to treat wounds and other trauma to tissues by attracting immune cells to the injured location. Chemotactic molecules of the present invention can also attract fibroblasts, which can be used to treat wounds.

It is also contemplated that a polynucleotide or polypeptide of the present invention may inhibit chemotactic activity. These molecules could also be used to treat disorders. Thus, a polynucleotide or polypeptide of the present invention could be used as an inhibitor of chemotaxis.

Binding Activity

A polypeptide of the present invention may be used to screen for molecules that bind to the polypeptide or for molecules to which the polypeptide binds. The binding of the polypeptide and the molecule may activate (agonist), increase, inhibit (antagonist), or decrease activity of the polypeptide or the molecule bound. Examples of such molecules include antibodies, oligonucleotides, proteins (e.g., receptors), or small molecules.

Preferably, the molecule is closely related to the natural ligand of the polypeptide, e.g., a fragment of the ligand, or a natural substrate, a ligand, a structural or functional mimetic. (See, Coligan et al., Current Protocols in Immunology 1(2): Chapter 5 (1991).) Similarly, the molecule can be closely related to the natural receptor to which the polypeptide binds, or at least, a fragment of the receptor capable of being bound by the polypeptide (e.g., active site). In either case, the molecule can be rationally designed using known techniques.

Preferably, the screening for these molecules involves producing appropriate cells which express the polypeptide, either as a secreted protein or on the cell membrane. Preferred cells include cells from mammals, yeast, Drosophila, or E. coli. Cells expressing the polypeptide (or cell membrane containing the expressed polypeptide) are then preferably contacted with a test compound potentially containing the molecule to observe binding, stimulation, or inhibition of activity of either the polypeptide or the molecule.

The assay may simply test binding of a candidate compound to the polypeptide, wherein binding is detected by a label, or in an assay involving competition with a labeled competitor. Further, the assay may test whether the candidate compound results in a signal generated by binding to the polypeptide.

Alternatively, the assay can be carried out using cell-free preparations, polypeptide/molecule affixed to a solid support, chemical libraries, or natural product mixtures. The assay may also simply comprise the steps of mixing a candidate compound with a solution containing a polypeptide, measuring polypeptide/molecule activity or binding, and comparing the polypeptide/molecule activity or binding to a standard.

Preferably, an ELISA assay can measure polypeptide level or activity in a sample (e.g., biological sample) using a monoclonal or polyclonal antibody. The antibody can measure polypeptide level or activity by either binding, directly or indirectly, to the polypeptide or by competing with the polypeptide for a substrate.

All of these above assays can be used as diagnostic or prognostic markers. The molecules discovered using these assays can be used to treat disease or to bring about a particular result in a patient (e.g., blood vessel growth) by activating or inhibiting the polypeptide/molecule. Moreover, the assays can discover agents which may inhibit or enhance the production of the polypeptide from suitably manipulated cells or tissues.

Therefore, the invention includes a method of identifying compounds which bind to a polypeptide of the invention comprising the steps of: (a) incubating a candidate binding compound with a polypeptide of the invention; and (b) determining if binding has occurred. Moreover, the invention includes a method of identifying agonists/antagonists comprising the steps of: (a) incubating a candidate compound with a polypeptide of the invention, (b) assaying a biological activity, and (b) determining if a biological activity of the polypeptide has been altered.

Other Activities

A polypeptide or polynucleotide of the present invention may also increase or decrease the differentiation or proliferation of embryonic stem cells, besides, as discussed above, hematopoietic lineage.

A polypeptide or polynucleotide of the present invention may also be used to modulate mammalian characteristics, such as body height, weight, hair color, eye color, skin, percentage of adipose tissue, pigmentation, size, and shape (e.g., cosmetic surgery). Similarly, a polypeptide or polynucleotide of the present invention may be used to modulate mammalian metabolism affecting catabolism, anabolism, processing, utilization, and storage of energy.

A polypeptide or polynucleotide of the present invention may be used to change a mammal's mental state or physical state by influencing biorhythms, caricadic rhythms, depression (including depressive disorders), tendency for violence, tolerance for pain, reproductive capabilities (preferably by Activin or Inhibin-like activity), hormonal or endocrine levels, appetite, libido, memory, stress, or other cognitive qualities.

A polypeptide or polynucleotide of the present invention may also be used as a food additive or preservative, such as to increase or decrease storage capabilities, fat content, lipid, protein, carbohydrate, vitamins, minerals, cofactors or other nutritional components.

### Other Preferred Embodiments

Other preferred embodiments of the claimed invention include an isolated nucleic acid molecule comprising a nucleotide sequence which is at least 95% identical to a sequence of at least about 50 contiguous nucleotides in the nucleotide sequence of SEQ ID NO:X wherein X is any integer as defined in Table 1.

Also preferred is a nucleic acid molecule wherein said sequence of contiguous nucleotides is included in the nucleotide sequence of SEQ ID NO:X in the range of positions beginning with the nucleotide at about the position of the 5′ Nucleotide of the Clone Sequence and ending with the nucleotide at about the position of the 3′ Nucleotide of the Clone Sequence as defined for SEQ ID NO:X in Table 1.

Also preferred is a nucleic acid molecule wherein said sequence of contiguous nucleotides is included in the nucleotide sequence of SEQ ID NO:X in the range of positions beginning with the nucleotide at about the position of the 5′ Nucleotide of the Start Codon and ending with the nucleotide at about the position of the 3′ Nucleotide of the Clone Sequence as defined for SEQ ID NO:X in Table 1.

Similarly preferred is a nucleic acid molecule wherein said sequence of contiguous nucleotides is included in the nucleotide sequence of SEQ ID NO:X in the range of positions beginning with the nucleotide at about the position of the 5′ Nucleotide of the First Amino Acid of the Signal Peptide and ending with the nucleotide at about the position of the 3′ Nucleotide of the Clone Sequence as defined for SEQ ID NO:X in Table 1.

Also preferred is an isolated nucleic acid molecule comprising a nucleotide sequence which is at least 95% identical to a sequence of at least about 150 contiguous nucleotides in the nucleotide sequence of SEQ ID NO:X.

Further preferred is an isolated nucleic acid molecule comprising a nucleotide sequence which is at least 95% identical to a sequence of at least about 500 contiguous nucleotides in the nucleotide sequence of SEQ ID NO:X.

A further preferred embodiment is a nucleic acid molecule comprising a nucleotide sequence which is at least 95% identical to the nucleotide sequence of SEQ ID NO:X beginning with the nucleotide at about the position of the 5′ Nucleotide of the First Amino Acid of the Signal Peptide and ending with the nucleotide at about the position of the 3′ Nucleotide of the Clone Sequence as defined for SEQ ID NO:X in Table 1.

A further preferred embodiment is an isolated nucleic acid molecule comprising a nucleotide sequence which is at least 95% identical to the complete nucleotide sequence of SEQ ID NO:X.

Also preferred is an isolated nucleic acid molecule which hybridizes under stringent hybridization conditions to a nucleic acid molecule, wherein said nucleic acid molecule which hybridizes does not hybridize under stringent hybridization conditions to a nucleic acid molecule having a nucleotide sequence consisting of only a residues or of only T residues.

Also preferred is a composition of matter comprising a DNA molecule which comprises a human cDNA clone identified by a cDNA Clone Identifier in Table 1, which DNA molecule is contained in the material deposited with the American Type Culture Collection and given the ATCC™ Deposit Number shown in Table 1 for said cDNA Clone Identifier.

Also preferred is an isolated nucleic acid molecule comprising a nucleotide sequence which is at least 95% identical to a sequence of at least 50 contiguous nucleotides in the nucleotide sequence of a human cDNA clone identified by a cDNA Clone Identifier in Table 1, which DNA molecule is contained in the deposit given the ATCC™ Deposit Number shown in Table 1.

Also preferred is an isolated nucleic acid molecule, wherein said sequence of at least 50 contiguous nucleotides is included in the nucleotide sequence of the complete open reading frame sequence encoded by said human cDNA clone.

Also preferred is an isolated nucleic acid molecule comprising a nucleotide sequence which is at least 95% identical to sequence of at least 150 contiguous nucleotides in the nucleotide sequence encoded by said human cDNA clone.

A further preferred embodiment is an isolated nucleic acid molecule comprising a nucleotide sequence which is at least 95% identical to sequence of at least 500 contiguous nucleotides in the nucleotide sequence encoded by said human cDNA clone.

A further preferred embodiment is an isolated nucleic acid molecule comprising a nucleotide sequence which is at least 95% identical to the complete nucleotide sequence encoded by said human cDNA clone.

A further preferred embodiment is a method for detecting in a biological sample a nucleic acid molecule comprising a nucleotide sequence which is at least 95% identical to a sequence of at least 50 contiguous nucleotides in a sequence selected from the group consisting of: a nucleotide sequence of SEQ ID NO:X wherein X is any integer as defined in Table 1; and a nucleotide sequence encoded by a human cDNA clone identified by a cDNA Clone Identifier in Table 1 and contained in the deposit with the ATCC™ Deposit Number shown for said cDNA clone in Table 1; which method comprises a step of comparing a nucleotide sequence of at least one nucleic acid molecule in said sample with a sequence selected from said group and determining whether the sequence of said nucleic acid molecule in said sample is at least 95% identical to said selected sequence.

Also preferred is the above method wherein said step of comparing sequences comprises determining the extent of nucleic acid hybridization between nucleic acid molecules in said sample and a nucleic acid molecule comprising said sequence selected from said group. Similarly, also preferred is the above method wherein said step of comparing sequences is performed by comparing the nucleotide sequence determined from a nucleic acid molecule in said sample with said sequence selected from said group. The nucleic acid molecules can comprise DNA molecules or RNA molecules.

A further preferred embodiment is a method for identifying the species, tissue or cell type of a biological sample which method comprises a step of detecting nucleic acid molecules in said sample, if any, comprising a nucleotide sequence that is at least 95% identical to a sequence of at least 50 contiguous nucleotides in a sequence selected from the group consisting of: a nucleotide sequence of SEQ ID NO:X wherein X is any integer as defined in Table 1; and a nucleotide sequence encoded by a human cDNA clone identified by a cDNA Clone Identifier in Table 1 and contained in the deposit with the ATCC™ Deposit Number shown for said cDNA clone in Table 1.

The method for identifying the species, tissue or cell type of a biological sample can comprise a step of detecting nucleic acid molecules comprising a nucleotide sequence in a panel of at least two nucleotide sequences, wherein at least one sequence in said panel is at least 95% identical to a sequence of at least 50 contiguous nucleotides in a sequence selected from said group.

Also preferred is a method for diagnosing in a subject a pathological condition associated with abnormal structure or expression of a gene encoding a secreted protein identified in Table 1, which method comprises a step of detecting in a biological sample obtained from said subject nucleic acid molecules, if any, comprising a nucleotide sequence that is at least 95% identical to a sequence of at least 50 contiguous nucleotides in a sequence selected from the group consisting of: a nucleotide sequence of SEQ ID NO:X wherein X is any integer as defined in Table 1; and a nucleotide sequence encoded by a human cDNA clone identified by a cDNA Clone Identifier in Table 1 and contained in the deposit with the ATCC™ Deposit Number shown for said cDNA clone in Table 1.

The method for diagnosing a pathological condition can comprise a step of detecting nucleic acid molecules comprising a nucleotide sequence in a panel of at least two nucleotide sequences, wherein at least one sequence in said panel is at least 95% identical to a sequence of at least 50 contiguous nucleotides in a sequence selected from said group.

Also preferred is a composition of matter comprising isolated nucleic acid molecules wherein the nucleotide sequences of said nucleic acid molecules comprise a panel of at least two nucleotide sequences, wherein at least one sequence in said panel is at least 95% identical to a sequence of at least 50 contiguous nucleotides in a sequence selected from the group consisting of: a nucleotide sequence of SEQ ID NO:X wherein X is any integer as defined in Table 1; and a nucleotide sequence encoded by a human cDNA clone identified by a cDNA Clone Identifier in Table 1 and contained in the deposit with the ATCC™ Deposit Number shown for said cDNA clone in Table 1. The nucleic acid molecules can comprise DNA molecules or RNA molecules.

Also preferred is an isolated polypeptide comprising an amino acid sequence at least 90% identical to a sequence of at least about 10 contiguous amino acids in the amino acid sequence of SEQ ID NO:Y wherein Y is any integer as defined in Table 1.

Also preferred is a polypeptide, wherein said sequence of contiguous amino acids is included in the amino acid sequence of SEQ ID NO:Y in the range of positions beginning with the residue at about the position of the First Amino Acid of the Secreted Portion and ending with the residue at about the Last Amino Acid of the Open Reading Frame as set forth for SEQ ID NO:Y in Table 1.

Also preferred is an isolated polypeptide comprising an amino acid sequence at least 95% identical to a sequence of at least about 30 contiguous amino acids in the amino acid sequence of SEQ ID NO:Y.

Further preferred is an isolated polypeptide comprising an amino acid sequence at least 95% identical to a sequence of at least about 100 contiguous amino acids in the amino acid sequence of SEQ ID NO:Y.

Further preferred is an isolated polypeptide comprising an amino acid sequence at least 95% identical to the complete amino acid sequence of SEQ ID NO:Y.

Further preferred is an isolated polypeptide comprising an amino acid sequence at least 90% identical to a sequence of at least about 10 contiguous amino acids in the complete amino acid sequence of a secreted protein encoded by a human cDNA clone identified by a cDNA Clone Identifier in Table 1 and contained in the deposit with the ATCC™ Deposit Number shown for said cDNA clone in Table 1.

Also preferred is a polypeptide wherein said sequence of contiguous amino acids is included in the amino acid sequence of a secreted portion of the secreted protein encoded by a human cDNA clone identified by a cDNA Clone Identifier in Table 1 and contained in the deposit with the ATCC™ Deposit Number shown for said cDNA clone in Table I.

Also preferred is an isolated polypeptide comprising an amino acid sequence at least 95% identical to a sequence of at least about 30 contiguous amino acids in the amino acid sequence of the secreted portion of the protein encoded by a human cDNA clone identified by a cDNA Clone Identifier in Table 1 and contained in the deposit with the ATCC™ Deposit Number shown for said cDNA clone in Table 1.

Also preferred is an isolated polypeptide comprising an amino acid sequence at least 95% identical to a sequence of at least about 100 contiguous amino acids in the amino acid sequence of the secreted portion of the protein encoded by a human cDNA clone identified by a cDNA Clone Identifier in Table 1 and contained in the deposit with the ATCC™ Deposit Number shown for said cDNA clone in Table 1.

Also preferred is an isolated polypeptide comprising an amino acid sequence at least 95% identical to the amino acid sequence of the secreted portion of the protein encoded by a human cDNA clone identified by a cDNA Clone Identifier in Table 1 and contained in the deposit with the ATCC™ Deposit Number shown for said cDNA clone in Table 1.

Further preferred is an isolated antibody which binds specifically to a polypeptide comprising an amino acid sequence that is at least 90% identical to a sequence of at least 10 contiguous amino acids in a sequence selected from the group consisting of: an amino acid sequence of SEQ ID NO:Y wherein Y is any integer as defined in Table 1; and a complete amino acid sequence of a protein encoded by a human cDNA clone identified by a cDNA Clone Identifier in Table 1 and contained in the deposit with the ATCC™ Deposit Number shown for said cDNA clone in Table 1.

Further preferred is a method for detecting in a biological sample a polypeptide comprising an amino acid sequence which is at least 90% identical to a sequence of at least 10 contiguous amino acids in a sequence selected from the group consisting of: an amino acid sequence of SEQ ID NO:Y wherein Y is any integer as defined in Table 1; and a complete amino acid sequence of a protein encoded by a human cDNA clone identified by a cDNA Clone Identifier in Table 1 and contained in the deposit with the ATCC™ Deposit Number shown for said cDNA clone in Table 1; which method comprises a step of comparing an amino acid sequence of at least one polypeptide molecule in said sample with a sequence selected from said group and determining whether the sequence of said polypeptide molecule in said sample is at least 90% identical to said sequence of at least 10 contiguous amino acids.

Also preferred is the above method wherein said step of comparing an amino acid sequence of at least one polypeptide molecule in said sample with a sequence selected from said group comprises determining the extent of specific binding of polypeptides in said sample to an antibody which binds specifically to a polypeptide comprising an amino acid sequence that is at least 90% identical to a sequence of at least 10 contiguous amino acids in a sequence selected from the group consisting of: an amino acid sequence of SEQ ID NO:Y wherein Y is any integer as defined in Table 1; and a complete amino acid sequence of a protein encoded by a human cDNA clone identified by a cDNA Clone Identifier in Table 1 and contained in the deposit with the ATCC™ Deposit Number shown for said cDNA clone in Table 1.

Also preferred is the above method wherein said step of comparing sequences is performed by comparing the amino acid sequence determined from a polypeptide molecule in said sample with said sequence selected from said group.

Also preferred is a method for identifying the species, tissue or cell type of a biological sample which method comprises a step of detecting polypeptide molecules in said sample, if any, comprising an amino acid sequence that is at least 90% identical to a sequence of at least 10 contiguous amino acids in a sequence selected from the group consisting of: an amino acid sequence of SEQ ID NO:Y wherein Y is any integer as defined in Table 1; and a complete amino acid sequence of a secreted protein encoded by a human cDNA clone identified by a cDNA Clone Identifier in Table 1 and contained in the deposit with the ATCC™ Deposit Number shown for said cDNA clone in Table 1.

Also preferred is the above method for identifying the species, tissue or cell type of a biological sample, which method comprises a step of detecting polypeptide molecules comprising an amino acid sequence in a panel of at least two amino acid sequences, wherein at least one sequence in said panel is at least 90% identical to a sequence of at least 10 contiguous amino acids in a sequence selected from the above group.

Also preferred is a method for diagnosing in a subject a pathological condition associated with abnormal structure or expression of a gene encoding a secreted protein identified in Table 1, which method comprises a step of detecting in a biological sample obtained from said subject polypeptide molecules comprising an amino acid sequence in a panel of at least two amino acid sequences, wherein at least one sequence in said panel is at least 90% identical to a sequence of at least 10 contiguous amino acids in a sequence selected from the group consisting of: an amino acid sequence of SEQ ID NO:Y wherein Y is any integer as defined in Table 1; and a complete amino acid sequence of a secreted protein encoded by a human cDNA clone identified by a cDNA Clone Identifier in Table 1 and contained in the deposit with the ATCC™ Deposit Number shown for said cDNA clone in Table 1.

In any of these methods, the step of detecting said polypeptide molecules includes using an antibody.

Also preferred is an isolated nucleic acid molecule comprising a nucleotide sequence which is at least 95% identical to a nucleotide sequence encoding a polypeptide wherein said polypeptide comprises an amino acid sequence that is at least 90% identical to a sequence of at least 10 contiguous amino acids in a sequence selected from the group consisting of: an amino acid sequence of SEQ ID NO:Y wherein Y is any integer as defined in Table 1; and a complete amino acid sequence of a secreted protein encoded by a human cDNA clone identified by a cDNA Clone Identifier in Table 1 and contained in the deposit with the ATCC™ Deposit Number shown for said cDNA clone in Table 1.

Also preferred is an isolated nucleic acid molecule, wherein said nucleotide sequence encoding a polypeptide has been optimized for expression of said polypeptide in a prokaryotic host.

Also preferred is an isolated nucleic acid molecule, wherein said polypeptide comprises an amino acid sequence selected from the group consisting of: an amino acid sequence of SEQ ID NO:Y wherein Y is any integer as defined in Table 1; and a complete amino acid sequence of a secreted protein encoded by a human cDNA clone identified by a cDNA Clone Identifier in Table 1 and contained in the deposit with the ATCC™ Deposit Number shown for said cDNA clone in Table 1.

Further preferred is a method of making a recombinant vector comprising inserting any of the above isolated nucleic acid molecule into a vector. Also preferred is the recombinant vector produced by this method. Also preferred is a method of making a recombinant host cell comprising introducing the vector into a host cell, as well as the recombinant host cell produced by this method.

Also preferred is a method of making an isolated polypeptide comprising culturing this recombinant host cell under conditions such that said polypeptide is expressed and recovering said polypeptide. Also preferred is this method of making an isolated polypeptide, wherein said recombinant host cell is a eukaryotic cell and said polypeptide is a secreted portion of a human secreted protein comprising an amino acid sequence selected from the group consisting of: an amino acid sequence of SEQ ID NO:Y beginning with the residue at the position of the First Amino Acid of the Secreted Portion of SEQ ID NO:Y wherein Y is an integer set forth in Table 1 and said position of the First Amino Acid of the Secreted Portion of SEQ ID NO:Y is defined in Table 1; and an amino acid sequence of a secreted portion of a protein encoded by a human cDNA clone identified by a cDNA Clone Identifier in Table 1 and contained in the deposit with the ATCC™ Deposit Number shown for said cDNA clone in Table 1. The isolated polypeptide produced by this method is also preferred.

Also preferred is a method of treatment of an individual in need of an increased level of a secreted protein activity, which method comprises administering to such an individual a pharmaceutical composition comprising an amount of an isolated polypeptide, polynucleotide, or antibody of the claimed invention effective to increase the level of said protein activity in said individual.

Having generally described the invention, the same will be more readily understood by reference to the following examples, which are provided by way of illustration and are not intended as limiting.

### EXAMPLES

### Example 1

### Isolation of a Selected cDNA Clone from the Deposited Sample

Each cDNA clone in a cited ATCC™ deposit is contained in a plasmid vector. Table 1 identifies the vectors used to construct the cDNA library from which each clone was isolated. In many cases, the vector used to construct the library is a phage vector from which a plasmid has been excised. The table immediately below correlates the related plasmid for each phage vector used in constructing the cDNA library. For example, where a particular clone is identified in Table 1 as being isolated in the vector “Lambda Zap,” the corresponding deposited clone is in “pBLUESCRIPT™.”

| Vector Used to Construct Library | Corresponding Deposited Plasmid |
|---|---|
| Lambda Zap | pBLUESCRIPT ™ (pBS) |
| Uni-Zap XR | pBLUESCRIPT ™ (pBS) |
| Zap Express | pBK |
| lafmid BA | plafmid BA |
| pSport1 | pSport1 |
| pCMVSport 2.0 | pCMVSport 2.0 |
| pCMVSport 3.0 | pCMVSport 3.0 |
| pCR ® 2.1 | pCR ® 2.1 |

Vectors Lambda Zap (U.S. Pat. Nos. 5,128,256 and 5,286,636), Uni-Zap XR (U.S. Pat. Nos. 5,128,256 and 5,286,636), Zap Express (U.S. Pat. Nos. 5,128,256 and 5,286,636), pBLUESCRIPT™ (pBS) (Short, J. M. et al., Nucleic Acids Res. 16: 7583-7600 (1988); Alting-Mees, M. A. and Short, J. M., Nucleic Acids Res. 17: 9494 (1989)) and pBK (Alting-Mees, M. A. et al., Strategies 5: 58-61 (1992)) are commercially available from Stratagene Cloning Systems, Inc., 11011 N. Torrey Pines Road, La Jolla, Calif., 92037. pBS contains an ampicillin resistance gene and pBK contains a neomycin resistance gene. Both can be transformed into E. coli strain XL-1 Blue, also available from STRATAGENE™. pBS comes in 4 forms SK+, SK−, KS+ and KS. The S and K refers to the orientation of the polylinker to the T7 and T3 primer sequences which flank the polylinker region (“S” is for SacI and “K” is for KpnI which are the first sites on each respective end of the linker). “+” or “−” refer to the orientation of the f1 origin of replication (“ori”), such that in one orientation, single stranded rescue initiated from the fl ori generates sense strand DNA and in the other, antisense.

Vectors pSport1, pCMVSport 2.0 and pCMVSport 3.0, were obtained from Life Technologies, Inc., P.O. Box 6009, Gaithersburg, Md. 20897. All Sport vectors contain an ampicillin resistance gene and may be transformed into E. coli strain DH10B, also available from Life Technologies. (See, for instance, Gruber, C. E., et al., Focus 15: 59 (1993).) Vector lafmid BA (Bento Soares, Columbia University, NY) contains an ampicillin resistance gene and can be transformed into E. coli strain XL-1 Blue. Vector pCR® 2.1, which is available from Invitrogen, 1600 Faraday Avenue, Carlsbad, Calif. 92008, contains an ampicillin resistance gene and may be transformed into E. coli strain DH10B, available from Life Technologies. (See, for instance, Clark, J. M., Nuc. Acids Res. 16: 9677-9686 (1988) and Mead, D. et al., Bio/Technology 9: (1991).) Preferably, a polynucleotide of the present invention does not comprise the phage vector sequences identified for the particular clone in Table 1, as well as the corresponding plasmid vector sequences designated above.

The deposited material in the sample assigned the ATCC™ Deposit Number cited in Table 1 for any given cDNA clone also may contain one or more additional plasmids, each comprising a cDNA clone different from that given clone. Thus, deposits sharing the same ATCC™ Deposit Number contain at least a plasmid for each cDNA clone identified in Table 1. Typically, each ATCC™ deposit sample cited in Table 1 comprises a mixture of approximately equal amounts (by weight) of about 50 plasmid DNAs, each containing a different cDNA clone; but such a deposit sample may include plasmids for more or less than 50 cDNA clones, up to about 500 cDNA clones.

Two approaches can be used to isolate a particular clone from the deposited sample of plasmid DNAs cited for that clone in Table 1. First, a plasmid is directly isolated by screening the clones using a polynucleotide probe corresponding to SEQ ID NO:X.

Particularly, a specific polynucleotide with 30-40 nucleotides is synthesized using an Applied Biosystems DNA synthesizer according to the sequence reported. The oligonucleotide is labeled, for instance, with ³²P-γ-ATP using T4 polynucleotide kinase and purified according to routine methods. (E.g., Maniatis et al., Molecular Cloning: a Laboratory Manual, Cold Spring Harbor Press, Cold Spring, N.Y. (1982).) The plasmid mixture is transformed into a suitable host, as indicated above (such as XL-1 Blue (STRATAGENE™)) using techniques known to those of skill in the art, such as those provided by the vector supplier or in related publications or patents cited above. The transformants are plated on 1.5% agar plates (containing the appropriate selection agent, e.g., ampicillin) to a density of about 150 transformants (colonies) per plate. These plates are screened using Nylon membranes according to routine methods for bacterial colony screening (e.g., Sambrook et al., Molecular Cloning: a Laboratory Manual, 2nd Edit., (1989), Cold Spring Harbor Laboratory Press, pages 1.93 to 1.104), or other techniques known to those of skill in the art.

Alternatively, two primers of 17-20 nucleotides derived from both ends of the SEQ ID NO:X (i.e., within the region of SEQ ID NO:X bounded by the 5′ NT and the 3′ NT of the clone defined in Table 1) are synthesized and used to amplify the desired cDNA using the deposited cDNA plasmid as a template. The polymerase chain reaction is carried out under routine conditions, for instance, in 25 μl of reaction mixture with 0.5 μg of the above cDNA template. a convenient reaction mixture is 1.5-5 mM MgCl₂, 0.01% (w/v) gelatin, 20 μM each of dATP, dCTP, dGTP, dTTP, 25 pmol of each primer and 0.25 Unit of Taq polymerase. Thirty five cycles of PCR (denaturation at 94° C. for 1 min; annealing at 55° C. for 1 min; elongation at 72° C. for 1 min) are performed with a Perkin-Elmer Cetus automated thermal cycler. The amplified product is analyzed by agarose gel electrophoresis and the DNA band with expected molecular weight is excised and purified. The PCR product is verified to be the selected sequence by subcloning and sequencing the DNA product.

Several methods are available for the identification of the 5′ or 3′ non-coding portions of a gene which may not be present in the deposited clone. These methods include but are not limited to, filter probing, clone enrichment using specific probes, and protocols similar or identical to 5′ and 3′ “RACE” protocols which are well known in the art. For instance, a method similar to 5′ RACE is available for generating the missing 5′ end of a desired full-length transcript. (Fromont-Racine et al., Nucleic Acids Res. 21(7): 1683-1684 (1993).)

Briefly, a specific RNA oligonucleotide is ligated to the 5′ ends of a population of RNA presumably containing full-length gene RNA transcripts. a primer set containing a primer specific to the ligated RNA oligonucleotide and a primer specific to a known sequence of the gene of interest is used to PCR amplify the 5′ portion of the desired full-length gene. This amplified product may then be sequenced and used to generate the full length gene.

This above method starts with total RNA isolated from the desired source, although poly-A+ RNA can be used. The RNA preparation can then be treated with phosphatase if necessary to eliminate 5′ phosphate groups on degraded or damaged RNA which may interfere with the later RNA ligase step. The phosphatase should then be inactivated and the RNA treated with tobacco acid pyrophosphatase in order to remove the cap structure present at the 5′ ends of messenger RNAs. This reaction leaves a 5′ phosphate group at the 5′ end of the cap cleaved RNA which can then be ligated to an RNA oligonucleotide using T4 RNA ligase.

This modified RNA preparation is used as a template for first strand cDNA synthesis using a gene specific oligonucleotide. The first strand synthesis reaction is used as a template for PCR amplification of the desired 5′ end using a primer specific to the ligated RNA oligonucleotide and a primer specific to the known sequence of the gene of interest. The resultant product is then sequenced and analyzed to confirm that the 5′ end sequence belongs to the desired gene.

### Example 2

### Isolation of Genomic Clones Corresponding to a Polynucleotide

A human genomic P1 library (Genomic Systems, Inc.) is screened by PCR using primers selected for the cDNA sequence corresponding to SEQ ID NO:X., according to the method described in Example 1. (See also, Sambrook.)

### Example 3

### Tissue Distribution of Polypeptide

Tissue distribution of mRNA expression of polynucleotides of the present invention is determined using protocols for Northern blot analysis, described by, among others, Sambrook et al. For example, a cDNA probe produced by the method described in Example 1 is labeled with P³² using the REDIPRIME™ DNA labeling system (Amersham Life Science), according to manufacturer's instructions. After labeling, the probe is purified using CHROMA SPIN-100™ column (CLONTECH™ Laboratories, Inc.), according to manufacturer's protocol number PT1200-1. The purified labeled probe is then used to examine various human tissues for mRNA expression.

Multiple Tissue Northern (MTN) blots containing various human tissues (H) or human immune system tissues (IM) (CLONTECH™) are examined with the labeled probe using EXPRESSHYB™ hybridization solution (CLONTECH™) according to manufacturer's protocol number PT1190-1. Following hybridization and washing, the blots are mounted and exposed to film at −70° C. overnight, and the films developed according to standard procedures.

### Example 4

### Chromosomal Mapping of the Polynucleotides

An oligonucleotide primer set is designed according to the sequence at the 5′ end of SEQ ID NO:X. This primer preferably spans about 100 nucleotides. This primer set is then used in a polymerase chain reaction under the following set of conditions: 30 seconds, 95° C.; 1 minute, 56° C.; 1 minute, 70° C. This cycle is repeated 32 times followed by one 5 minute cycle at 70° C. Human, mouse, and hamster DNA is used as template in addition to a somatic cell hybrid panel containing individual chromosomes or chromosome fragments (Bios, Inc). The reactions is analyzed on either 8% polyacrylamide gels or 3.5% agarose gels. Chromosome mapping is determined by the presence of an approximately 100 bp PCR fragment in the particular somatic cell hybrid.

### Example 5

### Bacterial Expression of a Polypeptide

A polynucleotide encoding a polypeptide of the present invention is amplified using PCR oligonucleotide primers corresponding to the 5′ and 3′ ends of the DNA sequence, as outlined in Example 1, to synthesize insertion fragments. The primers used to amplify the cDNA insert should preferably contain restriction sites, such as BamHI and XbaI, at the 5′ end of the primers in order to clone the amplified product into the expression vector. For example, BamHI and XbaI correspond to the restriction enzyme sites on the bacterial expression vector pQE-9. (Qiagen, Inc., Chatsworth, Calif.). This plasmid vector encodes antibiotic resistance (Amp^{r}), a bacterial origin of replication (ori), an IPTG-regulatable promoter/operator (P/O), a ribosome binding site (RBS), a 6-histidine tag (6-His), and restriction enzyme cloning sites.

The pQE-9 vector is digested with BamHI and XbaI and the amplified fragment is ligated into the pQE-9 vector maintaining the reading frame initiated at the bacterial RBS. The ligation mixture is then used to transform the E. coli strain M15/rep4 (Qiagen, Inc.) which contains multiple copies of the plasmid pREP4, which expresses the lad repressor and also confers kanamycin resistance (Kan^{r}). Transformants are identified by their ability to grow on LB plates and ampicillin/kanamycin resistant colonies are selected. Plasmid DNA is isolated and confirmed by restriction analysis.

Clones containing the desired constructs are grown overnight (O/N) in liquid culture in LB media supplemented with both Amp (100 ug/ml) and Kan (25 ug/ml). The O/N culture is used to inoculate a large culture at a ratio of 1:100 to 1:250. The cells are grown to an optical density 600 (O.D.⁶⁰⁰) of between 0.4 and 0.6. IPTG (Isopropyl-B-D-thiogalacto pyranoside) is then added to a final concentration of 1 mM. IPTG induces by inactivating the lad repressor, clearing the P/O leading to increased gene expression.

Cells are grown for an extra 3 to 4 hours. Cells are then harvested by centrifugation (20 mins at 6000×g). The cell pellet is solubilized in the chaotropic agent 6 Molar Guanidine HCl by stirring for 3-4 hours at 4° C. The cell debris is removed by centrifugation, and the supernatant containing the polypeptide is loaded onto a nickel-nitrilo-tri-acetic acid (“Ni-NTA”) affinity resin column (available from QIAGEN, Inc., supra). Proteins with a 6×His tag bind to the Ni-NTA resin with high affinity and can be purified in a simple one-step procedure (for details see: The QIAexpressionist (1995) QIAGEN, Inc., supra).

Briefly, the supernatant is loaded onto the column in 6 M guanidine-HCl, pH 8, the column is first washed with 10 volumes of 6 M guanidine-HCl, pH 8, then washed with 10 volumes of 6 M guanidine-HCl pH 6, and finally the polypeptide is eluted with 6 M guanidine-HCl, pH 5.

The purified protein is then renatured by dialyzing it against phosphate-buffered saline (PBS) or 50 mM Na-acetate, pH 6 buffer plus 200 mM NaCl. Alternatively, the protein can be successfully refolded while immobilized on the Ni-NTA column. The recommended conditions are as follows: renature using a linear 6M-IM urea gradient in 500 mM NaCl, 20% glycerol, 20 mM Tris/HCl pH 7.4, containing protease inhibitors. The renaturation should be performed over a period of 1.5 hours or more. After renaturation the proteins are eluted by the addition of 250 mM immidazole. Immidazole is removed by a final dialyzing step against PBS or 50 mM sodium acetate pH 6 buffer plus 200 mM NaCl. The purified protein is stored at 4° C. or frozen at −80° C.

In addition to the above expression vector, the present invention further includes an expression vector comprising phage operator and promoter elements operatively linked to a polynucleotide of the present invention, called pHE4a. (ATCC™ Accession Number 209645, deposited on Feb. 25, 1998.) This vector contains: 1) a neomycinphosphotransferase gene as a selection marker, 2) an E. coli origin of replication, 3) a T5 phage promoter sequence, 4) two lac operator sequences, 5) a Shine-Delgarno sequence, and 6) the lactose operon repressor gene (lacIq). The origin of replication (oriC) is derived from pUC19 (LTI, Gaithersburg, Md.). The promoter sequence and operator sequences are made synthetically.

DNA can be inserted into the pHEa by restricting the vector with NdeI and XbaI, BamHI, XhoI, or Asp718, running the restricted product on a gel, and isolating the larger fragment (the stuffer fragment should be about 310 base pairs). The DNA insert is generated according to the PCR protocol described in Example 1, using PCR primers having restriction sites for NdeI (5′ primer) and XbaI, BamHI, XhoI, or Asp718 (3′ primer). The PCR insert is gel purified and restricted with compatible enzymes. The insert and vector are ligated according to standard protocols.

The engineered vector could easily be substituted in the above protocol to express protein in a bacterial system.

### Example 6

### Purification of a Polypeptide from an Inclusion Body

The following alternative method can be used to purify a polypeptide expressed in E coli when it is present in the form of inclusion bodies. Unless otherwise specified, all of the following steps are conducted at 4-10° C.

Upon completion of the production phase of the E. coli fermentation, the cell culture is cooled to 4-10° C. and the cells harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells are then lysed by passing the solution through a microfluidizer (Microfuidics, Corp. or APV Gaulin, Inc.) twice at 4000-6000 psi. The homogenate is then mixed with NaCl solution to a final concentration of 0.5 M NaCl, followed by centrifugation at 7000×g for 15 mm. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 2-4 hours. After 7000×g centrifugation for 15 min., the pellet is discarded and the polypeptide containing supernatant is incubated at 4° C. overnight to allow further GuHCl extraction.

Following high speed centrifugation (30,000×g) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4° C. without mixing for 12 hours prior to further purification steps.

To clarify the refolded polypeptide solution, a previously prepared tangential filtration unit equipped with 0.16 lam membrane filter with appropriate surface area (e.g., Filtron), equilibrated with 40 mM sodium acetate, pH 6.0 is employed. The filtered sample is loaded onto a cation exchange resin (e.g., Poros HS-50, Perseptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 nm of the effluent is continuously monitored. Fractions are collected and further analyzed by SDS-PAGE.

Fractions containing the polypeptide are then pooled and mixed with 4 volumes of water. The diluted sample is then loaded onto a previously prepared set of tandem columns of strong anion (Poros HQ-50, Perseptive Biosystems) and weak anion (Poros CM-20, Perseptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A₂₈₀ monitoring of the effluent. Fractions containing the polypeptide (determined, for instance, by 16% SDS-PAGE) are then pooled.

The resultant polypeptide should exhibit greater than 95% purity after the above refolding and purification steps. No major contaminant bands should be observed from Commassie blue stained 16% SDS-PAGE gel when 5 μg of purified protein is loaded. The purified protein can also be tested for endotoxin/LPS contamination, and typically the LPS content is less than 0.1 ng/ml according to LAL assays.

### Example 7

### Cloning and Expression of a Polypeptide in a Baculovirus Expression System

In this example, the plasmid shuttle vector pA2 is used to insert a polynucleotide into a baculovirus to express a polypeptide. This expression vector contains the strong polyhedrin promoter of the Autographa californica nuclear polyhedrosis virus (AcMNPV) followed by convenient restriction sites such as BamHI, Xba I and Asp718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned polynucleotide.

Many other baculovirus vectors can be used in place of the vector above, such as pAc373, pVL941, and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170: 31-39 (1989).

Specifically, the cDNA sequence contained in the deposited clone, including the AUG initiation codon and the naturally associated leader sequence identified in Table 1, is amplified using the PCR protocol described in Example 1. If the naturally occurring signal sequence is used to produce the secreted protein, the pA2 vector does not need a second signal peptide. Alternatively, the vector can be modified (pA2 GP) to include a baculovirus leader sequence, using the standard methods described in Summers et al., “a Manual of Methods for Baculovirus Vectors and Insect Cell Culture Procedures,” Texas Agricultural Experimental Station Bulletin No. 1555 (1987).

The amplified fragment is isolated from a 1% agarose gel using a commercially available kit (“GENECLEAN™,” BIO 101 Inc., La Jolla, Calif.). The fragment then is digested with appropriate restriction enzymes and again purified on a 1% agarose gel.

The plasmid is digested with the corresponding restriction enzymes and optionally, can be dephosphorylated using calf intestinal phosphatase, using routine procedures known in the art. The DNA is then isolated from a 1% agarose gel using a commercially available kit (“GENECLEAN™” BIO 101 Inc., La Jolla, Calif.).

The fragment and the dephosphorylated plasmid are ligated together with T4 DNA ligase. E. coli HB101 or other suitable E coli hosts such as XL-1 Blue (Stratagene Cloning Systems, La Jolla, Calif.) cells are transformed with the ligation mixture and spread on culture plates. Bacteria containing the plasmid are identified by digesting DNA from individual colonies and analyzing the digestion product by gel electrophoresis. The sequence of the cloned fragment is confirmed by DNA sequencing.

Five μg of a plasmid containing the polynucleotide is co-transfected with 1.0 μg of a commercially available linearized baculovirus DNA (“BACULOGOLD™ baculovirus DNA”, Pharmingen, San Diego, Calif.), using the lipofection method described by Felgner et al., Proc. Natl. Acad. Sci. USA 84: 7413-7417 (1987). One μg of BACULOGOLD™ virus DNA and 5 μg of the plasmid are mixed in a sterile well of a microtiter plate containing 50 μl of serum-free Grace's medium (Life Technologies Inc., Gaithersburg, Md.). Afterwards, 10 μl LIPOFECTIN™ plus 90 μl Grace's medium are added, mixed and incubated for 15 minutes at room temperature. Then the transfection mixture is added drop-wise to Sf9 insect cells (ATCC™ CRL 1711) seeded in a 35 mm tissue culture plate with 1 mil Grace's medium without serum. The plate is then incubated for 5 hours at 27° C. The transfection solution is then removed from the plate and 1 ml of Grace's insect medium supplemented with 10% fetal calf serum is added. Cultivation is then continued at 27° C. for four days.

After four days the supernatant is collected and a plaque assay is performed, as described by Summers and Smith, supra. An agarose gel with “Blue Gal” (Life Technologies Inc., Gaithersburg) is used to allow easy identification and isolation of gal-expressing clones, which produce blue-stained plaques. (a detailed description of a “plaque assay” of this type can also be found in the user's guide for insect cell culture and baculovirology distributed by Life Technologies Inc., Gaithersburg, page 9-10.) After appropriate incubation, blue stained plaques are picked with the tip of a micropipettor (e.g., Eppendorf). The agar containing the recombinant viruses is then resuspended in a microcentrifuge tube containing 200 μl of Grace's medium and the suspension containing the recombinant baculovirus is used to infect Sf9 cells seeded in 35 mm dishes. Four days later the supernatants of these culture dishes are harvested and then they are stored at 4° C.

To verify the expression of the polypeptide, Sf9 cells are grown in Grace's medium supplemented with 10% heat-inactivated FBS. The cells are infected with the recombinant baculovirus containing the polynucleotide at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md.). After 42 hours, 5 μCi of ³⁵S-methionine and 5 μCi ³⁵S-cysteine (available from Amersham) are added. The cells are further incubated for 16 hours and then are harvested by centrifugation. The proteins in the supernatant as well as the intracellular proteins are analyzed by SDS-PAGE followed by autoradiography (if radiolabeled).

Microsequencing of the amino acid sequence of the amino terminus of purified protein may be used to determine the amino terminal sequence of the produced protein.

### Example 8

### Expression of a Polypeptide in Mammalian Cells

The polypeptide of the present invention can be expressed in a mammalian cell. a typical mammalian expression vector contains a promoter element, which mediates the initiation of transcription of mRNA, a protein coding sequence, and signals required for the termination of transcription and polyadenylation of the transcript. Additional elements include enhancers, Kozak sequences and intervening sequences flanked by donor and acceptor sites for RNA splicing. Highly efficient transcription is achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter).

Suitable expression vectors for use in practicing the present invention include, for example, vectors such as pSVL and pMSG (PHARMACIA™, Uppsala, Sweden), pRSVcat (ATCC™ 37152), pSV2dhfr (ATCC™ 37146), pBC12MI (ATCC™ 67109), pCMVSport 2.0, and pCMVSport 3.0. Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells.

Alternatively, the polypeptide can be expressed in stable cell lines containing the polynucleotide integrated into a chromosome. The co-transfection with a selectable marker such as dhfr, gpt, neomycin, hygromycin allows the identification and isolation of the transfected cells.

The transfected gene can also be amplified to express large amounts of the encoded protein. The DHFR (dihydrofolate reductase) marker is useful in developing cell lines that carry several hundred or even several thousand copies of the gene of interest. (See, e.g., Alt, F. W., et al., J. Biol. Chem. 253: 1357-1370 (1978); Hamlin, J. L. and Ma, C., Biochem. et Biophys. Acta, 1097: 107-143 (1990); Page, M. J. and Sydenham, M. a., Biotechnology 9: 64-68 (1991).) Another useful selection marker is the enzyme glutamine synthase (GS) (Murphy et al., Biochem J. 227: 277-279 (1991); Bebbington et al., Bio/Technology 10: 169-175 (1992). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

Derivatives of the plasmid pSV2-dhfr (ATCC™ Accession No. 37146), the expression vectors pC4 (ATCC™ Accession No. 209646) and pC6 (ATCC™ Accession No. 209647) contain the strong promoter (LTR) of the Rous Sarcoma Virus (Cullen et al., Molecular and Cellular Biology, 438447 (March, 1985)) plus a fragment of the CMV-enhancer (Boshart et al., Cell 41: 521-530 (1985).) Multiple cloning sites, e.g., with the restriction enzyme cleavage sites BamHI, XbaI and Asp718, facilitate the cloning of the gene of interest. The vectors also contain the 3′ intron, the polyadenylation and termination signal of the rat preproinsulin gene, and the mouse DHFR gene under control of the SV40 early promoter.

Specifically, the plasmid pC6, for example, is digested with appropriate restriction enzymes and then dephosphorylated using calf intestinal phosphates by procedures known in the art. The vector is then isolated from a 1% agarose gel.

A polynucleotide of the present invention is amplified according to the protocol outlined in Example 1. If the naturally occurring signal sequence is used to produce the secreted protein, the vector does not need a second signal peptide. Alternatively, if the naturally occurring signal sequence is not used, the vector can be modified to include a heterologous signal sequence. (See, e.g., WO 96/34891.)

The amplified fragment is isolated from a 1% agarose gel using a commercially available kit (“GENECLEAN™,” BIO 101 Inc., La Jolla, Calif.). The fragment then is digested with appropriate restriction enzymes and again purified on a 1% agarose gel.

The amplified fragment is then digested with the same restriction enzyme and purified on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC6 using, for instance, restriction enzyme analysis.

Chinese hamster ovary cells lacking an active DHFR gene is used for transfection. Five μg of the expression plasmid pC6 is cotransfected with 0.5 μg of the plasmid pSVneo using LIPOFECTIN™ (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of metothrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 μM, 2 μM, 5 μM, 10 mM, 20 mM). The same procedure is repeated until clones are obtained which grow at a concentration of 100-200 μM. Expression of the desired gene product is analyzed, for instance, by SDS-PAGE and Western blot or by reversed phase HPLC analysis.

### Example 9

### Protein Fusions

The polypeptides of the present invention are preferably fused to other proteins. These fusion proteins can be used for a variety of applications. For example, fusion of the present polypeptides to His-tag, HA-tag, protein a, IgG domains, and maltose binding protein facilitates purification. (See Example 5; see also EP a 394,827; Traunecker, et al., Nature 331:84-86 (1988).) Similarly, fusion to IgG-1, IgG-3, and albumin increases the halflife time in vivo. Nuclear localization signals fused to the polypeptides of the present invention can target the protein to a specific subcellular localization, while covalent heterodimer or homodimers can increase or decrease the activity of a fusion protein. Fusion proteins can also create chimeric molecules having more than one function. Finally, fusion proteins can increase solubility and/or stability of the fused protein compared to the non-fused protein. All of the types of fusion proteins described above can be made by modifying the following protocol, which outlines the fusion of a polypeptide to an IgG molecule, or the protocol described in Example 5.

Briefly, the human Fc portion of the IgG molecule can be PCR amplified, using primers that span the 5′ and 3′ ends of the sequence described below. These primers also should have convenient restriction enzyme sites that will facilitate cloning into an expression vector, preferably a mammalian expression vector.

For example, if pC4 (Accession No. 209646) is used, the human Fc portion can be ligated into the BamHI cloning site. Note that the 3′ BamHI site should be destroyed. Next, the vector containing the human Fc portion is re-restricted with BamHI, linearizing the vector, and a polynucleotide of the present invention, isolated by the PCR protocol described in Example 1, is ligated into this BamHI site. Note that the polynucleotide is cloned without a stop codon, otherwise a fusion protein will not be produced.

If the naturally occurring signal sequence is used to produce the secreted protein, pC4 does not need a second signal peptide. Alternatively, if the naturally occurring signal sequence is not used, the vector can be modified to include a heterologous signal sequence. (See, e.g., WO 96/34891.)

Human IgG Fc region:

| | |
|---|---|
| GGGATCCGGAGCCCAAATCTTCTGACAAAACTCACACATGCCCACCGTGCCCAGCACCTGAATTCGAGGGTGCACCGTCAGTCTTC | (SEQ ID NO:1) |
| CTCTTCCCCCCAAAACCCAAGGACACCCTCATGATCTCCCGGACTCCTGAGGTCACATGCGTGGTGGTGGACGTAAGCCACGAAGA | |
| CCCTGAGGTCAAGTTCAACTGGTACGTGGACGGCGTGGAGGTGCATAATGCCAAGACAAAGCCGCGGGAGGAGCAGTACAACAGCA | |
| CGTACCGTGTGGTCAGCGTCCTCACCGTCCTGCACCAGGACTGGCTGAATGGCAAGGAGTACAAGTGCAAGGTCTCCAACAAAGCC | |
| CTCCCAACCCCCATCGAGAAAACCATCTCCAAAGCCAAAGGGCAGCCCCGAGAACCACAGGTGTACACCCTGCCCCCATCCCGGGA | |
| TGAGCTGACCAAGAACCAGGTCAGCCTGACCTGCCTGGTCAAAGGCTTCTATCCAAGCGACATCGCCGTGGAGTGGGAGAGCAATG | |
| GGCAGCCGGAGAACAACTACAAGACCACGCCTCCCGTGCTGGACTCCGACGGCTCCTTCTTCCTCTACAGCAAGCTCACCGTGGAC | |
| AAGAGCAGGTGGCAGCAGGGGAACGTCTTCTCATGCTCCGTGATGCATGAGGCTCTGCACAACCACTACACGCAGAAGAGCCTCTC | |
| CCTGTCTCCGGGTAAATGAGTGCGACGGCCGCGACTCTAGAGGAT | |

### Example 10

### Production of an Antibody from a Polypeptide

The antibodies of the present invention can be prepared by a variety of methods. (See, Current Protocols, Chapter 2.) For example, cells expressing a polypeptide of the present invention is administered to an animal to induce the production of sera containing polyclonal antibodies. In a preferred method, a preparation of the secreted protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or protein binding fragments thereof). Such monoclonal antibodies can be prepared using hybridoma technology. (Köhler et al., Nature 256: 495 (1975); Köhler et al., Eur. J. Immunol. 6: 511 (1976); Köhler et al., Eur. J. Immunol. 6: 292 (1976); Hammerling et al., in: Monoclonal Antibodies and T-Cell Hybridomas, Elsevier, N.Y., pp. 563-681 (1981).) In general, such procedures involve immunizing an animal (preferably a mouse) with polypeptide or, more preferably, with a secreted polypeptide-expressing cell. Such cells may be cultured in any suitable tissue culture medium; however, it is preferable to culture cells in Earle's modified Eagle's medium supplemented with 10% fetal bovine serum (inactivated at about 56° C.), and supplemented with about 10 g/l of nonessential amino acids, about 1,000 U/ml of penicillin, and about 100 μg/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP20), available from the ATCC™. After fusion, the resulting hybridoma cells are selectively maintained in HAT medium, and then cloned by limiting dilution as described by Wands et al. (Gastroenterology 80: 225-232 (1981).) The hybridoma cells obtained through such a selection are then assayed to identify clones which secrete antibodies capable of binding the polypeptide.

Alternatively, additional antibodies capable of binding to the polypeptide can be produced in a two-step procedure using anti-idiotypic antibodies. Such a method makes use of the fact that antibodies are themselves antigens, and therefore, it is possible to obtain an antibody which binds to a second antibody. In accordance with this method, protein specific antibodies are used to immunize an animal, preferably a mouse. The splenocytes of such an animal are then used to produce hybridoma cells, and the hybridoma cells are screened to identify clones which produce an antibody whose ability to bind to the protein-specific antibody can be blocked by the polypeptide. Such antibodies comprise anti-idiotypic antibodies to the protein-specific antibody and can be used to immunize an animal to induce formation of further protein-specific antibodies.

It will be appreciated that Fab and F(ab′)2 and other fragments of the antibodies of the present invention may be used according to the methods disclosed herein. Such fragments are typically produced by proteolytic cleavage, using enzymes such as papain (to produce Fab fragments) or pepsin (to produce F(ab′)2 fragments). Alternatively, secreted protein-binding fragments can be produced through the application of recombinant DNA technology or through synthetic chemistry.

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. (See, for review, Morrison, Science 229: 1202 (1985); Oi et al., BioTechniques 4: 214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312: 643 (1984); Neuberger et al., Nature 314: 268 (1985).)

### Example 11

### Production of Secreted Protein for High-Throughout Screening Assays

The following protocol produces a supernatant containing a polypeptide to be tested. This supernatant can then be used in the Screening Assays described in Examples 13-20.

First, dilute Poly-D-Lysine (644 587 Boehringer-Mannheim) stock solution (1 mg/ml in PBS) 1:20 in PBS (w/o calcium or magnesium 17-516F Biowhittaker) for a working solution of 50 ug/ml. Add 200 ul of this solution to each well (24 well plates) and incubate at RT for 20 minutes. Be sure to distribute the solution over each well (note: a 12-channel pipetter may be used with tips on every other channel). Aspirate off the Poly-D-Lysine solution and rinse with 1 ml PBS (Phosphate Buffered Saline). The PBS should remain in the well until just prior to plating the cells and plates may be poly-lysine coated in advance for up to two weeks.

Plate 293T cells (do not carry cells past P+20) at 2×10⁵ cells/well in 0.5 ml DMEM (Dulbecco's Modified Eagle Medium)(with 4.5 G/L glucose and L-glutamine (12-604F Biowhittaker))/10% heat inactivated FBS (14-503F Biowhittaker)/1× Penstrep(17-602E Biowhittaker). Let the cells grow overnight.

The next day, mix together in a sterile solution basin: 300 μl Lipofectamine (18324-012 Gibco/BRL) and 5 ml Optimem I (31985070 Gibco/BRL)/96-well plate. With a small volume multi-channel pipetter, aliquot approximately 2 μg of an expression vector containing a polynucleotide insert, produced by the methods described in Examples 8 or 9, into an appropriately labeled 96-well round bottom plate. With a multi-channel pipetter, add 50 μl of the Lipofectamine/Optimem 1 mixture to each well. Pipette up and down gently to mix. Incubate at RT 15-45 minutes. After about 20 minutes, use a multi-channel pipetter to add 1501 ul Optimem I to each well. As a control, one plate of vector DNA lacking an insert should be transfected with each set of transfections.

Preferably, the transfection should be performed by tag-teaming the following tasks. By tag-teaming, hands on time is cut in half, and the cells do not spend too much time on PBS. First, person a aspirates off the media from four 24-well plates of cells, and then person B rinses each well with 0.5-1 ml PBS. Person a then aspirates off PBS rinse, and person B, using a 12-channel pipetter with tips on every other channel, adds the 200 μl of DNA/Lipofectamine/Optimem I complex to the odd wells first, then to the even wells, to each row on the 24-well plates. Incubate at 37° C. for 6 hours.

While cells are incubating, prepare appropriate media, either 1% BSA in DMEM with 1× penstrep, or CHO-5 media (116.6 mg/L of CaCl2 (anhyd); 0.00130 mg/L CuSO₄-5H₂O; 0.050 mg/L of Fe(NO₃)₃-9H₂O; 0.417 mg/L of FeSO₄-7H₂O; 311.80 mg/L of Kcl; 28.64 mg/L of MgCl₂; 48.84 mg/L of MgSO₄; 6995.50 mg/L of NaCl; 2400.0 mg/L of NaHCO₃; 62.50 mg/L of NaH₂PO₄—H₂O; 71.02 mg/L of Na₂HPO4; 0.4320 mg/L of ZnSO₄-7H₂O; 0.002 mg/L of Arachidonic Acid; 1.022 mg/L of Cholesterol; 0.070 mg/L of DL-alpha-Tocopherol-Acetate; 0.0520 mg/L of Linoleic Acid; 0.010 mg/L of Linolenic Acid; 0.010 mg/L of Myristic Acid; 0.010 mg/L of Oleic Acid; 0.010 mg/L of Palmitric Acid; 0.010 mg/L of Palmitic Acid; 100 mg/L of Pluronic F-68; 0.010 mg/L of Stearic Acid; 2.20 mg/L of Tween 80; 4551 mg/L of D-Glucose; 130.85 mg/ml of L-Alanine; 147.50 mg/ml of L-Arginine-HCL; 7.50 mg/mil of L-Asparagine-H₂O; 6.65 mg/mil of L-Aspartic Acid; 29.56 mg/ml of L-Cystine-2HCL-H₂O; 31.29 mg/ml of L-Cystine-2HCL; 7.35 mg/ml of L-Glutamic Acid; 365.0 mg/ml of L-Glutamine; 18.75 mg/ml of Glycine; 52.48 mg/ml of L-Histidine-HCL-H₂O; 106.97 mg/ml of L-Isoleucine; 111.45 mg/ml of L-Leucine; 163.75 mg/ml of L-Lysine HCL; 32.34 mg/ml of L-Methionine; 68.48 mg/ml of L-Phenylalainine; 40.0 mg/ml of L-Proline; 26.25 mg/ml of L-Serine; 101.05 mg/ml of L-Threonine; 19.22 mg/ml of L-Tryptophan; 91.79 mg/ml of L-Tryrosine-2Na-2H₂O; 99.65 mg/ml of L-Valine; 0.0035 mg/L of Biotin; 3.24 mg/L of D-Ca Pantothenate; 11.78 mg/L of Choline Chloride; 4.65 mg/L of Folic Acid; 15.60 mg/L of i-Inositol; 3.02 mg/L of Niacinamide; 3.00 mg/L of Pyridoxal HCL; 0.031 mg/L of Pyridoxine HCL; 0.319 mg/L of Riboflavin; 3.17 mg/L of Thiamine HCL; 0.365 mg/L of Thymidine; and 0.680 mg/L of Vitamin B₁₂; 25 mM of HEPES Buffer; 2.39 mg/L of Na Hypoxanthine; 0.105 mg/L of Lipoic Acid; 0.081 mg/L of Sodium Putrescine-2HCL; 55.0 mg/L of Sodium Pyruvate; 0.0067 mg/L of Sodium Selenite; 20 μM of Ethanolamine; 0.122 mg/L of Ferric Citrate; 41.70 mg/L of Methyl-B-Cyclodextrin complexed with Linoleic Acid; 33.33 mg/L of Methyl-B-Cyclodextrin complexed with Oleic Acid; and 10 mg/L of Methyl-B-Cyclodextrin complexed with Retinal) with 2 mm glutamine and 1× penstrep. (BSA (81-068-3 Bayer) 100 gm dissolved in IL DMEM for a 10% BSA stock solution). Filter the media and collect 50 μl for endotoxin assay in 15 ml polystyrene conical.

The transfection reaction is terminated, preferably by tag-teaming, at the end of the incubation period. Person a aspirates off the transfection media, while person B adds 1.5 ml appropriate media to each well. Incubate at 37° C. for 45 or 72 hours depending on the media used: 1% BSA for 45 hours or CHO-5 for 72 hours.

On day four, using a 300 μl multichannel pipetter, aliquot 600 μl in one 1 ml deep well plate and the remaining supernatant into a 2 ml deep well. The supernatants from each well can then be used in the assays described in Examples 13-20.

It is specifically understood that when activity is obtained in any of the assays described below using a supernatant, the activity originates from either the polypeptide directly (e.g., as a secreted protein) or by the polypeptide inducing expression of other proteins, which are then secreted into the supernatant. Thus, the invention further provides a method of identifying the protein in the supernatant characterized by an activity in a particular assay.

### Example 12

### Construction of GAS Reporter Construct

One signal transduction pathway involved in the differentiation and proliferation of cells is called the Jaks-STATs pathway. Activated proteins in the Jaks-STATs pathway bind to gamma activation site “GAS” elements or interferon-sensitive responsive element (“ISRE”), located in the promoter of many genes. The binding of a protein to these elements alter the expression of the associated gene.

GAS and ISRE elements are recognized by a class of transcription factors called Signal Transducers and Activators of Transcription, or “STATs.” There are six members of the STATs family. Stat1 and Stat3 are present in many cell types, as is Stat2 (as response to IFN-alpha is widespread). Stat4 is more restricted and is not in many cell types though it has been found in T helper class 1, cells after treatment with IL-12. Stat5 was originally called mammary growth factor, but has been found at higher concentrations in other cells including myeloid cells. It can be activated in tissue culture cells by many cytokines.

The STATs are activated to translocate from the cytoplasm to the nucleus upon tyrosine phosphorylation by a set of kinases known as the Janus Kinase (“Jaks”) family. Jaks represent a distinct family of soluble tyrosine kinases and include Tyk2, Jak1, Jak2, and Jak3. These kinases display significant sequence similarity and are generally catalytically inactive in resting cells.

The Jaks are activated by a wide range of receptors summarized in the Table below. (Adapted from review by Schidler and Darnell, Ann. Rev. Biochem. 64: 621-51 (1995).) a cytokine receptor family, capable of activating Jaks, is divided into two groups: (a) Class 1 includes receptors for IL-2, IL-3, IL-4, IL-6, IL-7, IL-9, IL-11, IL-12, IL-15, Epo, PRL, GH, G-CSF, GM-CSF, LIF, CNTF, and thrombopoietin; and (b) Class 2 includes IFN-a, IFN-g, and IL-10. The Class 1 receptors share a conserved cysteine motif (a set of four conserved cysteines and one tryptophan) and a WSXWS motif (a membrane proxial region encoding Trp-Ser-Xxx-Trp-Ser (SEQ ID NO:2)).

Thus, on binding of a ligand to a receptor, Jaks are activated, which in turn activate STATs, which then translocate and bind to GAS elements. This entire process is encompassed in the Jaks-STATs signal transduction pathway.

Therefore, activation of the Jaks-STATs pathway, reflected by the binding of the GAS or the ISRE element, can be used to indicate proteins involved in the proliferation and differentiation of cells. For example, growth factors and cytokines are known to activate the Jaks-STATs pathway. (See Table below.) Thus, by using GAS elements linked to reporter molecules, activators of the Jaks-STATs pathway can be identified.

| | JAKs | | | | | |
|---|---|---|---|---|---|---|
| Ligand | tyk2 | Jak1 | Jak2 | Jak3 | STATS | GAS(elements) or ISRE |
| IFN family | | | | | | |
| IFN-a/B | + | + | − | − | 1, 2, 3 | ISRE |
| IFN-g | | + | + | − | 1 | GAS (IRF1 > Lys6 > IFP) |
| Il-10 | + | ? | ? | − | 1, 3 | |
| gp130 family | | | | | | |
| IL-6 (Pleiotrohic)+ | + | + | ? | 1, 3 | | GAS (IRF1 > Lys6 > IFP) |
| Il-11(Pleiotrohic)? | + | ? | ? | 1, 3 | | |
| OnM(Pleiotrohic)? | + | + | ? | 1, 3 | | |
| LIF(Pleiotrohic) ? | + | + | ? | 1, 3 | | |
| CNTF(Pleiotrohic) | −/+ | + | + | ? | 1, 3 | |
| G-CSF(Pleiotrohic) | ? | + | ? | ? | 1, 3 | |
| IL-12(Pleiotrohic) | + | − | + | + | 1, 3 | |
| g-C family | | | | | | |
| IL-2 (lymphocytes) | − | + | − | + | 1, 3, 5 | GAS |
| IL-4 (lymph/myeloid) | − | + | − | + | 6 | GAS (IRF1 = IFP >> Ly6)(IgH) |
| IL-7 (lymphocytes) | − | + | − | + | 5 | GAS |
| IL-9 (lymphocytes) | − | + | − | + | 5 | GAS |
| IL-13 (lymphocyte) | − | + | ? | ? | 6 | GAS |
| IL-15 | ? | + | ? | + | 5 | GAS |
| gp140 family | | | | | | |
| IL-3 (myeloid) | − | − | + | − | 5 | GAS (IRF1 > IFP >> Ly6) |
| IL-5 (myeloid) | − | − | + | − | 5 | GAS |
| GM-CSF (myeloid) | − | − | + | − | 5 | GAS |
| Growth hormone family | | | | | | |
| GH | ? | − | + | − | 5 | |
| PRL | ? | +/− | + | − | 1, 3, 5 | |
| EPO | ? | − | + | − | 5 | GAS(B-CAS > IRF1 = IFP >> Ly6) |
| Receptor Tyrosine Kinases | | | | | | |
| EGF | ? | + | + | − | 1, 3 | GAS (IRF1) |
| PDGF | ? | + | + | − | 1, 3 | |
| CSF-1 | ? | + | + | − | 1, 3 | GAS (not IRF1) |

To construct a synthetic GAS containing promoter element, which is used in the Biological Assays described in Examples 13-14, a PCR based strategy is employed to generate a GAS-SV40 promoter sequence. The 5′ primer contains four tandem copies of the GAS binding site found in the IRF1 promoter and previously demonstrated to bind STATs upon induction with a range of cytokines (Rothman et al., Immunity 1: 457-468 (1994).), although other GAS or ISRE elements can be used instead. The 5′ primer also contains 18 bp of sequence complementary to the SV40 early promoter sequence and is flanked with an XhoI site. The sequence of the 5′ primer is:

(SEQ ID NO:3)

5′:GCGCCTCGAGATTTCCCCGAAATCTAGATTTCCCCGAAATGATTTCCCCGAAATGATTTCCCCGAAATATCTGCCATCTCAATTAG:3′

The downstream primer is complementary to the SV40 promoter and is flanked with a Hind III site: 5′:GCGGCAAGCTTTTTGCAAAGCCTAGGC:3′ (SEQ ID NO:4)

PCR amplification is performed using the SV40 promoter template present in the B-gal:promoter plasmid obtained from CLONTECH™. The resulting PCR fragment is digested with XhoI/Hind III and subcloned into BLSK2-. (STRATAGENE™.) Sequencing with forward and reverse primers confirms that the insert contains the following sequence:

| | |
|---|---|
| 5′:CTCGAGATTTCCCCGAAATCTAGATTTCCCCGAAATGATTTCCCCCAAATGATTTCCCCGAAATATCTGCCATCTCAATTAGTCAG | (SEQ ID NO:5) |
| CAACCATAGTCCCCCCCCTAACTCCGCCCATCCCGCCCCTAACTCCGCCCAGTTCCGCCCATTCTCCGCCCCATGGCTGACTAATTTTT | |
| TTTATTTATGCAGAGGCCGAGGCCGCCTCGGCCTCTGAGCTATTCCAGAAGTAGTGAGGAGGCTTTTTTGGAGGCCTAGGCTTTTGCAA | |
| AAAGCTT:3′. | |

With this GAS promoter element linked to the SV40 promoter, a GAS:SEAP2 reporter construct is next engineered. Here, the reporter molecule is a secreted alkaline phosphatase, or “SEAP.” Clearly, however, any reporter molecule can be instead of SEAP, in this or in any of the other Examples. Well known reporter molecules that can be used instead of SEAP include chloramphenicol acetyltransferase (CAT), luciferase, alkaline phosphatase, B-galactosidase, green fluorescent protein (GFP), or any protein detectable by an antibody.

The above sequence confirmed synthetic GAS-SV40 promoter element is subcloned into the pSEAP-Promoter vector obtained from CLONTECH™ using HindIII and XhoI, effectively replacing the SV40 promoter with the amplified GAS:SV40 promoter element, to create the GAS-SEAP vector. However, this vector does not contain a neomycin resistance gene, and therefore, is not preferred for mammalian expression systems.

Thus, in order to generate mammalian stable cell lines expressing the GAS-SEAP reporter, the GAS-SEAP cassette is removed from the GAS-SEAP vector using SalI and NotI, and inserted into a backbone vector containing the neomycin resistance gene, such as pGFP-1 (CLONTECH™), using these restriction sites in the multiple cloning site, to create the GAS-SEAP/Neo vector. Once this vector is transfected into mammalian cells, this vector can then be used as a reporter molecule for GAS binding as described in Examples 13-14.

Other constructs can be made using the above description and replacing GAS with a different promoter sequence. For example, construction of reporter molecules containing NFK-B and EGR promoter sequences are described in Examples 15 and 16. However, many other promoters can be substituted using the protocols described in these Examples. For instance, SRE, IL-2, NFAT, or Osteocalcin promoters can be substituted, alone or in combination (e.g., GAS/NF-κB/EGR, GAS/NF-κB, II-2/NFAT, or NF-κB/GAS). Similarly, other cell lines can be used to test reporter construct activity, such as HELA (epithelial), HUVEC (endothelial), Reh (B-cell), Saos-2 (osteoblast), HUVAC (aortic), or Cardiomyocyte.

### Example 13

### High-Throughput Screening Assay for T-Cell Activity

The following protocol is used to assess T-cell activity by identifying factors, such as growth factors and cytokines, that may proliferate or differentiate T-cells. T-cell activity is assessed using the GAS/SEAP/Neo construct produced in Example 12. Thus, factors that increase SEAP activity indicate the ability to activate the Jaks-STATS signal transduction pathway. The T-cell used in this assay is Jurkat T-cells (ATCC™ Accession No. TIB-152), although Molt-3 cells (ATCC™ Accession No. CRL-1552) and Molt-4 cells (ATCC™ Accession No. CRL-1582) cells can also be used.

Jurkat T-cells are lymphoblastic CD4+Th1 helper cells. In order to generate stable cell lines, approximately 2 million Jurkat cells are transfected with the GAS-SEAP/neo vector using DMRIE-C (Life Technologies)(transfection procedure described below). The transfected cells are seeded to a density of approximately 20,000 cells per well and transfectants resistant to 1 mg/ml genticin selected. Resistant colonies are expanded and then tested for their response to increasing concentrations of interferon gamma. The dose response of a selected clone is demonstrated.

Specifically, the following protocol will yield sufficient cells for 75 wells containing 200 μl of cells. Thus, it is either scaled up, or performed in multiple to generate sufficient cells for multiple 96 well plates. Jurkat cells are maintained in RPMI+10% serum with 1% Pen-Strep. Combine 2.5 mls of OPTI-MEM™ (Life Technologies) with 10 μg of plasmid DNA in a T25 flask. Add 2.5 ml OPTI-MEM™ containing 50 μl of DMRIE-C and incubate at room temperature for 15-45 mins.

During the incubation period, count cell concentration, spin down the required number of cells (107 per transfection), and resuspend in OPTI-MEM™ to a final concentration of 10⁷ cells/ml. Then add 1 ml of 1×10⁷ cells in OPTI-MEM™ to T25 flask and incubate at 37° C. for 6 hrs. After the incubation, add 10 ml of RPMI+15% serum.

The Jurkat:GAS-SEAP stable reporter lines are maintained in RPMI+10% serum, 1 mg/ml Genticin, and 1% Pen-Strep. These cells are treated with supernatants containing a polypeptide as produced by the protocol described in Example 11.

On the day of treatment with the supernatant, the cells should be washed and resuspended in fresh RPMI+10% serum to a density of 500,000 cells per ml. The exact number of cells required will depend on the number of supernatants being screened. For one 96 well plate, approximately 10 million cells (for 10 plates, 100 million cells) are required.

Transfer the cells to a triangular reservoir boat, in order to dispense the cells into a 96 well dish, using a 12 channel pipette. Using a 12 channel pipette, transfer 200 μl of cells into each well (therefore adding 100,000 cells per well).

After all the plates have been seeded, 50 μl of the supernatants are transferred directly from the 96 well plate containing the supernatants into each well using a 12 channel pipette. In addition, a dose of exogenous interferon gamma (0.1, 1.0, 10 ng) is added to wells H9, H10, and H11 to serve as additional positive controls for the assay.

The 96 well dishes containing Jurkat cells treated with supernatants are placed in an incubator for 48 hrs (note: this time is variable between 48-72 hrs). 35 μl samples from each well are then transferred to an opaque 96 well plate using a 12 channel pipette. The opaque plates should be covered (using sellophene covers) and stored at −20° C. until SEAP assays are performed according to Example 17. The plates containing the remaining treated cells are placed at 4° C. and serve as a source of material for repeating the assay on a specific well if desired.

As a positive control, 100 Unit/ml interferon gamma can be used which is known to activate Jurkat T cells. Over 30 fold induction is typically observed in the positive control wells.

### Example 14

### High-Throughput Screening Assay Identifying Myeloid Activity

The following protocol is used to assess myeloid activity by identifying factors, such as growth factors and cytokines, that may proliferate or differentiate myeloid cells. Myeloid cell activity is assessed using the GAS/SEAP/Neo construct produced in Example 12. Thus, factors that increase SEAP activity indicate the ability to activate the Jaks-STATS signal transduction pathway. The myeloid cell used in this assay is U937, a pre-monocyte cell line, although TF-1, HL60, or KG1 can be used.

To transiently transfect U937 cells with the GAS/SEAP/Neo construct produced in Example 12, a DEAE-Dextran method (Kharbanda et. al., 1994, Cell Growth & Differentiation, 5: 259-265) is used. First, harvest 2×10e⁷ U937 cells and wash with PBS. The U937 cells are usually grown in RPMI 1640 medium containing 10% heat-inactivated fetal bovine serum (FBS) supplemented with 100 units/ml penicillin and 100 mg/ml streptomycin.

Next, suspend the cells in 1 ml of 20 mM Tris-HCl (pH 7.4) buffer containing 0.5 mg/ml DEAE-Dextran, 8 μg GAS-SEAP2 plasmid DNA, 140 mM NaCl, 5 mM KCl, 375 μM Na₂HPO₄.7H₂₀, 1 mM MgCl₂, and 675 μM CaCl₂. Incubate at 37° C. for 45 min.

Wash the cells with RPMI 1640 medium containing 10% FBS and then resuspend in 10 ml complete medium and incubate at 37° C. for 36 hr.

The GAS-SEAP/U937 stable cells are obtained by growing the cells in 400 μg/ml G418. The G418-free medium is used for routine growth but every one to two months, the cells should be re-grown in 400 μg/ml G418 for couple of passages.

These cells are tested by harvesting 1×10⁸ cells (this is enough for ten 96-well plates assay) and wash with PBS. Suspend the cells in 200 ml above described growth medium, with a final density of 5×10⁵ cells/ml. Plate 200 μl cells per well in the 96-well plate (or 1×10⁵ cells/well).

Add 50 μl of the supernatant prepared by the protocol described in Example 11. Incubate at 37° C. for 48 to 72 hr. As a positive control, 100 Unit/ml interferon gamma can be used which is known to activate U937 cells. Over 30 fold induction is typically observed in the positive control wells. SEAP assay the supernatant according to the protocol described in Example 17.

### Example 15

### High-Throughput Screening Assay Identifying Neuronal Activity

When cells undergo differentiation and proliferation, a group of genes are activated through many different signal transduction pathways. One of these genes, EGR1 (early growth response gene 1), is induced in various tissues and cell types upon activation. The promoter of EGR1 is responsible for such induction. Using the EGR1 promoter linked to reporter molecules, activation of cells can be assessed.

Particularly, the following protocol is used to assess neuronal activity in PC12 cell lines. PC12 cells (rat phenochromocytoma cells) are known to proliferate and/or differentiate by activation with a number of mitogens, such as TPA (tetradecanoyl phorbol acetate), NGF (nerve growth factor), and EGF (epidermal growth factor). The EGR1 gene expression is activated during this treatment. Thus, by stably transfecting PC12 cells with a construct containing an EGR promoter linked to SEAP reporter, activation of PC12 cells can be assessed.

The EGR/SEAP reporter construct can be assembled by the following protocol. The EGR-1 promoter sequence (−633 to +1)(Sakamoto K et al., Oncogene 6: 867-871 (1991)) can be PCR amplified from human genomic DNA using the following primers:

(SEQ ID NO:6)

5′ GCGCTCGAGGGATGACAGCGATAGAACCCCGG-3′

(SEQ ID NO:7)

5′ GCGAAGCTTCGCGACTCCCCGGATCCGCCTC-3′

Using the GAS:SEAP/Neo vector produced in Example 12, EGR1 amplified product can then be inserted into this vector. Linearize the GAS:SEAP/Neo vector using restriction enzymes XhoI/HindIII, removing the GAS/SV40 stuffer. Restrict the EGR1 amplified product with these same enzymes. Ligate the vector and the EGR1 promoter.

To prepare 96 well-plates for cell culture, two mls of a coating solution (1:30 dilution of collagen type I (Upstate Biotech Inc. Cat#08-115) in 30% ethanol (filter sterilized)) is added per one 10 cm plate or 50 ml per well of the 96-well plate, and allowed to air dry for 2 hr.

PC12 cells are routinely grown in RPMI-1640 medium (Bio Whittaker) containing 10% horse serum (JRH BIOSCIENCES, Cat. # 12449-78P), 5% heat-inactivated fetal bovine serum (FBS) supplemented with 100 units/ml penicillin and 100 μg/ml streptomycin on a precoated 10 cm tissue culture dish. One to four split is done every three to four days. Cells are removed from the plates by scraping and resuspended with pipetting up and down for more than 15 times.

Transfect the EGR/SEAP/Neo construct into PC12 using the Lipofectamine protocol described in Example 11. EGR-SEAP/PC12 stable cells are obtained by growing the cells in 300 μg/ml G418. The G418-free medium is used for routine growth but every one to two months, the cells should be re-grown in 300 μg/ml G418 for couple of passages.

To assay for neuronal activity, a 10 cm plate with cells around 70 to 80% confluent is screened by removing the old medium. Wash the cells once with PBS (Phosphate buffered saline). Then starve the cells in low serum medium (RPMI-1640 containing 1% horse serum and 0.5% FBS with antibiotics) overnight.

The next morning, remove the medium and wash the cells with PBS. Scrape off the cells from the plate, suspend the cells well in 2 ml low serum medium. Count the cell number and add more low serum medium to reach final cell density as 5×10⁵ cells/ml.

Add 200 μl of the cell suspension to each well of 96-well plate (equivalent to 1×10⁵ cells/well). Add 50 μl supernatant produced by Example 11, 37° C. for 48 to 72 hr. As a positive control, a growth factor known to activate PC12 cells through EGR can be used, such as 50 ng/μl of Neuronal Growth Factor (NGF). Over fifty-fold induction of SEAP is typically seen in the positive control wells. SEAP assay the supernatant according to Example 17.

### Example 16

### High-Throughput Screening Assay for T-Cell Activity

NF-κB (Nuclear Factor κB) is a transcription factor activated by a wide variety of agents including the inflammatory cytokines IL-1 and TNF, CD30 and CD40, lymphotoxin-alpha and lymphotoxin-beta, by exposure to LPS or thrombin, and by expression of certain viral gene products. As a transcription factor, NF-κB regulates the expression of genes involved in immune cell activation, control of apoptosis (NF-κB appears to shield cells from apoptosis), B and T-cell development, anti-viral and antimicrobial responses, and multiple stress responses.

In non-stimulated conditions, NF-κB is retained in the cytoplasm with I-κB (Inhibitor κB). However, upon stimulation, I-κB is phosphorylated and degraded, causing NF-κB to shuttle to the nucleus, thereby activating transcription of target genes. Target genes activated by NF-κB include IL-2, IL-6, GM-CSF, ICAM-1 and class 1 MHC.

Due to its central role and ability to respond to a range of stimuli, reporter constructs utilizing the NF-κB promoter element are used to screen the supernatants produced in Example 11. Activators or inhibitors of NF-κB would be useful in treating diseases. For example, inhibitors of NF-κB could be used to treat those diseases related to the acute or chronic activation of NF-κB, such as rheumatoid arthritis.

To construct a vector containing the NF-κB promoter element, a PCR based strategy is employed. The upstream primer contains four tandem copies of the NF-κB binding site (GGGGACTTTCCC) (SEQ ID NO:8), 18 bp of sequence complementary to the 5′ end of the SV40 early promoter sequence, and is flanked with an XhoI site:

| | |
|---|---|
| 5′:GCGGCCTCGAGGGGACTTTCCCGGGGACTTTCCGGGGACTTTCCGGGACTTTCCATCCTGCCATCTCAATTAG:3′ | (SEQ ID NO:9) |

The downstream primer is complementary to the 3′ end of the SV40 promoter and is flanked with a Hind III site: 5′:GCGGCAAGCTTTTTGCAAAGCCTAGGC:3′ (SEQ ID NO:4)

PCR amplification is performed using the SV40 promoter template present in the pB-gal:promoter plasmid obtained from CLONTECH™. The resulting PCR fragment is digested with XhoI and Hind III and subcloned into BLSK2-. (STRATAGENE™) Sequencing with the T7 and T3 primers confirms the insert contains the following sequence:

(SEQ ID NO:10)

5′:CTCGAGGGGACTTTCCCGGGGACTTTCCGGGGACTTTCCGGGACTTT
CCATCTGCCATCTCAATTAGTCAGCAACCATAGTCCCGCCCCTAACTCCG
CCCATCCCGCCCCTAACTCCGCCCAGTTCCGCCCATTCTCCGCCCCATGG
CTGACTAATTTTTTTTATTTATGCAGAGGCCGAGGCCGCCTCGGCCTCTG
AGCTATTCCAGAAGTAGTGAGGAGGCTTTTTTGGAGGCCTAGGCTTTTGC
AAAAAGCTT:3′

Next, replace the SV40 minimal promoter element present in the pSEAP2-promoter plasmid (CLONTECH™) with this NF-κB/SV40 fragment using XhoI and HindIII. However, this vector does not contain a neomycin resistance gene, and therefore, is not preferred for mammalian expression systems.

In order to generate stable mammalian cell lines, the NF-κB/SV40/SEAP cassette is removed from the above NF-κB/SEAP vector using restriction enzymes SalI and NotI, and inserted into a vector containing neomycin resistance. Particularly, the NF-κB/SV40/SEAP cassette was inserted into pGFP-1 (CLONTECH™), replacing the GFP gene, after restricting pGFP-1 with SalI and NotI.

Once NF-κB/SV40/SEAP/Neo vector is created, stable Jurkat T-cells are created and maintained according to the protocol described in Example 13. Similarly, the method for assaying supernatants with these stable Jurkat T-cells is also described in Example 13. As a positive control, exogenous TNF alpha (0.1, 1, 10 ng) is added to wells H9, H10, and H11, with a 5-10 fold activation typically observed.

### Example 17

### Assay for SEAP Activity

As a reporter molecule for the assays described in Examples 13-16, SEAP activity is assayed using the Tropix Phospho-light Kit (Cat. BP-400) according to the following general procedure. The Tropix Phospho-light Kit supplies the Dilution, Assay, and Reaction Buffers used below.

Prime a dispenser with the 2.5× Dilution Buffer and dispense 15 μl of 2.5× dilution buffer into Optiplates containing 35 μl of a supernatant. Seal the plates with a plastic sealer and incubate at 65° C. for 30 min. Separate the Optiplates to avoid uneven heating.

Cool the samples to room temperature for 15 minutes. Empty the dispenser and prime with the Assay Buffer. Add 50 μl Assay Buffer and incubate at room temperature 5 min. Empty the dispenser and prime with the Reaction Buffer (see the table below). Add 50 μl Reaction Buffer and incubate at room temperature for 20 minutes. Since the intensity of the chemiluminescent signal is time dependent, and it takes about 10 minutes to read 5 plates on luminometer, one should treat 5 plates at each time and start the second set 10 minutes later.

Read the relative light unit in the luminometer. Set H12 as blank, and print the results. An increase in chemiluminescence indicates reporter activity.

Reaction Buffer Formulation:

| # of plates | Rxn buffer diluent (ml) | CSPD (ml) |
|---|---|---|
| 10 | 60 | 3 |
| 11 | 65 | 3.25 |
| 12 | 70 | 3.5 |
| 13 | 75 | 3.75 |
| 14 | 80 | 4 |
| 15 | 85 | 4.25 |
| 16 | 90 | 4.5 |
| 17 | 95 | 4.75 |
| 18 | 100 | 5 |
| 19 | 105 | 5.25 |
| 20 | 110 | 5.5 |
| 21 | 115 | 5.75 |
| 22 | 120 | 6 |
| 23 | 125 | 6.25 |
| 24 | 130 | 6.5 |
| 25 | 135 | 6.75 |
| 26 | 140 | 7 |
| 27 | 145 | 7.25 |
| 28 | 150 | 7.5 |
| 29 | 155 | 7.75 |
| 30 | 160 | 8 |
| 31 | 165 | 8.25 |
| 32 | 170 | 8.5 |
| 33 | 175 | 8.75 |
| 34 | 180 | 9 |
| 35 | 185 | 9.25 |
| 36 | 190 | 9.5 |
| 37 | 195 | 9.75 |
| 38 | 200 | 10 |
| 39 | 205 | 10.25 |
| 40 | 210 | 10.5 |
| 41 | 215 | 10.75 |
| 42 | 220 | 11 |
| 43 | 225 | 11.25 |
| 44 | 230 | 11.5 |
| 45 | 235 | 11.75 |
| 46 | 240 | 12 |
| 47 | 245 | 12.25 |
| 48 | 250 | 12.5 |
| 49 | 255 | 12.75 |
| 50 | 260 | 13 |

### Example 18

### High-Throughput Screening Assay Identify the Changes in Small Molecule Concentration and Membrane Permeability

Binding of a ligand to a receptor is known to alter intracellular levels of small molecules, such as calcium, potassium, sodium, and pH, as well as alter membrane potential. These alterations can be measured in an assay to identify supernatants which bind to receptors of a particular cell. Although the following protocol describes an assay for calcium, this protocol can easily be modified to detect changes in potassium, sodium, pH, membrane potential, or any other small molecule which is detectable by a fluorescent probe.

The following assay uses Fluorometric Imaging Plate Reader (“FLIPR”) to measure changes in fluorescent molecules (Molecular Probes) that bind small molecules. Clearly, any fluorescent molecule detecting a small molecule can be used instead of the calcium fluorescent molecule, fluo-3, used here.

For adherent cells, seed the cells at 10,000-20,000 cells/well in a Co-star black 96-well plate with clear bottom. The plate is incubated in a CO₂ incubator for 20 hours. The adherent cells are washed two times in Biotek washer with 200 μl of HBSS (Hank's Balanced Salt Solution) leaving 100 μl of buffer after the final wash.

A stock solution of 1 mg/ml fluo-3 is made in 10% pluronic acid DMSO. To load the cells with fluo-3, 50 μl of 12 ug/ml fluo-3 is added to each well. The plate is incubated at 37° C. in a CO₂ incubator for 60 min. The plate is washed four times in the Biotek washer with HBSS leaving 100 μl of buffer.

For non-adherent cells, the cells are spun down from culture media. Cells are re-suspended to 2-5×10⁶ cells/ml with HBSS in a 50-ml conical tube. 4 μl of 1 mg/ml fluo-3 solution in 10% pluronic acid DMSO is added to each ml of cell suspension. The tube is then placed in a 37° C. water bath for 30-60 min. The cells are washed twice with HBSS, resuspended to 1×10⁶ cells/ml, and dispensed into a microplate, 100 μl/well. The plate is centrifuged at 1000 rpm for 5 min. The plate is then washed once in Denley CellWash with 200 μl, followed by an aspiration step to 100 μl final volume.

For a non-cell based assay, each well contains a fluorescent molecule, such as fluo-3. The supernatant is added to the well, and a change in fluorescence is detected.

To measure the fluorescence of intracellular calcium, the FLIPR is set for the following parameters: (1) System gain is 300-800 mW; (2) Exposure time is 0.4 second; (3) Camera F/stop is F/2; (4) Excitation is 488 nm; (5) Emission is 530 nm; and (6) Sample addition is 50 μl. Increased emission at 530 nm indicates an extracellular signaling event which has resulted in an increase in the intracellular Ca⁺⁺ concentration.

### Example 19

### High-Throughput Screening Assay Identifying Tyrosine Kinase Activity

The Protein Tyrosine Kinases (PTK) represent a diverse group of transmembrane and cytoplasmic kinases. Within the Receptor Protein Tyrosine Kinase RPTK) group are receptors for a range of mitogenic and metabolic growth factors including the PDGF, FGF, EGF, NGF, HGF and Insulin receptor subfamilies. In addition there are a large family of RPTKs for which the corresponding ligand is unknown. Ligands for RPTKs include mainly secreted small proteins, but also membrane-bound and extracellular matrix proteins.

Activation of RPTK by ligands involves ligand-mediated receptor dimerization, resulting in transphosphorylation of the receptor subunits and activation of the cytoplasmic tyrosine kinases. The cytoplasmic tyrosine kinases include receptor associated tyrosine kinases of the src-family (e.g., src, yes, Ick, lyn, fyn) and non-receptor linked and cytosolic protein tyrosine kinases, such as the Jak family, members of which mediate signal transduction triggered by the cytokine superfamily of receptors (e.g., the Interleukins, Interferons, GM-CSF, and Leptin).

Because of the wide range of known factors capable of stimulating tyrosine kinase activity, the identification of novel human secreted proteins capable of activating tyrosine kinase signal transduction pathways are of interest. Therefore, the following protocol is designed to identify those novel human secreted proteins capable of activating the tyrosine kinase signal transduction pathways.

Seed target cells (e.g., primary keratinocytes) at a density of approximately 25,000 cells per well in a 96 well LOPRODYNE™ Silent Screen Plates purchased from Nalge Nunc (Naperville, Ill.). The plates are sterilized with two 30 minute rinses with 100% ethanol, rinsed with water and dried overnight. Some plates are coated for 2 hr with 100 ml of cell culture grade type I collagen (50 mg/mil), gelatin (2%) or polylysine (50 mg/ml), all of which can be purchased from Sigma Chemicals (St. Louis, Mo.) or 10% MATRIGEL™ purchased from Becton Dickinson (Bedford, Mass.), or calf serum, rinsed with PBS and stored at 4° C. Cell growth on these plates is assayed by seeding 5,000 cells/well in growth medium and indirect quantitation of cell number through use of ALAMARBLUE™ as described by the manufacturer Alamar Biosciences, Inc. (Sacramento, Calif.) after 48 hr. Falcon plate covers #3071 from Becton Dickinson (Bedford, Mass.) are used to cover the LOPRODYNE™ Silent Screen Plates. Falcon Microtest III cell culture plates can also be used in some proliferation experiments.

To prepare extracts, A431 cells are seeded onto the nylon membranes of LOPRODYNE™ plates (20,000/200 ml/well) and cultured overnight in complete medium. Cells are quiesced by incubation in serum-free basal medium for 24 hr. After 5-20 minutes treatment with EGF (60 ng/ml) or 50 μl of the supernatant produced in Example 11, the medium was removed and 100 ml of extraction buffer ((20 mM HEPES pH 7.5, 0.15 M NaCl, 1% Triton X-100, 0.1% SDS, 2 mM Na3VO4, 2 mM Na4P207 and a cocktail of protease inhibitors (# 1836170) obtained from Boeheringer Mannheim (Indianapolis, Ind.) is added to each well and the plate is shaken on a rotating shaker for 5 minutes at 4° C. The plate is then placed in a vacuum transfer manifold and the extract filtered through the 0.45 mm membrane bottoms of each well using house vacuum. Extracts are collected in a 96-well catch/assay plate in the bottom of the vacuum manifold and immediately placed on ice. To obtain extracts clarified by centrifugation, the content of each well, after detergent solubilization for 5 minutes, is removed and centrifuged for 15 minutes at 4° C. at 16,000×g.

Test the filtered extracts for levels of tyrosine kinase activity. Although many methods of detecting tyrosine kinase activity are known, one method is described here.

Generally, the tyrosine kinase activity of a supernatant is evaluated by determining its ability to phosphorylate a tyrosine residue on a specific substrate (a biotinylated peptide). Biotinylated peptides that can be used for this purpose include PSK1 (corresponding to amino acids 6-20 of the cell division kinase cdc2-p34) and PSK2 (corresponding to amino acids 1-17 of gastrin). Both peptides are substrates for a range of tyrosine kinases and are available from Boehringer Mannheim.

The tyrosine kinase reaction is set up by adding the following components in order. First, add 10 μl of 5 μM Biotinylated Peptide, then 10 μl ATP/Mg₂₊ (5 mM ATP/50 mM MgCl₂), then 10 μl of 5× Assay Buffer (40 mM imidazole hydrochloride, pH7.3, 40 mM beta-glycerophosphate, 1 mM EGTA, 100 mM MgCl₂, 5 mM MnCl₂, 0.5 mg/ml BSA), then 5 μl of Sodium Vanadate (1 mM), and then 5 μl of water. Mix the components gently and preincubate the reaction mix at 30° C. for 2 min. Initial the reaction by adding 10 μl of the control enzyme or the filtered supernatant.

The tyrosine kinase assay reaction is then terminated by adding 10 μl of 120 mm EDTA and place the reactions on ice.

Tyrosine kinase activity is determined by transferring 50 μl aliquot of reaction mixture to a microtiter plate (MTP) module and incubating at 37° C. for 20 min. This allows the streptavadin coated 96 well plate to associate with the biotinylated peptide. Wash the MTP module with 300 ul/well of PBS four times. Next add 75 μl of anti-phospotyrosine antibody conjugated to horse radish peroxidase (anti-P-Tyr-POD(0.5 u/ml)) to each well and incubate at 37° C. for one hour. Wash the well as above.

Next add 100 μl of peroxidase substrate solution (Boehringer Mannheim) and incubate at room temperature for at least 5 mins (up to 30 min). Measure the absorbance of the sample at 405 nm by using ELISA reader. The level of bound peroxidase activity is quantitated using an ELISA reader and reflects the level of tyrosine kinase activity.

### Example 20

### High-Throughput Screening Assay Identifying Phosphorylation Activity

As a potential alternative and/or compliment to the assay of protein tyrosine kinase activity described in Example 19, an assay which detects activation (phosphorylation) of major intracellular signal transduction intermediates can also be used. For example, as described below one particular assay can detect tyrosine phosphorylation of the Erk-1 and Erk-2 kinases. However, phosphorylation of other molecules, such as Raf, JNK, p38 MAP, Map kinase kinase (MEK), MEK kinase, Src, Muscle specific kinase (MuSK), IRAK, Tec, and Janus, as well as any other phosphoserine, phosphotyrosine, or phosphothreonine molecule, can be detected by substituting these molecules for Erk-1 or Erk-2 in the following assay.

Specifically, assay plates are made by coating the wells of a 96-well ELISA plate with 0.1 ml of protein G (1 ug/ml) for 2 hr at room temp, (RT). The plates are then rinsed with PBS and blocked with 3% BSA/PBS for 1 hr at RT. The protein G plates are then treated with 2 commercial monoclonal antibodies (10 ng/well) against Erk-1 and Erk-2 (1 hr at RT) (Santa Cruz Biotechnology). (To detect other molecules, this step can easily be modified by substituting a monoclonal antibody detecting any of the above described molecules.) After 3-5 rinses with PBS, the plates are stored at 4° C. until use.

A431 cells are seeded at 20,000/well in a 96-well LOPRODYNE™ filterplate and cultured overnight in growth medium. The cells are then starved for 48 hr in basal medium (DMEM) and then treated with EGF (6 ng/well) or 50 μl of the supernatants obtained in Example 11 for 5-20 minutes. The cells are then solubilized and extracts filtered directly into the assay plate.

After incubation with the extract for 1 hr at RT, the wells are again rinsed. As a positive control, a commercial preparation of MAP kinase (10 ng/well) is used in place of A431 extract. Plates are then treated with a commercial polyclonal (rabbit) antibody (1 ug/ml) which specifically recognizes the phosphorylated epitope of the Erk-1 and Erk-2 kinases (1 hr at RT). This antibody is biotinylated by standard procedures. The bound polyclonal antibody is then quantitated by successive incubations with Europium-streptavidin and Europium fluorescence enhancing reagent in the Wallac DELFIA instrument (time-resolved fluorescence). An increased fluorescent signal over background indicates a phosphorylation.

### Example 21

### Method of Determining Alterations in a Gene Corresponding to a Polynucleotide

RNA isolated from entire families or individual patients presenting with a phenotype of interest (such as a disease) is be isolated. cDNA is then generated from these RNA samples using protocols known in the art. (See, Sambrook.) The cDNA is then used as a template for PCR, employing primers surrounding regions of interest in SEQ ID NO:X. Suggested PCR conditions consist of 35 cycles at 95° C. for 30 seconds; 60-120 seconds at 52-58° C.; and 60-120 seconds at 70° C., using buffer solutions described in Sidransky, D., et al., Science 252: 706 (1991).

PCR products are then sequenced using primers labeled at their 5′ end with T4 polynucleotide kinase, employing SequiTherm Polymerase. (Epicentre Technologies). The intron-exon borders of selected exons is also determined and genomic PCR products analyzed to confirm the results. PCR products harboring suspected mutations is then cloned and sequenced to validate the results of the direct sequencing.

PCR products is cloned into T-tailed vectors as described in Holton, T. A. and Graham, M. W., Nucleic Acids Research, 19: 1156 (1991) and sequenced with 17 polymerase (United States Biochemical). Affected individuals are identified by mutations not present in unaffected individuals.

Genomic rearrangements are also observed as a method of determining alterations in a gene corresponding to a polynucleotide. Genomic clones isolated according to Example 2 are nick-translated with digoxigenindeoxy-uridine 5′-triphosphate (Boehringer Manheim), and FISH performed as described in Johnson, Cg. et al., Methods Cell Biol. 35: 73-99 (1991). Hybridization with the labeled probe is carried out using a vast excess of human cot-1 DNA for specific hybridization to the corresponding genomic locus.

Chromosomes are counterstained with 4,6-diamino-2-phenylidole and propidium iodide, producing a combination of C- and R-bands. Aligned images for precise mapping are obtained using a triple-band filter set (Chroma Technology, Brattleboro, Vt.) in combination with a cooled charge-coupled device camera (Photometrics, Tucson, Ariz.) and variable excitation wavelength filters. (Johnson, Cv. et al., Genet. Anal. Tech. Appl., 8: 75 (1991).) Image collection, analysis and chromosomal fractional length measurements are performed using the ISee Graphical Program System. (Inovision Corporation, Durham, N.C.) Chromosome alterations of the genomic region hybridized by the probe are identified as insertions, deletions, and translocations. These alterations are used as a diagnostic marker for an associated disease.

### Example 22

### Method of Detecting Abnormal Levels of a Polypeptide in a Biological Sample

A polypeptide of the present invention can be detected in a biological sample, and if an increased or decreased level of the polypeptide is detected, this polypeptide is a marker for a particular phenotype. Methods of detection are numerous, and thus, it is understood that one skilled in the art can modify the following assay to fit their particular needs.

For example, antibody-sandwich ELISAs are used to detect polypeptides in a sample, preferably a biological sample. Wells of a microtiter plate are coated with specific antibodies, at a final concentration of 0.2 to 10 μg/ml. The antibodies are either monoclonal or polyclonal and are produced by the method described in Example 10. The wells are blocked so that non-specific binding of the polypeptide to the well is reduced.

The coated wells are then incubated for >2 hours at RT with a sample containing the polypeptide. Preferably, serial dilutions of the sample should be used to validate results. The plates are then washed three times with deionized or distilled water to remove unbounded polypeptide.

Next, 50 μl of specific antibody-alkaline phosphatase conjugate, at a concentration of 25-400 ng, is added and incubated for 2 hours at room temperature. The plates are again washed three times with deionized or distilled water to remove unbounded conjugate.

Add 75 μl of 4-methylumbelliferyl phosphate (MUP) or p-nitrophenyl phosphate (NPP) substrate solution to each well and incubate 1 hour at room temperature. Measure the reaction by a microtiter plate reader. Prepare a standard curve, using serial dilutions of a control sample, and plot polypeptide concentration on the X-axis (log scale) and fluorescence or absorbance of the Y-axis (linear scale). Interpolate the concentration of the polypeptide in the sample using the standard curve.

### Example 23

### Formulating a Polypeptide

The secreted polypeptide composition will be formulated and dosed in a fashion consistent with good medical practice, taking into account the clinical condition of the individual patient (especially the side effects of treatment with the secreted polypeptide alone), the site of delivery, the method of administration, the scheduling of administration, and other factors known to practitioners. The “effective amount” for purposes herein is thus determined by such considerations.

As a general proposition, the total pharmaceutically effective amount of secreted polypeptide administered parenterally per dose will be in the range of about 1 μg/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, the secreted polypeptide is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions containing the secreted protein of the invention are administered orally, rectally, parenterally, intracistemally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

The secreted polypeptide is also suitably administered by sustained-release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22: 547-556 (1983)), poly(2-hydroxyethyl methacrylate) (R. Langer et al., J. Biomed. Mater. Res. 15: 167-277 (1981), and R. Langer, Chem. Tech. 12: 98-105 (1982)), ethylene vinyl acetate (R. Langer et al.) or poly-D-(-)-3-hydroxybutyric acid (EP 133,988). Sustained-release compositions also include liposomally entrapped polypeptides. Liposomes containing the secreted polypeptide are prepared by methods known per se: DE 3,218,121; Epstein et al., Proc. Natl. Acad. Sci. USA 82: 3688-3692 (1985); Hwang et al., Proc. Natl. Acad. Sci. USA 77: 4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal secreted polypeptide therapy.

For parenteral administration, in one embodiment, the secreted polypeptide is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting the polypeptide uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

The secreted polypeptide is typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of polypeptide salts.

Any polypeptide to be used for therapeutic administration can be sterile. Sterility is readily accomplished by filtration through sterile filtration membranes (e.g., 0.2 micron membranes). Therapeutic polypeptide compositions generally are placed into a container having a sterile access port, for example, an intravenous solution bag or vial having a stopper pierceable by a hypodermic injection needle.

Polypeptides ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous polypeptide solution, and the resulting mixture is lyophilized. The infusion solution is prepared by reconstituting the lyophilized polypeptide using bacteriostatic Water-for-Injection.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the polypeptides of the present invention may be employed in conjunction with other therapeutic compounds.

### Example 24

### Method of Treating Decreased Levels of the Polypeptide

It will be appreciated that conditions caused by a decrease in the standard or normal expression level of a secreted protein in an individual can be treated by administering the polypeptide of the present invention, preferably in the secreted form. Thus, the invention also provides a method of treatment of an individual in need of an increased level of the polypeptide comprising administering to such an individual a pharmaceutical composition comprising an amount of the polypeptide to increase the activity level of the polypeptide in such an individual.

For example, a patient with decreased levels of a polypeptide receives a daily dose 0.1-100 μg/kg of the polypeptide for six consecutive days. Preferably, the polypeptide is in the secreted form. The exact details of the dosing scheme, based on administration and formulation, are provided in Example 23.

### Example 25

### Method of Treating Increased Levels of the Polypeptide

Antisense technology is used to inhibit production of a polypeptide of the present invention. This technology is one example of a method of decreasing levels of a polypeptide, preferably a secreted form, due to a variety of etiologies, such as cancer.

For example, a patient diagnosed with abnormally increased levels of a polypeptide is administered intravenously antisense polynucleotides at 0.5, 1.0, 1.5, 2.0 and 3.0 mg/kg day for 21 days. This treatment is repeated after a 7-day rest period if the treatment was well tolerated. The formulation of the antisense polynucleotide is provided in Example 23.

### Example 26

### Method of Treatment Using Gene Therapy

One method of gene therapy transplants fibroblasts, which are capable of expressing a polypeptide, onto a patient. Generally, fibroblasts are obtained from a subject by skin biopsy. The resulting tissue is placed in tissue-culture medium and separated into small pieces. Small chunks of the tissue are placed on a wet surface of a tissue culture flask, approximately ten pieces are placed in each flask. The flask is turned upside down, closed tight and left at room temperature over night. After 24 hours at room temperature, the flask is inverted and the chunks of tissue remain fixed to the bottom of the flask and fresh media (e.g., Ham's F12 media, with 10% FBS, penicillin and streptomycin) is added. The flasks are then incubated at 37° C. for approximately one week.

At this time, fresh media is added and subsequently changed every several days. After an additional two weeks in culture, a monolayer of fibroblasts emerge. The monolayer is trypsinized and scaled into larger flasks.

pMV-7 (Kirschmeier, P. T. et al., DNA, 7: 219-25 (1988)), flanked by the long terminal repeats of the Moloney murine sarcoma virus, is digested with EcoRI and HindIII and subsequently treated with calf intestinal phosphatase. The linear vector is fractionated on agarose gel and purified, using glass beads.

The cDNA encoding a polypeptide of the present invention can be amplified using PCR primers which correspond to the 5′ and 3′ end sequences respectively as set forth in Example 1. Preferably, the 5′ primer contains an EcoRI site and the 3′ primer includes a HindIII site. Equal quantities of the Moloney murine sarcoma virus linear backbone and the amplified EcoRI and HindIII fragment are added together, in the presence of T4 DNA ligase. The resulting mixture is maintained under conditions appropriate for ligation of the two fragments. The ligation mixture is then used to transform bacteria HB101, which are then plated onto agar containing kanamycin for the purpose of confirming that the vector has the gene of interest properly inserted.

The amphotropic pA317 or GP+am12 packaging cells are grown in tissue culture to confluent density in Dulbecco's Modified Eagles Medium (DMEM) with 10% calf serum (CS), penicillin and streptomycin. The MSV vector containing the gene is then added to the media and the packaging cells transduced with the vector. The packaging cells now produce infectious viral particles containing the gene (the packaging cells are now referred to as producer cells).

Fresh media is added to the transduced producer cells, and subsequently, the media is harvested from a 10 cm plate of confluent producer cells. The spent media, containing the infectious viral particles, is filtered through a millipore filter to remove detached producer cells and this media is then used to infect fibroblast cells. Media is removed from a sub-confluent plate of fibroblasts and quickly replaced with the media from the producer cells. This media is removed and replaced with fresh media. If the titer of virus is high, then virtually all fibroblasts will be infected and no selection is required. If the titer is very low, then it is necessary to use a retroviral vector that has a selectable marker, such as neo or his. Once the fibroblasts have been efficiently infected, the fibroblasts are analyzed to determine whether protein is produced.

The engineered fibroblasts are then transplanted onto the host, either alone or after having been grown to confluence on cytodex 3 microcarrier beads.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference. Further, the hard copy of the sequence listing submitted herewith and the corresponding computer readable form are both incorporated herein by reference in their entireties.

1. An isolated nucleic acid molecule comprising a polynucleotide having a nucleotide sequence at least 95% identical to a sequence selected from the group consisting of:
(a) a polynucleotide fragment of SEQ ID NO:X or a polynucleotide fragment of the cDNA sequence included in ATCC Deposit No:Z, which is hybridizable to SEQ ID NO:X;
(b) a polynucleotide encoding a polypeptide fragment of SEQ ID NO:Y or a polypeptide fragment encoded by the cDNA sequence included in ATCC Deposit No:Z, which is hybridizable to SEQ ID NO:X;
(c) a polynucleotide encoding a polypeptide domain of SEQ ID NO:Y or a polypeptide domain encoded by the cDNA sequence included in ATCC Deposit No:Z, which is hybridizable to SEQ ID NO:X;
(d) a polynucleotide encoding a polypeptide epitope of SEQ ID NO:Y or a polypeptide epitope encoded by the cDNA sequence included in ATCC Deposit No:Z, which is hybridizable to SEQ ID NO:X;
(e) a polynucleotide encoding a polypeptide of SEQ ID NO:Y or the cDNA sequence included in ATCC Deposit No:Z, which is hybridizable to SEQ ID NO:X, having biological activity;
(f) a polynucleotide which is a variant of SEQ ID NO:X;
(g) a polynucleotide which is an allelic variant of SEQ ID NO:X;
(h) a polynucleotide which encodes a species homologue of the SEQ ID NO:Y;
(i) a polynucleotide capable of hybridizing under stringent conditions to any one of the polynucleotides specified in (a)-(h),
wherein said polynucleotide does not hybridize under stringent conditions to a nucleic acid molecule having a nucleotide sequence of only a residues or of only T residues.

2. The isolated nucleic acid molecule of claim 1, wherein the polynucleotide fragment comprises a nucleotide sequence encoding a secreted protein.

3. The isolated nucleic acid molecule of claim 1, wherein the polynucleotide fragment comprises a nucleotide sequence encoding the sequence identified as SEQ ID NO:Y or the polypeptide encoded by the cDNA sequence included in ATCC Deposit No:Z, which is hybridizable to SEQ ID NO:X.

4. The isolated nucleic acid molecule of claim 1, wherein the polynucleotide fragment comprises the entire nucleotide sequence of SEQ ID NO:X or the cDNA sequence included in ATCC Deposit No:Z, which is hybridizable to SEQ ID NO:X.

5. The isolated nucleic acid molecule of claim 2, wherein the nucleotide sequence comprises sequential nucleotide deletions from either the C-terminus or the N-terminus.

6. The isolated nucleic acid molecule of claim 3, wherein the nucleotide sequence comprises sequential nucleotide deletions from either the C-terminus or the N-terminus.

7. A recombinant vector comprising the isolated nucleic acid molecule of claim 1.

8. A method of making a recombinant host cell comprising the isolated nucleic acid molecule of claim 1.

9. A recombinant host cell produced by the method of claim 8.

10. The recombinant host cell of claim 9 comprising vector sequences.

11. An isolated polypeptide comprising an amino acid sequence at least 95% identical to a sequence selected from the group consisting of:
(a) a polypeptide fragment of SEQ ID NO:Y or the encoded sequence included in ATCC Deposit No:Z;
(b) a polypeptide fragment of SEQ ID NO:Y or the encoded sequence included in ATCC Deposit No:Z, having biological activity;
(c) a polypeptide domain of SEQ ID NO:Y or the encoded sequence included in ATCC Deposit No:Z;
(d) a polypeptide epitope of SEQ ID NO:Y or the encoded sequence included in ATCC Deposit No:Z;
(e) a secreted form of SEQ ID NO:Y or the encoded sequence included in ATCC Deposit No:Z;
(f) a full length protein of SEQ ID NO:Y or the encoded sequence included in ATCC Deposit No:Z;
(g) a variant of SEQ ID NO:Y;
(h) an allelic variant of SEQ ID NO:Y; or
(i) a species homologue of the SEQ ID NO:Y.

12. The isolated polypeptide of claim 11, wherein the secreted form or the full length protein comprises sequential amino acid deletions from either the C-terminus or the N-terminus.

13. An isolated antibody that binds specifically to the isolated polypeptide of claim 11.

14. A recombinant host cell that expresses the isolated polypeptide of claim 11.

15. A method of making an isolated polypeptide comprising:
(a) culturing the recombinant host cell of claim 14 under conditions such that said polypeptide is expressed; and
(b) recovering said polypeptide.

16. The polypeptide produced by claim 15.

17. A method for preventing, treating, or ameliorating a medical condition, comprising administering to a mammalian subject a therapeutically effective amount of the polynucleotide of claim 1.

18. A method of diagnosing a pathological condition or a susceptibility to a pathological condition in a subject comprising:
(a) determining the presence or absence of a mutation in the polynucleotide of claim 1; and
(b) diagnosing a pathological condition or a susceptibility to a pathological condition based on the presence or absence of said mutation.

19. A method of diagnosing a pathological condition or a susceptibility to a pathological condition in a subject comprising:
(a) determining the presence or amount of expression of the polypeptide of claim 11 in a biological sample; and
(b) diagnosing a pathological condition or a susceptibility to a pathological condition based on the presence or amount of expression of the polypeptide.

20. A method for preventing, treating, or ameliorating a medical condition, comprising administering to a mammalian subject a therapeutically effective amount of the polypeptide of claim 11.
